(19) United States
(12) Patent Application Publication
(10) Pub. No.: US 2023/0150909 A1
(43) Pub. Date: May 18, 2023

(54) PHENYLACETIC ACID DERIVATIVE, USE THEREFOR, AND PRODUCTION INTERMEDIATE THEREOF

(71) Applicant: SUMITOMO CHEMICAL COMPANY, LIMITED, Tokyo (JP)

(72) Inventors: Nao MAEHATA, Osaka-shi (JP); Keisuke ARAI, Takarazuka-shi (JP); Hiroto TAMASHIMA, Takarazuka-shi (JP); Toshiyuki HARADA, Takarazuka-shi (JP)

(73) Assignee: SUMITOMO CHEMICAL COMPANY, LIMITED, Chuo-ku (JP)

(21) Appl. No.: 17/759,526

(22) PCT Filed: Jan. 29, 2021
(86) PCT No.: PCT/JP2021/003394

§ 371 Date: Jul. 27, 2022

(30) Foreign Application Priority Data
Jan. 31, 2020 (JP) 2020-015186

(51) Int. Cl. C07C 57/32; C07C 229/36

(52) U.S. Cl. CPC C07C 57/32; C07C 229/36

(57) ABSTRACT

The present invention provides a compound having an excellent efficacy for controlling pests. According to the present invention, a compound represented by formula (I) [wherein Q represents a group represented by Q1, or a group represented by Q2 (where • represents a binding site to a benzene ring), E represents a C5-C6 cycloalkenyl group and so on, L represents an oxygen atom or NH, a combination of R¹ and n represents a combination where R¹ represents a hydrogen atom, and n is 1, and so on, and R² represents a methyl group and so on] or its N-oxide, or agriculturally acceptable salts thereof has an excellent efficacy for controlling pests.

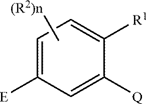

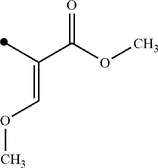

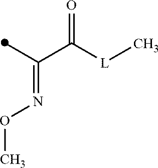

### TECHNICAL FIELD

This application claims priority to and the benefit of Japanese Patent Application No. 2020-015186 filed Jan. 31, 2020, the entire contents of which are incorporated herein by reference.

The present invention relates to a phenyl acetic acid derivative and preparation intermediate compound thereof

### BACKGROUND ART

Phenyl acetic acid derivatives are described in Patent document 1.

### Citation List

### Patent Document

Patent Document 1: EP patent publication No. 422597 A1

### SUMMARY OF THE INVENTION

### Problems to be Solved by Invention

An object of the present invention is to provide a compound having an excellent efficacy for controlling pests.

### Means to Solve Problems

The present inventor has studied to solve the problem and, as a result, found out that a compound represented by the below-mentioned formula (I) has an excellent efficacy for controlling pests.

That, the present invention is as follows. [1] A compound represented by formula (I):

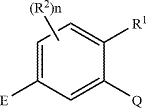

[wherein
Q represents a group represented by Q1, or a group represented by Q2 (where • represents a binding site to a benzene ring),

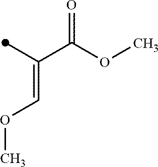

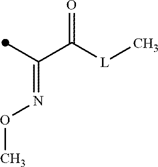

E represents a C5-C6 cycloalkenyl group which may be optionally substituted with one or more substituents selected from Group D, a C6-C10 aryl group, a five- to ten-membered aromatic heterocyclic group {the C6-C10 aryl group and the five- to ten- membered aromatic heterocyclic group may be optionally substituted by one or more substituents selected from Group A}, R⁷—C═C—, R⁵—O—N═C (R⁴) —, R⁸—N═C (R⁴) — or R⁶O—,
L represents an oxygen atom or NH,
a combination of R¹ and n represents a combination where R¹ represents a hydrogen atom, and n is 1; or
a combination where R¹ represents a C1-C3 chain hydrocarbon group which may be optionally substituted with one or more halogen atoms, a methoxy group, a cyclopropyl group, or a halogen atom, and n is 0,
R² represents a methyl group, a cyclopropyl group, or a halogen atom,
R⁴ represents a C1-C3 chain hydrocarbon group which may be optionally substituted with one or more halogen atoms, or a hydrogen atom,
R⁵ represents a C1-C6 chain hydrocarbon group which may be optionally substituted with one or more substituents selected from Group F, C3-C6 cycloalkyl group which may be optionally substituted with one or more substituents selected from Group D, a phenyl group, a five- to six-membered aromatic heterocyclic group {the phenyl group and the five- to six- membered aromatic heterocyclic group may be optionally substituted with one or more substituents selected from Group C}, or R¹⁸—CH₂—,
R⁷ represents a C1-C6 chain hydrocarbon group which may be optionally substituted with one or more substituents selected from Group F, a C3-C6 cycloalkyl group which may be optionally substituted with one or more substituents selected from Group D, or a five- to six- membered aromatic heterocyclic group {the five- to six- membered aromatic heterocyclic group may be optionally substituted with one or more substituents selected from Group C},
R⁶ represents a methyl group which is substituted with one or more substituents selected from Group F, a C2-C6 chain hydrocarbon group which may be optionally substituted with one or more substituents selected from Group F, a C3-C4 cycloalkyl group which may be optionally substituted with one or more substituents selected from Group D, or R¹³R⁴NC(O)—,
R⁸ and R¹³ are identical to or different from each other and represent a C1-C6 chain hydrocarbon group which may be optionally substituted with one or more substituents selected from Group F, a C3-C6 cycloalkyl group which may be optionally substituted with one or more substituents selected from Group D, a phenyl group, a five- to six-membered aromatic heterocyclic group {the phenyl group and the five- to six- membered aromatic heterocyclic group may be optionally substituted with one or more substituents selected from Group C}, or R¹⁰— (CH₂)ₘ—,
m is 1 or 2,
R¹⁰ and R¹⁸ are identical to or different from each other and represent a phenyl group, or a five- to six- membered aromatic heterocyclic group {the phenyl group and the five-to six- membered aromatic heterocyclic group may be optionally substituted with one or more substituents selected from Group C},
Group A is a group consisting of a C1-C6 chain hydrocarbon group which may be optionally substituted with one or more substituents selected from Group F, a C3-C6 cycloalkyl group which may be optionally substituted with one or more substituents selected from Group D, a C1-C6 alkylthio group which may be optionally substituted with one or more substituents selected from Group F, OR¹⁴, C(O)R¹¹, C(O)OR¹¹, NR¹¹R⁹, C(R⁹)═N—OR¹¹, a phenyl group, a five- to six-membered aromatic heterocyclic group {the phenyl group, and the five- to six- membered aromatic heterocyclic group may be optionally substituted with one or more substituents selected from Group E}, a halogen atom, a cyano group, and a nitro group,
R⁹ represents a C1-C3 chain hydrocarbon group which may be optionally substituted with one or more halogen atoms, or a hydrogen atom,
R¹¹ and R¹⁴ are identical to or different from each other and represent a C1-C6 chain hydrocarbon group which may be optionally substituted with one or more substituents selected from Group F, a C3-C6 cycloalkyl group which may be optionally substituted with one or more substituents selected from Group D, a phenyl group, or a five- to six-membered aromatic heterocyclic group {the phenyl group and the five- to six- membered aromatic heterocyclic group may be optionally substituted with one or more substituents selected from Group C},
Group C is a group consisting of a C1-C3 chain hydrocarbon group, a C3-C4 cycloalkyl group, a C1-C3 alkoxy group, and a C1-C3 alkylthio group {the C1-C3 chain hydrocarbon group, the C3-C4 cycloalkyl group, the C1-C3 alkoxy group, and the C1-C3 alkylthio group may be optionally substituted with one or more halogen atoms}, a halogen atom, a cyano group, a nitro group, and a hydroxy group,
Group D is a group consisting of a C1-C3 alkyl group which may be optionally substituted with one or more halogen atoms, a halogen atom, and a cyano group,
Group E is a group consisting of a C1-C3 chain hydrocarbon group, a C3-C4 cycloalkyl group, a C1-C3 alkoxy group, and C1-C3 alkylthio group {the C3-C4 cycloalkyl group, the C1-C3 alkoxy group, and the C1-C3 alkylthio group may be optionally substituted with one or more halogen atoms}, a halogen atom, a cyano group, a nitro group, and a hydroxy group, and
Group F is a group consisting of a C3-C4 cycloalkyl group, a C1-C3 alkoxy group, and a halogen atom] (hereinafter, referred to as “Compound N of the present invention”) or its N-oxide or agriculturally acceptable salts (hereinafter, Compound represented by formula (I), or its N-oxide or agriculturally acceptable salts are referred to as “Compound of the present invention”).

The compound according to [1] wherein
E represents a C5-C6 cycloalkenyl group which may be optionally substituted with one or more substituents selected from Group D, a phenyl group, a five- to six-membered aromatic heterocyclic group {the phenyl group and the five- to six- membered aromatic heterocyclic group may be optionally substituted with one or more substituents selected from Group H}, R⁷—C═C—, R⁵—O—N═C(R⁴)—, or R⁶O—,
R⁴ represents a C1-C3 alkyl group, or a hydrogen atom,
R⁵ represents a C1-C6 chain hydrocarbon group which may be optionally substituted with one or more substituents selected from Group F, a C3-C6 cycloalkyl group which may be optionally substituted with one or more halogen atoms, a phenyl group, a five- to six- membered aromatic heterocyclic group {the phenyl group and the five- to six- membered aromatic heterocyclic group may be optionally substituted with one or more substituents selected from Group G}, or R¹⁸—CH₂—,
R⁶ represents a C2-C6 which may be optionally substituted with one or more substituents selected from Group F, a C3-C4 cycloalkyl group which may be optionally substituted with one or more halogen atoms, or R¹³R⁴NC(O)—
R⁷ represents a C1-C6 chain hydrocarbon group which may be optionally substituted with one or more substituents selected from Group F, or a C3-C6 cycloalkyl group which may be optionally substituted with one or more halogen atoms,
R¹¹ and R¹³ are identical to or different from each other and represent a C1-C6 chain hydrocarbon group which may be optionally substituted with one or more substituents selected from Group F, or a C3-C6 cycloalkyl group which may be optionally substituted with one or more halogen atoms,
R¹⁴ represents a C1-C6 chain hydrocarbon group which may be optionally substituted with one or more substituents selected from Group F, a C3-C6 cycloalkyl group which may be optionally substituted with one or more halogen atoms, or a phenyl group which may be optionally substituted with one or more substituents selected from Group G,
R¹⁸ represents a phenyl group, or a five- to six-membered aromatic heterocyclic group {the phenyl group and the five- to six- membered aromatic heterocyclic group may be optionally substituted with one or more substituents selected from Group G},
Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group, and
Group H is a group consisting of a C1-C6 chain hydrocarbon group which may be optionally substituted with one or more substituents, a C3-C4 cycloalkyl group which may be optionally substituted with one or more halogen atoms, OR¹⁴, a halogen atom, a cyano group, C(R⁴)═N—OR¹¹, a phenyl group, and a five- to six- membered aromatic heterocyclic group {the phenyl group and the five- to six- membered aromatic heterocyclic group may be optionally substituted with one or more substituents selected from Group G}, or its N-oxide or agriculturally acceptable salts.

The compound according to [2] wherein
E represents R⁷—C═C—, R⁵—O—N═C(R⁴)—, R⁶O—, a phenyl group, a pyridyl group, a thienyl group, a furanyl group, a pyrimidinyl group, a thiadiazolyl group, or a pyrazolyl group {the phenyl group, the pyridyl group, the thienyl group, the furanyl group, the pyrimidinyl group, the thiadiazolyl group, and the pyrazolyl group may be optionally substituted with one or more substituents selected from Group I},
R⁵ represents a C1-C6 chain hydrocarbon group which may be optionally substituted with one or more substituents selected from Group F, or a C3-C4 cycloalkyl group which may be optionally substituted with one or more halogen atoms, or R¹⁸—CH₂—,
R⁶ represents a C2-C6 chain hydrocarbon group which may be optionally substituted with one or more halogen atoms,
R⁷ represents a C1-C6 chain hydrocarbon group which may be optionally substituted with one or more halogen atoms, or a C3-C4 cycloalkyl group which may be optionally substituted with one or more halogen atoms,
R¹¹ represents a C1-C3 alkyl group which may be optionally substituted with one or more halogen atoms,
R¹⁴ represents a C1-C6 chain hydrocarbon group which may be optionally substituted with one or more substituents selected from Group F, a C3-C4 cycloalkyl group which may be optionally substituted with one or more halogen atoms, or a phenyl group which may be optionally substituted with one or more substituents selected from Group G,
R¹⁸ represents a phenyl group which may be optionally substituted with one or more substituents selected from Group G, and
Group I is a group consisting of a C1-C6 chain hydrocarbon group which may be optionally substituted with one or more substituents selected from Group F, a cyclopropyl group, OR¹⁴, a halogen atom, a cyano group, C(R⁴)═N—OR¹¹, a phenyl group, a pyridyl group, and a pyrazolyl group {the phenyl group, the pyridyl group, and the pyrazolyl group may be optionally substituted with one or more substituents selected from Group G},
or its N-oxide or agriculturally acceptable salts.

The compound according to [2] wherein
E represents R⁷—C═C—, a phenyl group, a pyridyl group, a thienyl group, a furanyl group, a thiadiazolyl group, a pyrazolyl group {the phenyl group, the pyridyl group, the thienyl group, the furanyl group, the thiadiazolyl group, and the pyrazolyl group may be optionally substituted with one or more substituents selected from Group J}, or a pyrimidinyl group {the pyrimidinyl group may be optionally substituted with one or more substituents selected from Group K},
R⁷ represents a C1-C6 alkyl group which may be optionally substituted with one or more halogen atoms, or a cyclopropyl group,
Group J is a group consisting of a C1-C6 chain hydrocarbon group, a C1-C3 alkoxy group {the C1-C6 chain hydrocarbon group and the C1-C3 alkoxy group may be optionally substituted with one or more substituents selected from Group F}, a cyclopropyl group, a halogen atom, a cyano group, C (R⁴) ═M—OR¹¹, a phenyl group, a pyridyl group, and a pyrazolyl group {the phenyl group, the pyridyl group, and the pyrazolyl group may be optionally substituted with one or more substituents selected from Group G}, and
Group K is a group consisting of a C1-C3 alkyl group which may be optionally substituted with one or more halogen atoms, a phenoxy group which may be optionally substituted with one or more substituents selected from Group G, and a halogen atom,
or its N-oxide or agriculturally acceptable salts.

The compound according to any one of [1] to [4] wherein
R¹ represents a C1-C3 chain hydrocarbon group which may be optionally substituted with one or more halogen atoms, or a halogen atom, and
n is 0,
or its N-oxide or agriculturally acceptable salts.

The compound according to any one of [1] to [4] wherein
Q represents a group represented by Q1,
R¹ represents a methyl group, and
n is 0,
or its N-oxide or agriculturally acceptable salts.

An agricultural composition which comprises the compound according to any one of [1] to [6] or its N-oxide compound or an agriculturally acceptable salt thereof and an inert carrier.

A composition which comprises one or more ingredients selected from the group consisting of the following Groups (a), (b), (c) and (d) and the compound according to any one of [1] to [6] or its N-oxide compound or agriculturally acceptable salts:
Group (a) : a group consisting of insecticidal ingredients, miticidal ingredients, and nematicidal ingredients;
Group (b): fungicidal ingredients:
Group (c): plant growth modulating ingredients; and
Group (d): repellent ingredients.

A method for controlling pests which comprises applying an effective amount of the compound according to any one of [1] to [6] or its N-oxide compound or an agriculturally acceptable salt thereof or the composition according to [8] to a plant or soil.

A method for controlling soybean rust fungus having an amino acid substitution of F129L on mitochondrial cytochrome b protein by applying an effective amount of the compound according to any one of [1] to [6] or its N-oxide compound or an agriculturally acceptable salt thereof or the composition according to [8] to soybean or soils where soybean grows.

Use of the compound according to any one of [1] to [6] or its N-oxide compound or an agriculturally acceptable salt thereof or the composition according to [8] for controlling a pest.

A seed or vegetative reproductive organ carrying an effective amount of the compound according to any one of [1] to [6] or its N-oxide thereof, or the composition according to [8].

A compound represented by formula (II):

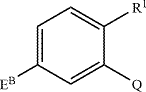

[wherein
a combination of Q and E^{B} represents a combination where Q represents a group represented by Q1, and E^{B} represents R¹²C(O)—, R¹²C(═N—OH) —, or a halogen atom, or,
a combination where Q represents a group represented by Q2, and E^{B} represents R¹²C(O) —, R¹²C(═N—OH) —, a bromine atom, or an iodine atom,
each of the group represented by Q1 and the group represented by Q2 represents the following group (where • represents a binding site to a benzene ring),

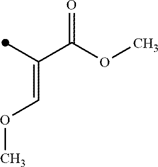

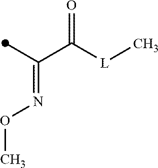

L represents an oxygen atom or NH,
R¹ represents a C1-C3 chain hydrocarbon group which may be optionally substituted with one or more halogen atoms, a methoxy group, a cyclopropyl group, or a halogen atom, and
R¹² represents a C1-C3 alkyl group]
(hereinafter, referred to as “Intermediate compound A”).

### Effect of Invention

The present invention can control pests.

### Mode for Carrying Out the Invention

The substituent(s) as described herein is/are explained.

The term “halogen atom” represents fluorine atom, chlorine atom, bromine atom, or iodine atom.

When the substituent has two or more halogen atoms, these halogen atoms may be identical to or different from each other.

The expression of “CX-CY” as used herein represents that the number of carbon atom is from X to Y. For example, the expression of “C1-C6” represents that the number of carbon atom is from 1 to 6.

The term “chain hydrocarbon group” represents an alkyl group, an alkenyl group, or an alkynyl group.

Examples of the “alkyl group” include methyl group, ethyl group, propyl group, isopropyl group, 1,1-dimethylpropyl group, 1,2-dimethylpropyl group, butyl group, sec-butyl group, tert-butyl group, pentyl group, hexyl group, and decyl group.

Examples of the “alkenyl group” include vinyl group, 1-propenyl group, 2-propenyl group, 1-methyl-1-propenyl group, 1-methyl-2-propenyl group, 1,2-dimethyl-1-propenyl group, 3-butenyl group, 4-pentenyl group, 5-hexenyl group, and 9-decenyl group.

Examples of the “alkynyl group” include ethynyl group, 1-propynyl group, 2-propynyl group, 1-methyl-2-propynyl group, 1,1-dimethyl-2-propynyl group, 2-butynyl group, 4-pentynyl group, 5-hexynyl group, and 9-decynyl group.

Examples of the “alkoxy group” include methoxy group, ethoxy group, propoxy group, isopropoxy group, butoxy group, tert-butoxy group, pentyloxy group, and hexyloxy group.

Examples of the “alkylthio group” includes methylthio group, ethylthio group, propylthio group, isopropylthio group, butylthio group, tert-butylthio group, pentylthio group, and hexylthio group.

Examples of the “cycloalkyl group” include cyclopropyl group, cyclobutyl group, cyclopentyl group, and cyclohexyl group.

Examples of the “cycloalkenyl group” include cyclopentenyl group, and cyclohexenyl group.

Examples of the “aryl group” include phenyl group, indenyl group, indanyl group, naphthyl group, and tetrahydronaphthyl group.

Examples of the “aromatic heterocyclic group” include pyrrolyl group, furyl group, thienyl group, pyrazolyl group, imidazolyl group, triazolyl group, tetrazolyl group, oxazolyl group, isoxazolyl group, thiazolyl group, isothiazolyl group, oxadiazolyl group, thiadiazolyl group, pyridyl group, pyridazinyl group, pyrimidinyl group, pyrazinyl group, triazinyl group, tetrazinyl group, indolyl group, indazolyl group, benzoimidazolyl group, imidazopyridyl group, benzothiophenyl group, benzofuranyl group, quinolyl group, isoquinolyl group, quinazolinyl group, and quinoxalinyl group.

The term(s) as described herein is/are explained.

The term of “soybean rust fungus having an amino acid substitution of F129L on mitochondrial cytochrome b protein” represents soybean rust fungus (scientific name: Phakopsora pachyrhizi) which shows a resistance against QoI fungicide by having a mutation in the mitochondrial cytochrome b gene encoding mitochondrial cytochrome protein and as a result of the mutation, causing amino acid substitution of F129L.

The compound of the present invention, and the intermediate compound A may be existed as one or more stereoisomers. Examples of the stereoisomer include enantiomer, diastereoisomer, atropisomer, and geometric isomer. Each stereoisomer, and stereoisomer mixture(s) in an arbitrary ratio of these stereoisomers are included.

Examples of the geometric isomer include the following structures.

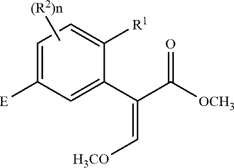

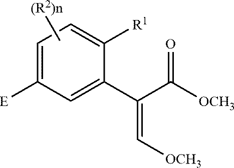

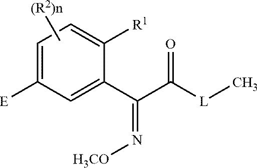

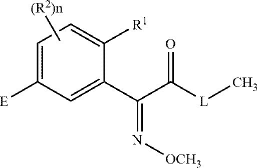

The compound of the present invention or its N-oxide may be mixed with an acid (such as hydrochloric acid, sulfuric acid, nitric acid, phosphoric acid, acetic acid, and benzoic acid) to form acid addition salts (such as hydrochloride salts, sulfates, nitrates, phosphates, acetates, and benzoates).

Embodiments of the compound N of the present invention include the following compounds.

[Embodiment 1] A compound N of the present invention wherein E represents R⁷—C═C—, and R⁷ represents a C1-C6 chain hydrocarbon group which may be optionally substituted with one or more substituents selected from Group F, or a C3-C6 cycloalkyl group which may be optionally substituted with one or more halogen atoms.

[Embodiment 2] A compound N of the present invention wherein E represents R⁷—C═C—, and R⁷ represents a C1-C6 chain hydrocarbon group which may be optionally substituted with one or more halogen atoms, or a C3-C4 cycloalkyl group which may be optionally substituted with one or more halogen atoms.

[Embodiment 3] A compound N of the present invention wherein E represents R⁷—C═C—, and R⁷ represents a C1-C6 chain hydrocarbon group, or C3-C4 cycloalkyl group.

[Embodiment 4] A compound N of the present invention wherein E represents R⁷—C═C—, and R⁷ represents C1-C6 alkyl group, or a cyclopropyl group.

[Embodiment 5] A compound N of the present invention wherein E represents a phenyl group which may be optionally substituted by one or more substituents selected from Group A.

[Embodiment 6] A compound N of the present invention wherein E represents a phenyl group which may be optionally substituted by one or more substituents selected from Group I.

[Embodiment 7] A compound N of the present invention wherein E represents a phenyl group which may be optionally substituted by one or more substituents selected from Group J.

[Embodiment 8] A compound N of the present invention wherein E represents a phenyl group which may be optionally substituted by one or more substituents selected from Group J1, and

Group J1 is a group consisting of a C1-C3 alkyl group, a C1-C3 alkoxy group {the C1-C3 alkyl group and the C1-C3 alkoxy group may be optionally substituted with one or more halogen atoms}, a halogen atom, a cyano group, and C(R⁹)═NOR¹¹.

[Embodiment 9] A compound N of the present invention wherein E represents a group represented by formula (III):

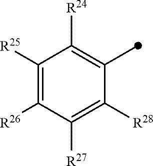

,
R²⁴ and R²⁸ are identical to or different from each other and represent a halogen atom, or a hydrogen atom,
R²⁵ and R²⁷ are identical to or different from each other and represent a C1-C3 alkyl group which may be optionally substituted with one or more halogen atoms, a C1-C3 alkoxy group which may be optionally substituted with one or more halogen atoms, C (O) R¹¹, C (R^{9x})═N—OR^{11x}, a halogen atom, a cyano group, or a hydrogen atom,
R^{9x} and R^{11x} are identical to or different from each other and represent a C1-C3 alkyl group,
R²⁶ represents a C1-C3 alkyl group which may be optionally substituted with one or more halogen atoms, a C1-C3 alkoxy group which may be optionally substituted with one or more halogen atoms, a halogen atom, or a hydrogen atom.

[Embodiment 10] The compound according to [Embodiment 9] wherein R²⁴, R²⁶, and R²⁸ are identical to or different from each other and represent a fluorine atom or a hydrogen atom, and

R²⁵ and R²⁷ are identical to or different from each other and represent C (R^{9x})═N—OR^{11x}, a halogen atom, a cyano group, or a hydrogen atom.

[Embodiment 11] A compound N of the present invention wherein E represents a five- to six- membered aromatic heterocyclic group which may be optionally substituted by one or more substituents selected from Group A.

[Embodiment 12] A compound N of the present invention wherein E represents a five- to six- membered aromatic heterocyclic group which may be optionally substituted with one or more substituents selected from Group H.

[Embodiment 13] A compound N of the present invention wherein E represents a five- to six- membered aromatic heterocyclic group which may be optionally substituted with one or more substituents selected from Group I.

[Embodiment 14] A compound N of the present invention wherein E represents a pyridyl group which may be optionally substituted with one or more substituents selected from Group J2, or a pyrimidinyl group which may be optionally substituted with one or more substituents selected from Group K,
Group J2 is a group consisting of a C1-C3 alkyl group, a C1-C3 alkoxy group {the C1-C3 alkyl group and the C1-C3 alkoxy group may be optionally substituted with one or more halogen atoms}, a halogen atom, and C (R^{9x}) ═N—OR^{11Y},
R^{11Y} represents a C1-C6 chain hydrocarbon group which may be optionally substituted with one or more substituents selected from Group F.

[Embodiment 15] A compound N of the present invention wherein E represents a pyridyl group which may be optionally substituted with one or more substituents selected from Group J3, or a pyrimidinyl group which may be optionally substituted with one or more substituents selected from Group K1,
Group J3 is a group consisting of a C1-C3 alkyl group, a C1-C3 alkoxy group {the C1-C3 alkyl group and the C1-C3 alkoxy group may be optionally substituted with one or more halogen atoms}, and a halogen atom,
Group K1 is a group consisting of a C1-C3 alkyl group which may be optionally substituted with one or more halogen atoms, a phenoxy group which may be optionally substituted with one or more substituents selected from Group G1, and a halogen atom, and
Group G1 is a group consisting of a cyano group and a halogen atom.

[Embodiment 16] A compound N of the present invention wherein E represents a thienyl group, a furanyl group, a thiadiazolyl group, or a pyrazolyl group {the thienyl group, the furanyl group, the thiadiazolyl group, and the pyrazolyl group may be optionally substituted with one or more substituents selected from Group J4},

Group J4 is a group consisting of a C1-C6 chain hydrocarbon group which may be optionally substituted with one or more substituents selected from Group F, a C1-C6 alkoxy group which may be optionally substituted with one or more halogen atoms, and a halogen atom.

[Embodiment 17] A compound N of the present invention wherein E represents a thienyl group, a furanyl group, a thiadiazolyl group, or a pyrazolyl group {the thienyl group, the furanyl group, the thiadiazolyl group, and the pyrazolyl group may be optionally substituted with one or more substituents selected from Group J5},

Group J5 is a group consisting of a C1-C6 chain hydrocarbon group which may be optionally substituted with one or more substituents selected from Group F, and a halogen atom.

[Embodiment 18] A compound N of the present invention wherein E represents a group represented by formula (IV-1), a group represented by formula (IV-2), a group represented by formula (IV-3), a group represented by formula (IV-4), a group represented by formula (IV-5), a group represented by formula (IV-6), or a group represented by formula (IV-7):

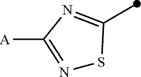

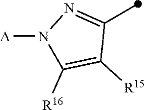

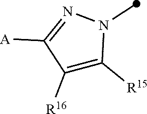

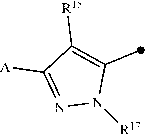

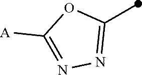

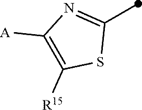

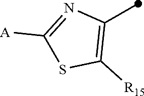

,
R¹⁵ and R¹⁶ are identical to or different from each other and represent a C1-C3 chain hydrocarbon group which may be optionally substituted with one or more halogen atoms, a halogen atom, or a hydrogen atom,
R¹⁷ represents a C1-C3 chain hydrocarbon group which may be optionally substituted with one or more halogen atoms, or a hydrogen atom, and
A represents a phenyl group or a pyridyl group {the phenyl group and the pyridyl group may be optionally substituted with one or more substituents selected from Group G} .

[Embodiment 19] A compound N of the present invention wherein E represents a group represented by formula (IV-1), a group represented by formula (IV-2), or a group represented by formula (IV-3), and A represents a phenyl group which may be optionally substituted with one or more halogen atoms.

[Embodiment 20] A compound N of the present invention wherein E represents a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), or a group represented by formula (IV-7).

[Embodiment 21] A compound N of the present invention wherein E represents a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), or a group represented by formula (IV-3), and A represents a phenyl group which may be optionally substituted with one or more halogen atoms.

[Embodiment 22] A compound N of the present invention wherein E represents R⁵—O—N═C(R⁴) —, R⁴ represents a C1-C3 alkyl group, or a hydrogen atom, R⁵ represents a C1-C6 chain hydrocarbon group which may be optionally substituted with one or more substituents selected from Group F, a C3-C6 cycloalkyl group which a phenyl group which may be optionally substituted with one or more halogen atoms, a phenyl group, a five- to six- membered aromatic heterocyclic group {the phenyl group and the five- to six- membered aromatic heterocyclic group may be optionally substituted with one or more substituents selected from Group G}, or R¹⁸—CH₂—, and R¹⁸ represents a phenyl group, or a five- to six- membered aromatic heterocyclic group {the phenyl group and the five-to six- membered aromatic heterocyclic group may be optionally substituted with one or more substituents selected from Group G}.

[Embodiment 23] A compound N of the present invention wherein E represents R⁵—O—N═C (R⁴)—, R⁴ represents a C1-C3 alkyl group, or a hydrogen atom, R⁵ represents a C1-C6 chain hydrocarbon group which may be optionally substituted with one or more substituents selected from Group F, a C3-C4 cycloalkyl group which may be optionally substituted with one or more halogen atoms, or R¹⁸—CH₂—, and R¹⁸ represents a phenyl group which may be optionally substituted with one or more substituents selected from Group G.

[Embodiment 24] A compound N of the present invention wherein E represents R⁵—O—N═C (R⁴) —, R⁴ represents a methyl group or a hydrogen atom, R⁵ represents a C1-C4 alkyl group which may be optionally substituted with one or more substituents selected from Group F, a C3-C4 cycloalkyl group, or R¹⁸—CH₂—, and R¹⁸ represents a phenyl group {the phenyl group may be optionally substituted with one or more substituents selected from a group consisting of a C1-C3 alkyl group which may be optionally substituted with one or more halogen atoms and a halogen atom}.

[Embodiment 25] A compound N of the present invention wherein E represents R⁶O—, and R⁶ represents a C2-C6 which may be optionally substituted with one or more substituents selected from Group F, a C3-C4 cycloalkyl group which may be optionally substituted with one or more halogen atoms, or R¹³R⁴NC (O) -.

[Embodiment 26] A compound N of the present invention wherein E represents R⁶O—, and R⁶ represents a C2-C6 chain hydrocarbon group which may be optionally substituted with one or more halogen atoms.

[Embodiment 27] A compound N of the present invention wherein E represents R⁶O—, and R⁶ represents a C2-C6 chain hydrocarbon group.

[Embodiment 28] A compound N of the present invention wherein E represents R⁶O—, and R⁶ represents a C2-C6 alkyl group.

[Embodiment 29] A compound N of the present invention wherein E represents a C5-C6 cycloalkenyl group which may be optionally substituted with one or more substituents selected from Group D.

[Embodiment 30] A compound N of the present invention wherein E represents a C5-C6 cycloalkenyl group which may be optionally substituted with one or more halogen atoms.

[Embodiment 31] A compound N of the present invention wherein E represents a C5-C6 cycloalkenyl group.

[Embodiment 32] A compound N of the present invention wherein E represents a phenyl group which may be optionally substituted by one or more substituents selected from Group A, or a five- to six- membered aromatic heterocyclic group which may be optionally substituted by one or more substituents selected from Group A.

[Embodiment 33] A compound N of the present invention wherein E represents a phenyl group which may be optionally substituted by one or more substituents selected from Group I, or a five- to six- membered aromatic heterocyclic group which may be optionally substituted with one or more substituents selected from Group H.

[Embodiment 34] A compound N of the present invention wherein E represents a phenyl group which may be optionally substituted by one or more substituents selected from Group J, or a five- to six- membered aromatic heterocyclic group which may be optionally substituted with one or more substituents selected from Group I.

[Embodiment 35] A compound N of the present invention wherein E represents a phenyl group which may be optionally substituted by one or more substituents selected from Group J1, or a five- to six- membered aromatic heterocyclic group which may be optionally substituted with one or more substituents selected from Group I.

[Embodiment 36] A compound N of the present invention wherein E represents a phenyl group which may be optionally substituted by one or more substituents selected from Group J1, a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), or a group represented by formula (IV-7).

[Embodiment 37] A compound N of the present invention wherein E represents a phenyl group which may be optionally substituted by one or more substituents selected from Group J1, a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), or a group represented by formula (IV-3), and A represents a phenyl group which may be optionally substituted with one or more halogen atoms.

[Embodiment 38] A compound N of the present invention wherein E represents a group represented by formula (III), a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), or a group represented by formula (IV-3), and A represents a phenyl group which may be optionally substituted with one or more halogen atoms.

[Embodiment 39] The compound according to [Embodiment 38] wherein R²⁴, R²⁶, and R²⁸ are identical to or different from each other and represent a fluorine atom or a hydrogen atom, and R²⁵, and R²⁷ are identical to or different from each other and represent C (R^{9x}) ═N—OR^{11x}, a halogen atom, a cyano group, or a hydrogen atom.

[Embodiment 40] A compound N of the present invention wherein E represents a phenyl group which may be optionally substituted by one or more substituents selected from Group A, a five- to six- membered aromatic heterocyclic group which may be optionally substituted by one or more substituents selected from Group A, or R⁷—C═C—, and R⁷ represents a C1-C6 chain hydrocarbon group which may be optionally substituted with one or more substituents selected from Group F, or a C3-C6 cycloalkyl group which may be optionally substituted with one or more halogen atoms.

[Embodiment 41] A compound N of the present invention wherein E represents a phenyl group which may be optionally substituted by one or more substituents selected from Group I, a five- to six- membered aromatic heterocyclic group which may be optionally substituted with one or more substituents selected from Group H, or R⁷—C═C—, and R⁷ represents a C1-C6 chain hydrocarbon group which may be optionally substituted with one or more halogen atoms, or a C3-C4 cycloalkyl group which may be optionally substituted with one or more halogen atoms.

[Embodiment 42] A compound N of the present invention wherein E represents a phenyl group which may be optionally substituted by one or more substituents selected from Group J, a five- to six- membered aromatic heterocyclic group which may be optionally substituted with one or more substituents selected from Group I, or R⁷—C═C—, and R⁷ represents a C1-C6 chain hydrocarbon group, or a C3-C4 cycloalkyl group.

[Embodiment 43] A compound N of the present invention wherein E represents a phenyl group which may be optionally substituted by one or more substituents selected from Group Jl, a five- to six- membered aromatic heterocyclic group which may be optionally substituted with one or more substituents selected from Group I, or R⁷—C═C—, and R⁷ represents a C1-C6 chain hydrocarbon group, or a C3-C4 cycloalkyl group.

[Embodiment 44] A compound N of the present invention wherein E represents a phenyl group which may be optionally substituted by one or more substituents selected from Group J1, a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), a group represented by formula (IV-7), or R⁷—C═C—, and R⁷ represents a C1-C6 chain hydrocarbon group, or a C3-C4 cycloalkyl group.

[Embodiment 45] A compound N of the present invention wherein E represents a phenyl group which may be optionally substituted by one or more substituents selected from Group J1, a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), or R⁷—C═C—, R⁷ represents a C1-C6 alkyl group, or a cyclopropyl group, and A represents a phenyl group which may be optionally substituted with one or more halogen atoms.

[Embodiment 46] A compound N of the present invention wherein E represents a group represented by formula (III), a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), or R⁷—C═C—, R⁷ represents a C1-C6 alkyl group, or a cyclopropyl group, and A represents a phenyl group which may be optionally substituted with one or more halogen atoms.

[Embodiment 47] The compound according to [Embodiment 46] wherein R²⁴, R²⁶, and R²⁸ are identical to or different from each other and represent a fluorine atom or a hydrogen atom, and R²⁵ and R²⁷ are identical to or different from each other and represent C (R^{9x}) —N—OR^{11x}, a halogen atom, a cyano group, or a hydrogen atom.

[Embodiment 48] A compound N of the present invention wherein E represents a phenyl group which may be optionally substituted by one or more substituents selected from Group A, a five- to six- membered aromatic heterocyclic group which may be optionally substituted by one or more substituents selected from Group A, a C5-C6 cycloalkenyl group which may be optionally substituted with one or more substituents selected from Group D, or R⁷—C═C—, and R⁷ represents a C1-C6 chain hydrocarbon group which may be optionally substituted with one or more substituents selected from Group F, or a C3-C6 cycloalkyl group which may be optionally substituted with one or more halogen atoms.

[Embodiment 49] A compound N of the present invention wherein E represents a phenyl group which may be optionally substituted by one or more substituents selected from Group I, a five- to six- membered aromatic heterocyclic group which may be optionally substituted with one or more substituents selected from Group H, a C5-C6 cycloalkenyl group which may be optionally substituted with one or more halogen atoms, or R⁷—C—C—, and R⁷ represents a C1-C6 chain hydrocarbon group which may be optionally substituted with one or more halogen atoms, or a C3-C4 cycloalkyl group which may be optionally substituted with one or more halogen atoms.

[Embodiment 50] A compound N of the present invention wherein E represents a phenyl group which may be optionally substituted by one or more substituents selected from Group J, a five- to six- membered aromatic heterocyclic group which may be optionally substituted with one or more substituents selected from Group I, a C5-C6 cycloalkenyl group which may be optionally substituted with one or more halogen atoms, or R⁷—C═C—, and R⁷ represents a C1-C6 chain hydrocarbon group, or a C3-C4 cycloalkyl group.

[Embodiment 51] A compound N of the present invention wherein E represents a phenyl group which may be optionally substituted by one or more substituents selected from Group J1, a five- to six- membered aromatic heterocyclic group which may be optionally substituted with one or more substituents selected from Group I, a C5-C6 cycloalkenyl group which may be optionally substituted with one or more halogen atoms, or R⁷—C═C—, and R⁷ represents a C1-C6 chain hydrocarbon group, or a C3-C4 cycloalkyl group.

[Embodiment 52] A compound N of the present invention wherein E represents a phenyl group which may be optionally substituted by one or more substituents selected from Group J1, a pyridyl group which may be optionally substituted with one or more substituents selected from Group J2, a pyrimidinyl group which may be optionally substituted with one or more substituents selected from Group K, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J4}, a group represented by formula (IV-1), a group represented by formula (IV-2), a group represented by formula (IV-3), a group represented by formula (IV-4), a group represented by formula (IV-5), a group represented by formula (IV-6), a group represented by formula (IV-7), a C5-C6 cycloalkenyl group, or R⁷—C═C—, and R⁷ represents a C1-C6 chain hydrocarbon group, or a C3-C4 cycloalkyl group.

[Embodiment 53] A compound N of the present invention wherein E represents a phenyl group which may be optionally substituted by one or more substituents selected from Group J1, a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), a C5-C6 cycloalkenyl group, or R⁷—C═C—, R⁷ represents a C1-C6 alkyl group, or a cyclopropyl group, and A represents a phenyl group which may be optionally substituted with one or more halogen atoms.

[Embodiment 54] A compound N of the present invention wherein E represents a group represented by formula (III), a pyridyl group which may be optionally substituted with one or more substituents selected from Group J3, a pyrimidinyl group which may be optionally substituted with one or more substituents selected from Group K1, a thienyl group, a furanyl group, a thiadiazolyl group, a pyrazolyl group {the thienyl group, the furanyl group, the thiadiazolyl group, and the pyrazolyl group may be optionally substituted with one or more substituents selected from Group J5}, a group represented by formula (IV-1), a group represented by formula (IV-2), a group represented by formula (IV-3), a C5-C6 cycloalkenyl group, or R⁷—C═C—, and R⁷ represents a C1-C6 alkyl group, or a cyclopropyl group, and A represents a phenyl group which may be optionally substituted with one or more halogen atoms.

[Embodiment 55] The compound according to [Embodiment 54] wherein R²⁴, R²⁶, and R²⁸ are identical to or different from each other and represent a fluorine atom or a hydrogen atom, and R²⁵ and R²⁷ are identical to or different from each other and represent C (R^{9x}) ═N—OR^{11x}, a halogen atom, a cyano group, or a hydrogen atom.

[Embodiment 56] A compound N of the present invention wherein E represents a phenyl group, a five- to six- membered aromatic heterocyclic group {the phenyl group, and the five-to six- membered aromatic heterocyclic group may be optionally substituted with one or more substituents selected from Group J1}, R⁷—C═C—, or R⁶O—, R⁷ represents a C1-C6 chain hydrocarbon group, or a C3-C4 cycloalkyl group, and R⁶ represents a C2-C6 which may be optionally substituted with one or more substituents selected from Group F.

[Embodiment 57] A compound N of the present invention wherein E represents a phenyl group, a five- to six- membered aromatic heterocyclic group {the phenyl group, and the five-to six- membered aromatic heterocyclic group may be optionally substituted with one or more substituents selected from Group J1}, R⁷—C═C—, or R⁶O—, R⁷ represents a C1-C6 chain hydrocarbon group, or a C3-C4 cycloalkyl group, and R⁶ represents a C2-C6 chain hydrocarbon group which may be optionally substituted with one or more halogen atoms.

[Embodiment 58] A compound N of the present invention wherein E represents a phenyl group, a five- to six- membered aromatic heterocyclic group {the phenyl group, and the five-to six- membered aromatic heterocyclic group may be optionally substituted with one or more substituents selected from Group J1} , R⁷—C═C—, or R⁶O—, R⁷ represents a C1-C6 chain hydrocarbon group, or a C3-C4 cycloalkyl group, and R⁶ represents a C2-C6 alkyl group.

[Embodiment 59] A compound N of the present invention wherein E represents a C5-C6 cycloalkenyl group, a phenyl group, a five- to six- membered aromatic heterocyclic group {the phenyl group, and the five- to six- membered aromatic heterocyclic group may be optionally substituted with one or more substituents selected from Group A1}, R⁷—C═C—, R⁵—ON═C(R⁴)—, or R⁶O—, R⁵ represents a C1-C6 chain hydrocarbon group which may be optionally substituted with one or more substituents selected from Group F, a C3-C6 cycloalkyl group which may be optionally substituted with one or more substituents selected from Group D, a phenyl group which may be optionally substituted with one or more substituents selected from Group C, or R¹⁸—CH₂—, R⁶ represents a methyl group which is substituted with one or more substituents selected from Group F, a C2-C6 chain hydrocarbon group which may be optionally substituted with one or more substituents selected from Group F, or a C3-C4 which may be optionally substituted with one or more substituents selected from Group D, R⁷ represents a C1-C6 chain hydrocarbon group which may be optionally substituted with one or more substituents selected from Group F, or a C3-C6 cycloalkyl group which may be optionally substituted with one or more substituents selected from Group D, Group A1 is a group consisting of a C1-C6 chain hydrocarbon group which may be optionally substituted with one or more substituents selected from Group F, a C3-C6 cycloalkyl group which may be optionally substituted with one or more substituents selected from Group D, OR^{14a} , C (O) R^{11a} , C (R⁹) ═N—OR^{11a} , a phenyl group, a five- to six- membered aromatic heterocyclic group {the phenyl group, and the five- to six- membered aromatic heterocyclic group may be optionally substituted with one or more substituents selected from Group E}, a halogen atom, a cyano group, and a nitro group, R^{11a} represents a C1-C6 chain hydrocarbon group which may be optionally substituted with one or more substituents selected from Group F, and R^{14a} represents a C1-C6 chain hydrocarbon group which may be optionally substituted with one or more substituents selected from Group F, a phenyl group, or a five- to six- membered aromatic heterocyclic group {the phenyl group, and the five- to six-membered aromatic heterocyclic group may be optionally substituted with one or more substituents selected from Group C} .

[Embodiment 60] A compound N of the present invention wherein E represents a C5-C6 cycloalkenyl group, a phenyl group, a five- to six- membered aromatic heterocyclic group {the phenyl group, and the five- to six- membered aromatic heterocyclic group may be optionally substituted with one or more substituents selected from Group A2}, R⁷—C═C—, R⁵—ON═C(R⁴)—, or R⁶O—, R⁴ represents a methyl group or a hydrogen atom, R⁵ represents a C1-C6 chain hydrocarbon group which may be optionally substituted with one or more substituents selected from Group F1, a C3-C6 cycloalkyl group, phenyl group, or R¹⁸—CH₂—, R⁶ represents (c—Pr)CH₂—, a C2-C6 chain hydrocarbon group, or a C3-C4 cycloalkyl group, R⁷ represents a C1-C6 chain hydrocarbon group, or a C3-C6 cycloalkyl group, R¹⁸ represents a phenyl group, or a pyridyl group {the phenyl group and the pyridyl group may be optionally substituted with one or more substituents selected from Group C1}, Group A2 is a group consisting of a C1-C6 chain hydrocarbon group which may be optionally substituted with one or more halogen atoms, a C3-C6 cycloalkyl group, OR^{14b}, a phenyl group which may be optionally substituted with one or more substituents selected from Group E, a halogen atom, and a cyano group, Group C1 is a group consisting of a C1-C3 chain hydrocarbon group which may be optionally substituted with one or more halogen atoms, and a halogen atom, Group F1 is a group consisting of a C3-C4 cycloalkyl group and a halogen atom, and R^{14b} represents a C1-C6 chain hydrocarbon group which may be optionally substituted with one or more halogen atoms, or a phenyl group which may be optionally substituted with one or more substituents selected from Group C1.

[Embodiment 61] A compound N of the present invention wherein E represents R⁵—O—N═C(R⁴)—, R⁴ represents a methyl group or a hydrogen atom, R⁵ represents a C1-C4 alkyl group which may be optionally substituted with one or more substituents selected from Group F1, a C3-C6 cycloalkyl group, or R¹⁸—CH₂—, and R¹⁸ represents a phenyl group, or a pyridyl group {the phenyl group, and the pyridyl group may be optionally substituted with one or more substituents selected from group C1}.

[Embodiment 62] The compound according any one of [Embodiment 1] to [Embodiment 61] or the compound N of the present invention wherein R¹ represents a C1-C3 chain hydrocarbon group which may be optionally substituted with one or more halogen atoms, or a halogen atom, and n is 0.

[Embodiment 63] The compound according any one of [Embodiment 1] to [Embodiment 61] or the compound N of the present invention wherein R¹ represents a methyl group, and n is 0.

[Embodiment 64] The compound according any one of [Embodiment 1] to [Embodiment 61] or the compound N of the present invention wherein R¹ represents a hydrogen atom, and n is 1.

[Embodiment 65] The compound according any one of [Embodiment 1] to [Embodiment 61] or the compound N of the present invention wherein R¹ represents a hydrogen atom, n is 1, and R² represents a methyl group or a halogen atom.

[Embodiment 66] The compound according any one of [Embodiment 1] to [Embodiment 61] or the compound N of the present invention wherein R¹ represents a hydrogen atom, n is 1, and R² represents a methyl group.

[Embodiment 67] The compound according any one of [Embodiment 1] to [Embodiment 61] or the compound N of the present invention wherein R¹ represents a hydrogen atom, n is 1, and R² represents a chlorine atom.

[Embodiment 68] The compound according any one of [Embodiment 1] to [Embodiment 61] or the compound N of the present invention wherein R¹ represents a C1-C3 chain hydrocarbon group which may be optionally substituted with one or more halogen atoms, or a halogen atom, n is 0, and Q represents a group represented by Q1.

[Embodiment 69] The compound according any one of [Embodiment 1] to [Embodiment 61] or the compound N of the present invention wherein R¹ represents a methyl group, n is 0, and Q represents a group represented by Q1.

[Embodiment 70] The compound according any one of [Embodiment 1] to [Embodiment 61] or the compound N of the present invention wherein R¹ represents a hydrogen atom, n is 1, and Q represents a group represented by Q1.

[Embodiment 71] The compound according any one of [Embodiment 1] to [Embodiment 61] or the compound N of the present invention wherein R¹ represents a hydrogen atom, n is 1, R² represents a methyl group or a halogen atom, and Q represents a group represented by Q1.

[Embodiment 72] The compound according any one of [Embodiment 1] to [Embodiment 61] or the compound N of the present invention wherein R¹ represents a hydrogen atom, n is 1, R² represents a methyl group, and Q represents a group represented by Q1.

[Embodiment 73] The compound according any one of [Embodiment 1] to [Embodiment 61] or the compound N of the present invention wherein R¹ represents a hydrogen atom, n is 1, R² represents a chlorine atom, and Q represents a group represented by Q1.

[Embodiment 74] The compound according any one of [Embodiment 1] to [Embodiment 61] or the compound N of the present invention wherein R¹ represents a C1-C3 chain hydrocarbon group which may be optionally substituted with one or more halogen atoms, or a halogen atom, n is 0, and Q represents a group represented by Q2.

[Embodiment 75] The compound according any one of [Embodiment 1] to [Embodiment 61] or the compound N of the present invention wherein R¹ represents a methyl group, n is 0, and Q represents a group represented by Q2.

[Embodiment 76] The compound according any one of [Embodiment 1] to [Embodiment 61] or the compound N of the present invention wherein R¹ represents a hydrogen atom, n is 1, and Q represents a group represented by Q2.

[Embodiment 77] The compound according any one of [Embodiment 1] to [Embodiment 61] or the compound N of the present invention wherein R¹ represents a hydrogen atom, n is 1, R² represents a methyl group or a halogen atom, and Q represents a group represented by Q2.

[Embodiment 78] The compound according any one of [Embodiment 1] to [Embodiment 61] or the compound N of the present invention wherein R¹ represents a hydrogen atom, n is 1, R² represents a methyl group, and Q represents a group represented by Q2.

[Embodiment 79] The compound according any one of [Embodiment 1] to [Embodiment 61] or the compound N of the present invention wherein R¹ represents a hydrogen atom, n is 1, R² represents a chlorine atom, and Q represents a group represented by Q2.

[Embodiment 80] A compound N of the present invention wherein E represents a C5-C6 cycloalkenyl group which may be optionally substituted with one or more substituents selected from Group D, a phenyl group, a five- to six-membered aromatic heterocyclic group {the phenyl group and the five- to six- membered aromatic heterocyclic group may be optionally substituted with one or more substituents selected from Group H}, R⁷—C═C—, R⁵—O—N═C (R⁴) —, or R⁶O—, R⁴ represents a C1-C3 alkyl group, or a hydrogen atom, R⁵ represents a C1-C6 chain hydrocarbon group which may be optionally substituted with one or more substituents selected from Group F, a C3-C6 cycloalkyl group which may be optionally substituted with one or more halogen atoms, a phenyl group, a five- to six- membered aromatic heterocyclic group {the phenyl group and the five- to six- membered aromatic heterocyclic group may be optionally substituted with one or more substituents selected from Group G}, or R¹⁸—CH₂—, R⁶ represents a C2-C6 which may be optionally substituted with one or more substituents selected from Group F, a C3-C4 cycloalkyl group which may be optionally substituted with one or more halogen atoms, or R¹³R⁴NC(O)—, R⁷ represents a C1-C6 chain hydrocarbon group which may be optionally substituted with one or more substituents selected from Group F, or a C3-C6 cycloalkyl group which may be optionally substituted with one or more halogen atoms, R¹¹ and R¹³ are identical to or different from each other and represent a C1-C6 chain hydrocarbon group which may be optionally substituted with one or more substituents selected from Group F, or a C3-C6 cycloalkyl group which may be optionally substituted with one or more halogen atoms, R¹⁴ represents a C1-C6 chain hydrocarbon group which may be optionally substituted with one or more substituents selected from Group F, a C3-C6 cycloalkyl group which may be optionally substituted with one or more halogen atoms, or a phenyl group which may be optionally substituted with one or more substituents selected from Group G, R¹⁸ represents a phenyl group, or a five- to six- membered aromatic heterocyclic group {the phenyl group and the five- to six-membered aromatic heterocyclic group may be optionally substituted with one or more substituents selected from Group G},

Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group, and

Group H is a group consisting of a C1-C6 chain hydrocarbon group which may be optionally substituted with one or more halogen atoms, a C3-C4 cycloalkyl group which may be optionally substituted with one or more halogen atoms, OR¹⁴, a halogen atom, a cyano group, C (R⁴) ═N—OR¹¹, a phenyl group, and a five- to six- membered aromatic heterocyclic group {the phenyl group and the five- to six- membered aromatic heterocyclic group may be optionally substituted with one or more substituents selected from Group G}.

[Embodiment 81] The compound wherein Group H is a group consisting of a C1-C6 chain hydrocarbon group which may be optionally substituted with one or more halogen atoms, a C3-C4 cycloalkyl group which may be optionally substituted with one or more halogen atoms, OR¹⁴, a halogen atom, a cyano group, C (R¹⁴) ═N—OR¹¹, a phenyl group, and a five- to six-membered aromatic heterocyclic group {the phenyl group and the five- to six- membered aromatic heterocyclic group may be optionally substituted with one or more substituents selected from Group G}.

Next, the process for preparing the compound of the present invention are explained.

### Process A

A compound represented by formula (A1) (hereinafter, referred to as “Compound (A1)”) can be prepared by reacting a compound represented by formula (B1) (hereinafter, referred to as “Compound (B1)”) with a compound represented by formula (M1) (hereinafter, referred to as “Compound (M1)”) in the presence of a palladium catalyst and a base.

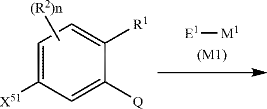

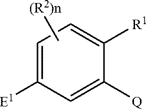

[wherein E¹ represents a C6-C10 aryl group, or a five- to ten- membered aromatic heterocyclic group {the C6-C10 aryl group and the five- to ten- membered aromatic heterocyclic group may be optionally substituted by one or more substituents selected from Group A}; M¹ represents B(OH)₂, or 4,4,5,5-tetramethyl-1,3,2-dioxaborolan-2-yl group; and X⁵¹ represents a leaving group such as chlorine atom, bromine atom, iodine atom, or triflyloxy group; and the other symbols are the same as defined above.

The reaction is usually carried out in a solvent. Examples of the solvent to be used in the reaction include hydrocarbons such as hexane, toluene, and xylene (hereinafter, collectively referred to as hydrocarbons); ethers such as methyl tert-butyl ether (hereinafter, referred to as MTBE), tetrahydrofuran (hereinafter, referred to as THF), dimethoxyethane (hereinafter, collectively referred to as ethers); halogenated hydrocarbons such as chloroform and chlorobenzene (hereinafter, collectively referred to as halogenated hydrocarbons); amides such as dimethylformamide (hereinafter, referred to as DMF) and N-methyl pyrrolidone (hereinafter, collectively referred to as amides); esters such as methyl acetate and ethyl acetate (hereinafter, collectively referred to as esters); nitriles such as acetonitrile and propionitrile (hereinafter, collectively referred to as nitriles); water; and mixed solvents of these’ two or more solvents.

Example of the palladium catalysts to be used in the reaction includes [1,1′-bis(diphenylphoshino)ferrocene]palladium (II) dichloride.

Examples of the base to be used in the reaction include organic bases such as triethylamine and pyridine (hereinafter, collectively referred to as organic bases); alkali metal carbonates such as sodium carbonates and potassium carbonates (hereinafter, collectively referred to as alkali metal carbonates); alkali metal hydrocarbonates such as sodium hydrocarbonate and potassium hydrocarbonate (hereinafter, collectively referred to as alkali metal hydrocarbonates); sodium fluoride, and tripotassium phosphate.

In the reaction, the compound (M1) is usually used within a range of 1 to 10 molar ratio(s), the palladium catalyst is usually used within a range of 0.01 to 1 molar ratio(s), and the base is usually used within a range of 1 to 10 molar ratio (s), as opposed to 1 mol of the compound (B1).

The reaction temperature is usually within a range of 0 to 150° C. The reaction period of the reaction is usually within a range of 0.1 to 120 hours.

When the reaction is completed, water is added to reaction mixtures, and the resulting mixtures are extracted with organic solvent(s), and the organic layers are worked up (such as drying and concentration) to isolate the compound (A1).

The compound (M1) is a known compound, or can be prepared according to a known method.

### Process B

The compound (A1) can be prepared by reacting a compound represented by formula (B2) (hereinafter, referred to as “Compound (B2)”) with a compound represented by formula (M2) (hereinafter, referred to as “Compound (M2)”) in the presence of a palladium catalyst and a base.

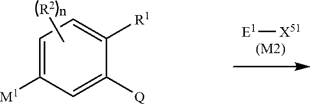

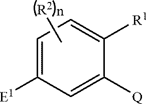

[wherein the symbols are the same as defined above.]

The reaction can be carried out by using the compound (M2) in place of the compound (B1) and using the compound (B2) in place of the compound (M1) according to the Process A.

The compound (M2) is a known compound, or can be prepared according to a known method.

### Process C

A compound represented by formula (A2) (hereinafter, referred to as “Compound (A2)”) can be prepared by reacting the compound (B1) with a compound represented by formula (M3) (hereinafter, referred to as “Compound (M3)”) in the presence of a metal catalyst and a base.

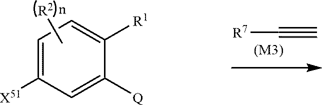

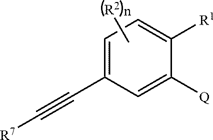

[wherein the symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the metal catalyst to be used in the reaction include bis(triphenylphosphine)palladium(II) dichloride (hereinafter, referred to as PdCl₂ (PPh₃)₂) and a copper (I) iodide.

The base to be used in the reaction includes the organic bases.

In the reaction, the compound (M3) is usually used within a range of 1 to 10 molar ratio(s), the metal catalyst is usually used within a range of 0.01 to 1 molar ratio(s), and the base is usually used within a range of 1 to 10 molar ratio(s), as opposed to 1 mol of the compound (B1).

The reaction temperature is usually within a range of 0 to 150° C. The reaction period of the reaction is usually within a range of 0.1 to 120 hours.

When the reaction is completed, water is added to the reaction mixtures, and the resulting mixtures are extracted with organic solvent(s), the organic layers are worked up (such as concentration and drying) to isolate the compound (A2).

The compound (M3) is a known compound, or can be prepared according to a known method.

### Process D

A compound represented by formula (A3) (hereinafter, referred to as “Compound (A3)”) can be prepared by reacting a compound represented by formula (B3) (hereinafter, referred to as “Compound (B3)”) with a compound represented by formula (M4) (hereinafter, referred to as “Compound (M4)”) or salts thereof.

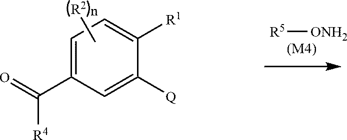

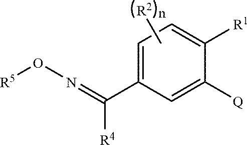

[wherein the symbols are the same as defined above.]

Examples of the salts of the compound (M4) include hydrochloride salts and sulfates.

The reaction is usually carried out in a solvent. Examples of the solvents to be used in the reaction include hydrocarbons, ethers, halogenated hydrocarbons, amides, esters, nitriles, alcohols (such as methanol, and ethanol), and mixed solvents of these two or more solvents.

A base may be used in the reaction as needed.

Examples of the bases to be used in the reaction include organic bases, alkali metal carbonates, alkali metal hydrocarbonates, sodium hydride and tripotassium phosphate.

In the reaction, the compound (M4) is usually used within a range of 1 to 10 molar ratio (s) , and the base is usually used within a range of 1 to 10 molar ratio (s), as opposed to 1 mol of the compound (B3).

The reaction temperature is usually within a range of 0 to 150° C. The reaction period of the reaction is usually within a range of 0.1 to 120 hours.

When the reaction is completed, water is added to the reaction mixtures, and the resulting mixtures are extracted with organic solvent(s), and the organic layers are worked up (such as concentration and drying) to isolate the compound (A3) .

Also the compound (A3) can be prepared according a method described in, for example, WO 1998/043949 A1.

The compound (M4) is a known compound, or can be prepared according to a known method.

### Process E

A compound represented by formula (A4) (hereinafter, referred to as “Compound (A4)”) can be prepared by reacting the compound (B3) with a compound represented by formula (M5) (hereinafter, referred to as “Compound (M5)”) or salts thereof.

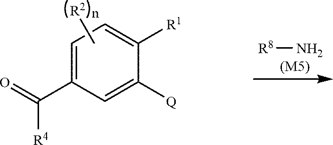

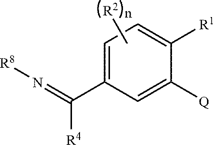

[wherein the symbols are the same as defined above.]

Examples of the salts of the compound (M5) include hydrochloride salts and sulfates.

The reaction can be carried out by using the compound (M5) in place of the compound (M4) according to the process D.

The compound (M5) is a known compound, or can be prepared according to a known method.

### Process F

The compound (A3) can be prepared by reacting a compound represented by formula (B4) (hereinafter, referred to as “Compound (B4)”) with a compound represented by formula (M6) (hereinafter, referred to as “Compound (M6)”) in the presence of a base.

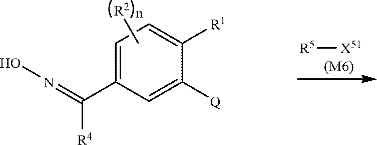

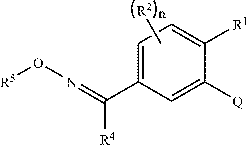

[wherein the symbols are the same as defined above.]

The reaction is usually carried out in a solvent. Examples of the solvents to be used in the reaction include hydrocarbons, ethers, halogenated hydrocarbons, amides, esters, nitriles, and mixed solvents of these two or more solvents.

Examples of the bases include organic bases, alkali metal carbonates, alkali metal hydrocarbonates, sodium hydride, and tripotassium phosphate.

In the reaction, the compound (M6) is usually used within a range of 1 to 10 molar ratio (s), and the base is usually used within a range of 1 to 10 molar ratio (s), as opposed to 1 mol of the compound (B4).

The reaction temperature is usually within a range of -20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A3) .

The compound (M6) is a known compound, or can be prepared according to a known method.

### Process H

A compound represented by formula (A5) (hereinafter, referred to as “Compound (A5)”) can be reacting the compound (B1) with a compound represented by formula (M8) (hereinafter, referred to as “Compound (M8)”) in the presence of a metal catalyst and a base.

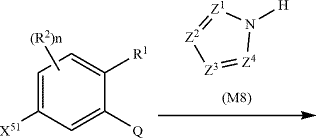

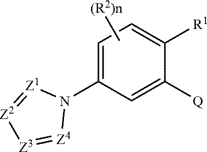

[wherein Z¹ represents a nitrogen atom or CR⁵¹; Z² represents a nitrogen atom or CR⁵²; Z³ represents a nitrogen atom or CR⁵³; and Z⁴ represents a nitrogen atom or CR⁵⁴ (with the proviso that the case where Z¹, Z², Z³, and Z⁴ are all nitrogen atoms is excluded); R⁵¹, R⁵², R⁵³, R⁵⁴ are identical to or different from each other and represent a C1-C6 chain hydrocarbon group which may be optionally substituted with one or more substituents selected from Group F, a C3-C6 cycloalkyl group which may be optionally substituted with one or more substituents selected from Group D, a C1-C6 alkylthio group which may be optionally substituted with one or more substituents selected from Group F, OR¹⁴, C(O)R¹¹, C (O)OR¹¹, NR¹¹R⁹, C (R⁹) ═N—OR¹¹, a phenyl group, a five- to six-membered aromatic heterocyclic group {the phenyl group, and the five- to six- membered aromatic heterocyclic group may be optionally substituted with one or more substituents selected from Group E}, a halogen atom, a cyano group, a nitro group, or a hydrogen atom; and the other symbols are the same as defined above.]

The reaction is usually carried out in a solvent. Examples of the solvents to be used in the reaction include ethers, hydrocarbons, amides, water, and mixed solvents of these two or more solvents.

Examples of the metal catalyst include copper catalysts (such as copper (I) iodide, copper (I) bromide, copper (I) chloride, copper (I) oxide, trifluoromethane sulfonate copper (I) benzene complex, tetrakis(acetonitrile) copper (I) hexafluorophoshate, and 2-thiophene carboxylate copper (I)); nickel catalysts (such as bis(cyclooctadine)nickel (0), and nickel (II) chloride).

Examples of the bases include organic bases, alkali metal carbonates, alkali metal hydrocarbonates, sodium fluoride, and tripotassium phosphate.

A ligand and/or an alkali metal halide may be used in the reaction as needed.

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethyletylenediamine, and N,N-dimethylglycine hydrochloride salt.

When the ligand is used in the reaction, the ligand is usually used within a range of 0.01 to 1 molar ratio(s), as opposed to 1 mol of the compound (B1).

Examples of the alkali metal halides include potassium fluoride, sodium fluoride, lithium chloride, and sodium chloride. When the alkali metal halide is used in the reaction, the alkali metal halide is usually used within a range of 0.1 to 5 molar ratio(s), as opposed to 1 mol of the compound (B1).

In the reaction, the compound (M8) is usually used within a range of 1 to 10 molar ratio(s), the metal catalyst is usually used within a range of 0.01 to 2 molar ratio(s), and the base is usually used within a range of 1 to 10 molar ratio(s), as opposed to 1 mol of the compound (B1).

The reaction temperature is usually within a range of -20 to 200° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A5) .

The compound (M8) is a known compound, or can be prepared according to a known method.

### Process I

A compound represented by formula (A6) (hereinafter, referred to as “Compound (A6)”) can be prepared by reacting a compound represented by formula (B5) (hereinafter, referred to as “Compound (B5)”) with a compound represented by formula (M9) (hereinafter, referred to as “Compound (M9)”) in the presence of a phosphines and an azodiester.

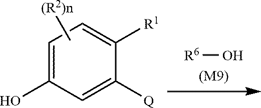

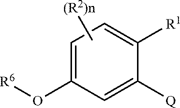

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixed solvents of these two or more solvents.

Examples of the phosphines include triphenylphosphine and trimethylphosphine.

Examples of the azodiesters include diethyl azodicarboxylate, diisopropyl azodicarboxylate, and bis (2-methoxyethyl) azodicarboxylate.

In the reaction, the compound (M9) is usually used within a range of 1 to 10 molar ratio(s), the phosphines is usually used within a range of 1 to 10 molar ratio(s), and the azodiester is usually used within a range of 1 to 10 molar ratio(s), as opposed to 1 mol of the compound (B5).

The reaction temperature is usually within a range of 0 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A6) .

The compound (M9) is a known compound, or can be prepared according to a known method.

### Process J

The compound (A6) can be prepared by reacting the compound (B5) with a compound represented by formula (M10) (hereinafter, referred to as “Compound (M10)”) in the presence of a base.

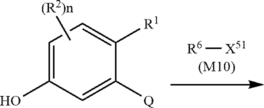

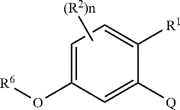

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixed solvents of these two or more solvents.

Examples of the bases include organic bases, alkali metal carbonates, alkali metal hydrocarbonates, sodium hydride, and tripotassium phosphate.

In the reaction, the compound (M10) is usually used within a range of 1 to 10 molar ratio (s), and the base is usually used within a range of 1 to 10 molar ratio(s), as opposed to 1 mol of the compound (B5).

The reaction temperature is usually within a range of -20 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A6) .

The compound (M10) is a known compound, or can be prepared according to a known method.

### Process K

A compound represented by formula (A7) (hereinafter, referred to as “Compound (A7)”) can be prepared by a step where a compound represented by formula (B6) (hereinafter, referred to as “Compound (B6)”) and a compound represented by formula (M11) (hereinafter, referred to as “Compound (M11)”) are reacted in the presence of a base to obtain a compound represented by (B7) (hereinafter, referred to as “Compound (B7)”) (hereinafter, referred to as Step (K-1)) and a step where the compound (B7) and a compound represented by formula (M12) (hereinafter, referred to as “Compound (M12)”) in the presence of a base (hereinafter, referred to as Step (K-2)).

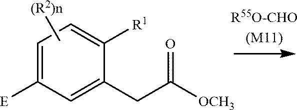

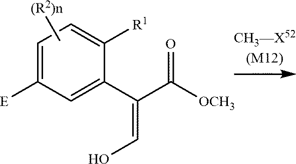

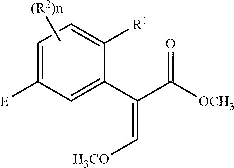

[wherein R⁵⁵ represents a C1-C4 alkyl group; X⁵² represents an iodine atom, a methoxysulfonyloxy group, a mesyloxy group, or a tosyl group; and the other symbols are the same defined above.]

A step (K-1) is usually carried out is a solvent. Examples of the solvent to be used in the reaction include ethers, amides, and mixed solvents of these two or more solvents.

Examples of the base to be used in the reaction include sodium hydride and alkali metal hydrides.

In the reaction, the compound (M11) is usually used within a range of 1 to 10 molar ratio (s), and the base is usually used within a range of 0.5 to 5 molar ratio(s), as opposed to 1 mol of the compound (B6).

The reaction period of the reaction is usually within a range of 5 minutes to 72 hours. The reaction temperature is usually within a range of -20 to 100° C.

When the reaction is completed, water is added to the reaction mixtures, and the resulting mixtures are extracted with organic solvent(s), and the organic layers are worked up (such as concentration and drying) to isolate the compound (B7) .

The compound (B6) and the compound (M11) are commercially available compounds, or can be prepared according to a known method.

A step (K-2) is usually carried out is a solvent. Examples of the solvent to be used in the reaction include hydrocarbons, ethers, halogenated hydrocarbons, amides, esters, nitriles and mixed solvents of these two or more solvents.

Examples of the base to be used in the reaction include organic bases, alkali metal carbonates, alkali metal hydrocarbonates, and sodium hydride.

In the reaction, the compound (M12) is usually used within a range of 1 to 10 molar ratio (s), and the base is usually used within a range of 1 to 20 molar ratio (s), as opposed to 1 mol of the compound (B7).

The reaction temperature is usually within a range of -20 to 100° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (A7) .

The compound (M12) is a known compound, or can be prepared according to a known method.

### Process L

A compound represented by formula (A8) (hereinafter, referred to as “Compound (A8)”) can be prepared by a step where a compound represented by formula (B8) (hereinafter, referred to as “Compound (B8)”) and a compound represented by formula (M13) (hereinafter, referred to as “Compound (M13)”) are reacted in the presence of a base to obtain a compound represented by formula (B9) (hereinafter, referred to as “Compound (B9)”) (hereinafter, referred to Step (L-1)) and a step where a compound (B9) and a compound (M12) are reacted in the presence of a base (hereinafter, referred to as Step (L-2)) in the presence of a base.

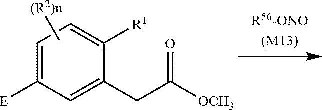

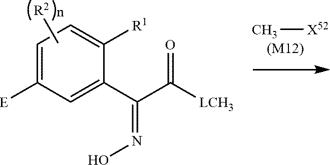

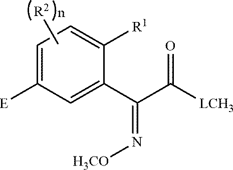

[wherein R⁵⁶ represents a t-butyl group, or an isopentyl group; and the other symbols are the same as defined above.]

A step (L-1) is usually carried out is a solvent. Examples of the solvent to be used in the reaction include ethers, amides, alcohols, and mixed solvents of these two or more solvents.

Examples of the base to be used in the reaction include sodium hydride and alkali metal alkoxides (such as sodium methoxide, sodium ethoxide, and potassium t-butoxide)

In the reaction, the compound (M13) is usually used within a range of 1 to 10 molar ratio (s), and the base is usually used within a range of 1 to 5 molar ratio (s), as opposed to 1 mol of the compound (B8).

The reaction period of the reaction is usually within a range of 5 minutes to 72 hours. The reaction temperature is usually within a range of -20 to 100° C.

When the reaction is completed, water is added to the reaction mixtures, and the resulting mixtures are extracted with organic solvent(s), and the organic layers are worked up (such as concentration and drying) to isolate the compound (B9).

The compound (B8) and the compound (M13) are commercially available compounds, or can be prepared according to a known method.

A step (L-2) can be carried out by using the compound (B9) in place of the compound (B7) according to the step (K-2) of the Process K.

### Process M

A compound represented by formula (A9) (hereinafter, referred to as “Compound (A9)”) can be prepared by reacting the compound (B1) with a compound represented by formula (M15) (hereinafter, referred to as “Compound (M15)”) in the presence of a palladium catalyst and a base.

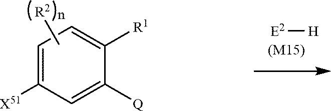

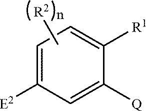

[wherein E² represents a C5-C6 cycloalkenyl group which may be optionally substituted with one or more substituents selected from Group D; and the other symbols are the same as defined above.]

The reaction is usually carried out in a solvent. Examples of the solvents to be used in the reaction include hydrocarbons, ethers, hydrocarbons, amides, water, and mixed solvents of these two or more solvents.

Examples of the catalyst to be used in the reaction include a palladium catalysts (such as tris(dibenzylideacetone)dipalladium (0), tetrakis(triphenylphosphine)palladium (0), palladium (II) acetate, and acetylacetone palladium (II).

Examples of the base to be used in the reaction include organic bases; alkali metal carbonates; and alkali metal hydrocarbonates.

A ligand and/or alkali metal halide may be used in the reaction as needed.

Examples of the ligand to be used in the reaction include tri-tert-butylphosphine tetrafluoroborate, triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethyletylenediamine, and N,N-dimethylglycine hydrochloride salt. When the ligand is used in the reaction, the ligand is usually used within a range of 0.01 to 1 molar ratio(s) as opposed to 1 mol of the compound (B1).

Examples of the alkali metal halides include potassium fluoride, sodium fluoride, lithium chloride, and sodium chloride. When the alkali metal halide is used in the reaction, the alkali metal halide is usually used within a range of 0.1 to 5 molar ratio(s), as opposed to 1 mol of the compound (B1).

In the reaction, the compound (M15) is usually used within a range of 1 to 10 molar ratio(s), the palladium catalyst is usually used within a range of 0.01 to 1 molar ratio(s), and the base is usually used within a range of 1 to 10 molar ratio (s), as opposed to 1 mol of the compound (B1).

The reaction temperature is usually within a range of 0 to 150° C. The reaction period of the reaction is usually within a range of 0.1 to 120 hours.

When the reaction is completed, water is added to reaction mixtures, and the reaction mixtures are extracted with organic solvent(s), and the organic layers are worked up (such as concentration and drying) to isolate the compound (A9) .

Also the compound (A9) can be prepared according to a method described in Chem. Commun., 2013, 49, 4794.

The compound (M15) is a known compound, or can be prepared according to a known method.

### Process N

A N-oxide of the compound represented by formula (I) can be prepared by reacting a compound represented by formula (I) and an oxidizing agent. The reaction can be carried out by a method described in U.S. Publication No. 2018/0009778 A1 or WO 2016/121970 A1.

### Reference Process 1

A compound represented by formula (B12) (hereinafter, referred to as “Compound (B12)”) can be prepared by a step where a compound represented by formula (B10) (hereinafter, referred to as “Compound (B10)”) and a compound represented by formula (M11) (hereinafter, referred to as “Compound (M11)”) are reacted in the presence of a base to obtain a compound represented by formula (B11) (hereinafter, referred to as “Compound (B11)”) (hereinafter, referred to as “Step (1-1)”), and a step where the compound (B11) and the compound (M12) are reacted in the presence of a base (hereinafter, referred to as “Step (1-2)”).

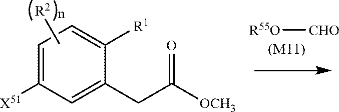

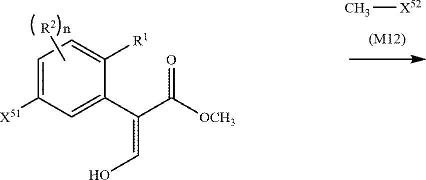

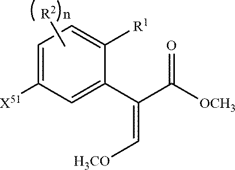

[wherein the symbols are the same as defined above.]

The step (1-1) can be carried out by using the compound (B10) in place of the compound (B6) according to the step (K-1) of the Process K.

The step (1-2) can be carried out by using the compound (B11) in place of the compound (B7) according to the step (K-2) of the Process K.

### Reference Process 2

A compound represented by formula (B15) (hereinafter, referred to as “Compound (B15)”) can be prepared by a step where a compound represented by formula (B13) (hereinafter, referred to as “Compound (B13)”) and the compound (M13) are reacted in the presence of a base to obtain a compound represented by formula (B14) (hereinafter, referred to as “Compound (B14)”) (hereinafter, referred to as “Step (2-1)”), and a step where the compound (B14) and the compound (M12) are reacted in the presence of a base (hereinafter, referred to as “Step (2-2)”).

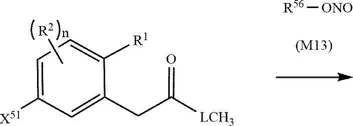

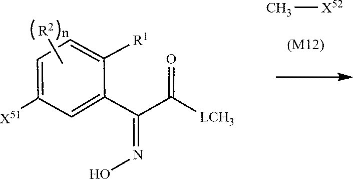

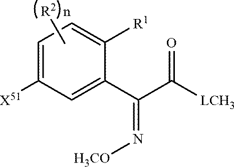

[wherein the symbols are the same as defined above.]

The step (2-1) can be carried out by using the compound (B13) in place of the compound (B8) according to the step (L-1) of the process L.

The step (2-2) can be carried out by using the compound (B14) in place of the compound (B7) according to the step (K-2) of the process K.

### Reference Process 3

A compound represented by formula (B16) (hereinafter, referred to as “Compound (B16)”) can be prepared reacting the compound (B1) with bis(pinacolato)diboron in the presence of a base and a palladium catalyst.

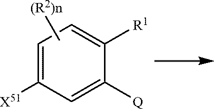

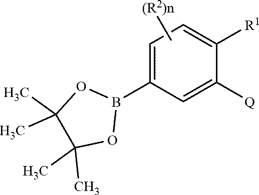

[wherein the symbols are the same defined above.]

The reaction is usually carried out in a solvent. Examples of the solvent to be used in the reaction include hydrocarbons, ethers, halogenated hydrocarbons, amides, esters, sulfoxides (such as dimethylsulfoxide (hereinafter, referred to as DMSO)) (hereinafter, referred to as sulfoxides), nitriles and mixed solvents of these two or more solvents.

Examples of the base to be used in the reaction include organic bases, alkali metal carbonates, alkali metal hydrocarbonates and tripotassium phosphate.

Examples of the palladium catalyst include [1,1′-bis(diphenylphosphino)ferrocene]palladium (II) dichloride.

In the reaction, bis(pinacolato)diboron is usually used within a range of 1 to 5 molar ratio(s), the base is usually used within a range of 1 to 5 molar ratio (s), and the palladium catalyst is usually used within a range of 0.01 to 0.5 molar ratios, as opposed to 1 mol of the compound (B1).

The reaction temperature is usually within a range of 0 to 150° C. The reaction period of the reaction is usually within a range of 0.1 to 48 hours.

When the reaction is completed, water is added to the reaction mixtures, and the resulting mixtures are extracted with organic solvent(s), and the organic layers are worked up (such as concentration and drying) to isolate the compound (B16) .

The compound (B5) can be prepared by oxidizing the compound (B16).

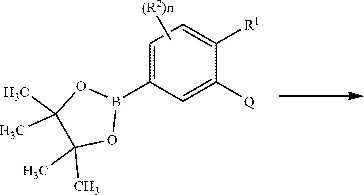

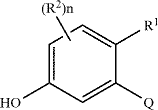

[wherein the symbols are the same as defined above.]

The reaction is usually carried out in a solvent. Examples of the solvent to be used in the reaction include hydrocarbons, ethers, halogenated hydrocarbons, amides, esters, nitriles, alcohols, water, and mixed solvents of these two or more solvents.

Examples of the oxidizing agent to be used in the reaction include metha chloroperbenzoic acid, and aqueous hydrogen peroxide solution.

When the aqueous hydrogen peroxide solution is used as an oxidizing agent, a base may be added as needed.

Examples of the base include alkali metal hydroxides.

In the reaction, the oxidizing agent is usually used within a range of 1 to 5 molar ratio(s) as opposed to 1 mol of the compound (B16).

When a base is used in the reaction, the base is usually used within a range of 0.1 to 5 molar ratios as opposed to 1 mol of the compound (B16).

The reaction temperature is usually within a range of -20 to 120° C. The reaction period of the reaction is usually within a range of 0.1 to 48 hours.

When the reaction is completed, water and a reducing agent (such as sodium thiosulfate) are added to the reaction mixture, and the resulting mixture was extracted with organic solvent(s), and the organic layers are worked up (for example, drying and concentration) to isolate the compound (B5).

### Reference Process 5

A compound represented by formula (B17) (hereinafter, referred to as “Compound (B17)”) can be prepared by reacting the compound (B1) with a compound represented by formula (M14) (hereinafter, referred to as “Compound (M14)”).

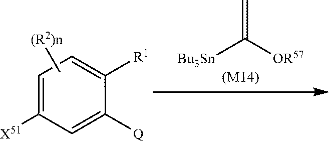

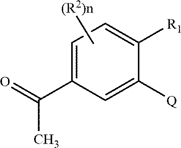

[wherein R⁵⁷ represents a methyl group, or ethyl group, and the other symbols are the same as defined above.]

The reaction can be carried out according to the method described in, for example, WO 2016/123253 A1.

### Reference Process 6

The compound (B4) can be prepared by reacting the compound (B3) with hydroxylamine or salts thereof.

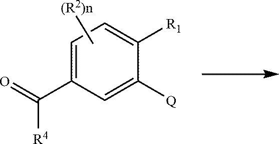

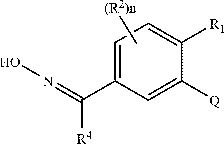

[wherein the symbols are the same as defined above.]

Examples of the salts of hydroxylamine include hydrochloride salts and sulfates.

The reaction can be carried out by using hydroxylamine in place of the compound (M4) according to the process D.

The compound of the present invention may be mixed or combined with one or more ingredients selected from a group consisting of the following Group (a), Group (b), Group (c), and Group (d), (hereinafter, referred to as “Present ingredient”).

The above-mentioned mixing or combining represents a use of the compound of the present invention and the present ingredient at same time, separately or at certain intervals.

When the compound of the present invention and the present ingredient are used at the same time, the compound of the present invention and the present ingredient may be contained in separate formulations respectively, or may be contained in the same one formulation.

One aspect of the present invention is a composition comprising one or more ingredients selected from Group (a), Group (b), Group (c) and Group (d) as well as the compound of the present invention (hereinafter, referred to as Composition A).

Group (a) is a group consisting of
each active ingredient as Acetylcholinesterase inhibitors (for example, carbamate insecticides, or organophosphorus insecticides), GABA-gated chloride channel blockers (for example, phenylpyrazole insecticides), Sodium channel modulators (for example, pyrethroid insecticides), Nicotinic acetylcholine receptor competitive modulators (for example, neonicotinoid insecticides), Nicotinic acetylcholine receptor allosteric modulators, Glutamatergic chlorine ion channel allosteric modulators (for example, macrolide insecticides), Juvenile hormone mimic, Multisite inhibitors, chordotonal organ TRPV channel modulators, Mites growth inhibitors, Mitochondria ATP biosynthetic enzyme inhibitors, Uncouplers of oxidative phosphorylation, Nicotinic acetylcholine receptor channel blocker (for example, Nereistoxin insecticides), Chitin synthesis inhibitors, Molting inhibitors, Ecdysone receptor agonist, Octopamine receptor agonist, Inhibitors of Mitochondrial electron transport system complex I, II, III and IV, Voltage-dependent sodium channel blockers, Acetyl CoA carboxylase inhibitor, Ryanodine receptor modulator (for example, Diamide insecticides), Chordotonal organ modulators, Microbial pesticides; and
the other insecticidal, miticidal or nematicidal active ingredients.

These ingredients are classified as a class based on the action mechanism of IRAC.

Group (b) is a group consisting of
Nucleic acid synthesis inhibitors (for example, Phenylamide fungicides, or Acylamino acid fungicides), cell division and cytoskeleton inhibitors (for example, MBC fungicides),
Respiratory inhibitors (for example, QoI fungicides or Qil fungicides), Amino acid synthesis and protein synthesis inhibitors (for example, anilinopyridine fungicides), Signal transduction inhibitors, Lipid synthesis and membrane synthesis inhibitors, sterol biosynthesis inhibitors (for example, DMI fungicides such as triazole), cell wall synthesis inhibitors, Melanin synthesis inhibitors, Plant defense inducers, Other action point contact active fungicides, Microbial fungicides, and the other fungicidal ingredients. These are classified as a class based on the action mechanism of FRAC.

Group (c) is a plant growth modulating ingredient group (including Mycorrhizal fungi, and Root nodule bacteria).

Group (d) is a repellent ingredient group.

Examples of the combination of the present ingredient and the compound of the present invention are described below. For example, alanycarb + SX represents a combination of alanycarb and SX. The symbol of “SX” represents any one of the compound of the present invention selected from the Compound Class SX1 to the Compound Class SX316. Also, all of the below-mentioned present active ingredient are known ingredients, and are commercially available or may be produced by the known method. If the present ingredient is a bacterium, it is available from the bacterial authority depository. The numerical number in bracket represents a CAS RN (Register Trademark).

Combinations of the present ingredient of the above Group (a) and the compound of the present invention:

abamectin + SX, acephate + SX, acequinocyl + SX, acetamiprid + SX, acetoprole + SX, acrinathrin + SX, acynonapyr + SX, afidopyropen + SX, afoxolaner + SX, alanycarb + SX, aldicarb + SX, allethrin + SX, alpha-cypermethrin + SX, alpha-endosulfan + SX, aluminium phosphide + SX, amitraz + SX, azadirachtin + SX, azamethiphos + SX, azinphos-ethyl + SX, azinphos-methyl + SX, azocyclotin + SX, bark of Celastrus angulatus + SX, bendiocarb + SX, benfluthrin + SX, benfuracarb + SX, bensultap + SX, benzoximate + SX, benzpyrimoxan + SX, beta-cyfluthrin + SX, beta-cypermethrin + SX, bifenazate + SX, bifenthrin + SX, bioallethrin + SX, bioresmethrin + SX, bistrifluron + SX, borax + SX, boric acid + SX, broflanilide + SX, bromopropylate + SX, buprofezin + SX, butocarboxim + SX, butoxycarboxim + SX, cadusafos + SX, calcium phosphide + SX, carbaryl + SX, carbofuran + SX, carbosulfan + SX, cartap hydrochloride + SX, cartap + SX, chinomethionat + SX, chlorantraniliprole + SX, chlordane + SX, chlorethoxyfos + SX, chlorfenapyr + SX, chlorfenvinphos + SX, chlorfluazuron + SX, chlormephos + SX, chloropicrin + SX, chlorpyrifos + SX, chlorpyrifos-methyl + SX, chromafenozide + SX, clofentezine + SX, clothianidin + SX, concanamycin A + SX, coumaphos + SX, cryolite + SX, cyanophos + SX, cyantraniliprole + SX, cyclaniliprole + SX, cyclobutrifluram + SX, cycloprothrin + SX, cycloxaprid + SX, cyenopyrafen + SX, cyetpyrafen + SX, cyflumetofen + SX, cyfluthrin + SX, cyhalodiamide + SX, cyhalothrin + SX, cyhexatin + SX, cypermethrin + SX, cyphenothrin + SX, cyromazine + SX, dazomet + SX, deltamethrin + SX, demeton-S-methyl + SX, diafenthiuron + SX, diazinon + SX, dichlorvos + SX, dicloromezotiaz + SX, dicofol + SX, dicrotophos + SX, diflovidazin + SX, diflubenzuron + SX, dimefluthrin + SX, dimethoate + SX, dimethylvinphos + SX, dimpropyridaz + SX, dinotefuran + SX, disodium octaborate + SX, disulfoton + SX, DNOC(2-methyl-4,6-dinitrophenol) + SX, doramectin + SX, dried leaves of Dryopteris filix-mas + SX, emamectin-benzoate + SX, empenthrin + SX, endosulfan + SX, EPN(O-ethyl O-(4-nitrophenyl) phenylphosphonothioate) + SX, epsilon-metofluthrin + SX, epsilon-momfluorothrin + SX, esfenvalerate + SX, ethiofencarb + SX, ethion + SX, ethiprole + SX, ethoprophos + SX, etofenprox + SX, etoxazole + SX, extract of Artemisia absinthium + SX, extract of Cassia nigricans + SX, extract of clitoria ternatea + SX, extract of Symphytum officinale + SX, extracts or simulated blend of Chenopodium ambrosioides + SX, extract of Tanacetum vulgare + SX, extract of Urtica dioica + SX, extract of Viscum album + SX, famphur + SX, fenamiphos + SX, fenazaquin + SX, fenbutatin oxide + SX, fenitrothion + SX, fenobucarb + SX, fenoxycarb + SX, fenpropathrin + SX, fenpyroximate + SX, fenthion + SX, fenvalerate + SX, fipronil + SX, flometoquin + SX, flonicamid + SX, fluacrypyrim + SX, fluazaindolizine + SX, fluazuron + SX, flubendiamide + SX, flucycloxuron + SX, flucythrinate + SX, fluensulfone + SX, flufenoprox + SX, flufenoxuron + SX, flufiprole + SX, flumethrin + SX, flupentiofenox + SX, flupyradifurone + SX, flupyrimin + SX, fluralaner + SX, fluvalinate + SX, fluxametamide + SX, formetanate + SX, fosthiazate + SX, furamethrin + SX, furathiocarb + SX, gamma-cyhalothrin + SX, GS-omega/kappa HXTX-Hv1a peptide + SX, halfenprox + SX, halofenozide + SX, heptafluthrin + SX, heptenophos + SX, hexaflumuron + SX, hexythiazox + SX, potassium salt of hop beta acid + SX, hydramethylnon + SX, hydroprene + SX, imicyafos + SX, imidacloprid + SX, imidaclothiz + SX, imiprothrin + SX, indoxacarb + SX, isocycloseram + SX, isofenphos + SX, isoprocarb + SX, isopropyl-O-(methoxyaminothiophosphoryl) salicylate + SX, isoxathion + SX, ivermectin + SX, kadethrin + SX, kappa-tefluthrin + SX, kappa-bifenthrin + SX, kinoprene + SX, lambda-cyhalothrin + SX, lenoremycin + SX, lepimectin + SX, lime sulfur + SX, lotilaner + SX, lufenuron + SX, machine oil + SX, malathion + SX, mecarbam + SX, meperfluthrin + SX, metaflumizone + SX, metam + SX, methamidophos + SX, methidathion + SX, methiocarb + SX, methomyl + SX, methoprene + SX, methoxychlor + SX, methoxyfenozide + SX, methyl bromide + SX, metofluthrin + SX, metolcarb + SX, metoxadiazone + SX, mevinphos + SX, milbemectin + SX, milbemycin oxime + SX, momfluorothrin + SX, monocrotophos + SX, moxidectin + SX, naled + SX, neem oil + SX, nicotine + SX, nicotine-sulfate + SX, nitenpyram + SX, novaluron + SX, noviflumuron + SX, oil of the seeds of Chenopodium anthelminticum + SX, omethoate + SX, oxamyl + SX, oxazosulfyl + SX, oxydemeton-methyl + SX, parathion + SX, parathion-methyl + SX, permethrin + SX, phenothrin + SX, phenthoate + SX, phorate + SX, phosalone + SX, phosmet + SX, phosphamidon + SX, phosphine + SX, phoxim + SX, pirimicarb + SX, pirimiphos-methyl + SX, prallethrin + SX, profenofos + SX, profluthrin + SX, propargite + SX, propetamphos + SX, propoxur + SX, propylene glycol alginate + SX, prothiofos + SX, pyflubumide + SX, pymetrozine + SX, pyraclofos + SX, pyrethrins + SX, pyridaben + SX, pyridalyl + SX, pyridaphenthion + SX, pyrifluquinazone + SX, pyrimidifen + SX, pyriminostrobin + SX, pyriprole + SX, pyriproxyfen + SX, quinalphos + SX, resmethrin + SX, rotenone + SX, ryanodine + SX, sarolaner + SX, selamectin + SX, sigma-cypermethrin + SX, silafluofen + SX, sodium borate + SX, sodium metaborate + SX, spinetoram + SX, spinosad + SX, spirodiclofen + SX, spiromesifen + SX, spiropidion + SX, spirotetramat + SX, sulfluramid + SX, sulfotep + SX, sulfoxaflor + SX, sulfur + SX, sulfuryl fluoride + SX, tartar emetic + SX, tau-fluvalinate + SX, tebufenozide + SX, tebufenpyrad + SX, tebupirimfos + SX, teflubenzuron + SX, tefluthrin + SX, temephos + SX, terbufos + SX, terpene constituents of the extract of chenopodium ambrosioides near ambrosioides + SX, tetrachlorantraniliprole + SX, tetrachlorvinphos + SX, tetradifon + SX, tetramethrin + SX, tetramethylfluthrin + SX, tetraniliprole + SX, theta-cypermethrin + SX, thiacloprid + SX, thiamethoxam + SX, thiocyclam + SX, thiodicarb + SX, thiofanox + SX, thiometon + SX, thiosultap-disodium + SX, thiosultap-monosodium + SX, tioxazafen + SX, tolfenpyrad + SX, tralomethrin + SX, transfluthrin + SX, triazamate + SX, triazophos + SX, trichlorfon + SX, triflumezopyrim + SX, triflumuron + SX, trimethacarb + SX, tyclopyrazoflor + SX, vamidothion + SX, wood extract of Quassia amara + SX, XMC (3,5-dimethylphenyl N-methylcarbamate) + SX, xylylcarb + SX, zeta-cypermethrin + SX, zinc phosphide + SX, 4-[5-(3,5-dichlorophenyl)-5-(trifluoromethyl)-4,5-dihydro-1,2-oxazol-3-yl]-2-methyl-N-(1-oxothietan-3-yl)benzamide (1241050-20-3) + SX, 3-methoxy-N-(5-{5-(trifluoromethyl)-5-[3-(trifluoromethyl)phenyl]-4,5-dihydro-1,2-oxazol-3-yl}indan-1-yl)propanamide (1118626-57-5) + SX, 4-chloro-5-[2,2-difluoro-2-(3,4,5-trifluorophenyl)ethoxy]-2-methylphenyl 2,2,2-trifluoroethyl sulfoxide (1632218-00-8) + SX, 4-fluoro-5-[2,2-difluoro-2-(3,4,5-trifluorophenyl)ethoxy]-2-methylphenyl 2,2,2-trifluoroethyl sulfoxide (1632217-98-1) + SX, 2-({2-fluoro-4-methyl-5-[(2,2,2-trifluoroethyl)sulfinyl]phenyl}imino)-3-(2,2,2-trifluoroethyl)-1,3-thiazolidin-4-one (1445683-71-5) + SX, (3R)-3-(2-chlorothiazol-5-yl)-8-methyl-7-oxo-6-phenyl-2,3-dihydrothiazolo[3,2-a]pyrimidin-4-ium-5-olate (2249718-27-0) + SX, BT crop protein CrylAb + SX, BT crop protein CrylAc + SX, BT crop protein CrylFa + SX, BT crop protein CrylA.105 + SX, BT crop protein Cry2Ab + SX, BT crop protein Vip3A + SX, BT crop protein Cry3A + SX, BT crop protein Cry3Ab + SX, BT crop protein Cry3Bb + SX, BT crop protein Cry34Ab1/Cry35Ab1 + SX, Adoxophyes orana granulosis virus strain BV-0001 + SX, Anticarsia gemmatalis mNPV + SX, Autographa californica mNPV + SX, Cydia pomonella GV strain V15 + SX, Cydia pomonella GV strain V22 + SX, Cryptophlebia leucotreta GV+ SX, Dendrolimus punctatus cypovirus + SX, Helicoverpa armigera NPV strain BV-0003 + SX, Helicoverpa zea NPV + SX, Lymantria dispar NPV + SX, Mamestra brassicae NPV + SX, Mamestra configurata NPV + SX, Neodiprion abietis NPV + SX, Neodiprion lecontei NPV + SX, Neodiprion sertifer NPV + SX, Nosema locustae + SX, Orgyia pseudotsugata NPV + SX, Pieris rapae GV + SX, Plodia interpunctella GV + SX, Spodoptera exigua mNPV + SX, Spodoptera littoralis mNPV + SX, Spodoptera litura NPV + SX, Arthrobotrys dactyloides + SX, Bacillus firmus strain GB-126 + SX, Bacillus firmus strain I-1582 + SX, Bacillus megaterium + SX, Bacillus sp. strain AQ175 + SX, Bacillus sp. strain AQ177 + SX, Bacillus sp. strain AQ178 + SX, Bacillus sphaericus strain 2362 + SX, Bacillus sphaericus strain ABTS1743 + SX, Bacillus sphaericus Serotype strain H5a5b + SX, Bacillus thuringiensis strain AQ52 + SX, Bacillus thuringiensis strain BD#32 + SX, Bacillus thuringiensisstrain CR-371 + SX, Bacillus thuringiensis subsp. Aizawai strain ABTS-1857 + SX, Bacillus thuringiensis subsp. Aizawai strain AM65-52 + SX, Bacillus thuringiensis subsp. Aizawai strain GC-91 + SX, Bacillus thuringiensis subsp. Aizawai Serotype strain H-7 + SX, Bacillus thuringiensis subsp. Kurstaki strain ABTS351 + SX, Bacillus thuringiensis subsp. Kurstaki strain BMP123 + SX, Bacillus thuringiensis subsp. Kurstaki strain EG234 + SX, Bacillus thuringiensis subsp. Kurstaki strain EG7841 + SX, Bacillus thuringiensis subsp. Kurstaki strain EVB113-19 + SX, Bacillus thuringiensis subsp. Kurstaki strain F810 + SX, Bacillus thuringiensis subsp. Kurstaki strain HD-1 + SX, Bacillus thuringiensis subsp. Kurstaki strain PB54 + SX, Bacillus thuringiensis subsp. Kurstaki strain SA-11 + SX, Bacillus thuringiensis subsp. Kurstaki strain SA-12 + SX, Bacillus thuringiensis subsp. Tenebriosis strain NB176 + SX, Bacillus thuringiensis subsp. Thuringiensis strain MPPL002 + SX, Bacillus thuringiensis subsp. morrisoni + SX, Bacillus thuringiensis var. colmeri + SX, Bacillus thuringiensis var. darmstadiensis strain 24-91 + SX, Bacillus thuringiensis var. dendrolimus + SX, Bacillus thuringiensis var. galleriae + SX, Bacillus thuringiensis var. israelensis strain BMP144 + SX, Bacillus thuringiensis var. israelensis serotype strain H-14 + SX, Bacillus thuringiensis var. japonensis strain buibui + SX, Bacillus thuringiensis var. san diego strain M-7 + SX, Bacillus thuringiensis var. 7216 + SX, Bacillus thuringiensis var. aegypti + SX, Bacillus thuringiensis var. T36 + SX, Beauveria bassiana strain ANT-03 + SX, Beauveria bassiana strain ATCC74040 + SX, Beauveria bassiana strain GHA + SX, Beauveria brongniartii + SX, Burkholderia rinojensis strain A396 + SX, Chromobacterium subtsugae strain PRAA4-1T + SX, Dactyllela ellipsospora + SX, Dectylaria thaumasia + SX, Hirsutella minnesotensis + SX, Hirsutella rhossiliensis + SX, Hirsutella thompsonii + SX, Lagenidium giganteum + SX, Lecanicillium lecanii strain KV01 + SX, Lecanicillium lecanii conidia of strain DAOM198499 + SX, Lecanicillium lecanii conidia of strain DAOM216596 + SX, Lecanicillium muscarium strain Ve6 + SX, Metarhizium anisopliae strain F52 + SX, Metarhizium anisopliae var. acridum + SX, Metarhizium anisopliae var. anisopliae BIPESCO 5/F52 + SX, Metarhizium flavoviride + SX, Monacrosporium phymatopagum + SX, Paecilomyces fumosoroseus Apopka strain 97 + SX, Paecilomyces lilacinus strain 251 + SX, Paecilomyces tenuipes strain T1 + SX, Paenibacillus popilliae + SX, Pasteuria nishizawae strain Pn1 + SX, Pasteuria penetrans + SX, Pasteuria usgae + SX, Pasteuria thoynei + SX, Serratia entomophila + SX, Verticillium chlamydosporium + SX, Verticillium lecani strain NCIM1312 + SX, 2-chloro-N-cyclopropyl-5-{1-[2,6-dichloro-4-(1,1,1,2,3,3,3-heptafluoropropan-2-yl)phenyl]-1H-pyrazol-4-yl}-N-methylpyridine-3-carboxamide (1771741-86-6) + SX, N-{4-chloro-3-[ (1-cyanocyclopropyl)carbamoyl]phenyl}-1-methyl-4-(methanesulfonyl)-3-(1,1,2,2,2-pentafluoroethyl)-1H-pyrazole-3-carboxamide (1400768-21-9) + SX, 11-(4-chloro-2,6-dimethylphenyl)-12-hydroxy-1,4-dioxa-9-azadispiro[4.2.4.2]tetradec-11-en-10-one (907187-07-9) + SX, 3-(4′-fluoro-2,4-dimethyl[1,1′-biphenyl]-3-yl)-4-hydroxy-8-oxa-1-azaspiro[4.5]dec-3-en-2-one (1031385-91-7) + SX, N-[3-chloro-1-(pyridin-3-yl)-1H-pyrazol-4-yl]-2-(methanesulfonyl)propanamide (2396747-83-2) + SX, 2-isopropyl-5-[(3,4,4-trifluoro-3-buten-1-yl)sulfonyl]-1,3,4-thiadiazole (2058052-95-0) + SX, 1,4-dimethyl-2-[2-(3-pyridinyl)-2H-indazol-5-yl]-1,2,4-triazolidine-3,5-dione (2171099-09-3) + SX, cyproflanilide + SX, N-((2-fluoro-4-[(2S,3S)-2-hydroxy-3-(3,4,5-trichlorophenyl)-3-(trifluoromethyl)pyrrolidin-1-yl]phenyl}methyl)cyclopropanecarboxamide + SX.

Combinations of the present ingredient of the above Group (b) and the compound of the present invention:

acibenzolar-S-methyl + SX, aldimorph + SX, ametoctradin + SX, aminopyrifen + SX, amisulbrom + SX, anilazine + SX, azaconazole + SX, azoxystrobin + SX, basic copper sulfate + SX, benalaxyl + SX, benalaxyl-M + SX, benodanil + SX, benomyl + SX, benthiavalicarb + SX, benthiavalicarb-isopropyl + SX, benzovindiflupyr + SX, binapacryl + SX, biphenyl + SX, bitertanol + SX, bixafen + SX, blasticidin-S + SX, Bordeaux mixture + SX, boscalid + SX, bromothalonil + SX, bromuconazole + SX, bupirimate + SX, captafol + SX, captan + SX, carbendazim + SX, carboxin + SX, carpropamid + SX, chinomethionat + SX, chitin + SX, chloroneb + SX, chlorothalonil + SX, chlozolinate + SX, colletochlorin B + SX, copper(II) acetate + SX, copper(II) hydroxide + SX, copper oxychloride + SX, copper(II) sulfate + SX, coumoxystrobin + SX, cyazofamid + SX, cyflufenamid + SX, cymoxanil + SX, cyproconazole + SX, cyprodinil + SX, dichlobentiazox + SX, dichlofluanid + SX, diclocymet + SX, diclomezine + SX, dicloran + SX, diethofencarb + SX, difenoconazole + SX, diflumetorim + SX, dimethachlone + SX, dimethirimol + SX, dimethomorph + SX, dimoxystrobin + SX, diniconazole + SX, diniconazole-M + SX, dinocap + SX, dipotassium hydrogenphosphite + SX, dipymetitrone + SX, dithianon + SX, dodecylbenzenesulphonic acid bisethylenediamine copper(II) salt + SX, dodemorph + SX, dodine + SX, edifenphos + SX, enoxastrobin + SX, epoxiconazole + SX, etaconazole + SX, ethaboxam + SX, ethirimol + SX, etridiazole + SX, extract from Melaleuca alternifolia + SX, extract from Reynoutria sachalinensis + SX, extract from the cotyledons of lupine plantlets (“BLAD”) + SX, extract of Allium sativum + SX, extract of Equisetum arvense + SX, extract of Tropaeolum majus + SX, famoxadone + SX, fenamidone + SX, fenaminstrobin + SX, fenarimol + SX, fenbuconazole + SX, fenfuram + SX, fenhexamid + SX, fenoxanil + SX, fenpiclonil + SX, fenpicoxamid + SX, fenpropidin + SX, fenpropimorph + SX, fenpyrazamine + SX, fentin acetate + SX, fentin chloride + SX, fentin hydroxide + SX, ferbam + SX, ferimzone + SX, florylpicoxamid + SX, fluazinam + SX, flubeneteram + SX, fludioxonil + SX, flufenoxystrobin + SX, fluindapyr + SX, flumorph + SX, fluopicolide + SX, fluopyram + SX, fluopimomide + SX, fluoroimide + SX, fluoxapiprolin + SX, fluoxastrobin + SX, fluquinconazole + SX, flusilazole + SX, flusulfamide + SX, flutianil + SX, flutolanil + SX, flutriafol + SX, fluxapyroxad + SX, folpet + SX, fosetyl + SX, fosetyl-aluminium + SX, fuberidazole + SX, furalaxyl + SX, furametpyr + SX, guazatine + SX, hexaconazole + SX, hymexazole + SX, imazalil + SX, imibenconazole + SX, iminoctadine + SX, iminoctadine triacetate + SX, inpyrfluxam + SX, iodocarb + SX, ipconazole + SX, ipfentrifluconazole + SX, ipflufenoquin + SX, iprobenfos + SX, iprodione + SX, iprovalicarb + SX, isofetamid + SX, isoflucypram + SX, isoprothiolane + SX, isopyrazam + SX, isotianil + SX, kasugamycin + SX, kresoxim-methyl + SX, laminarin + SX, leaves and bark of Quercus + SX, mancozeb + SX, mandestrobin + SX, mandipropamid + SX, maneb + SX, mefentrifluconazole + SX, mepanipyrim + SX, mepronil + SX, meptyldinocap + SX, metalaxyl + SX, metalaxyl-M + SX, metconazole + SX, methasulfocarb + SX, metiram + SX, metominostrobin + SX, metrafenone + SX, metyltetraprole + SX, mineral oils + SX, myclobutanil + SX, naftifine + SX, nuarimol + SX, octhilinone + SX, ofurace + SX, orysastrobin + SX, oxadixyl + SX, oxathiapiprolin + SX, oxine-copper + SX, oxolinic acid + SX, oxpoconazole + SX, oxpoconazole fumarate + SX, oxycarboxin + SX, oxytetracycline + SX, pefurazoate + SX, penconazole + SX, pencycuron + SX, penflufen + SX, penthiopyrad + SX, phenamacril + SX, phosphorous acid + SX, phthalide + SX, picarbutrazox + SX, picoxystrobin + SX, piperalin + SX, polyoxins + SX, potassium hydrogencarbonate + SX, potassium dihydrogenphosphite + SX, probenazole + SX, prochloraz + SX, procymidone + SX, propamidine + SX, propamocarb + SX, propiconazole + SX, propineb + SX, proquinazid + SX, prothiocarb + SX, prothioconazole + SX, pydiflumetofen + SX, pyraclostrobin + SX, pyrametostrobin + SX, pyraoxystrobin + SX, pyrapropoyne + SX, pyraziflumid + SX, pyrazophos + SX, pyribencarb + SX, pyributicarb + SX, pyridachlometyl + SX, pyrifenox + SX, pyrimethanil + SX, pyrimorph + SX, pyriofenone + SX, pyrisoxazole + SX, pyroquilon + SX, Quillaja extract + SX, quinconazole + SX, quinofumelin + SX, quinoxyfen + SX, quintozene + SX, Saponins of Chenopodium quinoa + SX, sedaxane + SX, silthiofam + SX, simeconazole + SX, sodium hydrogencarbonate + SX, spiroxamine + SX, streptomycin + SX, sulfur + SX, tebuconazole + SX, tebufloquin + SX, teclofthalam + SX, tecnazene + SX, terbinafine + SX, tetraconazole + SX, thiabendazole + SX, thifluzamide + SX, thiophanate + SX, thiophanate-methyl + SX, thiram + SX, thymol + SX, tiadinil + SX, tolclofos-methyl + SX, tolfenpyrad + SX, tolprocarb + SX, tolylfluanid + SX, triadimefon + SX, triadimenol + SX, triazoxide + SX, triclopyricarb + SX, tricyclazole + SX, tridemorph + SX, trifloxystrobin + SX, triflumizole + SX, triforine + SX, triticonazole + SX, validamycin + SX, valifenalate + SX, vinclozolin + SX, yellow mustard powder + SX, zinc thiazole + SX, zineb + SX, ziram + SX, zoxamide + SX, N′-[4-({3-[(4-chlorophenyl)methyl]-1,2,4-thiadiazol-5-yl}oxy)-2,5-dimethylphenyl]-N-ethyl-N-methylmethanimidamide (1202781-91-6) + SX, N′-{4-[(4,5-dichlorothiazol-2-yl)oxy]-2,5-dimethylphenyl}-N-ethyl-N-methylmethanimidamide (929908-57-6) + SX, N′-(2,5-dimethyl-4-phenoxyphenyl)-N-ethyl-N-methylmethanimidamide (1052688-31-9) + SX, N′-[5-choro-4-(2-fluorophenoxy)-2-methylphenyl)-N-ethyl-N-methylmethanimidamide (2055589-28-9) + SX, N′-[2-choro-4-(2-fluorophenoxy)-5-methylphenyl)-N-ethyl-N-methylmethanimidamide (2055756-21-1) + SX, N′-[4-(1-hydroxy-1-phenyl-2,2,2-trifluoroethyl)-2-methyl-5-methoxyphenyl)-N-isopropyl-N-methylmethanimidamide (2101814-55-3) + SX, N′-[5-bromo-6-(1-methyl-2-propoxyethoxy)-2-methylpyridin-3-yl)-N-ethyl-N-methylmethanimidamide (1817828-69-5) + SX, 4-(2-bromo-4-fluorophenyl)-N-(2-chloro-6-fluorophenyl)-1,3-dimethyl-1H-pyrazol-5-amine (1362477-26-6) + SX, 2-[6-(3-fluoro-4-methoxyphenyl)-5-methylpyridin-2-yl]quinazoline (1257056-97-5) + SX, 5-fluoro-4-imino-3-methyl-1-tosyl-3,4-dihydropyrimidin-2(1H)-one (1616664-98-2) + SX, ethyl (2Z)-3-amino-2-cyano-3-phenylacrylate (39491-78-6) + SX, N-[(2-chlorothiazol-5-yl)methyl]-N-ethyl-6-methoxy-3-nitropyridin-2-amine (1446247-98-8) + SX, 5-(4-chlorobenzyl)-2-(chloromethyl)-2-methyl-1-(1H-1,2,4-triazol-1-ylmethyl)cyclopentan-1-ol (1394057-11-4) + SX, (1R, 2S, 5S)-5-(4-chlorobenzyl)-2-(chloromethyl)-2-methyl-1-(1H-1,2,4-triazol-1-ylmethyl)cyclopentan-1-ol (1801930-06-2) + SX, (1S, 2R, 5R)-5-(4-chlorobenzyl)-2-(chloromethyl)-2-methyl-1-(1H-1,2,4-triazol-1-ylmethyl)cyclopentan-1-ol (1801930-07-3) + SX, 2-(chloromethyl)-5-(4-fluorobenzyl)-2-methyl-1-(1H-1,2,4-triazol-1-ylmethyl)cyclopentan-1-ol (1394057-13-6) + SX, (1R, 2S, 5S)-2-(chloromethyl)-5-(4-fluorobenzyl)-2-methyl-1-(1H-1,2,4-triazol-1-ylmethyl)cyclopentan-1-ol (1801930-08-4) + SX, (1S, 2R, 5R)-2-(chloromethyl)-5-(4-fluorobenzyl)-2-methyl-1-(1H-1,2,4-triazol-1-ylmethyl)cyclopentan-1-ol (1801930-09-5) + SX, methyl 3-[(4-chlorophenyl)methyl]-2-hydroxy-1-methyl-2-(1H-1,2,4-triazol-1-ylmethyl)cyclopentan-1-carboxylate (1791398-02-1) + SX, 1-(2,4-difluorophenyl)-2-(1H-1,2,4-triazol-1-yl)-1-[1-(4-bromo-2,6-difluorophenoxy)cyclopropyl]ethanol (2019215-86-0) + SX, 1-(2,4-difluorophenyl)-2-(1H-1,2,4-triazol-1-yl)-1-[1-(4-chloro-2,6-difluorophenoxy)cyclopropyl]ethanol (2019215-84-8) + SX, 1-[2-(1-chlorocyclopropyl)-3-(2-fluorophenyl)-2-hydroxypropyl]-1H-imidazole-5-carbonitrile (2018316-13-5) + SX, 1-[2-(1-chlorocyclopropyl)-3-(2,3-difluorophenyl)-2-hydroxypropyl]-1H-imidazole-5-carbonitrile (2018317-25-2) + SX, 4-({6-[2-(2,4-difluorophenyl)-1,1-difluoro-2-hydroxy-3-(1H-1,2,4-triazol-1-yl)propyl]pyridin-3-yl}oxy)benzonitrile (2046300-61-0) + SX, 2-[6-(4-bromophenoxy)-2-(trifluoromethyl)pyridin-3-yl]-1-(1H-1,2,4-triazol-1-yl)propan-2-ol (2082661-43-4) + SX, 2-[6-(4-chlorophenoxy)-2-(trifluoromethyl)pyridin-3-yl]-1-(1H-1,2,4-triazol-1-yl)propan-2-ol (2082660-27-1) + SX, methyl ({2-methyl-5-[1-(4-methoxy-2-methylphenyl)-1H-pyrazol-3-yl]phenyl}methyl)carbamate (1605879-98-8) + SX, 2-(difluoromethyl)-N-[1,1,3-trimethyl-2,3-dihydro-1H-inden-4-yl]pyridine-3-carboxamide (1616239-21-4) + SX, 2-(difluoromethyl)-N-[3-ethyl-1,1-dimethyl-2,3-dihydro-1H-inden-4-yl]pyridine-3-carboxamide (1847460-02-9) + SX, 2-(difluoromethyl)-N-[3-propyl-1,1-dimethyl-2,3-dihydro-1H-inden-4-yl]pyridine-3-carboxamide (1847460-05-2) + SX, (2E,3Z)-5-{ [1-(4-chlorophenyl)-1H-pyrazol-3-yl]oxy}-2-(methoxyimino)-N,3-dimethylpent-3-enamide (1445331-27-0) + SX, (2E, 3Z)-5-{[1-(2,4-dichlorophenyl)-1H-pyrazol-3-yl]oxy}-2-(methoxyimino)-N,3-dimethylpent-3-enamide (1445331-54-3) + SX, 5-chloro-4-({2-[6-(4-chlorophenoxy)pyridin-3-yl]ethyl}amino)-6-methylpyrimidine (1605340-92-8) + SX, N-(1-benzyl-1,3-dimethylbutyl)-8-fluoroquinoline-3-carboxamide (2132414-04-9) + SX, N-(1-benzyl-3,3,3-trifluoro-1-methylpropyl)-8-fluoroquinoline-3-carboxamide (2132414-00-5) + SX, 4,4-dimethyl-2-({4-[5-(trifluoromethyl)-1,2,4-oxadiazol-3-yl]phenyl}methyl)isoxazolidin-3-one (2098918-25-1) + SX, 5,5-dimethyl-2-({4-[5-(trifluoromethyl)-1,2,4-oxadiazol-3-yl]phenyl}methyl)isoxazolidin-3-one (2098918-26-2) + SX, Agrobacterium radiobactor strain K1026 + SX, Agrobacterium radiobactor strain K84 + SX, Bacillus amyloliquefaciens (Aveo (trademark) EZ Nematicide) + SX, Bacillus amyloliquefaciens strain AT332 + SX, Bacillus amyloliquefaciens strain B3 + SX, Bacillus amyloliquefaciens strain D747 + SX, Bacillus amyloliquefaciens strain DB101 + SX, Bacillus amyloliquefaciens strain DB102 + SX, Bacillus amyloliquefaciens strain GB03 + SX, Bacillus amyloliquefaciens strain FZB24 + SX, Bacillus amyloliquefaciens strain FZB42 + SX, Bacillus amyloliquefaciens strain IN937a + SX, Bacillus amyloliquefaciens strain MBI600 + SX, Bacillus amyloliquefaciens strain QST713 + SX, Bacillus amyloliquefaciens isolate strain B246 + SX, Bacillus amyloliquefaciens strain F727 + SX, Bacillus amyloliquefaciens subsp. plantarum strain D747 + SX, Bacillus licheniformis strain HB-2 + SX, Bacillus licheniformis strain SB3086 + SX, Bacillus pumilus strain AQ717 + SX, Bacillus pumilus strain BUF-33 + SX, Bacillus pumilus strain GB34 + SX, Bacillus pumilus strain QST2808 + SX, Bacillus simplex strain CGF2856 + SX, Bacillus subtilis strain AQ153 + SX, Bacillus subtilis strain AQ743 + SX, Bacillus subtilis strain BU1814 + SX, Bacillus subtilis strain D747 + SX, Bacillus subtilis strain DB101 + SX, Bacillus subtilis strain FZB24 + SX, Bacillus subtilis strain GB03 + SX, Bacillus subtilis strain HAI0404 + SX, Bacillus subtilis strain IAB/BS03 + SX, Bacillus subtilis strain MBI600 + SX, Bacillus subtilis strain QST30002/AQ30002 + SX, Bacillus subtilis strain QST30004/AQ30004 + SX, Bacillus subtilis strain QST713 + SX, Bacillus subtilis strain QST714 + SX, Bacillus subtilis var. Amyloliquefaciens strain FZB24 + SX, Bacillus subtilis strain Y1336 + SX, Burkholderia cepacia + SX, Burkholderia cepacia type Wisconsin strain J82 + SX, Burkholderia cepacia type Wisconsin strain M54 + SX, Candida oleophila strain O + SX, Candida saitoana + SX, Chaetomium cupreum + SX, Clonostachys rosea + SX, Coniothyrium minitans strain CGMCC8325 + SX, Coniothyrium minitans strain CON/M/91-8 + SX, cryptococcus albidus + SX, Erwinia carotovora subsp. carotovora strain CGE234M403 + SX, Fusarium oxysporum strain Fo47 + SX, Gliocladium catenulatum strain J1446 + SX, Paenibacillus polymyxa strain AC-1 + SX, Paenibacillus polymyxa strain BS-0105 + SX, Pantoea agglomerans strain E325 + SX, Phlebiopsis gigantea strain VRA1992 + SX, Pseudomonas aureofaciens strain TX-1 + SX, Pseudomonas chlororaphis strain 63-28 + SX, Pseudomonas chlororaphis strain AFS009 + SX, Pseudomonas chlororaphis strain MA342 + SX, Pseudomonas fluorescens strain 1629RS + SX, Pseudomonas fluorescens strain A506 + SX, Pseudomonas fluorescens strain CL145A + SX, Pseudomonas fluorescens strain G7090 + SX, Pseudomonas sp. strain CAB-02 + SX, Pseudomonas syringae strain 742RS + SX, Pseudomonas syringae strain MA-4 + SX, Pseudozyma flocculosa strain PF-A22UL + SX, Pseudomonas rhodesiae strain HAI-0804 + SX, Pythium oligandrum strain DV74 + SX, Pythium oligandrum strain M1 + SX, Streptomyces griseoviridis strain K61 + SX, Streptomyces lydicus strain WYCD108US + SX, Streptomyces lydicus strain WYEC108 + SX, Talaromyces flavus strain SAY-Y-94-01 + SX, Talaromyces flavus strain V117b + SX, Trichoderma asperellum strain ICC012 + SX, Trichoderma asperellum SKT-1 + SX, Trichoderma asperellum strain T25 + SX, Trichoderma asperellum strain T34 + SX, Trichoderma asperellum strain TV1 + SX, Trichoderma atroviride strain CNCM 1-1237 + SX, Trichoderma atroviride strain LC52 + SX, Trichoderma atroviride strain IMI 206040 + SX, Trichoderma atroviride strain SC1 + SX, Trichoderma atroviride strain SKT-1 + SX, Trichoderma atroviride strain T11 + SX, y strain ICC080 + SX, Trichoderma harzianum strain 21 + SX, Trichoderma harzianum strain DB104 + SX, Trichoderma harzianum strain DSM 14944 + SX, Trichoderma harzianum strain ESALQ-1303 + SX, Trichoderma harzianum strain ESALQ-1306 + SX, Trichoderma harzianum strain IIHR-Th-2 + SX, Trichoderma harzianum strain ITEM908 + SX, Trichoderma harzianum strain kd + SX, Trichoderma harzianum strain MO1 + SX, Trichoderma harzianum strain SF + SX, Trichoderma harzianum strain T22 + SX, Trichoderma harzianum strain T39 + SX, Trichoderma harzianum strain T78 + SX, Trichoderma harzianum strain TH35 + SX, Trichoderma polysporum strain IMI206039 + SX, trichoderma stromaticum + SX, Trichoderma virens strain G-41 + SX, Trichoderma virens strain GL-21 + SX, Trichoderma viride + SX, Variovorax paradoxus strain CGF4526 + SX, Harpin protein + SX, N-acetyl-2-(ethanesulfonyl)-N-[2-(methoxycarbonyl)-4-(trifluoromethoxy)phenyl]-4-(trifluoromethyl)benzamide (2043675-28-9) + SX, (2S,3S)-3-(2-methylphenyl)butan-2-yl N-[(3-acetoxy-4-methoxypyridin-2-yl)carbonyl]-L-alaninate (2376210-00-1) + SX, (2S,3S)-3-(4-fluoro-2-methylphenyl)butan-2-yl N-[(3-acetoxy-4-methoxypyridin-2-yl)carbonyl]-L-alaninate + SX, (2S,3S)-3-(4-methoxy-2-methylphenyl)butan-2-yl N-[(3-acetoxy-4-methoxypyridin-2-yl)carbonyl]-L-alaninate + SX, (2S,3S)-3-(2,4-dimethylphenyl)butan-2-yl N-[(3-acetoxy-4-methoxypyridin-2-yl)carbonyl]-L-alaninate (2376209-13-9) + SX, (2S,3S)-3-(2-methylphenyl)butan-2-yl N-({3-[(2-methylpropanoyl)oxy]-4-methoxypyridin-2-yl}carbonyl)-L-alaninate (2376210-02-3) + SX, (2S,3S)-3-(4-fluoro-2-methylphenyl)butan-2-yl N-({3-[(2-methylpropanoyl)oxy]-4-methoxypyridin-2-yl}carbonyl)-L-alaninate + SX, (2S,3S)-3-(4-methoxy-2-methylphenyl)butan-2-yl N-({3-[(2-methylpropanoyl)oxy]-4-methoxypyridin-2-yl}carbonyl)-L-alaninate (2376209-40-2) + SX, (2S,3S)-3-(2,4-dimethylphenyl)butan-2-yl N-({3-[ (2-methylpropanoyl)oxy]-4-methoxypyridin-2-yl}carbonyl)-L-alaninate (2376209-15-1) + SX, N′-(2-choro-4-phenoxy-5-methylphenyl)-N-ethyl-N-methylmethanimidamide (2062599-39-5) + SX, (2S,3S)-3-(2-methylphenyl)butan-2-yl N-[(3-hydroxy-4-methoxypyridin-2-yl)carbonyl]-L-alaninate + SX, (2S,3S)-3-(4-fluoro-2-methylphenyl)butan-2-yl N-[(3-hydroxy-4-methoxypyridin-2-yl)carbonyl]-L-alaninate + SX, (2S,3S)-3-(4-methoxy-2-methylphenyl)butan-2-yl N-[(3-hydroxy-4-methoxypyridin-2-yl)carbonyl]-L-alaninate + SX, (2S,3S)-3-(2,4-dimethylphenyl)butan-2-yl N-[(3-hydroxy-4-methoxypyridin-2-yl)carbonyl]-L-alaninate + SX, (2S,3S)-3-(4-fluoro-2-methylphenyl)-4-methylpentan-2-yl N-[(3-acetoxy-4-methoxypyridin-2-yl)carbonyl]-L-alaninate + SX, (2S,3S)-3-(2-fluoro-4-methylphenyl)-4-methylpentan-2-yl N-[(3-acetoxy-4-methoxypyridin-2-yl)carbonyl]-L-alaninate + SX, (25,35)-3-(2,4-difluorophenyl)-4-methylpentan-2-yl N-[(3-acetoxy-4-methoxypyridin-2-yl)carbonyl]-L-alaninate + SX, (25,35)-3-(2,4-dimethylphenyl)-4-methylpentan-2-yl N-[(3-acetoxy-4-methoxypyridin-2-yl)carbonyl]-L-alaninate + SX, (2S,3S)-3-(4-fluoro-2-methoxylphenyl)-4-methylpentan-2-yl N-[(3-acetoxy-4-methoxypyridin-2-yl)carbonyl]-L-alaninate + SX, (2S,3S)-3-(4-fluoro-2-methylphenyl)-4-methylpentan-2-yl N-{ [3-(acetoxymethoxy)-4-methoxypyridin-2-yl]carbonyl}-L-alaninate + SX, (2S,3S)-3-(2-fluoro-4-methylphenyl)-4-methylpentan-2-yl N-{[3-(acetoxymethoxy)-4-methoxypyridin-2-yl]carbonyl}-L-alaninate + SX, (2S,3S)-3-(2,4-difluorophenyl)-4-methylpentan-2-yl N-{[3-(acetoxymethoxy)-4-methoxypyridin-2-yl]carbonyl}-L-alaninate + SX, (2S,3S)-3-(2,4-dimethylphenyl)-4-methylpentan-2-yl N-{[3-(acetoxymethoxy)-4-methoxypyridin-2-yl]carbonyl}-L-alaninate + SX, (2S,3S)-3-(4-fluoro-2-methoxylphenyl)-4-methylpentan-2-yl N-{[3-(acetoxymethoxy)-4-methoxypyridin-2-yl]carbonyl}-L-alaninate + SX, (2S,3S)-3-(4-fluoro-2-methylphenyl)-4-methylpentan-2-yl N-({3-[(isobutyryloxy)methoxy]-4-methoxypyridin-2-yl}carbonyl)-L-alaninate + SX, (2S,3S)-3-(2-fluoro-4-methylphenyl)-4-methylpentan-2-yl N-({3-[(isobutyryloxy)methoxy]-4-methoxypyridin-2-yl}carbonyl)-L-alaninate + SX, (2S,3S)-3-(2,4-difluorophenyl)-4-methylpentan-2-yl N-({3-[(isobutyryloxy)methoxy]-4-methoxypyridin-2-yl}carbonyl)-L-alaninate + SX, (2S,3S)-3-(2,4-dimethylphenyl)-4-methylpentan-2-yl N-({3-[(isobutyryloxy)methoxy]-4-methoxypyridin-2-yl}carbonyl)-L-alaninate + SX, (2S,3S)-3-(4-fluoro-2-methoxylphenyl)-4-methylpentan-2-yl N-({3-[(isobutyryloxy)methoxy]-4-methoxypyridin-2-yl}carbonyl)-L-alaninate + SX, (2S,3S)-3-(4-fluoro-2-methylphenyl)-4-methylpentan-2-yl N-[(3-hydroxy-4-methoxypyridin-2-yl)carbonyl]-L-alaninate + SX, (2S,3S)-3-(2-fluoro-4-methylphenyl)-4-methylpentan-2-yl N-[(3-hydroxy-4-methoxypyridin-2-yl)carbonyl]-L-alaninate + SX, (2S,3S)-3-(2,4-difluorophenyl)-4-methylpentan-2-yl N-[(3-hydroxy-4-methoxypyridin-2-yl)carbonyl]-L-alaninate + SX, (2S,3S)-3-(2,4-dimethylphenyl)-4-methylpentan-2-yl N-[(3-hydroxy-4-methoxypyridin-2-yl)carbonyl]-L-alaninate + SX, (2S,3S)-3-(4-fluoro-2-methoxylphenyl)-4-methylpentan-2-yl N-[(3-hydroxy-4-methoxypyridin-2-yl)carbonyl]-L-alaninate + SX, 3-(4-bromo-7-fluoroindol-1-yl)butan-2-yl N-[(3-hydroxy-4-methoxypyridin-2-yl)carbonyl]-L-alaninate + SX, 3-(7-bromoindol-1-yl)butan-2-yl N-[(3-hydroxy-4-methoxypyridin-2-yl)carbonyl]-L-alaninate + SX, 3-(7-bromo-4-fluoroindol-1-yl)butan-2-yl N-[(3-hydroxy-4-methoxypyridin-2-yl)carbonyl]-L-alaninate + SX, 3-(3,5-dichloropyridin-2-yl)butan-2-yl N-[(3-hydroxy-4-methoxypyridin-2-yl)carbonyl]-L-alaninate + SX, 3-(3,5-dichloropyridin-2-yl)butan-2-yl N-{[3-(acetoxymethoxy)-4-methoxypyridin-2-yl]carbonyl}-L-alaninate + SX, (1S)-1-[1-(naphthalen-1-yl)cyclopropyl]ethyl N-[(3-hydroxy-4-methoxypyridin-2-yl)carbonyl]-L-alaninate + SX, (1S)-1-[1-(naphthalen-1-yl)cyclopropyl]ethyl N-[(3-acetoxy-4-methoxypyridin-2-yl)carbonyl]-L-alaninate + SX, (1S)-1-[1-(naphthalen-1-yl)cyclopropyl]ethyl N-{[3-(acetoxymethoxy)-4-methoxypyridin-2-yl)carbonyl]-L-alaninate + SX, chloroinconazide + SX, N-ethyl-2-methyl-N-({4-[5-(trifluoromethyl)-1,2,4-oxadiazol-3-yl]phenyl}methyl)propanamide + SX, N,2-dimethoxy-N-({4-[5-(trifluoromethyl)-1,2,4-oxadiazol-3-yl]phenyl}methyl)propanamide + SX, N-methoxy-N-({4-[5-(trifluoromethyl)-1,2,4-oxadiazol-3-yl]phenyl}methyl)cyclopropanecarboxamide + SX, N-methoxy-N′-methyl-N-({4-[5-(trifluoromethyl)-1,2,4-oxadiazol-3-yl]phenyl}methyl)urea + SX, N′-ethyl-N-methoxy-N-({4-[5-(trifluoromethyl)-1,2,4-oxadiazol-3-yl]phenyl}methyl)urea + SX, N,N′-dimethoxy-N-({4-[5-(trifluoromethyl)-1,2,4-oxadiazol-3-yl]phenyl}methyl)urea + SX, N-(2-fluorophenyl)-4-[5-(trifluoromethyl)-1,2,4-oxadiazol-3-yl]benzamide + SX, N-[2-(difluoromethoxy)phenyl]-4-[5-(trifluoromethyl)-1,2,4-oxadiazol-3-yl]benzamide + SX, N-[2-(difluoromethoxy)-4-fluorophenyl]-4-[5-(trifluoromethyl)-1,2,4-oxadiazol-3-yl]benzamide + SX.

Combinations of the present ingredient of the above Group (c) and the compound of the present invention:

1-methylcyclopropene + SX, 1,3-diphenylurea + SX, 2,3,5-triiodobenzoic acid + SX, IAA ((1H-indol-3-yl)acetic acid) + SX, IBA (4-(1H-indol-3-yl)butyric acid) + SX, MCPA (2-(4-chloro-2-methylphenoxy)acetic acid) + SX, MCPB (4-(4-chloro-2-methylphenoxy)butyric acid) + SX, 4-CPA (4-chlorophenoxyacetic acid) + SX, 5-aminolevulinic acid hydrochloride + SX, 6-benzylaminopurine + SX, abscisic acid + SX, AVG (aminoethoxyvinylglycine) + SX, ancymidol + SX, butralin + SX, calcium carbonate + SX, calcium chloride + SX, calcium formate + SX, calcium peroxide + SX, calcium polysulfide + SX, calcium sulfate + SX, chlormequat-chloride + SX, chlorpropham + SX, choline chloride + SX, cloprop + SX, cyanamide + SX, cyclanilide + SX, daminozide + SX, decan-1-ol + SX, dichlorprop + SX, dikegulac + SX, dimethipin + SX, diquat + SX, ethephon + SX, ethychlozate + SX, flumetralin + SX, flurprimidol + SX, forchlorfenuron + SX, formononetin + SX, Gibberellin A + SX, Gibberellin A3 + SX, inabenfide + SX, Kinetin + SX, lipochitooligosaccharide SP104 + SX, maleic hydrazide + SX, mefluidide + SX, mepiquat-chloride + SX, oxidized glutathione + SX, pacrobutrazol + SX, pendimethalin + SX, prohexandione-calcium + SX, prohydrojasmon + SX, pyraflufen-ethyl + SX, sintofen + SX, sodium 1-naphthaleneacetate + SX, sodium cyanate + SX, streptmycin + SX, thidiazuron + SX, triapenthenol + SX, Tribufos + SX, trinexapac-ethyl + SX, uniconazole-P + SX, 2-(naphthalen-1-yl)acetamide + SX, [4-oxo-4-(2-phenylethyl)amino]butyric acid + SX, methyl 5-(trifluoromethyl)-benzo[b]thiophen-2-carboxylate + SX, 3-[(6-chloro-4-phenylquinazolin-2-yl)amino]propan-1-ol + SX, Claroideoglomus etunicatum + SX, Claroideoglomus claroideum + SX, Funneliformis mosseae + SX, Gigaspora margarita + SX, Gigaspora rosea + SX, Glomus aggregatum + SX, Glomus deserticola + SX, Glomus monosporum + SX, Paraglomus brasillianum + SX, Rhizophagus clarus + SX, Rhizophagus intraradices RTI-801 + SX, Rhizophagus irregularis DAOM 197198 + SX, Azorhizobium caulinodans + SX, Azospirillum amazonense + SX, Azospirillum brasilense XOH + SX,Azospirillum brasilense Ab-V5 + SX, Azospirillum brasilense Ab-V6 + SX, Azospirillum caulinodans + SX, Azospirillum halopraeferens + SX, Azospirillum irakense + SX, Azospirillum lipoferum + SX, Bradyrhizobium elkanii SEMIA 587 + SX, Bradyrhizobium elkanii SEMIA 5019 + SX, Bradyrhizobium japonicum TA-11 + SX, Bradyrhizobium japonicum USDA 110 + SX, Bradyrhizobium liaoningense + SX, Bradyrhizobium lupini + SX, Delftia acidovorans RAY209 + SX, Mesorhizobium ciceri + SX, Mesorhizobium huakii + SX, Mesorhizobium loti + SX, Rhizobium etli + SX, Rhizobium galegae + SX, Rhizobium leguminosarum bv. Phaseoli + SX, Rhizobium leguminosarum bv. Trifolii + SX, Rhizobium leguminosarum bv. Viciae + SX, Rhizobium trifolii + SX, Rhizobium tropici + SX, Sinorhizobium fredii + SX, Sinorhizobium meliloti + SX, Zucchini Yellow Mosaik Virus weak strain + SX.

Combinations of the present ingredient of the above Group (d) and the compound of the present invention: anthraquinone + SX, deet + SX, icaridin + SX.

The ratio of the compound of the present invention to the Present ingredient includes, but not limited thereto, as a ratio by weight (the compound of the present invention : the present ingredient) 1,000:1 to 1:1,000, 500:1 to 1:500, 100:1 to 1:100, 50:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10. 1:20, and 1:50, and the others.

The compound of the present invention has control efficacy against pests. Examples of the pests include phytopathogenic microorganism, harmful arthropods (such as harmful insects and harmful mites), harmful nematodes, and harmful mollusks.

The compound of the present invention can control plant diseases which are caused by phytopathogenic microorganism such as fungi, Oomycete, Phytomyxea, and bacteria and the others. Examples of the fungi include Ascomycota, Basidiomycota, Blasocladiomycota, Chytridiomycota, Mucoromycota, and Olpidiomycota. Specific examples thereof include the followings. The descriptions in a parenthesis indicates an academic name of phytopathogenic microorganism that causes each of the disease.

Rice diseases:
blast (Pyricularia oryzae), brown spot (Cocnliobolus miyabeanus), sheath blight (Rhizoctonia solani), bakanae disease (Gibberella fujikuroi), downy mildew (Sclerophthora macrospora), false blast and head blight (Epicoccum nigrum), and seedling blight (Trichoderma viride, Rhizopus oryzae);
Wheat diseases:
powdery mildew (Blumeria graminis), fusarium blight (Fusarium graminearum, Fusarium avenaceum, Fusarium culmorum, Microdochium nivale), stripe rust (Puccinia striiformis), stem rust (Puccinia graminis), leaf rust (Puccinia recondita), snow mould (Microdochium nivale, Microdochium majus), typhula snow blight (Typhula incarnata, Typhula ishikariensis), loose smut (Ustilago tritici), stinking smut (Tilletia caries, Tilletia controversa), eyespot (Pseudocercosporella herpotrichoides), leaf blotch (Septoria tritici), glume blotch (Stagonospora nodorum), tan spot (Pyrenophora tritici-repentis), rhizoctonia seeding blight (Rhizoctonia solani), take-all disease (Gaeumannomyces graminis), and blast (Pyricularia graminis-tritici);
Barley diseases:
powdery mildew (Blumeria graminis), fusarium head blight (Fusarium graminearum, Fusarium avenaceum, Fusarium culmorum, Microdochium nivale), stripe rust (Puccinia striiformis), stem rust (Puccinia graminis), dwarf leaf rust (Puccinia hordei), loose smut (Ustilago nuda), scald (Rhynchosporium secalis), net blotch (Pyrenophora teres), spot blotch (Cochliobolus sativus), stripe (Pyrenophora graminea), Ramularia disease (Ramularia collo-cygni), and rhizoctonia seeding blight (Rhizoctonia solani);
Corn diseases:
rust (Puccinia sorghi), southern rust (Puccinia polysora), northern leaf blight (Setosphaeria turcica), tropical rust (Physopella zeae), southern leaf blight (Cochliobolus heterostrophus), anthracnose (Colletotrichum graminicola), gray leaf spot (Cercospora zeae-maydis), eyespot (Kabatiella zeae), phaeosphaeria leaf spot (Phaeosphaeria maydis), diplodia rot (Stenocarpella maydis, Stenocarpella macrospora) ,
stalk rot (Fusarium graminearum, Fusarium verticilioides, Colletotrichum graminicola),
smut (Ustilago maydis), and Physoderma brown spot and Physoderma stalk rot (Physoderma maydis);
Cotton diseases:
anthracnose (Colletotrichum gossypii), grey mildew (Ramularia areola), alternaria leaf spot (Alternaria macrospora, Alternaria gossypii), and black root rot (Thielaviopsis basicola);
Coffee diseases:
rust (Hemileia vastatrix), and leaf spot (Cercospora coffeicola);
Rape seed diseases:
sclerotinia rot (Sclerotinia sclerotiorum), gray leaf spot (Alternaria brassicae), root rot (Phoma lingam), and light leaf spot (Pyrenopeziza brassicae);
Sugar cane diseases:
rust (Puccinia melanocephela, Puccinia kuehnii), and smut (Ustilago scitaminea);
Sunflower diseases:
rust (Puccinia helianthi), and downy mildew (Plasmopara halstedii);
Citrus diseases:
melanose (Diaporthe citri), scab (Elsinoe fawcetti), green mold (Penicillium digitatum), blue mold (Penicillium italicum), Phytophthora rot (Phytophthora parasitica, Phytophthora citrophthora), and aspergillus rot (Aspergillus niger);
Apple diseases:
blossom blight (Monilinia mali), valsa canker (Valsa ceratosperma), powdery mildew (Podosphaera leucotricha), alternaria leaf spot (Alternaria alternata apple pathotype), scab (Venturia inaequalis), bitter rot (Glomerella cingulata, Colletotrichum acutatum), blotch (Diplocarpon mali), ring rot (Botryosphaeria berengeriana), crown rot (Phytophtora cactorum), and rust (Gymnosporangium juniperi-virginianae, Gymnosporangium yamadae) ;
Pear diseases:
scab (Venturia nashicola, Venturia pirina), black spot (Alternaria alternata Japanese pear pathotype), and rust (Gymnosporangium haraeanum);
Peach diseases:
brown rot (Monilinia fructicola), scab (Cladosporium carpophilum), Phomopsis rot (Phomopsis sp.), and leaf curl (Taphrina deformans);
Grapes diseases:
anthracnose (Elsinoe ampelina), ripe rot (Glomerella cingulata, Colletotrichum acutatum), powdery mildew (Uncinula necator), rust (Phakopsora ampelopsidis), black rot (Guignardia bidwellii), and downy mildew (Plasmopara viticola);
Japanese persimmon diseases:
anthracnose (Gloeosporium kaki, Colletotrichum acutatum), and leaf spot (Cercospora kaki, Mycosphaerella nawae);
Fig diseases:
rust (Phakopsora nishidana));
Diseases of gourd family:
anthracnose (Colletotrichum lagenarium), powdery mildew (Sphaerotheca fuliginea), gummy stem blight (Didymella bryoniae), Corynespora leaf spot (Corynespora cassiicola), fusarium wilt (Fusarium oxysporum), downy mildew (Pseudoperonospora cubensis), phytophthora rot (Phytophthora capsici), and damping-off (Pythium sp.);
Tomato diseases:
early blight (Alternaria solani), leaf mold (Cladosporium fulvum), Cercospora leaf mold (Pseudocercospora fuligena), late blight (Phytophthora infestans), and powdery mildew (Leveillula taurica);
Eggplant diseases:
brown spot (Phomopsis vexans), and powdery mildew (Erysiphe cichoracearum);
Cruciferous vegetables diseases:
alternaria leaf spot (Alternaria japonica), white spot (Cercosporella brassicae), clubroot (Plasmodiophora brassicae), downy mildew (Peronospora parasitica), and white rust (Albugo candida);
Welsh onion disease:
rust (Puccinia allii);
Soybean diseases:
purple stain (Cercospora kikuchii), sphaceloma scab (Elsinoe glycines), pod and stem blight (Diaporthe phaseolorum var. sojae), rust (Phakopsora pachyrhizi), target spot (Corynespora cassiicola), anthracnose (Colletotrichum glycines, Colletotrichum truncatum), Rhizoctonia rot (Rhizoctonia solani), septoria brown spot (Septoria glycines), Cercospora leaf spot (Cercospora sojina), stem rot (Sclerotinia sclerotiorum), powdery mildew (Microsphaera diffusa), phytophthora stem and root rot (Phytophthora sojae), downy mildew (Peronospora manshurica), sudden death syndrome (Fusarium virguliforme), red crown rot (Calonectria ilicicola), and Diaporthe/Phomopsis complex (Diaporthe longicolla) ;
Kidney bean diseases:
stem rot (Sclerotinia sclerotiorum), rust (Uromyces appendiculatus), angular leaf spot (Phaeoisariopsis griseola), and anthracnose (Colletotrichum lindemuthianum), and Fusarium root-rot (Fusarium solani);
Peanut diseases:
leaf spot (Cercospora personata), brown leaf spot (Cercospora arachidicola), southern blight (Sclerotium rolfsii), and Cylindrocladium black rot (Calonectria ilicicola) ;
Garden pea disease:
powdery mildew (Erysiphe pisi), and root rot (Fusarium solani);
Potato diseases:
early blight (Alternaria solani), late blight (Phytophthora infestans), Pink rot (Phytophthora erythroseptica), powdery scab (Spongospora subterranea f. sp. subterranea), verticillium wilt (Verticillium albo-atrum, Verticillium dahliae,Verticillium nigrescens), dry rot (Fusarium solani), and potato wart (Synchytrium endobioticum);
Strawberry disease:
powdery mildew (Sphaerotheca humuli);
Tea diseases:
net blister blight (Exobasidium reticulatum), white scab (Elsinoe leucospila), gray blight (Pestalotiopsis sp.), and anthracnose (Colletotrichum theae-sinensis) ;
Tobacco diseases:
brown spot (Alternaria longipes), anthracnose (Colletotrichum tabacum), blue mold (Peronospora tabacina), and black shank (Phytophthora nicotianae);
Sugar beet diseases:
cercospora leaf spot (Cercospora beticola), leaf blight (Thanatephorus cucumeris), root rot (Thanatephorus cucumeris), and aphanomyces root rot (Aphanomyces cochlioides), and rust (Uromyces betae);
Rose diseases:
black spot (Diplocarpon rosae), and powdery mildew (Sphaerotheca pannosa);
Chrysanthemum diseases:
leaf blight (Septoria chrysanthemi-indici) , and white rust (Puccinia horiana);
Onion diseases:
botrytis leaf blight (Botrytis cinerea, Botrytis byssoidea, Botrytis squamosa), gray-mold neck rot (Botrytis allii), and small sclerotial neck rot (Botrytis squamosa);
Various crops diseases:
Botrytis rot (Botrytis cinerea), sclerotinia rot (Sclerotinia sclerotiorum), seedling blight (Pythium aphanidermatum, Pythium irregulare, Pythium ultimum) ;
Japanese radish disease:
alternaria leaf spot (Alternaria brassicicola);
Turfgrass diseases:
dollar spot (Sclerotinia homoeocarpa), brown patch and large patch (Rhizoctonia solani), and pythium bligt (Pythium aphanidermatum) ;
Banana disease:
Sigatoka disease (Mycosphaerella fijiensis, Mycosphaerella musicola) ;
Lentils disease:
ascochyta blight (Ascochyta lentis);
Chickpea disease:
ascochyta blight (Ascochyta rabiei);
Green pepper disease:
anthracnose (Colletotrichum scovillei);
Mango disease:
anthracnose (Colletotrichum acutatum);
Fuit trees diseases:
white root rot (Rosellinia necatrix), and violet root rot (Helicobasidium mompa);
Postharvest diseases of fruits (for example, apple and pear): Mucor rot diseases (Mucor piriformis);
Seed diseases or diseases in the early stages of the growth of various plants caused by Aspergillus spp., Penicillium spp., Fusarium spp., Gibberella spp., Tricoderma spp., Thielaviopsis spp., Rhizopus spp., Mucor spp., Corticium spp., Phoma spp., Rhizoctonia spp. or Diplodia spp.; and the like;
Viral diseases:
Lettuce big-vein disease transmitted by Olpidium brassicae, and viral diseases of several crops transmitted by Polymixa spp. (e.g. Polymyxa betae and Polymyxa graminis);
Diseases caused by bacteria:
bacterial seedling blight of rice (Burkholderia plantarii), bacterial spot of cucumber (Pseudomonas syringae pv. Lachrymans), bacterial wilt of eggplant (Ralstonia solanacearum), canker of citrus (Xanthomonas citri), bacterial soft rot of Chinese cabbage (Erwinia carotovora), scab of potato (Streptomyces scabiei), Goss’s wilt of corn (Clavibacter michiganensis), Pierce’s disease of grapes, olive and peach (Xylella fastidiosa), andcrown gall of Rosacead plants such as apple, peach, cherries (Agrobacterium tumefaciens).

Examples of harmful arthropods, harmful nematodes, and harmful mollusks include the followings.

Hemiptera pests:
from the family Delphacidae, for example, small brown planthopper (Laodelphax striatella), brown planthopper (Nilaparvata lugens), white-backed planthopper (Sogatella furcifera), corn planthopper (Peregrinus maidis), cereal leafhopper (Javesella pellucida), sugarcane leafhopper (Perkinsiella saccharicida), and Tagosodes orizicolus;
from the family Cicadellidae, for example, green rice leafhopper (Nephotettix cincticeps), green paddy leafhopper (Nephotettix virescens), rice leafhopper (Nephotettix nigropictus), zigzag-striped leafhopper (Recilia dorsalis), tea green leafhopper (Empoasca onukii), potato leafhopper (Empoasca fabae), corn leafhopper (Dalbulus maidis), and rice leafhopper (Cofana spectra);
from the family Cercopidae, for example, Mahanarva posticata, and Mahanarva fimbriolata;
from the family Aphididae, for example, bean aphid (Aphis fabae), soybean aphid (Aphis glycines), cotton aphid (Aphis gossypii), green apple aphid (Aphis pomi), apple aphid (Aphis spiraecola), green peach aphid (Myzus persicae), leaf-curling plum aphid (Brachycaudus helichrysi), cabbage aphid (Brevicoryne brassicae), rosy apple aphid (Dysaphis plantaginea), false cabbage aphid (Lipaphis erysimi), potato aphid (Macrosiphum euphorbiae), foxglove aphid (Aulacorthum solani), lettuce aphid (Nasonovia ribisnigri), grain aphid (Rhopalosiphum padi), corn aphid (Rhopalosiphum maidis), brown citrus aphid (Toxoptera citricida), mealy plum aphid (Hyalopterus pruni), cane aphid (Melanaphis sacchari), black rice root aphid (Tetraneura nigriabdominalis), sugarcane cottony aphid (Ceratovacuna lanigera), and apple woolly aphid (Eriosoma lanigerum);
from the family Phylloxeridae, for example, grapevine phylloxera (Daktulosphaira vitifoliae), Pecan phylloxera (Phylloxera devastatrix), Pecan leaf phylloxera (Phylloxera notabilis), and Southern pecan leaf phylloxera (Phylloxera russellae);
from the family Adelgidae, for example, hemlock woolly aphid (Adelges tsugae), balsam woolly aphid (Adelges piceae), and Aphrastasia pectinatae;
from the family Pentatomidae, for example, black rice bug (Scotinophara lurida), Malayan rice black bug (Scotinophara coarctata), common green stink bug (Nezara antennata), white-spotted spined bug (Eysarcoris aeneus), lewis spined bug (Eysarcoris lewisi), white-spotted bug (Eysarcoris ventralis), Eysarcoris annamita, brown marmorated stink bug (Halyomorpha halys), green plant bug (Nezara viridula), brown stink bug (Euschistus heros), red banded stink bug (Piezodorus guildinii), Oebalus pugnax, and Dichelops melacanthus;
from the family Cydnidae, for example, Burrower brown bug (Scaptocoris castanea);
from the family Alydidae, for example, bean bug (Riptortus pedestris), corbett rice bug (Leptocorisa chinensis), and rice bug (Leptocorisa acuta);
from the family Coreidae, for example, Cletus punctiger, and Australian leaf-footed bug (Leptoglossus australis);
from the family Lygaeidae, for example, oriental chinch bug (Caverelius saccharivorus), seed bug (Togo hemipterus), and chinch bug (Blissus leucopterus);
from the family Miridae, for example, rice leaf bug (Trigonotylus caelestialium), sorghum plant bug (Stenotus rubrovittatus), wheat leaf bug (Stenodema calcarata), and American tarnished plant bug (Lygus lineolaris);
from the family Aleyrodidae, for example, greenhouse whitefly (Trialeurodes vaporariorum), tobacco whitefly (Bemisia tabaci), citrus whitefly (Dialeurodes citri), citrus spiny whitefly (Aleurocanthus spiniferus), tea spiny whitefly (Aleurocanthus camelliae), and Pealius euryae;
from the family Diaspididae, for example, Abgrallaspis cyanophylli, red scale (Aonidiella aurantii), San José scale (Diaspidiotus perniciosus), white peach scale (Pseudaulacaspis pentagona), arrowhead scale (Unaspis yanonensis), and citrus snow scale (Unaspis citri);
from the family Coccidae, for example, pink wax scale (Ceroplastes rubens);
from the family Margarodidae, for example, fluted scale (Icerya purchasi) and seychelles fluted scale (Icerya seychellarum) ;
from the family Pseudococcidae, for example, solanum mealybug (Phenacoccus solani), cotton mealybug (Phenacoccus solenopsis), Japanese mealybug (Planococcus kraunhiae), white peach scale (Pseudococcus comstocki), citrus mealybug (Planococcus citri), currant mealybug (Pseudococcus calceolariae), long-tailed mealybug (Pseudococcus longispinus), and tuttle mealybug (Brevennia rehi);
from the family Psyllidae, for example, citrus psylla (Diaphorina citri), two-spotted citrus psyllid (Trioza erytreae), pear sucker (Cacopsylla pyrisuga), Cacopsylla chinensis, potato psyllid (Bactericera cockerelli), and Pear psylla (Cacopsylla pyricola);
from the family Tingidae, for example, sycamore lace bug (Corythucha ciliata), aster tingid (Corythucha marmorata), Japanese pear lace bug (Stephanitis nashi), and azalea lace bug (Stephanitis pyrioides);
from the family Cimicidae, for example, common bed bug (Cimex lectularius);
from the family Cicadidae, for example, Giant Cicada (Quesada gigas);
from Triatoma spp., for example, Triatoma infestans; and the others.

Lepidoptera pests:
from the family Crambidae, for example, rice stem borer (Chilo suppressalis), Darkheaded stem borer (Chilo polychrysus), white stem borer (Scirpophaga innotata), yellow paddy borer (Scirpophaga incertulas), Rupela albina, rice leaf roller (Cnaphalocrocis medinalis), Marasmia patnalis, rice leaf roller (Marasmia exigua), cotton leaf roller (Notarcha derogata), corn borer (Ostrinia furnacalis), European corn borer (Ostrinia nubilalis), cabbage webworm (Hellula undalis), grape leafroller (Herpetogramma luctuosale), bluegrass webworm (Pediasia teterrellus), rice case-worm (Nymphula depunctalis), and Sugarcane borer (Diatraea saccharalis);
from the family Pyralidae, for example, lesser cornstalk borer (Elasmopalpus lignosellus) mealworm moth (Plodia interpunctella), and persimmon bark borer (Euzophera batangensis);
from the family Noctuidae, for example, cotton worm (Spodoptera litura), beet armyworm (Spodoptera exigua), rice armyworm (Mythimna separata), cabbage moth (Mamestra brassicae), pink borer (Sesamia inferens), grass armyworm (Spodoptera mauritia), green rice caterpillar (Naranga aenescens), Spodoptera frugiperda, true armyworm (Spodoptera exempta), black cutworm (Agrotis ipsilon), beet worm (Autographa nigrisigna), rice looper (Plusia festucae), soybean looper (Chrysodeixis includens), Trichoplusia spp., Heliothis spp. (such as tobacco budworm (Heliothis virescens)), Helicoverpa spp. (such as tobacco budworm (Helicoverpa armigera) and, corn earworm (Helicoverpa zea)), velvetbean caterpillar (Anticarsia gemmatalis), cotton leafworm (Alabama argillacea), and hop vine borer (Hydraecia immanis);
from the family Pieridae, for example, common cabbage worm (Pieris rapae);
from the family Tortricidae, for example, oriental fruit moth (Grapholita molesta), Grapholita dimorpha, soybean moth (Leguminivora glycinivorella), Matsumuraeses azukivora, summer fruit tortrix (Adoxophyes orana fasciata), smaller tea tortrix (Adoxophyes honmai), Japanese tea tortrix (Homona magnanima), apple tortrix (Archips fuscocupreanus), codling moth (Cydia pomonella), sugarcane shoot borer (Tetramoera schistaceana), bean shoot borer (Epinotia aporema), and citrus fruit borer (Ecdytolopha aurantiana);
from the family Gracillariidae, for example, tea leaf roller (Caloptilia theivora), and Asiatic apple leaf miner (Phyllonorycter ringoniella);
from the family Carposinidae, for example, peach fruit moth (Carposina sasakii);
from the family Lyonetiidae, for example, Coffee leaf miner (Leucoptera coffeella), peach leaf miner (Lyonetia clerkella), and Lyonetia prunifoliella;
from the family Lymantriidae, for example, Lymantria spp. (such as gypsy moth (Lymantria dispar)) and, Euproctis spp. (such as tea lymantriid (Euproctis pseudoconspersa));
from the family Plutellidae, for example, diamondback moth (Plutella xylostella);
from the family Gelechiidae, for example, peach worm (Anarsia lineatella), sweetpotato leaf folder (Helcystogramma triannulella), pink bollworm (Pectinophora gossypiella), potato moth (Phthorimaea operculella), and Tuta absoluta;
from the family Arctiidae, for example, American white moth (Hyphantria cunea);
from the family Castniidae, for example, giant sugarcane borer (Telchin licus);
from the family Cossidae, for example, Cossus insularis;
from the family Geometridae, for example, Ascotis selenaria;
from the family Limacodidae, for example, blue-striped nettle grub (Parasa lepida);
from the family Stathmopodidae, for example, persimmon fruit moth (Stathmopoda masinissa);
from the family Sphingidae, for example, tobacco hornworm (Acherontia lachesis);
from the family Sesiidae, for example, Nokona feralis, cherry borer (Synanthedon hector), and Synanthedon tenuis;
from the family Hesperiidae, for example, rice skipper (Parnara guttata);
from the family Tineidae, for example, casemaking clothes moth (Tinea translucens), and common clothes moth (Tineola bisselliella);
and the others.

Thysanoptera pests:
from the family Thripidae, for example, western flower thrips (Frankliniella occidentalis), oriental thrips (Thrips palmi), yellow tea thrips (Scirtothrips dorsalis), onion thrips (Thrips tabaci), eastern flower thrips (Frankliniella intonsa), rice thrips (Stenchaetothrips biformis), and Echinothrips americanus;
from the family Phlaeothripidae, for example, aculeated rice thrips (Haplothrips aculeatus);
and the others.

Diptera pests:
from the family Anthomyiidae, for example, seedcorn maggot (Delia platura), onion maggot (Delia antiqua) , and beet leaf miner (Pegomya cunicularia);
from the family Ulidiidae, for example, sugarbeet root maggot (Tetanops myopaeformis);
from the family Agromyzidae, for example, rice leaf miner (Agromyza oryzae), tomato leaf miner (Liriomyza sativae), chrysanthemum leaf miner (Liriomyza trifolii), and pea leafminer (Chromatomyia horticola);
from the family Chloropidae, for example, rice stem maggot (Chlorops oryzae);
from the family Tephritidae, for example, melon fly (Bactrocera cucurbitae), oriental fruit fly (Bactrocera dorsalis), Malaysian fruit fly (Bactrocera latifrons), olive fruit fly (Bactrocera oleae), Queensland fruit fly (Bactrocera tryoni), Mediterranean fruit fly (Ceratitis capitata) , apple maggot (Rhagoletis pomonella), and Japanese cherry fruit fly (Rhacochlaena japonica);
from the family Ephydridae, for example, smaller rice leaf miner (Hydrellia griseola), whorl maggot (Hydrellia philippina), and paddy stem maggot (Hydrellia sasakii);
from the family Drosophilidae, for example, cherry drosophila (Drosophila suzukii);
from the family Phoridae, for example, Megaselia spiracularis;
from the family Psychodidae, for example, Clogmia albipunctata;
from the family Sciaridae, for example, Bradysia difformis;
from the family Cecidomyiidae, for example, hessian fly (Mayetiola destructor) and, paddy gall fly (Orseolia oryzae);
from the family Diopsidae, for example, Diopsis macrophthalma;
from the family Tipulidae, for example, rice crane fly (Tipula aino), common cranefly (Tipula oleracea), and European cranefly (Tipula paludosa);
from the family Culicidae, for example, southern house mosquito (Culex pipiens pallens), dengue mosquito (Aedes aegypti), Asian tiger mosquito (Aedes albopictus), Chinese malaria mosquito (Anopheles hyracanus sinensis), Culex quinquefasciatus, Culex pipiens molestus Forskal , and brown house mosquito (Culex quinquefasciatus);
from the family Simulidae, for example, Prosimulium yezoensis, and Simulium ornatum;
from the family Tabanidae, for example, Tabanus trigonus;
from the family Muscidae, for example, house fly (Musca domestica), false stable fly (Muscina stabulans), biting house fly (Stomoxys calcitrans), and buffalo fly (Haematobia irritans);
from the family Tabanidae, for example, Tabanus trigonus;
from the family Calliphoridae;
from the family Sarcophagidae;
from the family Chironomidae, for example, Chironomus plumosus, Chironomus yoshimatsui, and Glyptotendipes tokunagai;
from the family Fannidae;
and the others.

Coleoptera pests:
from the family Chrysomelidae, for example, western corn rootworm (Diabrotica virgifera virgifera), southern corn rootworm (Diabrotica undecimpunctata howardi), northern corn rootworm (Diabrotica barberi), Mexican corn rootworm (Diabrotica virgifera zeae), banded cucumber beetle (Diabrotica balteata), cucurbit beetle (Diabrotica speciosa), bean leaf beetle (Cerotoma trifurcata), barley leaf beetle (Oulema melanopus), cucurbit leaf beetle (Aulacophora femoralis), striped flea beetle (Phyllotreta striolata), cabbage flea beetle (Phyllotreta cruciferae), western black flea beetle (Phyllotreta pusilla), cabbage stem flea beetle (Psylliodes chrysocephala), Colorado potato beetle (Leptinotarsa decemlineata), rice leaf beetle (Oulema oryzae), grape colaspis (Colaspis brunnea), corn flea beetle (Chaetocnema pulicaria), sweet-potato flea beetle (Chaetocnema confinis), potato flea beetle (Epitrix cucumeris), rice leaf beetle (Dicladispa armigera), southern corn leaf beetle (Myochrous denticollis), Laccoptera quadrimaculata, and tobacco flea beetle (Epitrix hirtipennis);
from the family Carabidae, for example, Seedcorn beetle (Stenolophus lecontei), and slender seedcorn beetle (Clivina impressifrons);
from the family Scarabaeidae, for example, cupreus chafer (Anomala cuprea), soybean beetle (Anomala rufocuprea), Anomala albopilosa, Japanese beetle (Popillia japonica), yellowish elongate chafer (Heptophylla picea), European Chafer (Rhizotrogus majalis), Tomarus gibbosus, Holotrichia spp., Phyllophaga spp. (such as June beetle (Phyllophaga crinita)), and Diloboderus spp. (such as Diloboderus abderus);
from the family Curculionidae, for example, coffee bean weevil (Araecerus coffeae), sweet-potato weevil (Cylas formicarius), West Indian sweet-potato weevil (Euscepes postfasciatus), alfalfa weevil (Hypera postica), maize wevil (Sitophilus zeamais), rice plant weevil (Echinocnemus squameus), rice water weevil (Lissorhoptrus oryzophilus), Rhabdoscelus lineaticollis, boll weevil (Anthonomus grandis), nunting billbug (Sphenophorus venatus), southern corn billbug (Sphenophorus callosus), soybean stalk weevil (Sternechus subsignatus), sugarcane weevil (Sphenophorus levis), rusty gourd-shaped weevil (Scepticus griseus), brown gourd-shaped weevil (Scepticus uniformis), Mexican bean weevil (Zabrotes subfasciatus), pine beetle (Tomicus piniperda),coffee berry borer (Hypothenemus hampei), Aracanthus spp. (such as Aracanthus mourei), and cotton root borer (Eutinobothrus brasiliensis);
from the family Tenebrionidae, for example, red meal beetle (Tribolium castaneum), and mason beetle (Tribolium confusum);
from the family Coccinellidae, for example, twenty-eight-spotted ladybird (Epilachna vigintioctopunctata);
from the family Bostrychidae, for example, common powder-post beetle (Lyctus brunneus);
from the family Ptinidae;
from the family Cerambycidae, for example, citrus long-horned beetle (Anoplophora malasiaca) and, Migdolus fryanus;
from the family Elateridae, for example, Melanotus okinawensis, barley wireworm (Agriotes fuscicollis), Melanotus legatus, Anchastus spp., Conoderus spp., Ctenicera spp., Limonius spp., and Aeolus spp.;
from the family Staphylinidae, for example, Paederus fuscipes;
from the family Dermestidae, for example, varied carpet beetle (Anthrenus verbasci) and, hide beetle (Dermestes maculates);
from the family Anobiidae, for example, tobacco beetle (Lasioderma serricorne), and biscuit beetle (Stegobium paniceum);
and the others.

Orthoptera pests:
from the family Acrididae, for example, oriental migratory locust (Locusta migratoria), Moroccan locust (Dociostaurus maroccanus), Australian plague locust (Chortoicetes terminifera), red locust (Nomadacris septemfasciata), brown locust (Locustana pardalina), tree locust (Anacridium melanorhodon), Italian locust (Calliptamus italicus), differential grasshopper (Melanoplus differentialis), two-striped grasshopper (Melanoplus bivittatus), migratory grasshopper (Melanoplus sanguinipes), red-legged grasshopper (Melanoplus femurrubrum), clear-winged grasshopper (Camnula pellucida), desert locust (Schistocerca gregaria), Yellow-winged locust (Gastrimargus musicus), ,spur-throated locust (Austracris guttulosa), , Japanese grasshopper (Oxya yezoensis), rice grasshopper (Oxya japonica), and Bombay locust (Patanga succincta);
from the family Gryllotalpidae, for example, oriental mole cricket (Gryllotalpa orientalis);
from the family Gryllidae, for example, house cricket (Acheta domestica), and emma field cricket (Teleogryllus emma);
from the family Tettigoniidae, for example, mormon cricket (Anabrus simplex);
and the others.

Hymenoptera pests:
from the family Tenthredinidae, for example, beet sawfly (Athalia rosae), and Nippon cabbage sawfly (Athalia japonica);
from the family Formicidae, for example, Solenopsis spp. (such as red imported fire ant (Solenopsis invicta) and, tropical fire ant (Solenopsis geminata)), Atta spp. (such as brown leaf-cutting ant (Atta capiguara)), Acromyrmex spp., Paraponera clavata, black house ant (Ochetellus glaber), little red ant (Monomorium pharaonis), Argentine ant (Linepithema humile), Formica japonica, Pristomyrmex punctutus, Pheidole noda, big-headed ant (Pheidole megacephala), Camponotus spp. (such as Camponotus japonicus, and Camponotus obscuripes), Pogonomyrmex spp. (such as western harvester ant (Pogonomyrmex occidentalis)), Wasmania spp. (such as Wasmania auropunctata), and long-legged ant (Anoplolepis gracilipes);
from the family Vespidae, for example, Asian giant hornet (Vespa mandarinia japonica), Vespa simillima, Vespa analis Fabriciusi, Asian hornet (Vespa velutina), and Polistes jokahamae;
from the family Siricidae, for example, pine wood wasp (Urocerus gigas);
from the family Bethylidae;
and the others.

Blattodea pests:
from the family Blattellidae, for example, German cockroach (Blattella germanica);
from the family Blattidae, for example, smoky-brown cockroach (Periplaneta fuliginosa), American cockroach (Periplaneta americana), brown cockroach (Periplaneta brunnea), and black cockroach (Blatta orientalis);
from the family Termitidae, for example, Japanese termite (Reticulitermes speratus), Formosan termite (Coptotermes formosanus), western drywood termite (Incisitermes minor), Cryptotermes domesticus, Odontotermes formosanus, Neotermes koshunensis, Glyptotermes satsumensis, Glyptotermes nakajimai, Glyptotermes fuscus, Hodotermopsis sjostedti, Coptotermes guangzhouensis, Reticulitermes amamianus, Reticulitermes miyatakei, Reticulitermes kanmonensis, Nasutitermes takasagoensis, Pericapritermes nitobei, Sinocapritermes mushae, and Cornitermes cumulans;
and the others.

Siphonaptera pests:
from the family Pulicidae, for example, human flea (Pulex irritans), cat flea (Ctenocephalides felis), dog flea (Ctenocephalides canis), oriental rat flea (Xenopsylla cheopis), and chicken flea (Echidnophaga gallinacea);
from the family Hectopsyllidae, for example, chigoe flea (Tunga penetrans); and
from the family Ceratophyllidae, for example, European rat flea (Nosopsyllus fasciatus);
and the others.

Psocodae pests:
from the family Pediculidae, for example, head louse (Pediculus humanus capitis);
from the family Pthiridae, for example, crab louse (Pthirus pubis);
from the family Haematopinidae, for example, short-nosed cattle louse (Haematopinus eurysternus), pig louse (Haematopinus suis);
from the family Linognathidae, for example, blue cattle louse (Linognathus vituli), sheep face louse (Linognathus ovillus), capillate louse (Solenopotes capillatus);
from the family Bovicoliidae, for example, Bovicola bovis, sheep louse (Bovicola ovis), Bovicola breviceps, Damalinia forficula, Werneckiella spp.;
from the family Trichodectidae, for example, dog biting louse (Trichodectes canis), cat louse (Felicola subrostratus);
from the family Menoponidae, for example, chicken biting louse (Menopon gallinae), chicken body louse (Menacanthus stramineus), Trinoton spp.;
from the family Trimenoponidae, for example, Cummingsia spp. ;
from the family Trogiidae, for example, Trogium pulsatorium;
from the family Liposcelidaeor Liposcelididae, for example, book louse (Liposcelis subfuscas), Liposcelis bostrychophilus, Liposcelis simulans, Liposcelis divinatorius, Liposcelis entomophila.

Thysanura:

from the family Lepismatidae, oriental silverfish (Ctenolepisma villosa), moth fish (Lepisma saccharina), and the like.

Acari pests:
from the family Tetranychidae, for example, common red spider mite (Tetranychus urticae), kanzawa spider mite (Tetranychus kanzawai), red spider mite (Tetranychus evansi), citrus red mite (Panonychus citri), fruit-tree red spider mite (Panonychus ulmi), and Oligonychus spp.;
from the family Eriophyidae, for example, Japanese citrus rust mite (Aculops pelekassi), Phyllocoptruta citri, tomato mite (Aculops lycopersici), purple mite (Calacarus carinatus), tea rust mite (Acaphylla theavagrans), Eriophyeschibaensis, apple bud mite (Aculus schlechtendali), Aceria diospyri, Aceria tosichella, and Shevtchenkella sp.;
from the family Tarsonemidae, for example, broad mite (Polyphagotarsonemus latus);
from the family Tenuipalpidae, for example, Brevipalpus phoenicis;
from the family Tuckerellidae;
from the family Ixodidae, for example, Haemaphysalis longicornis, Haemaphysalis flava, Dermacentor taiwanensis, American dog tick (Dermacentor variabilis), Dermacentor andersoni, Ixodes ovatus, Ixodes persulcatus, Ixodes ricinus, black-legged tick (Ixodes scapularis), lone star tick (Amblyomma americanum), Amblyomma maculatum, cattle tick (Boophilus microplus), Boophilus annulatus, and brown dog tick (Rhipicephalus sanguineus);
from the family Acaridae, for example, cereal mite (Tyrophagus putrescentiae)and grassland mite (Tyrophagus similis);
from the family Pyroglyphidae, for example, American house dust mite (Dermatophagoides farinae), and European house dust mite (Dermatophagoides pteronyssinus);
from the family Cheyletidae, for example, Cheyletus eruditus, Cheyletus malaccensis, Cheyletus moorei, and Cheyletiella yasguri;
from the family Sarcoptidae, for example, ear mange mite (Otodectes cynotis), and itch mite (Sarcoptes scabiei);
from the family Demodicidae, for example, dog follicle mite (Demodex canis);
from the family Listrophoridae;
from the family Haplochthoniidae;
from the family Macronyssidae, for example, tropical rat mite (Ornithonyssus bacoti), and feather mite (Ornithonyssus sylviarum);
from the family Dermanyssidae, for example, bird mite (Dermanyssus gallinae);
from the family Trombiculidae, for example, Leptotrombidium akamushi;
and the others.

Araneae pests:
from the family Eutichuridae, for example, Cheiracanthium japonicum;
from the family Theridiidae, for example, red-back spider (Latrodectus hasseltii);
and the others.

Polydesmida pests:

from the family Paradoxosomatidae, for example, flat-backed millipede (Oxidus gracilis), and Nedyopus tambanus; and the others.

Isopoda pests:
from the family Armadillidiidae, for example, common pill bug (Armadillidium vulgare);
and the others.

Chilopoda pests:
from the family Scutigeridae, for example, Thereuonema hilgendorfi;
from the family Scolopendridae, for example, giant tropical centipede (Scolopendra subspinipes);
from the family Ethopolidae, for example, Bothropolys rugosus;
and the others.

Gastropoda pests:
from the family Limacidae, for example, tree slug (Limax marginatus), and garden tawny slug (Limax flavus);
from the family Philomycidae, for example, Meghimatium bilineatum;
from the family Ampullariidae, for example, golden apple snail (Pomacea canaliculata);
from the family Lymnaeidae, for example, Austropeplea ollula;
and the others.

Nematoda pests:
from the family Aphelenchoididae, for example, rice white-tip nematode (Aphelenchoides besseyi);
from the family Pratylenchidae, for example, root lesion nematode (Pratylenchus coffeae), Pratylenchus brachyurus, California meadow nematode (Pratylenchus neglectus), and Radopholus similis;
from the family Heteroderidae, for example, javanese root-knot nematode (Meloidogyne javanica), southern root-knot nematode (Meloidogyne incognita), northern root-knot nematode (Meloidogyne hapla), soybean cyst nematode (Heterodera glycines), potato cyst nematode (Globodera rostochiensis), and white potato cyst nematode (Globodera pallida);
from the family Hoplolaimidae, for example, Rotylenchulus reniformis;
from the family Anguinidae, for example, strawberry bud nematode (Nothotylenchus acris), and stem nematode (Ditylenchus dipsaci);
from the family Tylenchulidae, for example, citrus nematode (Tylenchulus semipenetrans);
from the family Longidoridae, for example, dagger nematode (Xiphinema index);
from the family Trichodoridae;
from the family Parasitaphelenchidae, for example, pine wilt disease (Bursaphelenchus xylophilus);
and the others.

As a target, the harmful arthropods such as harmful insects, harmful mites, harmful mollusks, and harmful nematodes may be the harmful arthropods such as harmful insects, harmful mites, harmful mollusks, and harmful nematodes, each of which has a reduced agent-sensitivity to or a developed agent-resistance to an insecticide, a miticide, a molluscicide, and a nematicide, respectively.

The compound of the present invention, or the composition A is usually mixed with an inert carrier such as solid carrier, liquid carrier or gaseous carrier, and if necessary, adding surfactants and the other auxiliary agents for formulation, to formulate into emulsifiable concentrates, oil solutions, dust formulations, granules, wettable powders, water dispersible granules, flowables, dry flowables, microcapsules, aerosols, poison baits, resin formulations, shampoo formulations, paste-like formulations, foams, carbon dioxide formulations, and tablets and the others. Such formulations may be processed into mosquito repellent coils, electric mosquito repellent mats, liquid mosquito formulations, smoking agents, fumigants, sheet formulations, spot-on formulations or formulations for oral treatment. These formulations comprise usually 0.0001 to 95% by weight of the present compound, the compound of the present invention or the composition A.

Examples of the solid carrier to be used in the formulation include fine powders or granules of clays (for example, kaolin clay, diatomaceous earth, bentonite, or acid white clay), dry silica, wet silica, talcs, ceramics, other inorganic minerals (for example, sericite, quartz, sulfur, active carbon, or calcium carbonate) or chemical fertilizers (for example, ammonium sulfate, ammonium phosphate, ammonium nitrate, urea, or ammonium chloride) and the others; as well as synthetic resins (for example, polyester resins such as polypropylene, polyacrylonitrile, polymethyl methacrylate or polyethylene terephthalate; nylon resins (for example, nylon-6, nylon-11, or nylon-66); polyamide resins; polyvinyl chloride, polyvinylidene chloride, vinyl chloride-propylene copolymers, and the others).

Examples of the liquid carriers include water; alcohols (for example, methanol, ethanol, isopropyl alcohol, butanol, hexanol, benzyl alcohol, ethylene glycol, propylene glycol, or phenoxy ethanol); ketones (for example, acetone, methyl ethyl ketone, or cyclohexanone); aromatic hydrocarbons (for example, toluene, xylene, ethyl benzene, dodecyl benzene, phenyl xylyl ethane, or methylnaphthalene); aliphatic hydrocarbons (for example, hexane, cyclohexane, kerosene, or light oil); esters (for example, ethyl acetate, butyl acetate, isopropyl myristate, ethyl oleate, diisopropyl adipate, diisobutyl adipate, or propylene glycol monomethyl ether acetate); nitriles (for example, acetonitrile, or isobutyronitrile); ethers (for example, diisopropyl ether, 1,4-dioxane, 1,2-dimethoxyethane, diethyleneglycol dimethyl ether, diethylene glycol monomethyl ether, propylene glycol monomethyl ether, dipropylene glycol monomethyl ether, or 3-methoxy-3-methyl-1-butanol); amides (for example, DMF, or N,N-dimethylacetamide); sulfoxides (for example, dimethyl sulfoxide); propylene carbonate; and vegetable oils (for example, soybean oil or cottonseed oil).

Examples of gaseous carrier include fluorocarbon, butane gas, liquefied petroleum gas (LPG), dimethyl ether, and carbon dioxide gas.

Examples of the surfactants include nonionic surfactants such as polyoxyethylenated alkyl ethers, polyoxyethylenated alkyl aryl ethers, and polyethylene glycol fatty acid esters; and anionic surfactants such as alkyl sulfonates, alkylbenzene sulfonates and alkyl sulfates.

Examples of the other auxiliary agents for formulation include a binder, a dispersant, a colorant and a stabilizer. Specific examples include casein, gelatin, polysaccharides (for example, starch, gum arabic, cellulose derivatives and alginic acid), lignin derivatives, bentonite, water-soluble synthetic polymers (for example, polyvinyl alcohol, polyvinyl pyrrolidone and polyacrylic acids), acidic isopropyl phosphate, 2,6-di-tert-butyl-4-methylphenol, and a mixture of 2-tert-butyl-4-methoxyphenol and 3-tert-butyl-4-methoxyphenol. Specific examples thereof include Nimbus (registered trademark), Assist (registered trademark), Aureo (registered trademark), Iharol (registered trademark), Silwet L-77 (registered trademark), BreakThru (registered trademark), SundanceII (registered trademark), Induce (registered trademark), Penetrator (registered trademark), AgriDex (registered trademark), Lutensol A8 (registered trademark), NP-7 (registered trademark), Triton (registered trademark), Nufilm (registered trademark), Emulgator NP7 (registered trademark), Emulad (registered trademark), TRITON X 45 (registered trademark), AGRAL 90 (registered trademark), AGROTIN (registered trademark), ARPON (registered trademark), EnSpray N (registered trademark), and BANOLE (registered trademark), and the others.

Examples of base material of the resin formulation include polyvinyl chloride polymers, polyurethane and the others, and a plasticizer such as phthalate esters (for example, dimethyl phthalate, dioctyl phthalate), adipic acid esters and stearic acid may be added to these base materials, if necessary. The resin formulation can be prepared by mixing the compound of the present invention with the above-mentioned base material, kneading the mixture, followed by molding it by injection molding, extrusion molding or pressure molding and the like. The resultant resin formulation can be subjected to further molding or cutting procedure and the like, if necessary, to be processed into shapes such as a plate, film, tape, net or string shape. These resin formulations can be processed into animal collars, animal ear tags, sheet products, trap strings, gardening supports and other products.

Examples of a base material for the poison baits include bait ingredients such as grain powder, vegetable oil, saccharide and crystalline cellulose, and if necessary, with addition of antioxidants such as dibutylhydroxytoluene and nordihydroguaiaretic acid, preservatives such as dehydroacetic acid, accidental ingestion inhibitors for children and pets such as a chili powder, insect attraction fragrances such as cheese flavor, onion flavor and peanut oil.

The plants as used herein include entire plant, foliages, flowers, ears, fruits, stems, branches, tree canopies, seeds, vegetative reproductive organs, and seedlings.

The vegetative reproductive organs represent a part of plant which have the ability to grow when the part is separated from the body and placed in soil, among the roots, stems, leaves and the like of the plant. Examples of the vegetative reproductive organs include tuberous root, creeping root, bulb, corm or solid bulb, tuber, rhizome, stolon, rhizophore, cane cuttings, propagule, and vine cutting. Here the stolon is also called runner, propagule is also called bulbils, which is divided into broad bud and bulblets. The vines represent shoots (generic name for leaves and stems) of sweet potato and Japanese yam. Discoid stem, corm, tuber, rhizome, stem fragments, rhizophore and tuberous root are also collectively referred to bulbs. For example, though a cultivation of potato begins by planting tubers in soil, the tubers used are generally called seed potatoes.

Examples of a method of controlling pests by applying an effective amount of the compound of the present invention, or the composition A to soil include a method of applying an effective amount of the compound of the present invention, or the composition A to soil before planting plants or after planting plants.

More specific examples of the method for controlling harmful arthropods include planting hole treatment (spraying into planting holes, soil mixing after planting hole treatment), plant foot treatment (plant foot spraying, soil mixing after plant foot treatment, irrigation at plant foot, plant foot treatment at a later seeding raising stage), planting furrow treatment (planting furrow spraying, soil mixing after planting furrow treatment), planting row treatment (planting row spraying, soil mixing after planting row treatment, planting row spraying at a growing stage), planting row treatment at the time of sowing (planting row spraying at the time of sowing, soil mixing after planting row treatment at the time of sowing), broadcast treatment (overall soil surface spraying, soil mixing after broadcast treatment), side-article treatment, treatment of water surface (application to water surface, application to water surface after flooding), other soil spraying treatment (spraying of a granular formulation on leaves at a growing stage, spraying under a canopy or around a tree stem, spraying on the soil surface, mixing with surface soil, spraying into seed holes, spraying on the ground surfaces of furrows, spraying between plants), other irrigation treatment (soil irrigation, irrigation at a seedling raising stage, drug solution injection treatment, irrigation of a plant part just above the ground, drug solution drip irrigation, chemigation), seedling raising box treatment (spraying into a seedling raising box, irrigation of a seedling raising box, flooding into a seedling raising box with drug solution), seedling raising tray treatment (spraying on a seedling raising tray, irrigation of a seedling raising tray, flooding into a seedling raising tray with drug solution), seedbed treatment (spraying on a seedbed, irrigation of a seedbed, spraying on a lowland rice nursery, immersion of seedlings), seedbed soil incorporation treatment (mixing with seedbed soil, mixing with seedbed soil before sowing, spraying at sowing before covering with soils, spraying at sowing after covering with soils, mixing with covering soil, and other treatment (mixing with culture soil, plowing under, mixing with surface soil, mixing with soil at the place where raindrops fall from a canopy, treatment at a planting position, spraying of a granule formulation on flower clusters, mixing with a paste fertilizer).

Examples of the application to seeds (or seed treatments) include an application of the compound of the present invention or the composition A to seeds or vegetative reproductive organs, and specific examples thereof include spraying treatment in which a suspension of the compound of the present invention or the composition A is sprayed onto seed surface or the vegetative reproductive organ surface in the form of mist; smearing treatment in which the compound of the present invention or the composition A is coated a surface of seeds or the vegetative reproductive organ; a soaking treatment in which the seeds are soaked into the solution of the compound of the present invention or the composition A for a certain time; and a method for coating the seeds or the vegetative reproductive organ with a carrier containing the compound of the present invention or the composition A (film coating treatment, pellet coating treatment). Examples of the above-described vegetative reproductive organ include particularly seed potato.

When the composition A is applied to seeds or vegetative reproductive organs, the composition A may be also applied to seeds or vegetative reproductive organs as a single formulation, or the composition A may be applied to seeds or vegetative reproductive organs as a divided plural of formulations by a plurality of times. Examples or the method in which the composition A is applied as a divided plural of formulations by a plurality of times include, for exmaple, a method in which the formulations comprising as an active component the compound of the present invention only are applied, and seeds or vegetative reproductive organs are air dried, followed by applying the formulations comprising the present ingredient: and a method in which the formulations comprising as an active component the compound of the present invention and the present ingredients are applied, and seeds or vegetative reproductive organs are air dried, followed by applying the formulations comprising the present ingredients other than the already-applied present ingredients, are included.

As used herein, seeds or vegetative reproductive organs carrying the compound of the present invention or the composition A means seeds or vegetative reproductive organs in the state where the compound of the present invention or the composition A is adhered to a surface of the seeds or the vegetative reproductive organ. The above-described seeds or vegetative reproductive organs carrying the compound of the present invention or the composition A may be adhered by any other materials that are different from the compound of the present invention or the composition A before or after being adhered the compound of the present invention or the composition A to the seeds or vegetative reproductive organs.

Also, when the composition A is adhered in a form of layer(s) to a surface of seeds or vegetative reproductive organ, the layer(s) is/are composed of one layer or a plural of layers. Also, when a plural layers are formed, each of the layer may be composed of a layer comprising one or more active ingredients, or a combination of a layer comprising one or more active ingredients and a layer not comprising an active ingredient.

Seeds or vegetative reproductive organs carrying the compound of the present invention or the composition A can be obtained, for example, by applying the formulations comprising the compound of the present invention or the composition A by the above-described application method to seeds to seeds or vegetative reproductive organs.

When the compound of the present invention, or the composition A is applied for harmful arthropods control in agricultural fields, the application dose thereof is usually within a range of 1 to 10,000 g g of the compound of the present invention per 10,000 m². In the case of being applied to seeds or vegetative reproductive organs, the dose of application dose thereof is usually within a range of 0.001 to 100 g of the compound of the present invention per 1 Kg of seeds or vegetative reproductive organs. When the compound of the present invention, or the composition A is formulated into an emulsifiable concentrate, a wettable powder or a flowable etc., they are usually applied by diluting them with water so as to make an effective concentration of the active ingredients 0.01 to 10,000 ppm, and the dust formulation or the granular formulation, etc., is usually applied as itself without diluting them.

Also, the resin preparation which is processed into a sheet or a string may be applied by winding a plant with a sheet or a string of the resin preparation, putting a string of the resin preparation around a crop so that the plant is surrounded by the string, or laying a sheet of the resin preparation on the soil surface near the root of a plant.

When the compound of the present invention, or the composition A is used to control harmful arthropods that live inside a house, the application dose as an amount of the compound of the present invention is usually within a range from 0.01 to 1,000 mg per 1 m² of an area to be treated, in the case of using it on a planar area. In the case of using it spatially, the application dose as an amount of the compound of the present invention is usually within a range from 0.01 to 500 mg per 1 m³ of the space to be treated. When the compound of the present invention or the composition A is formulated into emulsifiable concentrates, wettable powders, flowables or the others, such formulations are usually applied after diluting it with water in such a way that a concentration of the active ingredient is within a range from 0.1 to 10,000 ppm. In the case of being formulated into oil solutions, aerosols, smoking agents, poison baits and the others, such formulations are used as itself without diluting it.

The compound of the present invention, or the composition A may be used as an agent for controlling harmful arthropods in agricultural lands such as paddy fields, fields, turfs, and orchards. Examples of the plants to be applied include the followings.
corn, rice, wheat, barley, rye, oat, sorghum, cotton, soybean, peanut, buckwheat, beet, rapeseed, sunflower, sugar cane, tobacco,
solanaceous vegetables (for example, eggplant, tomato, pimento, pepper, or potato),
cucurbitaceous vegetables (for example, cucumber, pumpkin, zucchini, water melon, or melon),
cruciferous vegetables (for example, Japanese radish, white turnip, horseradish, kohlrabi, Chinese cabbage, cabbage, leaf mustard, broccoli, or cauliflower),
asteraceous vegetables (for example, burdock, crown daisy, artichoke, or lettuce),
liliaceous vegetables (for example, green onion, onion, garlic, or asparagus),
ammiaceous vegetables (for example, carrot, parsley, celery, or parsnip),
chenopodiaceous vegetables (for example, spinach, or Swiss chard),
lamiaceous vegetables (for example, Perilla frutescens, mint, or basil),
strawberry, sweet potato, Dioscorea japonica, colocasia, pomaceous fruits (for example, apple, pear, Japanese pear, Chinese quince, or quince),
stone fleshy fruits (for example, peach, plum, nectarine, Prunus mume, cherry fruit, apricot, or prune),
citrus fruits (for example, Citrus unshiu, orange, lemon, lime, or grapefruit),
nuts (for example, chestnut, walnuts, hazelnuts, almond, pistachio, cashew nuts, or macadamia nuts),
berry fruits (for example, blueberry, cranberry, blackberry or raspberry),
grape, kaki persimmon, olive, Japanese plum, banana, coffee, date palm, coconuts, tea, mulberry, ornamental foliage plants, woodland plants, lawns, pastures.

The above-mentioned plants may include genetically modified crops.

### EXAMPLES

Hereinafter, the present invention is explained in more detail by Preparation Examples, Reference Preparation Examples, Formulation Examples, and Test Examples, however, the present invention should not be limited to only the these Examples.

As used herein, Me represents methyl group, Et represents ethyl group, Pr represents propyl group, i-Pr represents isopropyl group, Bu represents butyl group, i-Bu represents isobutyl group, t-Bu represents t-butyl group, Pen represents pentyl group, c-Pr represents cyclopropyl group, c-Bu represents cyclobutyl group, c-Pen represents cyclopentyl group, c-Hex represents cyclohexyl group, and Ph represents phenyl group.

Firstly, Preparation examples of the compound of the present invention are described.

When a physical property of a compound is measured by a liquid chromatography / mass spectrometry analysis (hereinafter, referred to as LCMS), a measured molecular ion value [M + H]⁺ or [M—H]^{—} and a retention time (hereinafter, referred to as RT) is described. The measured condition for liquid chromatography (hereinafter, referred to as LC) and mass spectrometry (hereinafter, referred to as MS) is described below.

### LC Condition

Column: L-column2 ODS, inner diameter 4.6 mm, length 30 m m, particle size 3 µm (Chemicals Evaluation and Research Institute, Japan)

UV measurement wavelength: 254 nm

Mobile phase: A solution: 0.1 % aqueous formic acid solution,

B solution: 0.1 % formic acid in acetonitrile

Flow Rate: 2.0 mL/min

Pump: LC-20AD (manufacturing SHIMAZU) 2 umps (high pressure gradient)

Gradient condition: The following concentration gradient shown in [Table LC1] is liquid-transferred.

TABLE LC1

| Time (min) | A solution (%) | B solution (%) |
|---|---|---|
| 0.01 | 90 | 10 |
| 2.00 | 0 | 100 |
| 4.00 | 0 | 100 |
| 4.01 | 90 | 10 |

### MS Condition

Detector: LCMS-2020 (manufacturing SHIMADZU)

Ionization method: DUIS

### Reference Prepration Example 1

To a mixture of methyl 2-(5-bromo-2-methylphenyl)acetate (which was prepared by the method described in WO 2019/016269 A1) 19.4 g, methyl formate 14.7 mL, and dimethoxyethane 150 mL was added tert-butoxy potassium 19.7 g at 0° C., and the mixture was stirred at room temperature for 1 hour. To the resulting mixture was added 1 N hydrochloric acid, and the mixture was extracted with MTBE. The resulting mixture was dried over anhydrous magnesium sulfate, and concentrated under reduced pressure. To the resulting residue was added DMF 100 mL, and the mixture was stirred, and thereto were added potassium carbonate 9.9 g, and dimethyl sulfate 6.8 mL at 0° C. successively, and the mixture was stirred at room temperature for 5 hours. Water was added to the resulting mixture, and the mixture was extracted with MTBE. The resulting organic layer was dried over anhydrous magnesium sulfate, and concentrated under reduced pressure. The resulting residue was subjected to a silica gel column chromatography (ethyl acetate : hexane= 1 : 4) to obtain the intermediate compound 1 represented by the following formula 14.2 g.

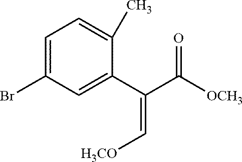 [SMILES: CO/C=C(/C(=O)OC)c1cc(Br)ccc1C]

Intermediate compound 1: ¹H-NMR (CDCl₃) δ: 7.56 (1 H, s), 7.33 (1 H, dd), 7.24 (1 H, d), 7.09 (1 H, d), 3.84 (3 H, s), 3.70 (3 H, s), 2.12 (3 H, s).

### Reference Preparation Example 1-1

The compounds which were prepared according to the Reference Prepration Exmaple 1 and their physical property value are shown below.

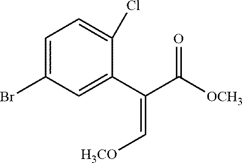 [SMILES: CO/C=C(/C(=O)OC)c1cc(Br)ccc1Cl]

Intermediate compound 1-2: ¹H-NMR (CDCl₃) δ: 7.57 (1 H, s), 7.38-7.35 (2 H, m), 7.29-7.26 (1 H, m), 3.87 (3 H, s), 3.72 (3 H, s) .

### Reference Preparation Example 1-2

The compounds which can be prepared according to the Reference Prepration Exmaple 1 are shown below.

A compound represented by formula (M1-a):

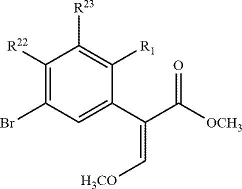

wherein a combination of R¹, R²² and R²³ represent any combinations described in [Table A1].

TABLE A1

| Intermediate compound | R¹ | R²² | R²³ |
|---|---|---|---|
| 1-3 | F | H | H |
| 1-5 | Et | H | H |
| 1-6 | c-Pr | H | H |
| 1-7 | H | Cl | H |
| 1-9 | H | H | Cl |

### Reference Preparation Example 1-3

The compounds which were prepared according to the Reference Preparation Example 1 and their physical property value are shown below.

The compounds represented by formula (M1-a) wherein a combination of R¹, R²² and R²³ represent any combinations described in [Table A14].

TABLE A14

| Intermediate compound | R¹ | R²² | R²³ |
|---|---|---|---|
| 1-4 | OMe | H | H |
| 1-8 | H | Me | H |
| 1-10 | H | H | Me |

Intermediate compound 1-4: : ¹H-NMR (CDCl₃) δ: 7.50 (1 H, s), 7.37 (1 H, dd), 7.28 (1 H, d), 6.78 (1 H, d), 3.83 (3 H, s), 3.76 (3 H, s), 3.69 (3 H, s).
Intermediate compound 1-8: ¹H-NMR (CDCl₃) δ: 7.54 (1 H, s), 7.51 (1 H, s), 7.23-7.15 (2 H, m), 3.86 (3 H, s), 3.74 (3 H, s), 2.38 (3 H, s).
Intermediate compound 1-10: ¹H-NMR (CDCl₃) δ: 7.54 (1 H, s), 7.27 (1 H, s), 7.24 (1 H, s), 7.05 (1 H, s), 3.86 (3 H, s), 3.74 (3 H, s), 2.33 (3 H, s).

### Reference Preparation Example 2

A mixture of 3.3 g of the intermediate compound 1, bis(pinacolato)diboron 4.7 g, [1,1′-bis(diphenylphosphino)ferrocene]palladium (II) dichloride 0.85 g, tripotassium phosphate 7.4 g, and dimethoxyethane 30 mL was stirred at 80° C. for 10 hours. The resulting mixture was cooled to room temperature, and concentrated under reduced pressure. The resulting residue was subjected to a silica gel column chromatography (ethyl acetate : hexane= 1 : 4) to obtain the intermediate compound 2 represented by the following formula.

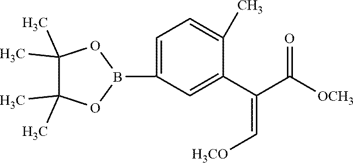 [SMILES: CO/C=C(/C(=O)OC)c1cc(B2OC(C)(C)C(C)(C)O2)ccc1C]

Intermediate compound 2: ¹H-NMR (CDCl₃) δ: 7.67 (1 H, dd), 7.55 (1 H, s), 7.54 (1 H, d), 7.23 (1 H, d), 3.80 (3 H, s), 3.68 (3 H, s), 2.19 (3 H, s), 1.32 (12 H, s).

### Reference Preparation Example 3

A mixture of 5.0 g of the intermediate compound 1, bis(triphenylphosphine)palladium(II) dichloride 0.61 g, tributyl(1-ethoxyvinyl)tin 7.0 mL, and 1,4-dioxane 30 mL was stirred at 100° C. for 5 hours. To the resulting mixture was added 1 N hydrochloric acid, and the mixture was stirred at room temperature for 30 minutes. The resulting mixture was concentrated under reduced pressure and extracted with ethyl acetate. The resulting organic layers were concentrated under reduced pressure and the residue was subjected to a silica gel column chromatography (ethyl acetate : hexane= 2 : 3) to obtain the intermediate compound 3 represented by the following formula 3.41 g.

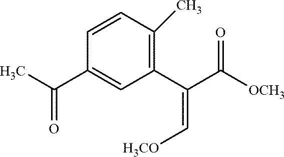 [SMILES: CO/C=C(/C(=O)OC)c1cc(C(C)=O)ccc1C]

Intermediate compound 3: ¹H-NMR (CDCl₃) δ: 7.82 (1 H, dd), 7.71 (1 H, d), 7.60 (1 H, s), 7.31 (1 H, d), 3.84 (3 H, s), 3.71 (3 H, s), 2.57 (3 H, s), 2.24 (3 H, s).

### Reference Preparation Example 4

To a mixture of 1.0 g of the intermediate compound 3, hydroxylamine hydrochloride salt 0.28 g, and methanol 12 mL was added pyridine 0.64 mL at 0° C., and the mixture was stirred at room temperature for 2.5 hours. The resulting mixture was diluted with ethyl acetate, and washed with water and saturated brine successively. The resulting organic layers were dried over anhydrous sodium sulfate, and concentrated under reduced pressure. The precipitated out solids were washed with a mixed solvent of ethyl acetate and hexane (2 : 3) to obtain the intermediate compound 4 represented by the following formula 0.91 g.

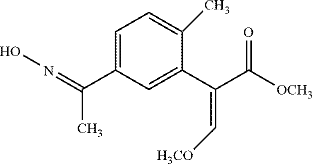 [SMILES: CO/C=C(/C(=O)OC)c1cc(C(C)=NO)ccc1C]

Intermediate compound 4: ¹—H—NMR (CDCl₃) δ: 8.78 (1 H, s), 7.59 (1 H, s), 7.48 (1 H, dd), 7.38 (1 H, d), 7.23 (1 H, d), 3.83 (3 H, s), 3.70 (3 H, s), 2.26 (3 H, s), 2.19 (3 H, s).

### Reference Preparation Example 4-1

The compounds which were prepared according to the Reference Preparation Example 4 and their physical property value are shown below.

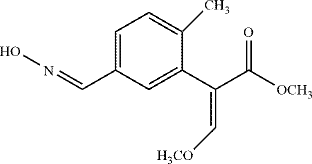 [SMILES: CO/C=C(/C(=O)OC)c1cc(C=NO)ccc1C]

Intermediate compound 4-2: ¹H—NMR (CDCl₃) δ: 9.01 (1 H, br s), 8.11 (1 H, s), 7.59 (1 H, s), 7.45 (1 H, dd), 7.31 (1 H, d), 7.23 (1 H, d), 3.81 (3 H, s), 3.70 (3 H, s), 2.19 (3 H, s).

### Reference Preparation Example 5

To a mixture of methyl 2-(5-bromo-2-methylphenyl)acetate (which was prepared by the method described in WO 2019/016269 A1) 20.0 g, isoamyl nitrite 13.2 mL, and THF 160 mL was added sodium methoxide (28 % in methanol) 25.4 mL, and the mixture was stirred at room temperature for 7 hours. To the resulting mixture was added 1N hydrochloric acid, and the mixture was extracted with ethyl acetate. The resulting organic layers were washed with water and saturated brine successively, dried over sodium sulfate, and concentrated under reduced pressure. The resulting residue was subjected to a silica gel column chromatography (ethyl acetate : hexane = 1:4) to obtain the intermediate compound 5 represented by the following formula 6.51 g.

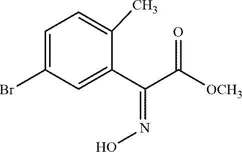

Intermediate compound 5: ¹H—NMR (CDCl₃) δ: 9.25 (1 H, s), 7.45 (1 H, dd), 7.29 (1 H, d), 7.15 (1 H, d), 3.87 (3 H, s), 2.17 (3 H, s) .

### Reference Preparation Example 6

To a mixture of 6.51 g of the intermediate compound 5, potassium carbonate 4.0 g, and DMF 60 mL was added dimethyl sulfate 2.7 mL at 0° C., and the mixture was stirred at room temperature for 2 hours. To the resulting mixture was added aqueous ammonium chloride solution, and the mixture was extracted with MTBE. The resulting organic layers were washed with water and saturated brine successively, dried over anhydrous sodium sulfate, and concentrated under reduced pressure. The resulting residue was subjected to a silica gel column chromatography (ethyl acetate : hexane = 1:9) to obtain the intermediate compound 6 represented by the following formula 4.83 g.

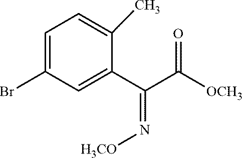 [SMILES: CO/N=C(/C(=O)OC)c1cc(Br)ccc1C]

Intermediate compound 6: ¹H—NMR (CDCl₃ δ: 7.42 (1 H, dd), 7.23 (1 H, d), 7.12 (1 H, d), 4.06 (3 H, s), 3.88 (3 H, s), 2.13 (3 H, s).

### Reference Preparation Example 7

To a mixture of 2.0 g of the intermediate compound 6, and methanol 20 mL was added methylamine (9.8 M in methanol) 1.2 mL, and the mixture was stirred at room temperature overnight. Water was added to the resulting mixture, and the mixture was extracted with ethyl acetate. The resulting organic layers were dried over anhydrous sodium sulfate, and concentrated under reduced pressure. The resulting residue was subjected to a silica gel column chromatography to obtain the intermediate compound represented 7 by the following formula 1.99 g.

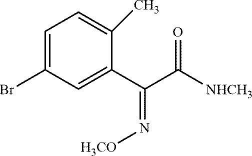 [SMILES: CNC(=O)/C(=N/OC)c1cc(Br)ccc1C]

Intermediate compound 7: ¹H-NMR (CDCl₃) δ: 7.40 (1 H, dd), 7.22 (1 H, d), 7.10 (1 H, d), 6.78 (1 H, br s), 3.97 (3 H, s), 2.94 (3 H, d), 2.12 (3 H, s).

### Reference Preparation Example 8

A mixture of 4.5 g of the intermediate compound 2, methachloro perbenzoic acid 4.0 g, acetonitrile 20 mL, and ethanol 20 mL, and water 20 mL was stirred at room temperature for 15 minutes. To the resulting mixture was added aqueous sodium thiosulfate solution and aqueous sodium hydrogencarbonate solution, and the mixture was stirred at room temperature for 30 minutes. The resulting mixture was concentrated under reduced pressure and extracted with ethyl acetate. The resulting organic layers were concentrated under reduced pressure to obtain the intermediate compound 8 represented by the following formula 4.5 g.

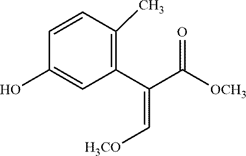 [SMILES: CO/C=C(/C(=O)OC)c1cc(O)ccc1C]

Intermediate compound 8: ¹H—NMR (CDCl₃) δ: 7.54 (1 H, s), 7.08 (1 H, d), 6.71 (1 H, dd), 6.60 (1 H, d), 3.83 (3 H, s), 3.70 (3 H, s), 2.09 (3 H, s).

### Preparation Example 1

A mixture of 0.30 g of the intermediate compound 1, PdCl₂ (PPh₃)₂ 0.04 g, 1-pentyne 0.29 g, tetrabutyl ammonium fluoride (1 mol/L in THF) 3.2 mL and THF 3 mL was stirred at 80° C. under nitrogen atmosphere for 3 hours. To the resulting mixture was added aqueous saturated sodium bicarbonate solution, and the mixture was extracted with ethyl acetate. The resulting organic layers were dried over sodium sulfate, and concentrated under reduced pressure. The residue was subjected to a silica gel column chromatography (ethyl acetate : hexane = 1:2) to obtain the compound of the present invention 1-3 represented by the following formula 0.22 g.

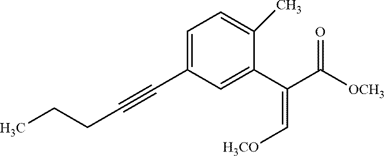 [SMILES: CCCC#Cc1ccc(C)c(/C(=C\OC)C(=O)OC)c1]

Compound of the present invention 1-3: ¹H—NMR (CDCl₃) δ: 7.55 (1 H, s), 7.25-7.23 (1 H, m), 7.15-7.12 (2 H, m), 3.82 (3 H, s), 3.69 (3 H, s), 2.36 (2 H, t), 2.15 (3 H, s), 1.65-1.58 (2 H, m), 1.03 (3 H, t).

### Preparation Example 1-1

The compounds which were prepared according to the Preparation Example 1 and their physical property value are shown below.

A compound of the present invention 1-2 represented by formula (1a):

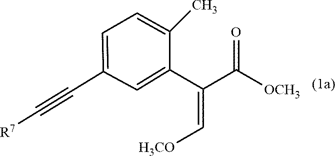

wherein R⁷ represents a substituents described below.

Compound of the present invention 1-2 (R⁷ — c—Pr) :¹H—NMR (CDCl₃) δ: 7.54 (1 H, s), 7.23-7.21 (1 H, m), 7.13-7.10 (2 H, m), 3.81 (3 H, s), 3.68 (3 H, s), 2.14 (3 H, s), 1.46-1.39 (1 H, m), 0.85-0.75 (4 H, m).

### Preparation Example 1-2

The compounds which were prepared according to the Preparation Example 1 and their physical property value are shown below.

A compound represented by formula (2a):

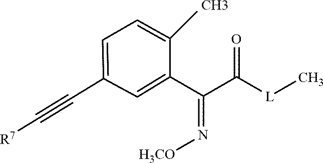

wherein a combination of R⁷ and L represents any combinations described in [Table A9].

TABLE A9

| Compound of the present invention | R⁷ | L |
|---|---|---|
| 1-4 | c-Pr | O |
| 1-5 | t-Bu | O |
| 1-6 | c-Pr | NH |
| 1-7 | t-Bu | NH |

Compound of the present invention 1-4: ¹H—NMR (CDCl₃) δ: 7.30 (1 H, dd), 7.14 (1 H, d), 7.12 (1 H, d), 4.04 (3 H, s), 3.86 (3 H, s), 2.15 (3 H, s), 1.46-1.38 (1 H, m), 0.88-0.81 (2 H, m), 0.80-0.75 (2 H, m).

Compound of the present invention 1-5: ¹H—NMR (CDCl₃) δ: 7.31 (1 H, dd), 7.14 (1 H, d), 7.12 (1 H, d), 4.04 (3 H, s), 3.86 (3 H, s), 2.15 (3 H, s), 1.29 (9 H, s).

Compound of the present invention 1-6: ¹H—NMR (CDCl₃) δ: 7.28 (1 H, dd), 7.13 (1 H, d), 7.11 (1 H, d), 6.71 (1 H, br s), 3.95 (3 H, s), 2.92 (3 H, d), 2.15 (3 H, s), 1.45-1.36 (1 H, m), 0.87-0.79 (2 H, m), 0.79-0.72 (2 H, m).

Compound of the present invention 1-7: ¹H—NMR (CDCl₃) δ: 7.29 (1 H, dd), 7.14-7.11 (2 H, m), 6.72 (1 H, br s), 3.95 (3 H, s), 2.92 (3 H, d), 2.15 (3 H, s), 1.28 (9 H, s).

A compound represented by formula (3a):

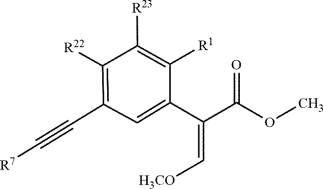

wherein a combination of R¹, R⁷, R²² and R²³ represents any combinations described in [Table A11].

TABLE A11

| Compound of the present invention | R¹ | R⁷ | R22 | R²³ |
|---|---|---|---|---|
| 1-1 | Cl | c-Pr | H | H |
| 1-8 | Cl | t-Bu | H | H |
| 1-9 | OMe | c-Pr | H | H |
| 1-10 | OMe | t-Bu | H | H |
| 1-11 | H | c-Pr | H | Me |
| 1-12 | H | t-Bu | H | Me |
| 1-13 | H | c-Pr | Me | H |

Compound of the present invention 1-1: ¹H—NMR (CDCl₃) δ: 7.55 (1 H, s), 7.31-7.28 (1 H, m), 7.24-7.21 (2 H, m), 3.84 (3 H, s), 3.69 (3 H, s), 1.46-1.38 (1 H, m), 0.89-0.75 (4 H, m).

Compound of the present invention 1-8: ¹H—NMR (CDCl₃) δ: 7.55 (1 H, s), 7.32-7.28 (1 H, m), 7.25-7.22 (2 H, m), 3.84 (3 H, s), 3.69 (3 H, s), 1.29 (9 H, s).

Compound of the present invention 1-9: ¹H—NMR (CDCl₃) δ: 7.49 (1 H, s), 7.31 (1 H, dd), 7.19 (1 H, d), 6.80 (1 H, d), 3.81 (3 H, s), 3.77 (3 H, s), 3.67 (3 H, s), 1.45-1.38 (1 H, m), 0.85-0.74 (4 H, m).

Compound of the present invention 1-10: ¹H—NMR (CDCl₃) δ: 7.50 (1 H, s), 7.31 (1 H, dd), 7.19 (1 H, d), 6.80 (1 H, d), 3.81 (3 H, s), 3.77 (3 H, s), 3.67 (3 H, s), 1.29 (9 H, s).

Compound of the present invention 1-11: ¹H—NMR (CDCl₃) δ: 7.52 (1 H, s), 7.13 (1 H, s), 7.11 (1 H, s), 7.01 (1 H, s), 3.84 (3 H, s), 3.72 (3 H, s), 2.30 (3 H, s), 1.47-1.39 (1 H, m), 0.87-0.81 (2 H, m), 0.81-0.75 (2 H, m).

Compound of the present invention 1-12: ¹H—NMR (CDCl₃) δ: 7.53 (1 H, s), 7.14-7.11 (2 H, m), 7.01-6.98 (1 H, m), 3.84 (3 H, s), 3.72 (3 H, s), 2.30 (3 H, s), 1.29 (9 H, s).

Compound of the present invention 1-13: ¹H—NMR (CDCl₃) δ: 7.52 (1 H, s), 7.30 (1 H, d), 7.17-7.07 (2 H, m), 3.84 (3 H, s), 3.72 (3 H, s), 2.37 (3 H, s), 1.52-1.43 (1 H, m), 0.90-0.83 (2 H, m), 0.82-0.75 (2 H, m).

### Preparation Example 2

A mixture of 0.50 g of the intermediate compound 1, 3-acetylphenylboronic acid 0.43 g, [1,1′-bis(diphenylphosphino)ferrocene]palladium (II) dichloride 0.13 g, tripotassium phosphate 1.12 g, dimethoxyethane 10 mL and water 0.1 mL was stirred at 80° C. under nitrogen atmosphere for 5 hours. The resulting mixture was cooled to room temperature, and then concentrated under reduced pressure. The residue was subjected to a silica gel column chromatography (ethyl acetate : hexane = 1:2) obtain the compound of the present invention 2-1 by the following formula 0.55 g.

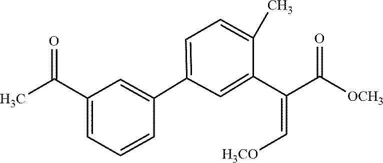 [SMILES: CO/C=C(/C(=O)OC)c1cc(-c2cccc(C(C)=O)c2)ccc1C]

Compound of the present invention 2-1: ¹H—NMR (CDCl₃) δ: 8.17 (1 H, t), 7.90 (1 H, dq), 7.79 (1 H, dq,), 7.62 (1 H, s), 7.53-7.48 (2 H, m), 7.38 (1 H, d), 7.33 (1 H, d), 3.85 (3 H, s), 3.73 (3 H, s), 2.65 (3 H, s), 2.24 (3 H, s).

### Preparation Example 2-1

The compounds which were prepared according to the Preparation Example 2 and their physical property value are shown below.

A compound represented by formula (Ib):

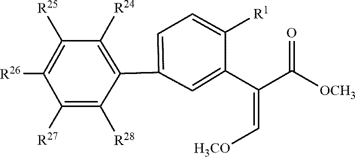

wherein a combination of R¹, R²⁴, R²⁵, R²⁶, R²⁷, and R²⁸ represents any combinations described in [Table A2].

TABLE A2

| Compound of the present invention | R¹ | R²⁴ | R²⁵ | R²⁶ | R²⁷ | R²⁸ |
|---|---|---|---|---|---|---|
| 2-2 | Me | H | H | CF₃ | H | H |
| 2-3 | Me | H | H | H | H | H |
| 2-4 | Me | F | H | H | F | H |
| 2-5 | Me | H | F | H | F | H |
| 2-6 | Me | H | F | F | F | H |
| 2-7 | Me | F | H | H | H | H |
| 2-8 | Me | H | F | H | H | H |
| 2-9 | Me | H | H | OMe | H | H |
| 2-10 | Me | F | H | H | H | F |
| 2-11 | Me | H | H | F | H | H |
| 2-12 | Me | H | H | Cl | H | H |
| 2-13 | Me | H | H | Me | H | H |
| 2-14 | Me | H | CF₃ | H | H | H |
| 2-15 | Me | H | Cl | H | H | H |
| 2-16 | Me | H | CF₃ | H | H | F |
| 2-17 | Me | H | Cl | H | H | F |
| 2-18 | Me | H | CN | H | H | H |
| 2-19 | Me | H | C(═NOMe)Me | H | H | H |
| 2-20 | Cl | H | H | H | H | H |
| 2-21 | Cl | F | H | H | H | H |
| 2-22 | Me | F | F | H | H | H |
| 2-23 | Me | F | H | H | H | OMe |
| 2-24 | Me | F | H | H | H | Cl |

Compound of the present invention 2-2: ¹H—NMR (CDCl₃) δ: 7.70-7.64 (4 H, m), 7.62 (1 H, s), 7.47 (1 H), 7.36-7.32 (2 H, m), 3.85 (3 H, s), 3.73 (3 H, s), 2.24 (3 H, s).

Compound of the present invention 2-3: ¹H—NMR (CDCl₃) δ: 7.61 (1 H, s), 7.60-7.56 (2 H, m), 7.47-7.39 (3 H, m), 7.35-7.29 (3 H, m), 3.84 (3 H, s), 3.72 (3 H, s), 2.22 (3 H, s).

Compound of the present invention 2-4: ¹H—NMR (CDCl₃) δ: 7.60 (1 H, s), 7.41 (1 H, dt), 7.33-7.29 (2 H, m) , 7.17-7.13 (1 H, m), 7.10-7.04 (1 H, m), 7.00-6.92 (1 H, m), 3.85 (3 H, s), 3.72 (3 H, s), 2.23 (3 H, s).

Compound of the present invention 2-5: ¹H—NMR (CDCl₃) δ: 7.61 (1 H, s), 7.41 (1 H, dd), 7.32-7.30 (2 H, m), 7.12-7.08 (2 H, m), 6.79-6.72 (1 H, m), 3.85 (3 H, s), 3.72 (3 H, s), 2.22 (3 H, s) .

Compound of the present invention 2-6: ¹H—NMR (CDCl₃) δ: 7.61 (1 H, s), 7.36 (1 H, dd), 7.32-7.30 (1 H, m), 7.26-7.25 (1 H, m), 7.18 (2 H, dd), 3.86 (3 H, s), 3.73 (3 H, s), 2.23 (3 H, s).

Compound of the present invention 2-7: ¹H—NMR (CDCl₃)δ: 7.60 (1 H, s), 7.47-7.42 (2 H, m), 7.31-7.25 (3 H, m), 7.20-7.10 (2 H, m), 3.84 (3 H, s), 3.72 (3 H, s), 2.23 (3 H, s).

Compound of the present invention 2-8: ¹H—NMR (CDCl₃) δ: 7.61 (1 H, s), 7.44 (1 H, dd), 7.37-7.29 (5 H, m), 7.02-6.97 (1 H, m), 3.85 (3 H, s), 3.72 (3 H, s), 2.22 (3 H, s).

Compound of the present invention 2-9: ¹H—NMR (CDCl₃) δ: 7.60 (1 H, s), 7.53-7.49 (2 H, m), 7.41 (1 H, dd), 7.30 (1 H, d), 7.28-7.26 (1 H, m), 6.97-6.93 (2 H, m), 3.84 (3 H, s), 3.83 (3 H, s), 3.72 (3 H, s), 2.21 (3 H, s) .

Compound of the present invention 2-10: ¹H—NMR (CDCl₃) δ: 7.59 (1 H, s), 7.35-7.30 (2 H, m), 7.27-7.19 (2 H, m), 6.99-6.91 (2 H, m), 3.82 (3 H, s), 3.71 (3 H, s), 2.23 (3 H, s).

Compound of the present invention 2-11: ¹H—NMR (CDCl₃) δ: 7.60 (1 H, s), 7.54-7.51 (2 H, m), 7.40 (1 H, dd), 7.29-7.27 (2 H, m), 7.11-7.06 (2 H, m), 3.83 (3 H, s), 3.71 (3 H, s), 2.21 (3 H, s).

Compound of the present invention 2-12: ¹H—NMR (CDCl₃) δ: 7.60 (1 H, s), 7.51-7.48 (2 H, m), 7.42-7.27 (5 H, m), 3.81 (3 H, s), 3.71 (3 H, s), 2.21 (3 H, s).

Compound of the present invention 2-13: ¹H—NMR (CDCl₃) δ: 7.60 (1 H, s), 7.49-7.43 (3 H, m), 7.33-7.21 (4 H, m), 3.83 (3 H, s), 3.71 (3 H, s), 2.38 (3 H, s), 2.21 (3 H, s).

Compound of the present invention 2-14: ¹H—NMR (CDCl₃) δ: 7.82 (1 H, s), 7.76 (1 H, d), 7.62 (1 H, s), 7.57-7.50 (2 H, m), 7.46 (1 H, dd), 7.35-7.32 (2 H, m), 3.85 (3 H, s), 3.73 (3 H, s), 2.24 (3 H, s).

Compound of the present invention 2-15: ¹H—NMR (CDCl₃) δ: 7.61 (1 H, s), 7.56 (1 H, t), 7.47-7.42 (2 H, m), 7.35-7.26 (4 H, m), 3.84 (3 H, s), 3.72 (3 H, s), 2.22 (3 H, s).

Compound of the present invention 2-16: ¹H—NMR (CDCl₃) δ: 7.72 (1 H, d), 7.61 (1 H, s), 7.57-7.53 (1 H, m), 7.43 (1 H, d), 7.34-7.30 (2 H, m), 7.23-7.21 (1 H, m), 3.85 (3 H, s), 3.72 (3 H, s), 2.24 (3 H, s).

Compound of the present invention 2-17: ¹H—NMR (CDCl₃) δ: 7.60 (1 H, s), 7.44-7.39 (2 H, m), 7.31-7.28 (2 H, m), 7.25-7.21 (1 H, m), 7.09-7.04 (1 H, m), 3.85 (3 H, s), 3.72 (3 H, s), 2.23 (3 H, s).

Compound of the present invention 2-18: ¹H—NMR (CDCl₃) δ: 7.86 (1 H, t), 7.81 (1 H, dq), 7.62 (1 H, s), 7.59 (1 H, dt), 7.51 (1 H, t), 7.43 (1 H, dd), 7.34-7.32 (2 H, m), 3.86 (3 H, s), 3.73 (3 H, s), 2.24 (3 H, s).

Compound of the present invention 2-19: ¹H—NMR (CDCl₃) δ: 7.83 (1 H, t), 7.61 (1 H, s), 7.60-7.55 (2 H, m), 7.47 (1 H, dd), 7.40 (1 H, t), 7.35 (1 H, d), 7.30 (1 H, d), 4.01 (3 H, s), 3.83 (3 H, s), 3.72 (3 H, s), 2.26 (3 H, s), 2.23 (3 H, s).

Compound of the present invention 2-20: ¹H—NMR (CDCl₃) δ: 7.61 (1 H, s), 7.58-7.55 (2 H, m), 7.48-7.32 (6 H, m), 3.86 (3 H, s), 3.73 (3 H, s).

Compound of the present invention 2-21: ¹H—NMR (CDCl₃) δ: 7.60 (1 H, s), 7.50-7.41 (4 H, m), 7.34-7.29 (1 H, m), 7.22-7.12 (2 H, m), 3.87 (3 H, s), 3.73 (3 H, s).

Compound of the present invention 2-22: ¹H—NMR (CDCl₃) δ: 7.60 (1 H, s), 7.42 (1 H, dt), 7.32-7.30 (2 H, m), 7.22-7.18 (1 H, m), 7.12-7.08 (2 H, m), 3.85 (3 H, s), 3.72 (3 H, s), 2.23 (3 H, s).

Compound of the present invention 2-23: ¹H—NMR (CDCl₃) δ: 7.58 (1 H, s), 7.29-7.28 (2 H, m), 7.24-7.19 (2 H, m), 6.79-6.74 (2 H, m), 3.83 (3 H, s), 3.77 (3 H, s), 3.71 (3 H, s), 2.22 (3 H, s).

Compound of the present invention 2-24: ¹H—NMR (CDCl₃) δ: 7.59 (1 H, s), 7.34-7.28 (2 H, m), 7.25-7.20 (2 H, m), 7.15-7.14 (1 H, m), 7.08-7.03 (1 H, m), 3.84 (3 H, s), 3.71 (3 H, s), 2.25 (3 H, s).

A compound represented by formula (1c):

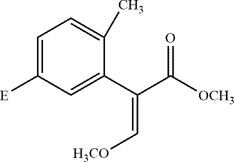

wherein E represents a substituent described in [Table A3].

TABLE A3

| Compound of the present invention | E |
|---|---|
| 3-1 | 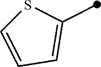 |
| 3-2 | 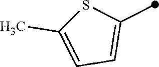 |
| 3-3 | 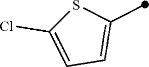 |
| 3-4 | 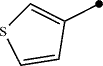 |
| 3-5 | 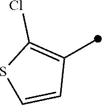 |
| 3-6 | 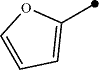 |
| 3-7 | 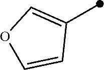 |

Compound of the present invention 3-1: ¹H—NMR (CDCl₃) δ: 7.60 (1 H, s), 7.46 (1 H, dd), 7.35 (1 H, d,), 7.25-7.22 (3 H, m), 7.05 (1 H, dd), 3.84 (3 H, s), 3.71 (3 H, s), 2.19 (3 H, s). the compound of the present invention 3-2 : ¹H—NMR (CDCl₃) δ: 7.59 (1 H, s), 7.39 (1 H, dd), 7.29 (1 H, d), 7.20 (1 H, d), 7.04 (1 H, d), 6.70-6.68 (1 H, m), 3.83 (3 H, s), 3.71 (3 H, s), 2.49 (3 H, d), 2.17 (3 H, s).

Compound of the present invention 3-3: ¹H—NMR (CDCl₃) δ: 7.59 (1 H, s), 7.35 (1 H, dd), 7.25-7.21 (2 H, m), 7.01 (1 H, d), 6.85 (1 H, d), 3.84 (3 H, s), 3.71 (3 H, s), 2.18 (3 H, s).

Compound of the present invention 3-4: ¹H—NMR (CDCl₃) δ: 7.60 (1H, s), 7.45 (1 H, dd), 7.40-7.39 (1 H, m), 7.37-7.34 (3 H, m), 7.26-7.24 (1 H, m), 3.84 (3 H, s), 3.71 (3 H, s), 2.20 (3 H, s) .

Compound of the present invention 3-5: ¹H—NMR (CDCl₃) δ: 7.59 (1 H, s), 7.44 (1 H, dd), 7.33 (1 H, d), 7.29 (1 H, d), 7.11 (1 H, d), 7.05 (1 H, d), 3.84 (3 H, s), 3.72 (3 H, s), 2.22 (3 H, s).

Compound of the present invention 3-6: ¹H—NMR (CDCl₃) δ: 7.59 (1 H, s), 7.52 (1 H, dd), 7.43-7.42 (2 H, m), 7.26-7.23 (1 H, m), 6.58 (1 H, d), 6.44 (1 H, dd), 3.83 (3 H, s), 3.71 (3 H, s), 2.19 (3 H, s).

Compound of the present invention 3-7: ¹H—NMR (CDCl₃) δ: 7.69-7.68 (1 H, m), 7.59 (1 H, s), 7.45-7.44 (1 H, m), 7.34 (1 H, dd), 7.24-7.22 (2 H, m), 6.68-6.67 (1 H, m), 3.83 (3 H, s), 3.71 (3 H, s), 2.19 (3 H, s).

### Preparation Example 2-2

The compounds which can be prepared according to the Preparation Example 2 are shown below.

A compound represented by formula (1b) wherein a combination of R¹, R²⁴, R²⁵, R²⁶, R²⁷, and R²⁸ represents any combinations described in [Table A10].

TABLE A10

| Compound of the present invention | R¹ | R²⁴ | R²⁵ | R²⁶ | R²⁷ | R²⁸ |
|---|---|---|---|---|---|---|
| 2-28 | Me | OMe | H | H | H | H |
| 2-29 | Me | Me | H | H | H | H |
| 2-30 | Me | H | c-Pr | H | H | H |
| 2-31 | Me | H | OPh | H | H | H |

### Preparation Example 2-3

The compounds which were prepared according to the Preparation Example 2 and their physical property value are shown below.

A compound represented by formula (1b) wherein a combination of R¹, R²⁴, R²⁵, R²⁶, R²⁷, and R²⁸ represents any combinations described in [Table A12].

TABLE A12

| Compound of the present invention | R¹ | R²⁴ | R²⁵ | R²⁶ | R²⁷ | R²⁸ |
|---|---|---|---|---|---|---|
| 2-25 | Me | F | F | H | H | F |
| 2-26 | Me | F | F | H | F | F |
| 2-27 | Me | F | F | H | F | H |
| 2-32 | Me | H | F | F | H | H |
| 2-33 | Me | F | H | F | H | H |
| 2-34 | Me | F | F | F | H | H |
| 2-35 | Me | F | F | F | F | F |
| 2-36 | Cl | H | H | F | H | H |
| 2-37 | Cl | H | Cl | H | H | H |
| 2-38 | Cl | H | F | H | H | H |
| 2-39 | OMe | H | H | F | H | H |
| 2-40 | OMe | F | H | H | H | H |
| 2-43 | Me | H | OMe | H | H | H |

Compound of the present invention 2-25: ¹H—NMR (CDCl₃) δ: 7.60 (1 H, s), 7.33 (2 H, s), 7.24 (1 H, s), 7.12-7.04 (1 H, m), 6.92-6.86 (1 H, m), 3.85 (3 H, s), 3.72 (3 H, s), 2.24 (3 H, s).

Compound of the present invention 2-26: ¹H—NMR (CDCl₃) δ: 7.60 (1 H, s), 7.36-7.33 (2 H, m), 7.24 (1 H, s), 7.07-6.98 (1 H, m), 3.85 (3 H, s), 3.72 (3 H, s), 2.25 (3 H, s).

Compound of the present invention 2-27: ¹H—NMR (CDCl₃) δ: 7.60 (1 H, s), 7.40 (1 H, d), 7.33-7.25 (2 H, m), 6.97-6.84 (2 H, m), 3.85 (3 H, s), 3.72 (3 H, s), 2.24 (3 H, s).

Compound of the present invention 2-32: ¹H—NMR (CDCl₃) δ: 7.61 (1 H, s), 7.41-7.34 (2 H, m), 7.31-7.26 (3 H, m), 7.22-7.15 (1 H, m), 3.85 (3 H, s), 3.72 (3 H, s), 2.22 (3 H, s).

Compound of the present invention 2-33: ¹H—NMR (CDCl₃) δ: 7.60 (1 H, s), 7.44-7.36 (2 H, m), 7.31-7.26 (2 H, m), 6.94-6.85 (2 H, m), 3.84 (3 H, s), 3.72 (3 H, s), 2.22 (3 H, s).

Compound of the present invention 2-34: ¹H—NMR (CDCl₃) δ: 7.60 (1 H, s), 7.41-7.30 (2 H, m), 7.26-7.24 (1 H, m), 7.18-7.12 (1 H, m), 7.03-6.96 (1 H, m), 3.85 (3 H, s), 3.72 (3 H, s), 2.23 (3 H, s).

Compound of the present invention 2-35: ¹H—NMR (CDCl₃) δ: 7.60 (1 H, s), 7.36-7.27 (2 H, m), 7.20 (1 H, s), 3.85 (3 H, s), 3.72 (3 H, s), 2.25 (3 H, s).

Compound of the present invention 2-36: ¹H—NMR (CDCl₃) δ: 7.61 (1 H, s), 7.55-7.38 (5 H, m), 7.14-7.08 (2 H, m), 3.87 (3 H, s), 3.74 (3 H, s).

Compound of the present invention 2-37: ¹H—NMR (CDCl₃) δ: 7.62 (1 H, s), 7.55 (1 H, t), 7.51-7.41 (4 H, m), 7.38-7.30 (2 H, m), 3.87 (3 H, s), 3.74 (3 H, s).

Compound of the present invention 2-38: ¹H—NMR (CDCl₃) δ: 7.62 (1 H, s), 7.50-7.33 (5 H, m), 7.29-7.24 (1 H, m), 7.07-7.01 (1 H, m), 3.87 (3 H, s), 3.74 (3 H, s).

Compound of the present invention 2-39: ¹H—NMR (CDCl₃) δ: 7.55 (1 H, s), 7.53-7.44 (3 H, m), 7.36 (1 H, d), 7.09 (2 H, t), 6.98 (1 H, d), 3.83 (3 H, s), 3.83 (3 H, s), 3.72 (3 H, s).

Compound of the present invention 2-40: ¹H—NMR (CDCl₃) δ: 7.54 (1 H, s), 7.54-7.50 (1 H, m), 7.44 (1 H, td), 7.39 (1 H, t), 7.30-7.24 (1 H, m), 7.17 (1 H, td), 7.12 (1 H, m), 6.99 (1 H, d), 3.84 (3 H, s), 3.83 (3 H, s), 3.72 (3 H, s).

Compound of the present invention 2-43: ¹H—NMR (CDCl₃) δ: 7.61 (1 H, s), 7.45 (1 H, dd), 7.34-7.28 (3 H, m), 7.18 (1 H, dt) , 7.12 (1 H, t), 6.86 (1 H, dd), 3.85 (3 H, s), 3.84 (3 H, s), 3.72 (3 H, s), 2.22 (3 H, s).

A compound represented by formula (2b):

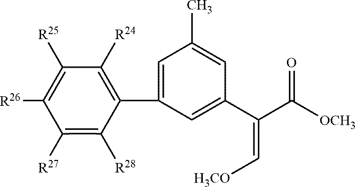

wherein a combination of R²⁴, R²⁵, R²⁶, R²⁷, and R²⁸ represents any combinations described in [Table A13].

TABLE A13

| Compound of the present invention | R²⁴ | R²⁵ | R²⁶ | R²⁷ | R²⁸ |
|---|---|---|---|---|---|
| 2-41 | H | H | F | H | H |
| 2-42 | F | H | H | H | H |

Compound of the present invention 2-41: ¹H—NMR (CDCl₃) δ: 7.59 (1 H, s), 7.56-7.50 (2 H, m), 7.28 (2 H, d), 7.14-7.07 (3 H, m), 3.87 (3 H, s), 3.75 (3 H, s), 2.42 (3 H, s).

Compound of the present invention 2-42: ¹H—NMR (CDCl₃) δ: 7.58 (1 H, s), 7.44 (1 H, td), 7.33-7.27 (3 H, m), 7.21-7.10 (3 H, m), 3.86 (3 H, s), 3.75 (3 H, s), 2.42 (3 H, s).

### Preparation Example 3

A mixture of 0.30 g of the intermediate compound 2, 2-bromopyridine 0.21 g, [1,1′-bis(diphenylphosphino)ferrocene]palladium (II) dichloride 0.07 g, tripotassium phosphate 0.27 g, dimethoxyethane 6 mL and water 0.1 mL was stirred at 80° C. under nitrogen atmosphere for 5 hours. The resulting mixture was cooled to room temperature, and then concentrated under reduced pressure. The residue was subjected to a silica gel column chromatography (ethyl acetate : hexane = 1:3) to obtain the compound of the present invention 4-1 represented by the following formula 0.15 g.

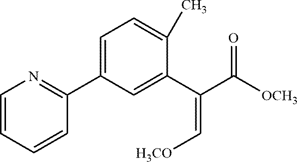 [SMILES: CO/C=C(/C(=O)OC)c1cc(-c2ccccn2)ccc1C]

Compound of the present invention 4-1: ¹H—NMR (CDCl₃) δ: 8.66 (1 H, td), 7.85 (1 H, dd), 7.76 (1 H, d), 7.72-7.70 (2 H, m), 7.60 (1 H, s), 7.33 (1 H, d), 7.20-7.16 (1 H, m), 3.83 (3 H, s), 3.70 (3 H, s), 2.23 (3 H, s).

### Preparation Example 3-1

The compounds which were prepared according to the Preparation Example 3 and their physical property value are shown below.

A compound represented by formula (1d):

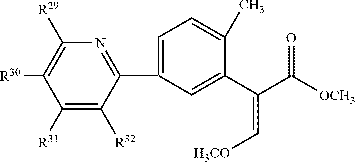

wherein a combination of R²⁹, R³⁰, R³¹, _{and} R³² represents any combinations described in [Table A4].

TABLE A4

| Compound of the present invention | R²⁹ | R³⁰ | R³¹ | R³² |
|---|---|---|---|---|
| 4-2 | Me | H | H | H |
| 4-3 | F | H | H | H |
| 4-4 | CF₃ | H | H | H |
| 4-5 | H | F | H | H |
| 4-6 | H | H | H | F |
| 4-7 | H | CF₃ | H | H |
| 4-8 | OMe | H | H | H |

Compound of the present invention 4-2: ¹H—NMR (CDCl₃) δ: 7.84 (1 H, dd), 7.73 (1 H, d), 7.61-7.57 (2 H, m), 7.49 (1 H, d), 7.31 (1 H, d), 7.04 (1 H, d), 3.82 (3 H, s), 3.70 (3 H, s), 2.60 (3 H, s), 2.22 (3 H, s).

Compound of the present invention 4-3: ¹H—NMR (CDCl₃) δ: 7.86 (1 H, dd), 7.83-7.77 (2 H, m), 7.61-7.59 (2 H, m), 7.32 (1 H, d), 6.81 (1 H, dd), 3.83 (3 H, s), 3.71 (3 H, s), 2.23 (3 H, s). Compound of the present invention 4-4: ¹H—NMR (CDCl₃) δ: 7.94 (1 H, dd), 7.90-7.84 (2 H, m), 7.80 (1 H, d), 7.62 (1 H, s), 7.55 (1 H, dd), 7.35 (1 H, d), 3.84 (3 H, s), 3.71 (3 H, s), 2.24 (3 H, s).

Compound of the present invention 4-5: ¹H—NMR (CDCl₃) δ: 8.51 (1 H, d), 7.80 (1 H,dd), 7.71-7.68 (2 H, m), 7.60 (1 H, s), 7.45-7.40 (1 H, m), 7.32 (1 H, d), 3.83 (3 H, s), 3.70 (3 H, s), 2.23 (3 H, s).

Compound of the present invention 4-6: ¹H—NMR (CDCl₃) δ: 8.48 (1 H, td), 7.86 (1 H, dt), 7.77 (1 H, t), 7.60 (1 H, s), 7.43 (1 H, ddd), 7.33 (1 H, d), 7.22-7.18 (1 H, m), 3.81 (3 H, s), 3.69 (3 H, s), 2.24 (3 H, s).

Compound of the present invention 4-7: ¹H—NMR (CDCl₃) δ: 8.91 (1 H, dd), 7.94-7.89 (2 H, m), 7.83-7.80 (2 H, m), 7.62 (1 H, s), 7.36 (1 H, d), 3.83 (3 H, s), 3.71 (3 H, s), 2.25 (3 H, s). Compound of the present invention 4-8: ¹H-NMR (CDCl₃) δ: 7.92 (1 H, dd), 7.76 (1 H, d), 7.61-7.57 (2 H, m), 7.31 (2 H, dd), 6.64 (1 H, dd), 4.02 (3 H, s), 3.83 (3 H, s), 3.71 (3 H, s), 2.23 (3 H, s).

The compound represented by formula (1c) wherein E represents a substituent described in [Table A5] . [0137]

TABLE A5

| Compound present of invention the | E |
|---|---|
| 5-1 | 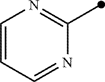 |
| 5-2 | 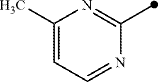 |
| 5-3 | 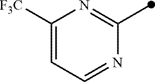 |
| 5-4 | 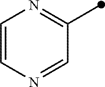 |
| 5-5 | 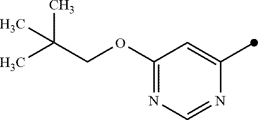 |
| 5-6 | 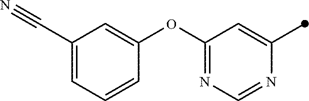 |
| 5-7 | 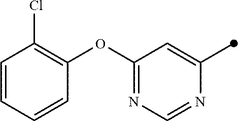 |
| 5-8 | 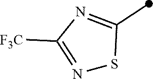 |
| 5-9 | 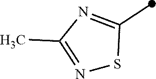 |
| 5-10 | 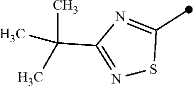 |
| 5-11 | 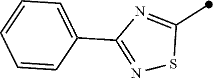 |
| 5-12 | 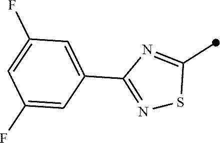 |
| 5-13 | 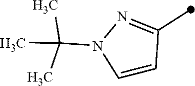 |
| 5-14 | 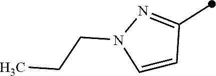 |
| 5-15 | 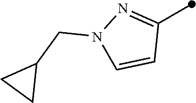 |

TABLE A5 (Continued)

| | |
|---|---|
| 5-16 | 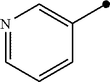 |
| 5-17 | 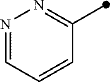 |
| 5-18 | 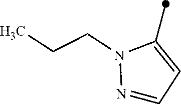 |
| 5-19 | 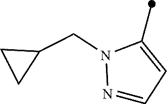 |
| 5-20 | 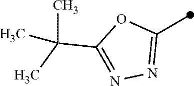 |

Compound of the present invention 5-1: ¹H—NMR (CDCl₃) δ: 8.77 (2 H, d), 8.31 (1 H, d), 8.20 (1 H, s), 7.61 (1 H, s), 7.35 (1 H, d), 7.15-7.11 (1 H, m), 3.82 (3 H, s), 3.70 (3 H, s), 2.25 (3 H, s) .

Compound of the present invention 5-2: ¹H—NMR (CDCl₃) δ: 8.61 (1 H, d), 8.31 (1 H, dd), 8.19 (1 H, d), 7.60 (1 H, s), 7.33 (1 H, d), 7.00 (1 H, d), 3.82 (3 H, s), 3.69 (3 H, s), 2.56 (3 H, s), 2.24 (3 H, s).

Compound of the present invention 5-3: ¹H—NMR (CDCl₃) δ: 8.99 (1 H, d), 8.37 (1 H, dd), 8.25 (1 H, d), 7.63 (1 H, d), 7.45 (1 H, d), 7.37 (1 H, t), 3.84 (3 H, s), 3.71 (3 H, s), 2.26 (3 H, s).

Compound of the present invention 5-4: ¹H—NMR (CDCl₃) δ: 9.01 (1 H, d), 8.60 (1 H, dd), 8.46 (1 H, d), 7.89 (1 H, dd), 7.79 (1 H, d), 7.62 (1 H, s), 7.37 (1 H, d), 3.85 (3 H, s), 3.72 (3 H, s), 2.25 (3 H, s).

Compound of the present invention 5-5: ¹H—NMR (CDCl₃) δ: 8.79 (1 H, d), 7.91 (1 H, dd), 7.81 (1 H, d), 7.61 (1 H, s), 7.34 (1 H, d), 7.11 (1 H, d), 4.07 (2 H, s), 3.84 (3 H, s), 3.71 (3 H, s), 2.24 (3 H, s), 1.05 (9 H, s).

Compound of the present invention 5-6: ¹H—NMR (CDCl₃) δ: 8.80 (1 H, d), 7.95 (1 H,dd), 7.85 (1 H, d), 7.63-7.31 (7 H, m), 3.85 (3 H, s), 3.72 (3 H, s), 2.27 (3 H, s).

Compound of the present invention 5-7: ¹H—NMR (CDCl₃) δ: 8.79 (1 H,s), 7.94 (1 H, dd), 7.89-7.85 (2 H, m), 7.62 (1 H,s), 7.52-7.45 (2 H, m), 7.41-7.34 (3 H, m), 3.85 (3 H, s), 3.71 (3 H, s), 2.26 (3 H, s).

Compound of the present invention 5-8: ¹H—NMR (CDCl₃) δ: 7.84 (1 H, d), 7.75 (1 H, s), 7.63 (1 H, s), 7.38 (1 H, d), 3.86 (3 H, s), 3.72 (3 H, s), 2.27 (3 H, s).

Compound of the present invention 5-9: ¹H-NMR (CDCl₃) δ: 7.78 (1 H, dd), 7.70 (1 H, d), 7.61 (1 H, s), 7.34 (1 H, d), 3.85 (3 H, s), 3.71 (3 H, s), 2.71 (3 H, s), 2.24 (3 H, s).

Compound of the present invention 5-10: ¹H—NMR (CDCl₃) δ: 7.80 (1 H, dd), 7.69 (1 H, d), 7.62 (1 H, s), 7.32 (1 H, d), 3.85 (3 H, s), 3.72 (3 H, s), 2.24 (3 H, s), 1.48 (9 H, s). Compound of the present invention 5-11: ¹H—NMR (CDCl₃) δ: 8.39-8.37 (2 H, m), 7.89 (1 H, dd) , 7.80 (1 H, d), 7.64 (1 H, s), 7.52-7.47 (3 H, m), 7.37 (1 H, d), 3.86 (3 H, s), 3.73 (3 H, s), 2.26 (3 H, s).

Compound of the present invention 5-12: ¹H—NMR (CDCl₃) δ: 7.93-7.86 (3 H, m), 7.79 (1 H, d), 7.65 (1 H, s), 7.38 (1 H, d), 6.92 (1 H, tt) , 3.87 (3 H, s), 3.74 (3 H, s), 2.27 (3 H, s).

Compound of the present invention 5-13: ¹H—NMR (CDCl₃) δ: 7.67 (1 H, dd), 7.58 (1 H, s), 7.54 (1 H, d), 7.49 (1 H, d), 7.22 (1 H, d), 6.46 (1 H, d), 3.81 (3 H, s), 3.69 (3 H, s), 2.18 (3 H, s), 1.61 (9 H, s).

Compound of the present invention 5-14: ¹H—NMR (CDCl₃) δ: 7.64 (1 H, dd), 7.57 (1 H, s), 7.54 (1 H, d), 7.36 (1 H, d), 7.25-7.22 (1 H, m), 6.49 (1 H, d), 4.10 (2 H, t), 3.81 (3 H, s), 3.69 (3 H, s), 2.19 (3 H, s), 1.96-1.87 (2 H, m), 0.93 (3 H, t). Compound of the present invention 5-15: ¹H—NMR (CDCl₃) δ: 7.59 (1 H, s), 7.53 (1 H, d), 7.32-7.27 (1 H, m), 7.24 (1 H, dd), 7.15 (1 H, d), 6.27 (1 H, d), 4.00 (2 H, d), 3.85 (3 H, s), 3.72 (3 H, s), 2.23 (3 H, s), 1.24-1.18 (1 H, m), 0.50-0.45 (2 H, m), 0.22-0.18 (2 H, m).

Compound of the present invention 5-16: ¹H—NMR (CDCl₃) δ: 8.84 (1 H, dd), 8.55 (1 H, dd), 7.88-7.85 (1 H, m), 7.62 (1 H, s), 7.45 (1 H, dd), 7.35-7.31 (3 H, m), 3.85 (3 H, s), 3.73 (3 H, s), 2.24 (3 H, s).

Compound of the present invention 5-17: ¹H—NMR (CDCl₃) δ: 9.12 (1 H, dd), 7.96 (1 H, dd), 7.87-7.83 (2 H, m), 7.62 (1 H, s), 7.49 (1 H, dd), 7.39 (1 H, d), 3.84 (3 H, s), 3.71 (3 H, s), 2.26 (3 H, s).

Compound of the present invention 5-18: ¹H—NMR (CDCl₃) δ: 7.60 (1 H, s), 7.52 (1 H, d), 7.30 (1 H, d), 7.23 (1 H, dd), 7.14 (1 H, d), 6.25 (1 H, d), 4.10-4.06 (2 H, m), 3.85 (3 H, s), 3.72 (3 H, s), 2.24 (3 H, s), 1.89-1.80 (2 H, m), 0.84 (3 H, t).

Compound of the present invention 5-19: ¹H—NMR (CDCl₃) (5: 7.64 (1 H, dd), 7.58 (1 H, s), 7.55-7.54 (1 H, m), 7.50 (1 H, d), 7.24 (1 H, d), 6.51 (1 H, d), 4.02 (2 H, d), 3.81 (3 H, s), 3.69 (3 H, s), 2.19 (3 H, s), 1.35-1.30 (1 H, m), 0.67-0.62 (2 H, m), 0.40-0.36 (2 H, m) .

Compound of the present invention 5-20: ¹H—NMR (CDCl₃) δ: 7.89 (1 H, dd), 7.77 (1 H, d), 7.61 (1 H, s), 7.35 (1 H, d), 3.85 (3 H, s), 3.71 (3 H, s), 2.24 (3 H, s), 1.47 (9 H, s).

### Preparation Example 4

A mixture of 0.30 g of the intermediate compound 1, pyrazole 0.1 mL, copper (I) iodide 0.20 g, potassium carbonate 0.29 g, trans-N,N-dimethylcyclohexane-1,2-diamine 0.17 mL and DMF 10 mL was stirred at 120° C. under nitrogen atmosphere for 5 hours. The resulting mixture was cooled to room temperature, and then concentrated under reduced pressure. The resulting residue was subjected to a silica gel column chromatography (hexane : ethyl acetate = 1:4) to obtain the compound of the present invention 6-1 represented by the following formula 0.20 g.

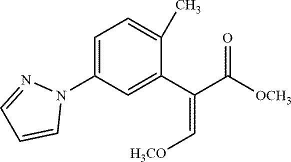 [SMILES: CO/C=C(/C(=O)OC)c1cc(-n2cccn2)ccc1C]

Compound of the present invention 6-1: ¹H—NMR (CDCl₃) δ: 7.89 (1 H, d), 7.69 (1 H, d), 7.60 (1 H, s), 7.53 (1 H, dd), 7.45 (1 H, d), 7.29 (1 H, d), 6.43 (1 H, t), 3.84 (3 H, s), 3.71 (3 H, s), 2.20 (3 H, s).

### Preparation Example 4-1

The compounds which were prepared according to the Preparation Example 4 and their physical property value are shown below.

A compound represented by formula (If):

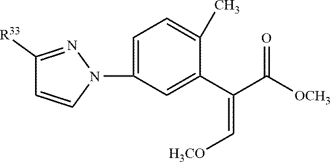

wherein R³³ represents a substituent described in [Table A6] .

TABLE A6

| Compound of the present invention | R³³ |
|---|---|
| 6-2 | c-Pr |
| 6-3 | t-Bu |
| 6-4 | Me |
| 6-5 | CF₃ |
| 6-6 | 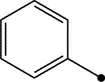 |
| 6-7 | 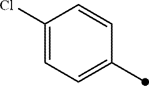 |
| 6-8 | 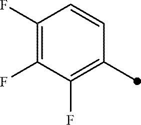 |

Compound of the present invention 6-2: ¹H—NMR (CDCl₃) δ: 7.74 (1 H, d), 7.58 (1 H, s), 7.48 (1 H, dd), 7.40 (1 H, d), 7.24 (1 H, s), 6.05 (1 H, d) , 3.83 (3 H, s), 3.70 (3 H, s), 2.18 (3 H, s) , 2.05-2.00 (1 H, m), 0.98-0.93 (2 H, m), 0.80-0.76 (2 H, m).

Compound of the present invention 6-3: ¹H—NMR (CDCl₃) δ: 7.74 (1 H, d), 7.59 (1 H, s), 7.51 (1 H, dd), 7.39 (1 H, d), 7.26-7.24 (1 H, m), 6.27 (1 H, d), 3.83 (3 H,s), 3.70 (3 H, s), 2.18 (3 H, s), 1.36 (9 H, s).

Compound of the present invention 6-4: ¹H—NMR (CDCl₃) δ: 7.77 (1 H, d), 7.58 (1 H, s), 7.48 (1 H, dd), 7.41 (1 H, d), 7.27-7.25 (1 H, m), 6.21 (1 H, d), 3.83 (3 H, s), 3.70 (3 H, s), 2.37 (3 H, s), 2.19 (3 H, s).

Compound of the present invention 6-5: ¹H—NMR (CDCl₃) δ: 7.90 (1 H, d), 7.60 (y s), 7.54 (1 H, dd), 7.45 (1 H, d), 7.32 (1 H, d), 6.68 (1 H, d), 3.85 (3 H, s), 3.71 (3 H, s), 2.22 (3 H, s) . Compound of the present invention 6-6: ¹H—NMR (CDCl₃) δ: 7.92-7.89 (3 H, m), 7.62-7.60 (2 H, m), 7.52 (1 H, d), 7.44-7.37 (2 H, m), 7.34-7.29 (2 H, m), 6.74 (1 H, d), 3.85 (3 H, s), 3.72 (3 H, s), 2.21 (3 H, s).

Compound of the present invention 6-7: ¹H—NMR (CDCl₃) δ: 7.91 (1 H, d), 7.84 (2 H, dt) , 7.61 (1 H, s), 7.59 (1 H, dd), 7.51 (1 H, d), 7.38 (2 H, dt), 7.31 (1 H, d), 6.71 (1 H, d), 3.85 (3 H, s), 3.72 (3 H, s), 2.21 (3 H, s).

Compound of the present invention 6-8: ¹H—NMR (CDCl₃) δ: 7.94 (1 H, d), 7.90-7.84 (1 H, m), 7.61 (1 H, s), 7.59 (1 H, dd), 7.50 (1 H, d), 7.31 (1 H, d), 7.06-6.99 (1 H, m), 6.85 (1 H, dd), 3.85 (3 H, s), 3.72 (3 H, s), 2.22 (3 H, s).

### Preparation Example 5

To a mixture of 1-(4-chlorophenyl)-1H-pyrazole-3-ol 0.95 g and chloroform 10 mL were added trifluoroacetic anhydride 0.88 mL and triethylamine 0.74 mL at 0° C. successively, and the mixture was stirred at room temperature for 1 hour. Water was added to the resulting mixture, and the mixture was extracted with ethyl acetate. The resulting organic layers were washed with water and saturated brine successively, dried over anhydrous sodium sulfate, and concentrated under reduced pressure. To the resulting residue were added 0.8 g of the intermediate compound 2, [1,1′-bis(diphenylphoshino)ferrocene]palladium (II) dichloride 0.18 g, tripotassium phosphate 1.53 g, and dimethoxyethane 10 mL under nitrogen atmosphere successively, and the mixture was stirred at 80° C. for 9 hours. To the resulting mixture was added ethyl acetate, and the mixture was washed with water and saturated brine successively. The resulting organic layers were dried over anhydrous sodium sulfate, and concentrated under reduced pressure. The resulting residue was subjected to a silica gel column chromatography (ethyl acetate : hexane = 1:4) to obtain the compound of the present invention 7-1 represented by the following formula 0.12 g.

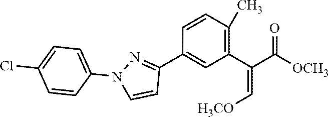 [SMILES: CO/C=C(/C(=O)OC)c1cc(-c2ccn(-c3ccc(Cl)cc3)n2)ccc1C]

Compound of the present invention 7-1: ¹H—NMR (CDCl₃) δ: 7.82 (1 H, d), 7.71 (1 H, dd), 7.66-7.64 (3 H, m), 7.59 (1 H, s), 7.38-7.35 (2 H, m), 7.27 (1 H, d), 6.69 (1 H, d), 3.78 (3 H, s), 3.69 (3 H, s), 2.21 (3 H, s).

### Preparation Example 6

To a mixture of 0.25 g of the intermediate compound 3, O-benzylhydroxyamine hydrochloride salt 0.16 g, and methanol 3 mL was added pyridine 0.16 mL at 0° C., and the mixture was stirred at room temperature for 4 hours. The resulting mixture was diluted with ethyl acetate, and washed with water and saturated brine successively. The resulting organic layers were dried over anhydrous sodium sulfate, and concentrated under reduced pressure. The resulting residue was subjected to a silica gel column chromatography (ethyl acetate : hexane = 1:4) to obtain the compound of the present invention 8-1 represented by the following formula 0.35 g.

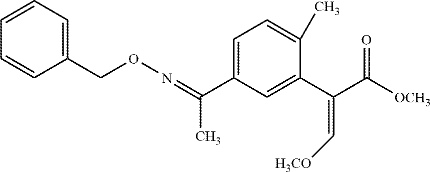 [SMILES: CO/C=C(/C(=O)OC)c1cc(C(C)=NOCc2ccccc2)ccc1C]

Compound of the present invention 8-1: ¹H—NMR (CDCl₃) δ: 7.57 (1 H, s), 7.52 (1 H, dd), 7.38 (6 H, m), 7.20 (1 H, d), 5.22 (2 H, s), 3.82 (3 H, s), 3.69 (3 H, s), 2.24 (3 H, s), 2.18 (3 H, s).

### Preparation Example 6-1

The compounds which were prepared according to the Preparation Example 6 and their physical property value are shown below.

A compound represented by formula (1h):

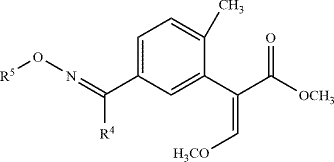

wherein a combination of R⁴ and R⁵ represents any combinations described in [Table A7].

TABLE A7

| Compound of the present invention | R⁴ | R⁵ |
|---|---|---|
| 8-2 | Me | CH₂-c-Pr |
| 8-3 | Me | CH₂-C = CH |
| 8-4 | H | CH₂—Ph |
| 8-5 | H | CH₂-c-Pr |
| 8-6 | Me | i-Pr |
| 8-7 | H | i-Pr |
| 8-8 | H | Me |
| 8-9 | H | Et |
| 8-10 | H | Pr |
| 8-11 | H | Pen |
| 8-12 | H | i-Bu |
| 8-13 | H | CH₂CF₂CF₃ |
| 8-14 | H | CH₂CF₃ |
| 8-15 | Me | Me |
| 8-16 | Me | Et |
| 8-17 | Me | Pen |
| 8-18 | Me | Ph |
| 8-19 | Et | Me |
| 8-20 | Et | Et |
| 8-21 | Me | c-Hex |

Compound of the present invention 8-2: ¹H—NMR (CDCl₃) δ: 7.57 (1 H, s), 7.51 (1 H, dd), 7.39 (1 H, d), 7.21 (1 H, d), 3.99 (2 H, d), 3.82 (3 H, d), 3.70 (3 H, d), 2.23 (3 H, s), 2.18 (3 H, s), 1.24-1.17 (1 H, m), 0.57-0.53 (2 H, m), 0.32-0.30 (2 H, m).

Compound of the present invention 8-3: ¹H—NMR (CDCl₃) δ: 7.58 (1 H, s), 7.53 (1 H, dd), 7.39 (1 H, d), 7.22 (1 H, d), 4.78 (2 H, d), 3.82 (3 H, s), 3.70 (3 H, s), 2.47 (1 H, t), 2.23 (3 H, s), 2.18 (3 H, s).

Compound of the present invention 8-4: ¹H—NMR (CDCl₃) δ: 8.10 (1 H, s), 7.57 (1 H, s), 7.47-7.29 (7 H, m), 7.22 (1 H, d), 5.19 (2 H, s), 3.82 (3 H, s), 3.69 (3 H, s), 2.18 (3 H, s).

Compound of the present invention 8-5: ¹H—NMR (CDCl₃) δ: 8.07 (1 H, s), 7.57 (1 H, s), 7.45 (1 H, dd), 7.30 (1 H, d), 7.22 (1 H, d), 3.97 (2 H, d), 3.83 (3 H, s), 3.70 (3 H, s), 2.18 (3 H, s), 1.23-1.16 (1 H, m), 0.60-0.54 (2 H, m), 0.34-0.29 (2 H, m).

Compound of the present invention 8-6: ¹H—NMR (CDCl₃) δ: 7.57 (1 H, s), 7.53 (1 H, dd), 7.38 (1 H, d), 7.20 (1 H, d), 4.46-4.40 (1 H, m), 3.82 (3 H, s), 3.70 (3 H, s), 2.19 (3 H, s), 2.18 (3 H, s), 1.29 (6 H, d).

Compound of the present invention 8-7: ¹H—NMR (CDCl₃) δ: 8.01 (1 H, s), 7.57 (1 H, s), 7.45 (1 H, dd), 7.30 (1 H, d), 7.22 (1 H, d), 4.46-4.40 (1 H, m), 3.83 (3 H, s), 3.70 (3 H, s), 2.18 (3 H, s), 1.28 (6 H, d).

Compound of the present invention 8-8: ¹H—NMR (CDCl₃ δ:8.02 (1 H, s), 7.57 (1 H, s), 7.44 (1 H, dd), 7.31 (1 H, d), 7.23 (1 H, d), 3.95 (3 H, s), 3.83 (3 H, s), 3.70 (3 H, s), 2.19 (3 H, s). Compound of the present invention 8-9: ¹H—NMR (CDCl₃) δ: 8.03 (1 H, s), 7.57 (1 H, s), 7.45 (1 H, d), 7.31 (1 H, s), 7.22 (1 H, d), 4.20 (2 H, q), 3.83 (3 H, s), 3.70 (3 H, s), 2.19 (3 H, s), 1.31 (3 H, t).

Compound of the present invention 8-10: ¹H—NMR (CDCl₃) δ: 8.04 (1 H, s), 7.57 (1 H, s), 7.45 (1 H, dd), 7.31 (1 H, d), 7.22 (1 H, d), 4.10 (2 H, t), 3.83 (3 H, s), 3.70 (3 H, s), 2.18 (3 H, s), 1.72 (2 H, td), 0.97 (3 H, t).

Compound of the present invention 8-11: ¹H—NMR (CDCl₃) δ: 8.03 (1 H, s), 7.57 (1 H, s), 7.45 (1 H, dd), 7.30 (1 H, d), 7.22 (1 H, d), 4.14 (2 H, t), 3.83 (3 H, s), 3.70 (3 H, s), 2.18 (3 H, s), 1.76-1.64 (2 H, m), 1.43-1.31 (4 H, m), 0.96-0.86 (3 H, m) .

Compound of the present invention 8-12: ¹H—NMR (CDCl₃)δ: 8.05 (1 H, s), 7.57 (1 H, s), 7.45 (1 H, dd), 7.30 (1 H, d), 7.22 (1 H, d), 3.91 (2 H, d), 3.83 (3 H, s), 3.70 (3 H, s), 2.18 (3 H, s), 2.09-1.98 (1 H, m), 0.95 (6 H, d).

Compound of the present invention 8-13: ¹H—NMR (CDCl₃) δ: 8.10 (1 H, s), 7.58 (1 H, s), 7.46 (1 H, dd), 7.30 (1 H, d), 7.25 (1 H, d), 4.64-4.55 (2 H, m), 3.84 (3 H, s), 3.71 (3 H, s), 2.20 (3 H, s).

Compound of the present invention 8-14: ¹H—NMR (CDCl₃) δ: 8.12 (1 H, s), 7.58 (1 H, s), 7.46 (1 H, dd), 7.30 (1 H, d), 7.24 (1 H, d), 4.50 (2 H, q), 3.84 (3 H, s), 3.70 (3 H, s), 2.20 (3 H, s).

Compound of the present invention 8-15: ¹H—NMR (CDCl₃) δ: 7.57 (1 H, s), 7.50 (1 H, dd), 7.39 (1 H, d), 7.21 (1 H, d), 3.97 (3 H, s), 3.82 (3 H, s), 3.69 (3 H, s), 2.19 (3 H, s), 2.18 (3 H, s).

Compound of the present invention 8-16: ¹H—NMR (CDCl₃) δ: 7.57 (1 H, s), 7.51 (1 H, dd), 7.39 (1 H, d), 7.21 (1 H, d), 4.22 (2 H, q), 3.82 (3 H, s), 3.70 (3 H, s), 2.20 (3 H, s), 2.18 (3 H, s), 1.31 (3 H, t) .

Compound of the present invention 8-17: ¹H—NMR (CDCl₃) δ: 7.57 (1 H, s), 7.51 (1 H, dd), 7.38 (1 H, d), 7.21 (1 H, d), 4.16 (2 H, t), 3.82 (3 H, s), 3.69 (3 H, s), 2.20 (3 H, s), 2.18 (3 H, s), 1.76-1.65 (2 H, m), 1.41-1.32 (4 H, m), 0.94-0.88 (3 H, m) .

Compound of the present invention 8-18: ¹H—NMR (CDCl₃) δ: 7.65 (1 H, dd), 7.60 (1 H, s), 7.52 (1 H, d), 7.35-7.25 (5 H, m), 7.04-6.99 (1 H, m), 3.84 (3 H, s), 3.71 (3 H, s), 2.42 (3 H, s), 2.21 (3 H, s).

Compound of the present invention 8-19: ¹H—NMR (CDCl₃) δ: 7.57 (1 H, s), 7.50 (1 H, dd), 7.38 (1 H, d), 7.21 (1 H, d), 3.95 (3 H, s), 3.81 (3 H, s), 3.69 (3 H, s), 2.71 (2 H, q), 2.18 (3 H, s), 1.13 (3 H, t).

Compound of the present invention 8-20: ¹H—NMR (CDCl₃) δ: 7.57 (1 H, s), 7.51 (1 H, dd), 7.38 (1 H, d), 7.21 (1 H, d), 4.21 (2 H, q), 3.82 (3 H, s), 3.70 (3 H, s), 2.72 (2 H, q), 2.18 (3 H, s), 1.30 (3 H, t), 1.13 (3 H, t).

Compound of the present invention 8-21: ¹H—NMR (CDCl₃) δ: 7.57 (1 H, s), 7.52 (1 H, dd), 7.38 (1 H, d), 7.20 (1 H, d), 4.21-4.14 (1 H, m), 3.82 (3 H, s), 3.69 (3 H, s), 2.20 (3 H, s), 2.18 (3 H, s), 2.03-1.93 (2 H, m), 1.79-1.69 (2 H, m), 1.58-1.28 (6 H, m).

### Preparation Example 7

A mixture of sodium hydride (60% in oil) 0.04 g and DMF 3 mL was stirred at 0° C. for 30 minutes. To the resulting mixture was added 0.25 g of the intermediate compound 4 under ice-cooling, and the mixture was stirred at room temperature for 4 hours. To the resulting mixture was added (bromomethyl)cyclobutene 0.16 mL at 0° C., and the mixture was stirred at room temperature for 2 hours. To the resulting mixture was added aqueous saturated ammonium chloride solution, and the mixture was extracted with ethyl acetate. The resulting organic layers were washed with water and saturated brine successively, dried over anhydrous sodium sulfate, and concentrated under reduced pressure. The resulting residue was subjected to a silica gel column chromatography (ethyl acetate : hexane = 1:4) to obtain the compound of the present invention 9-1 represented by the following formula 0.20 g.

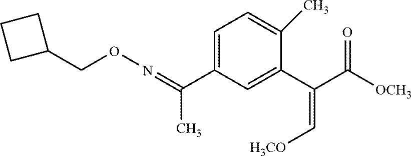 [SMILES: CO/C=C(/C(=O)OC)c1cc(C(C)=NOCC2CCC2)ccc1C]

Compound of the present invention 9-1: ¹H—NMR (CDCl₃) δ: 7.57 (1 H,s), 7.51 (1 H, d), 7.38 (1 H, s), 7.20 (1 H, d), 4.15 (2 H, d), 3.82 (3 H, s), 3.70 (3 H, s), 2.75-2.67 (1 H, m), 2.20 (3 H, s), 2.18 (3 H, s), 2.10-2.04 (2 H, m), 1.96-1.79 (4 H, m).

### Preparation Example 7-1

The compounds which were prepared according to the Preparation Example 7 and their physical property value are shown below.

The compounds represented by formula (1h) wherein a combination of R⁴ and R⁵ represents any combinations described in [Table A8].

TABLE A8

| Compound of the present invention | R⁴ | R⁵ |
|---|---|---|
| 9-2 | Me | 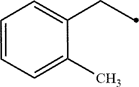 |
| 9-3 | Me | 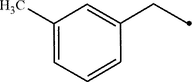 |
| 9-4 | Me | 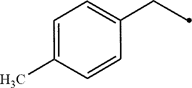 |
| 9-5 | Me | 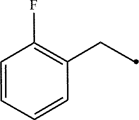 |
| 9-6 | Me | 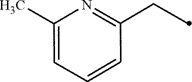 |

Compound of the present invention 9-2: ¹H—NMR (CDCl₃) δ: 7.57 (1 H, s), 7.52 (1 H, dd), 7.39-7.37 (2 H, m), 7.24-7.17 (4 H, m), 5.23 (2 H, s), 3.82 (3 H, s), 3.69 (3 H, s), 2.39 (3 H, s), 2.23 (3 H, s), 2.18 (3 H, s).

Compound of the present invention 9-3: ¹H—NMR (CDCl₃) δ: 7.57 (1 H, s), 7.52 (1 H, dd), 7.39 (1 H, d), 7.23-7.21 (4 H, m), 7.11 (1 H, d), 5.19 (2 H, s), 3.82 (3 H, s), 3.69 (3 H, s), 2.36 (3 H, s), 2.23 (3 H, s), 2.18 (3 H, s).

Compound of the present invention 9-4: ¹H—NMR (CDCl₃) δ: 7.57 (1 H, s), 7.52 (1 H, dd), 7.38 (1 H, d), 7.31 (2 H, d), 7.20 (1 H, d), 7.16 (2 H, d), 5.17 (2 H, s), 3.82 (3 H, s), 3.69 (3 H, s), 2.35 (3 H, s), 2.22 (3 H, s), 2.18 (3 H, s).

Compound of the present invention 9-5: ¹H—NMR (CDCl₃) δ: 7.57 (1 H, s), 7.52 (1 H, dd), 7.47-7.43 (1 H, m), 7.38 (1 H, d), 7.30-7.25 (1 H, m), 7.20 (1 H, d), 7.14-7.10 (1 H, m), 7.07-7.03 (1 H, m), 5.29 (2 H, s), 3.82 (3 H, s), 3.69 (3 H, s), 2.23 (3 H, s), 2.18 (3 H, s).

Compound of the present invention 9-6: ¹H—NMR (CDCl₃) δ:7.58-7.50 (3 H, m), 7.39 (1 H, d), 7.20 (2 H, dd), 7.04 (1 H, d), 5.32 (2 H, s), 3.81 (3 H, s), 3.69 (3 H, s), 2.56 (3 H, s), 2.30 (3 H, s), 2.17 (3 H, s).

### Preparation Example 8

A mixture of 0.45 g of the intermediate compound 8, butanol 0.23 g, bis(2-methoxyethyl) azocarboxylate 0.62 g, triphenylphosphine 0.64 g, and chloroform 5 mL was stirred at room temperature for 1 hour. The resulting mixture was concentrated under reduced pressure. The resulting residue was subjected to a silica gel column chromatography (ethyl acetate : hexane = 1:4) to obtain a compound of the present invention 10-1 represented by the following formula 0.21 g.

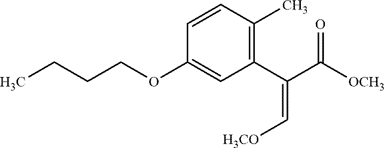 [SMILES: CCCCOc1ccc(C)c(/C(=C\OC)C(=O)OC)c1]

Compound of the present invention 10-1: ¹H—NMR (CDCl₃) δ: 7.54 (1 H, s), 7.11 (1 H, d), 6.77 (1 H, dd), 6.67 (1 H, d), 3.92 (2 H, t), 3.82 (3 H, s), 3.70 (3 H, s), 2.10 (3 H, s), 1.77-1.70 (2 H, m), 1.52-1.43 (2 H, m), 0.96 (3 H, t).

### Preparation Example 8-1

The compounds which were prepared according to the Preparation Example 8 and their physical property value are shown below.

The compound of the present invention 10-2 to the compound of the present invention 10-8, which corresponds to a compound represented by formula (1k):

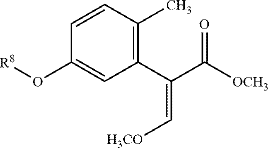

wherein R⁶ represents a substituent described below.

Compound of the present invention 10-2 (R⁶— Pr) : ¹H—NMR (CDCl₃) δ: 7.54 (1 H, s), 7.11 (1 H, d), 6.77 (1 H, dd), 6.67 (1 H, d), 3.88 (2 H, t), 3.82 (3 H, s), 3.70 (3 H, s), 2.10 (3 H, s), 1.83-1.74 (2 H, m), 1.02 (3 H, t).

Compound of the present invention 10-3 (R⁶— CH₂CHMe₂)— ¹H—NMR (CDCl₃) δ: 7.54 (1 H, s), 7.11 (1 H, d), 6.77 (1 H, dd), 6.66 (1 H, d), 3.82 (3 H, s), 3.70 (3 H, s), 3.68 (2 H, d), 2.10 (3 H, s), 2.09-2.00 (1 H, m), 1.00 (6 H, d).

Compound of the present invention 10-4 (R⁶— CH₂CH₃)— ¹H—NMR (CDCl₃) δ: 7.54 (1 H, s), 7.11 (1 H, d), 6.77 (1 H, dd), 6.67 (1 H, d), 4.00 (2 H, q), 3.82 (3 H, s), 3.70 (3 H, s), 2.10 (3 H, s), 1.38 (3 H, t).

Compound of the present invention 10-5 (R⁶— CH₂(c—Pr)): ¹H—NMR (CDCl₃) δ: 7.54 (1 H, s), 7.11 (1 H, d), 6.78 (1 H, dd), 6.67 (1 H, d), 3.82 (3 H, s), 3.76 (2 H, d), 3.70 (3 H, s), 2.10 (3 H, s), 1.29-1.23 (1 H, m), 0.6-0.60 (2 H, m), 0.34-0.30 (2 H, m) .

Compound of the present invention 10-6 (R⁶— CH₂CH₂OMe)— ¹H—NMR (CDCl₃) δ: 7.54 (1 H, s), 7.11 (1 H, d), 6.80 (1 H, dd), 6.70 (1 H, d), 4.10-4.08 (2 H, m), 3.82 (3 H, s), 3.74-3.70 (2 H, m), 3.69 (3 H, s), 3.44 (3 H, s), 2.10 (3 H, s).

Compound of the present invention 10-7 (R⁶— Pen): ¹H—NMR (CDCl₃) δ: 7.54 (1 H, s), 7.10 (1 H, d), 6.76 (1 H, dd), 6.66 (1 H, d), 3.91 (2 H, t), 3.82 (3 H, s), 3.69 (3 H, s), 2.09 (3 H, s), 1.80-1.71 (2 H, m), 1.46-1.31 (4 H, m), 0.91 (3 H, t).

Compound of the present invention 10-8 (R⁶— CH₂CH₂F)— ¹H—NMR (CDCl₃) δ: 7.55 (1 H, s), 7.13 (1 H, d), 6.80 (1 H, dd), 6.70 (1 H, d), 4.73 (2 H, dt), 4.18 (2 H, dt), 3.82 (3 H, s), 3.69 (3 H, s), 2.10 (3 H, s).

### Preparation Example 9

A mixture of 0.30 g of the intermediate compound 1, cyclohexene 0.43 g, acetylacetone palladium (II) 0.13 g, diisopropylamine 0.16 g, tetrafluoroboronic acid tri-tert-butyl phosphine 0.06 g, and N,N′-dimethylpropylene urea 3 mL was stirred at 130° C. for 5 hours. The resulting mixture was cooled to room temperature, and then concentrated under reduced pressure. The resulting residue was subjected to a silica gel column chromatography (ethyl acetate , hexane = 1:2) to obtain the compound 3-8 of the present invention represented by the following formula 0.20 g.

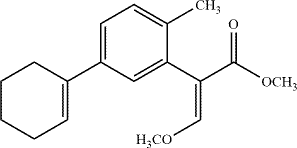 [SMILES: CO/C=C(/C(=O)OC)c1cc(C2=CCCCC2)ccc1C]

Compound of the present invention 3-8: LCMS: 287 [M+H]⁺, RT = 2.27 minutes

### Preparation Example 9-1

The compounds which were prepared according to the Preparation Example 9 and their physical property value are shown below.

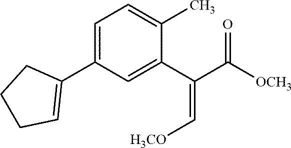 [SMILES: CO/C=C(/C(=O)OC)c1cc(C2=CCCC2)ccc1C]

Compound of the present invention 3-9: ¹H—NMR (CDCl₃) δ: 7.57 (1 H, s), 7.29 (1 H, dd), 7.18-7.15 (2 H, m), 6.12-6.10 (1 H, m), 3.81 (3 H, s), 3.69 (3 H, s), 2.70-2.65 (2 H, m), 2.53-2.47 (2 H, m), 2.16 (3 H, s), 2.02-1.97 (2 H, m) .

### Preparation Example 10

To a mixture of 0.25 g of the intermediate compound 8, potassium carbonate 0.23 g, and acetonitrile 5 mL was added dimethyl carbamoyl chloride 0.17 mL at 0° C. under nitrogen atmosphere, and the mixture was stirred at room temperature for 7 hours. To the resulting mixture was added aqueous saturated sodium hydrogencarbonate solution, and the mixture was extracted with ethyl acetate. The resulting organic layers were washed with water and saturated brine successively, dried over anhydrous sodium sulfate, and concentrated under reduced pressure. The resulting residue was subjected to a silica gel column chromatography (ethyl acetate : hexane = 1:3) to obtain a compound of the present invention 11-1 represented by the following formula 0.27 g.

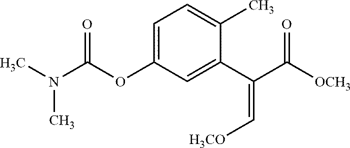 [SMILES: CO/C=C(/C(=O)OC)c1cc(OC(=O)N(C)C)ccc1C]

Compound of the present invention 11-1: ¹H—NMR (CDCl₃) δ: 7.55 (1 H, s), 7.18 (1 H, d), 6.99 (1 H, dd), 6.87 (1 H, d), 3.81 (3 H, s), 3.69 (3 H, s), 3.07 (3 H, s), 3.00 (3 H, s), 2.14 (3 H, s).

### Preparation Example 10-1

The compounds which were prepared according to the Preparation Example 10 and their physical property value are shown below.

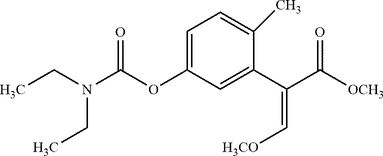 [SMILES: CCN(CC)C(=O)Oc1ccc(C)c(/C(=C\OC)C(=O)OC)c1]

Compound of the present invention 11-2: ¹H—NMR (CDCl₃) δ: 7.55 (1 H, s), 7.18 (1 H, d), 7.00 (1 H, dd), 6.88 (1 H, d), 3.81 (3 H, s), 3.69 (3 H, s), 3.46-3.32 (4 H, m), 2.14 (3 H, s), 1.26-1.15 (6 H, m). [0155]

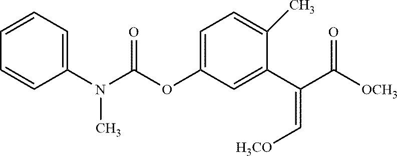 [SMILES: CO/C=C(/C(=O)OC)c1cc(OC(=O)N(C)c2ccccc2)ccc1C]

Compound of the present invention 11-3: ¹H—NMR (CDCl₃) δ: 7.53 (1 H, s), 7.41-7.31 (4 H, m), 7.28-7.21 (1 H, m), 7.17 (1 H, d), 7.02-6.97 (1 H, m), 6.85 (1 H, s), 3.80 (3 H, s), 3.67 (3 H, s), 3.41 (3 H, s), 2.13 (3 H, s).

Next, examples of the compounds of the present invention and the intermediate compounds which are prepared according to the Preparation Examples described in Working Examples and the Processes described herein are shown below.

A compound represented by formula (1A):

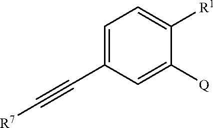

wherein Q represents Q1, R¹ represents a methyl group, and R⁷ represents any substituents selected from Group X (hereinafter, referred to as Compound class SX1).

Group X: a group consisting of Me,Et,Pr,i-Pr,c-Pr,c—Bu,c— Pen,c—Hex, (CH₂)₃CH₃,CH₂ CH(CH₃)₂,CH(CH₃)CH₂ CH₃,t—Bu,CH₂ c—Pr, (CH₂ ) ₄ CH₃, (CH₂ ) ₂ CH (CH₃ ) ₂ , CH (CH₃ ) (CH₂ ) ₂ CH₃ , CH (CH₂ CH₃ ) CH₂ C _{H3} , _{CH2 CH} (_{CH3} ) _{CH2 CH3} , _{C} (CH₃ ) ₂ CH₂ CH₃ , CH₂ t—Bu, CH₂ C—BU, CH₂ C — Pen,(CH₂)₂c—Pr,(1-methylcyclopropyl)methyl,(2-methylcyclopropyl) methyl, 1-cyclopropylethyl, (eH₂ ) ₃ c—Pr, (eH₂ ) ₅ eH_{3,} (eH₂ ) ₃ eH (eH3) ₂, (eH₂) ₂ eH (eH₃) eH₂ eH₃, eH₂ eH (eH₃) ( CH₂ ) ₂CH₃ , CH (CH₃ ) (CH₂ ) ₃CH₃ , CH₂ CH (CH₂ CH₃ ) ₂ , heptyl, CH₂ CH═CH₂ , C H2 CH═CHCH3 , CH2 CH-C (CH3 ) 2 , CH2 CH═CF2 , CH2 CH═CC12 , CH2 CH═CHCH2 CH ₃, CH₂CH═CH(CH₂)₂CH₃, CH₂C(CH₃)═CH₂, CH₂C(CH₃)═CHCH₃, CH₂C(CH₃) ═C(CH₃)₂, CH₂C(CH₃)═CHCH₂CH₃, CH₂CF═CH₂, CH₂CF═CHCH₃, CH₂CF═C(C H₃)₂, CH₂CF═CF₂ , CH₂CF═CHCH₂CH₃, CH₂CF═CH(CH₂)₂CH₃, CH₂CCl═CH₂, CH₂CCl═CHCH₃, CH₂CCl-C(CH₃)₂, CH₂CCl-CCl₂, CH₂CCl-CHCH₂CH₃, CH₂ CCl═CH(CH₂)₂CH₃, (CH₂)₂CH═CH₂, (CH₂)₂CH═CHCH₃, (CH₂)₂CH═CHCH₂C H₃, (CH₂)₂CH═C(CH₃)₂, (CH₂)₂C(CH₃)═CH₂, (CH₂)₂C(CH₃)═CHCH₃, (CH ₂)₃CH═CH₂, (CH₂)₃C(CH₃)═CH₂, (CH₂)₄CH═CH₂, CH₂C≡CH, CH₂C≡CCH3, C H₂C≡CCH₂CH₃, CH₂C≡Cc—Pr,(CH₂)₂C≡CH, (CH₂)₂C≡CCH₃, (CH₂)₂C≡CCH₂CH₃, (CH₂)₃C≡CH,(CH₂) ₃C≡CCH₃, CH₂Cl, CH₂Br, CH₂OCH₃, CH₂OCH₂CH₃, CH₂O(CH₂)₂CH₃, CHF₂, C F₃, CH₂CF₃, CH₂CHF₂, CH₂CH₂CHF₂, CH₂CH₂CF₃, CH₂CF₂CF₃, (CH₂)₂CF₂C F₃, CH₂(CF₂)₂CF_{3,} (CH₂)₂CF(CF₃)₂, (CH₂)₂(CF₂)₅CF₃, CF_{3,} CF₂CHF(C F₃)_{,} CF₂CHF(OCF₃), and CH₂CF₂CF₂H.

A compound (1A) wherein Q represents Q1, R¹ represents a chlorine atom, and R⁷ represents any substituent selected from Group X (hereinafter, referred to as Compound class SX2).

A compound (1A) wherein Q represents Q1, R¹ represents a fluorine atom, and R⁷ represents any substituent selected from Group X (hereinafter, referred to as Compound class SX3).

A compound (1A) wherein Q represents Q1, R¹ represents an ethyl group, and R⁷ represents any substituent selected from Group X (hereinafter, referred to as Compound class SX4) .

A compound (1A) wherein Q represents Q1, R¹ represents a cyclopropyl group, and R⁷ represents any substituent selected from Group X (hereinafter, referred to as Compound class SX5).

A compound (1A) wherein Q represents Q2, L represents an oxygen atom, R¹ represents a methyl group, and R⁷ represents any substituent selected from Group X (hereinafter, referred to as Compound class SX6).

A compound (1A) wherein Q represents Q2, L represents an oxygen atom, R¹ represents a chlorine atom, and R⁷ represents any substituent selected from Group X (hereinafter, referred to as Compound class SX7).

A compound (1A) wherein Q represents Q2, L represents NH, R¹ represents a methyl group, and R⁷ represents any substituent selected from Group X (hereinafter, referred to as Compound class SX8).

A compound (1A) wherein Q represents Q2, L represents NH, R¹ represents a chlorine atom, and R⁷ represents any substituent selected from Group X (hereinafter, referred to as Compound class SX9).

A compound represented by formula (1B):

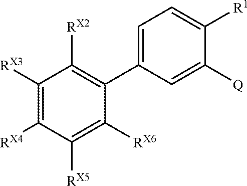

(hereinafter, referred to as “Compound (1B)”),
wherein Q represents Q1, R¹ represents a methyl group, and a combination of R^{X2}, R^{X3}, P^{X4}, R^{X5}, and R^{X6} represents any combinations described in Combination A (hereinafter, referred to as Compound class SX10).

Combination A consists of Substituent Nos. ZA1 to ZA312. The Substituent No. ZA1 to ZA312 represents a combination of R^{X2}, R^{X3}, R^{X4}, R^{X5}, and R^{X6} in the compound (1B), a compound represented by formula (1D), a compound represented by formula (1F), a compound represented by formula (1I), a compound represented by formula (1M), a compound represented by formula (1N), a compound represented by formula (1O), a compound represented by formula (1P), a compound represented by formula (1Q), a compound represented by formula (1R), a compound represented by formula (1S), a compound represented by formula (1T), a compound represented by formula (2C), and a compound represented by formula (2E), , which hereinafter, is referred to as [Substituent No.; R^{X2}, R^{X3}, R^{X4}, R^{X5}, R^{X6}]. For example, Substituent No. ZA2 represents a combination wherein R^{X2} represents a methyl group, and R^{X3}, R^{X4}, R^{X5}, and R^{X6} represent a hydrogen atom.

### Combination A

[ZA1;H, H, H, H, H], [ZA2; Me, H, H, H, H], [ZA3; F, H, H, H, H], [ZA4; Cl, H, H, H, H], [ZA5; OMe, H, H, H, H], [ZA6; CF₃, H, H, H, H], [ZA7; H, Me, H, H, H], [ZA8; H, Et, H, H, H], [ZA9; H, Pr, H, H, H], [ZA10; H, i-Pr, H, H, H], [ZA11; H, t-Bu, H, H, H], [ZA12; H, OMe, H, H, H], [ZA13; H, OEt, H, H, H], [ZA14; H, OPr, H, H, H], [ZA15; H, Oi-Pr, H, H, H], [ZA16; H, CF₃, H, H, H], [ZA17; H, CF₂H, H, H, H], [ZA18; H, CF H₂, H, H, H], [ZA19 ;H, F, H, H, H], [ZA20; H, Cl, H, H, H], [ZA21; H, Br, H, H, H], [ZA22; H, CN, H, H, H], [ZA23 ;H, Ph, H, H, H], [ZA24; H, OPh, H, H, H], [ZA25; H, c-Pr, H, H, H], [ZA26; H, c-Pen, H, H, H], [ZA27; H, c-Hex, H, H, H], [ZA28; H, H, Me, H, H], [ZA29; H, H, Et, H, H], [ZA30; H, H, Pr, H, H], [ZA31; H, H, i-Pr, H, H], [ZA32; H, H, t-Bu, H, H], [ZA33; H, H, OMe, H, H], [ZA34; H, H, OEt, H, H], [ZA35; H, H, OPr, H, H], [ZA36 ;H, H, Oi-Pr, H, H], [ZA37; H, H, CF₃, H, H], [ZA38; H, H, CF₂H, H, H], [ZA39; H, H, CF H₂, H, H], [ZA40; H, H, F, H, H], [ZA41;H, H, Cl, H, H], [ZA42; H, H, Br, H, H ], [ZA43; H, H, CN, H, H], [ZA44; H, H, Ph, H, H], [ZA45; H, H, OPh, H, H], [Z A46; H, H, c-Pr, H, H], [ZA47; H, H, c-Pen, H, H], [ZA48; H, H, c-Hex, H, H], [ZA49; H, H, H, H, F], [ZA50; Me, H, H, H, F], [ZA51; F, H, H, H, F ], [ZA52; Cl, H, H, H, F], [ZA53; H, Me, H, H, F], [ZA54; H, Et, H, H, F], [ZA 55; H, Pr, H, H, F], [ZA56; H, i-Pr, H, H, F], [ZA57; H, t-Bu, H, H, F], [ZA58; H, OMe, H, H, F], [ZA59; H, OEt, H, H, F], [ZA60;H,OPr ,H, H, F], [ZA61; H, Oi-Pr, H, H, F], [ZA62; H, CF₃, H, H, F], [ZA63; H, CF₂H, H, H, F], [ZA64; H, CF H₂, H, H, F], [ZA65; H, F, H, H, F], [ZA66; H, Cl, H, H, F], [ZA67; H, Br, H, H , F], [ZA68; H, CN, H, H, F], [ZA69; H, Ph, H, H, F], [ZA70; H, OPh, H, H, F], [ZA71; H, c-Pr, H, H, F], [ZA72; H, c-Pen, H, H, F], [ZA73; H, c-Hex, H, H, F], [ZA74; H, H, Me, H, F], [ZA75; H, H, Et, H, F], [ZA76; H, H, Pr , H, F], [ZA77; H, H, i-Pr, H, F], [ZA78; H, H, t-Bu, H, F], [ZA79; H, H, OMe, H, F], [ZA80; H, H, OEt, H, F], [ZA81; H, H, OPr , H, F], [ZA82; H, H, Oi-Pr, H, F], [ZA83; H, H, CF₃, H, F], [ZA84; H, H, CF₂H, H, F], [ZA85; H, H, CF H₂, H, F], [ZA86; H, H, F, H, F], [ZA87; H, H, Cl, H, F], [ZA88; H, H, Br, H, F ], [ZA89; H, H, CN, H, F], [ZA90; H, H, Ph, H, F], [ZA91; H, H, OPh, H, F], [Z A92; H, H, c-Pr, H, F], [ZA93; H, H, c-Pen, H, F], [ZA94; H, H, c-Hex, H, F], [ZA95; H, H, H, H, Cl], [ZA96; Me, H, H, H, Cl], [ZA97; F, H, H, H , Cl], [ZA98; Cl, H, H, H, Cl], [ZA99; H, Me, H, H, Cl], [ZA100; H, Et, H, H, Cl], [ZA101; H, Pr, H, H, Cl], [ZA102; H, i-Pr, H, H, Cl], [ZA103; H, t-Bu, H, H, Cl], [ZA104; H, OMe, H, H, Cl], [ZA105; H, OEt, H, H, Cl], [ZA106 ; H, OPr, H, H, Cl], [ZA107; H, Oi-Pr, H, H, Cl], [ZA108; H, CF_{3,} H, H, Cl], [ZA109; H, CF₂H, H, H, Cl], [ZA11 0; H, CFH₂, H, H, C1], [ZA111; H, F, H, H, C1], [ZA112; H, C1, H, H, C1], [ZA 113; H, Br, H, H, Cl], [ZA114; H, CN, H, H, Cl], [ZA115; H, Ph, H, H, Cl], [Z A116; H, OPh, H, H, Cl], [ZA117; H, c-Pr, H, H, Cl], [ZA118; H, H, H, F, F], [ZA119; F, H, H, F, F], [ZA120; H, H, M e, H, Cl], [ZA121; H, H, Et, H, Cl], [ZA122; H, H, Pr, H, Cl], [ZA123; H, H, i-Pr, H, Cl], [ZA124; H, H, t-Bu, H,Cl], [ZA125; H, H, OMe, H, Cl], [ZA126; H, H, OEt, H, Cl], [ZA127; H, H, OPr, H, Cl], [ZA128; H, H, Oi-Pr, H, Cl], [ZA129; H, H, CF₃, H, Cl], [ZA130; H, H, CF₂H, H, Cl], [ZA131; H, H, CFH₂, H, Cl], [ZA132;H, H, F, H, Cl], [ZA133; H, H, Cl, H, Cl], [ZA13 4; H, H, Br, H, Cl], [ZA135; H, H, CN, H, Cl], [ZA136; H, H, Ph, H, Cl], [ZA1 37; H, H, OPh, H, Cl], [ZA138; H, H, c-Pr, H, Cl], [ZA139; H, H, c-Pen, H, Cl], [ZA140; H, H, c-Hex, H, Cl], [ZA141; H, H, H, H, Me], [ZA142; Me, H, H, H, Me], [ZA143; F, H, H, H, Me], [ZA144; Cl, H, H, H, Me], [ZA145; H, Me, H, H, Me], [ZA146; H, E t, H, H, Me], [ZA147; H, Pr, H, H, Me], [ZA148; H, i-Pr, H, H, Me], [ZA149; H, t-Bu, H, H, Me], [ZA150; H, OMe, H, H, Me], [ZA151; H, OEt, H, H, Me], [ZA152; H, OPr, H, H, Me], [ZA153; H, Oi-Pr, H, H, Me], [ZA154; H, CF₃, H, H, Me], [ZA155; H, CF₂H, H, H, Me], [ZA15 6; H, CFH₂, H, H, Me], [ZA157; H, F, H, H, Me], [ZA158; H, Cl, H, H, Me], [ZA 159; H, Br, H, H, Me], [ZA160; H, CN, H, H, Me], [ZA161; H, Ph, H, H, Me], [Z A162; H, OPh, H, H, Me], [ZA163; H, c-Pr, H, H, Me], [ZA164; H, c-Pen, H, H, Me], [ZA165; H, c-Hex, H, H, Me], [ZA166; H, H, Me, H, Me], [ZA167; H, H, Et, H, Me], [ZA168; H, H, Pr, H, Me], [ZA169; H, H, i-Pr, H, Me], [ZA170; H, H, t-Bu, H, Me], [ZA171; H, H, OMe, H, Me], [ZA172; H, H, OEt, H, Me], [ZA173; H, H, OPr, H, Me], [ZA174; H, H, Oi-Pr, H, Me], [ZA175; H, H, CF₃, H, Me], [ZA176; H, H, CF₂H, H, Me], [ZA177; H, H, CFH₂,H, Me], [ZA178; H, H, F, H, Me], [ZAI79; H, H, Cl, H, Me], [ZA18 0; H, H, Br, H, Me], [ZA181; H, H, CN, H, Me], [ZA182; H, H, Ph, H, Me], [ZA1 83; H, H, OPh, H, Me], [ZA184; H, H, c-Pr, H, Me], [ZA185; H, H, c-Pen, H, Me], [ZA186; H, H, c-Hex, H, Me],[ZA187; H, H, H, H, OMe], [ZA188; Me, H, H, H, OMe], [ZA189; F, H, H, H, OMe], [ZA190; Cl, H, H, H, OMe], [ZA191; H, Me, H, H, OMe], [ZA19 2; H, Et, H, H, OMe], [ZA193; H, Pr, H, H, OMe], [ZA194; H, i-Pr, H, H, OMe], [ZA195; H, t-Bu, H, H, OMe], [ZA196; H, OMe, H, H, OMe], [ZA197; H, OEt, H, H, OMe], [ZA 198; H, OPr, H, H, OMe], [ZA199; H, Oi-Pr, H, H, OMe], [ZA200; H, CF₃, H, H, OMe], [ZA201; H, CF₂H, H, H, OMe], [ZA202; H, CFH₂, H, H, OMe], [ZA203; H, F, H, H, OMe], [ZA204; H, Cl, H, H, OMe], [ZA205; H, Br, H, H, OMe], [ZA206; H, C N, H, H, OMe], [ZA207; H, Ph, H, H, OMe], [ZA208; H, OPh, H, H, OMe], [ZA20 9; H, c-Pr, H, H, OMe], [ZA210; H, c-Pen, H, H, OMe], [ZA211; H, c-Hex, H, H, OMe], [ZA212; H, H, Me, H, OMe], [ZA213; H, H, Et, H, OMe], [ZA2 14; H, H, Pr, H, OMe], [ZA215; H, H, i-Pr, H, OMe], [ZA216; H, H, t-Bu, H, OMe], [ZA217; H, H, OMe, H, OMe], [ZA218; H, H, OEt, H, OMe], [ZA21 9; H, H, OPr, H, OMe],[ZA220; H, H, Oi-Pr, H, OMe], [ZA221; H, H, CF₃,H, OMe], [ZA222; H, H, CF₂H, H, OMe], [ZA2 23; H, H, CFH₂, H, OMe], [ZA224; H, H, F, H, OMe], [ZA225; H, H, Cl, H, OMe], [ZA226; H, H, Br, H, OMe], [ZA227; H, H, CN, H, OMe], [ZA228; H, H, Ph, H, OMe], [ZA229; H, H, OPh, H, OMe], [ZA230; H, H, c-Pr, H, OMe], [ZA231; H, H, c-Pen, H, OMe], [ZA232; H, H, c-Hex, H, OMe], [ZA233; H, H, H, H, CF₃, [ZA234; Me, H, H, H, CF₃], [ZA235; F, H, H, H, CF₃], [ZA236; Cl, H, H, H, CF₃], [ZA237; H, Me, H, H, CF₃], [ZA23 8; H, Et, H, H, CF₃], [ZA239; H, Pr, H, H, CF₃], [ZA240; H, i-Pr,H,H,CF_{3]}, [ZA241;H,t-Bu, H, H, CF₃], [ZA242; H, OMe, H, H, CF₃], [ZA243; H, OEt, H, H, CF₃], [ZA2 44; H, OPr, H, H, CF₃], [ZA245; H,Oi-Pr, H, H, CF₃], [ZA246; H, CF₃, H, H, CF₃], [ZA247; H, CF₂H, H, H, CF₃], [ZA 248; H, CFH₂, H, H, CF₃], [ZA249; H, F, H, H, CF₃], [ZA250; H, Cl, H, H, CF₃] [ZA251; H, Br, H, H, CF₃], [ZA252; H, CN, H, H, CF₃], [ZA253; H, Ph, H, H, CF₃], [ZA254; H, OPh, H, H, CF₃], [ZA255; H, c-Pr, H, H, CF₃], [ZA256; H, c-Pen, H, H, CF₃], [ZA257; H, c-Hex, H, H, CF₃], [ZA258; H, H, Me, H, CF₃], [ZA259; H, H, Et, H, CF₃], [ZA26 0; H, H, Pr, H, CF₃], [ZA261; H, H, i-Pr, H, CF₃], [ZA262; H, H, t-Bu, H, CF₃], [ZA263; H, H, OMe, H, CF₃], [ZA264; H, H, OEt, H, CF₃], [ZA265; H, H, OPr, H, CF₃], [ZA266; H, H, Oi-Pr, H, CF₃], [ZA267; H, H, CF₃, H, CF₃], [ZA268; H, H, CF₂H, H, CF₃], [ZA26 9; H, H, CFH₂, H, CF₃], [ZA270; H, H, F, H, CF₃], [ZA271; H, H, Cl, H, CF₃], [ ZA272; H, H, Br, H, CF₃], [ZA273; H, H, CN, H, CF₃], [ZA274; H, H, Ph, H, CF₃ ], [ZA275; H, H, OPh, H, CF₃], [ZA276; H, H, c-Pr, H, CF₃], [ZA277; H, H, c-Pen, H, CF₃], [ZA278; H, H, c-Hex, H, CF₃], [ZA279; H, F, F, H, H], [ZA280; H, F, H, F, H], [ZA281; H, F, F, F, H], [ZA282; F, F, F, H, H], [ZA283; F, F, H, F, H], [ZA284; F, H, F, H, F], [ZA285; F, F, F, F, F], [ZA286; F, F, H, F, F], [ZA287; H, OMe, H, OMe, H], [ZA288; H, F, Cl, H, H], [ZA289; H, F, Me, H, H], [ZA290; H, F, OMe, H, H], [ ZA291; H, F, CF₃, H, H], [ZA292; H, Cl, F, H, H], [ZA293; H, Cl, Cl, H, H], [ ZA294; H, Cl, Me, H, H], [ZA295; H, Cl, OMe, H, H], [ZA296; H, Cl, CF₃,H, H ], [ZA297; H, Me, F, H, H], [ZA298; H, Me, Cl, H, H], [ZA299; H, Me, Me, H, H ], [ZA300; H, Me, OMe, H, H], [ZA301; H, Me, CF₃, H, H], [ZA302; H, OMe, F, H, H], [ZA303; H, OMe, Cl, H, H], [ZA304; H, OMe, Me, H, H], [ZA305; H, OMe, OMe, H, H], [ZA306; H, OMe, CF₃, H, H], [ZA307; H, CF₃, F, H, H], [ZA308; H, CF₃, Cl, H, H],[ZA309; H, C F₃, F, H, H], [ZA310; H, CF₃, Cl, H, H], [ZA311; H, CF₃, F, H, H], [ZA312; H, Cl, H, Cl, H]

A compound (1B) wherein Q represents Q1, R¹ represents a chlorine atom, and a combination of R^{X2}, R^{X3}, R^{X4}, R^{X5}, and R^{X6} represents any combinations described in Combination A (hereinafter, referred to as Compound class SX11).

A compound (1B) wherein Q represents Q1, R¹ represents a fluorine atom, and a combination of R^{X2}, R^{X3}, R^{X4}, R^{X5}, and R^{X6} represents any combinations described in Combination A (hereinafter, referred to as Compound class SX12).

A compound (1B) wherein Q represents Q1, R¹ represents an ethyl group, and a combination of R^{X2}, R^{X3}, R^{X4}, R^{X5}, and R^{X6} represents any combinations described in Combination A (hereinafter, referred to as Compound class SX13).

A compound (1B) wherein Q represents Q1, R¹ represents a cyclopropyl group, and a combination of R^{X2}, R^{X3}, R^{X4}, R^{X5}, and R^{X6} represents any combinations described in Combination A (hereinafter, referred to as Compound class SX14).

A compound (1B) wherein Q represents Q2, L represents an oxygen atom, R¹ represents a methyl group, and a combination of R^{X2}, R^{X3}, R^{X4}, R^{X5}, and R^{X6} represents any combinations described in Combination A (hereinafter, referred to as Compound class SX15).

A compound (1B) wherein Q represents Q2, L represents an oxygen atom, R¹ represents a chlorine atom, and a combination of R^{X2}, R^{X3}, R^{X4}, R^{X5}, and R^{X6} represents any combinations described in Combination A (hereinafter, referred to as Compound class SX16).

A compound (1B) wherein Q represents Q2, L represents NH, R¹ represents a methyl group, and a combination of R^{X2}, R^{X3}, R^{X4}, R^{X5}, and R^{X6} represents any combinations described in Combination A (hereinafter, referred to as Compound class SX17) .

A compound (1B) wherein Q represents Q2, L represents NH, R¹ represents a chlorine atom, and a combination of R^{X2}, R^{X3}, R^{X4}, R^{X5}, and R^{X6} represents any combinations described in Combination A (hereinafter, referred to as Compound class SX18).

A compound represented by formula (1C):

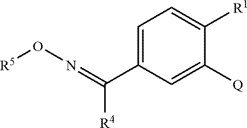

(hereinafter, referred to as “Compound (1C)”),
wherein R¹ represents a methyl group, R⁴ represents a methyl group, and R⁵ represents any substituents selected from Group X (hereinafter, referred to as Compound class SX19).

A compound (1C) wherein Q represents Q1, R¹ represents a chlorine atom, R⁴ represents a methyl group, and R⁵ represents any substituents selected from Group X (hereinafter, referred to as Compound class SX20).

A compound (1C) wherein Q represents Q1, R¹ represents a fluorine atom, R⁴ represents a methyl group, and R⁵ represents any substituents selected from Group X (hereinafter, referred to as Compound class SX21).

A compound (1C) wherein Q represents Q1, R¹ represents an ethyl group, R⁴ represents a methyl group, and R⁵ represents any substituents selected from Group X (hereinafter, referred to as Compound class SX22).

A compound (1C) wherein Q represents Q1, R¹ represents a cyclopropyl group, R⁴ represents a methyl group, and R⁵ represents any substituents selected from Group X (hereinafter, referred to as Compound class SX23).

A compound (1C) wherein Q represents Q1, R¹ represents a methyl group, R⁴ represents an ethyl group, and R⁵ represents any substituents selected from Group X (hereinafter, referred to as Compound class SX24).

A compound (1C) wherein Q represents Q1, R¹ represents a chlorine atom, R⁴ represents an ethyl group, and R⁵ represents any substituents selected from Group X (hereinafter, referred to as Compound class SX25).

A compound (1C) wherein Q represents Q1, R¹ represents a fluorine atom, R⁴ represents an ethyl group, and R⁵ represents any substituents selected from Group X (hereinafter, referred to as Compound class SX26).

A compound (1C) wherein Q represents Q1, R¹ represents an ethyl group, R⁴ represents an ethyl group, and R⁵ represents any substituents selected from Group X (hereinafter, referred to as Compound class SX27).

A compound (1C) wherein Q represents Q1, R¹ represents a cyclopropyl group, R⁴ represents an ethyl group, and R⁵ represents any substituents selected from Group X (hereinafter, referred to as Compound class SX28).

A compound (1C) wherein Q represents Q1, R¹ represents a methyl group, R⁴ represents a hydrogen atom, and R⁵ represents any substituents selected from Group X (hereinafter, referred to as Compound class SX29).

A compound (1C) wherein Q represents Q1, R¹ represents a chlorine atom, R⁴ represents a hydrogen atom, and R⁵ represents any substituents selected from Group X (hereinafter, referred to as Compound class SX30).

A compound (1C) wherein Q represents Q1, R¹ represents a fluorine atom, R⁴ represents a hydrogen atom, and R⁵ represents any substituents selected from Group X (hereinafter, referred to as Compound class SX31).

A compound (1C) wherein Q represents Q1, R¹ represents an ethyl group, R⁴ represents a hydrogen atom, and R⁵ represents any substituents selected from Group X (hereinafter, referred to as Compound class SX32).

A compound (1C) wherein Q represents Q1, R¹ represents a cyclopropyl group, R⁴ represents a hydrogen atom, and R⁵ represents any substituents selected from Group X (hereinafter, referred to as Compound class SX33).

A compound (1C) wherein Q represents Q2, L represents an oxygen atom, R¹ represents a methyl group, R⁴ represents a methyl group, and R⁵ represents any substituents selected from Group X (hereinafter, referred to as Compound class SX34) .

A compound (1C) wherein Q represents Q2, L represents an oxygen atom, R¹ represents a chlorine atom, R⁴ represents a methyl group, and R⁵ represents any substituents selected from Group X (hereinafter, referred to as Compound class SX35) .

A compound (1C) wherein Q represents Q2, L represents an oxygen atom, R¹ represents a methyl group, R⁴ represents a methyl group, and R⁵ represents any substituents selected from Group X (hereinafter, referred to as Compound class SX36).

A compound (1C) wherein Q represents Q2, L represents an oxygen atom, R¹ represents a chlorine atom, R⁴ represents an ethyl group, and R⁵ represents any substituents selected from Group X (hereinafter, referred to as Compound class SX37) .

A compound (1C) wherein Q represents Q2, L represents an oxygen atom, R¹ represents a methyl group, R⁴ represents a hydrogen atom, and R⁵ represents any substituents selected from Group X (hereinafter, referred to as Compound class SX38) .

A compound (1C) wherein Q represents Q2, L represents an oxygen atom, R¹ represents a chlorine atom, R⁴ represents a hydrogen atom, and R⁵ represents any substituents selected from Group X (hereinafter, referred to as Compound class SX39) .

A compound (1C) wherein Q represents Q2, L represents NH, R¹ represents a methyl group, R⁴ represents a methyl group, and R⁵ represents any substituents selected from Group X (hereinafter, referred to as Compound class SX40).

A compound (1C) wherein Q represents Q2, L represents NH, R¹ represents a chlorine atom, R⁴ represents a methyl group, and R⁵ represents any substituents selected from Group X (hereinafter, referred to as Compound class SX41).

A compound (1C) wherein Q represents Q2, L represents NH, R¹ represents a methyl group, R⁴ represents an ethyl group, and R⁵ represents any substituents selected from Group X (hereinafter, referred to as Compound class SX42).

A compound (1C) wherein Q represents Q2, L represents NH, R¹ represents a chlorine atom, R⁴ represents an ethyl group, and R⁵ represents any substituents selected from Group X (hereinafter, referred to as Compound class SX43).

A compound (1C) wherein Q represents Q2, L represents NH, R¹ represents a methyl group, R⁴ represents a hydrogen atom, and R⁵ represents any substituents selected from Group X (hereinafter, referred to as Compound class SX44).

A compound (1C) wherein Q represents Q2, L represents NH, R¹ represents a chlorine atom, R⁴ represents a hydrogen atom, and R⁵ represents any substituents selected from Group X (hereinafter, referred to as Compound class SX45).

A compound represented by formula (1D):

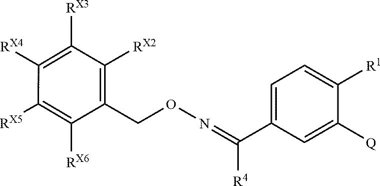

(hereinafter, referred to as “Compound (1D)”),
wherein Q represents Q1, R¹ represents a methyl group, R⁴ represents a methyl group, and a combination of R^{X2}, R^{x3}, R^{X4}, R^{x5}, and R^{X6} represents any combinations described in the combination A (hereinafter, referred to as Compound Class SX46) .

A compound (1D) wherein Q represents Q1, R¹ represents a methyl group, R⁴ represents a methyl group, and a combination of R^{x2}, R^{x3}, R^{x4}, R^{x5}, and R^{x6} represents any combinations described in the combination A (hereinafter, referred to as Compound Class SX47).

A compound (1D) wherein Q represents Q1, R¹ represents a fluorine atom, R⁴ represents a methyl group, and a combination of R^{X2}, R^{X3}, R^{X4}, R^{X5}, and R^{X6} represents any combinations described in the combination A (hereinafter, referred to as Compound Class SX48).

A compound (1D) wherein Q represents Q1, R¹ represents an ethyl group, R⁴ represents a methyl group, and a combination of R^{X2}, R^{x3}, R^{X4}, R^{x5}, and R^{X6} represents any combinations described in the combination A (hereinafter, referred to as Compound Class SX49).

A compound (1D) wherein Q represents Q1, R¹ represents a cyclopropyl group, R⁴ represents a methyl group, and a combination of R^{x2}, R^{x3}, R^{X4}, R^{X5}, and R^{X6} represents any combinations described in the combination A (hereinafter, referred to as Compound Class SX50).

A compound (1D) wherein Q represents Q1, R¹ represents a methyl group, R⁴ represents an ethyl group, and a combination of R^{X2}, R^{x3}, R^{x4}, R^{X5}, and R^{X6} represents any combinations described in the combination A (hereinafter, referred to as Compound Class SX51).

A compound (1D) wherein Q represents Q1, R¹ represents a chlorine atom, R⁴ represents an ethyl group, and a combination of R^{X2}, R^{x3}, R^{x4}, R^{x5}, and R^{X6} represents any combinations described in the combination A (hereinafter, referred to as Compound Class SX52).

A compound (1D) wherein Q represents Q1, R¹ represents a fluorine atom, R⁴ represents an ethyl group, and a combination of R^{x2}, R^{x3}, R^{X4}, R^{X5}, and R^{x6} represents any combinations described in the combination A (hereinafter, referred to as Compound Class SX53).

A compound (1D) wherein Q represents Q1, R¹ represents an ethyl group, R⁴ represents an ethyl group, and a combination of R^{X2}, R^{x3}, R^{x4}, R^{x5}, and R^{X6} represents any combinations described in the combination A (hereinafter, referred to as Compound Class SX54).

A compound (1D) wherein Q represents Q1, R¹ represents a cyclopropyl group, R⁴ represents an ethyl group, and a combination of R^{X2}, R^{X3}, R^{X4}, R^{x5}, and R^{X6} represents any combinations described in the combination A (hereinafter, referred to as Compound Class SX55).

A compound (1D) wherein Q represents Q1, R¹ represents a methyl group, R⁴ represents a hydrogen atom, and a combination of R^{x2}, R^{x3}, R^{X4},R^{X5}, and R^{x6} represents any combinations described in the combination A (hereinafter, referred to as Compound Class SX56).

A compound (1D) wherein Q represents Q1, R¹ represents a chlorine atom, R⁴ represents a hydrogen atom, and a combination of R^{X2}, R^{X3}, R^{X4}, R^{X5}, _{and} R^{X6} represents any combinations described in the combination A (hereinafter, referred to as Compound Class SX57).

A compound (1D) wherein Q represents Q1, R¹ represents a fluorine atom, R⁴ represents a hydrogen atom, and a combination of R^{X2}, R^{X3}, R^{X4}, R^{X5}, and R^{X6} represents any combinations described in the combination A (hereinafter, referred to as Compound Class SX58).

A compound (1D) wherein R¹ represents an ethyl group R⁴ represents a hydrogen atom, and a combination of R^{X2}, R^{x3}, R^{X4}, R^{X5}, and R^{X6} represents any combinations described in the combination A (hereinafter, referred to as Compound Class SX59) .

A compound (1D) wherein Q represents Q1, R¹ represents a cyclopropyl group, R⁴ represents a hydrogen atom, and a combination of R^{X2}, R^{X3}, R^{X4}, R^{X5}, and R^{X6} represents any combinations described in the combination A (hereinafter, referred to as Compound Class SX60).

A compound (1D) wherein Q represents Q2, L represents an oxygen atom, R¹ represents a methyl group, R⁴ represents a methyl group, and a combination of R^{X2}, R^{x3}, R^{X4}, R^{x5}, and R^{X6} represents any combinations described in the combination A (hereinafter, referred to as Compound Class SX61).

A compound (1D) wherein Q represents Q2, L represents an oxygen atom, R¹ represents a chlorine atom, R⁴ represents a methyl group, and a combination of R^{X2}, R^{X3}, R^{X4}, R^{X5}, and R^{X6} represents any combinations described in the combination A (hereinafter, referred to as Compound Class SX62).

A compound (1D) wherein Q represents Q2, L represents an oxygen atom, R¹ represents a methyl group, R⁴ represents an ethyl group, and a combination of R^{X2}, R^{X3}, R^{X4}, R^{X5}, and R^{x6} represents any combinations described in the combination A (hereinafter, referred to as Compound Class SX63).

A compound (1D) wherein Q represents Q2, L represents an oxygen atom, R¹ represents a chlorine atom, R⁴ represents an ethyl group, and a combination of R^{X2}, R^{X3}, R^{X4}, R^{X5}, and R^{X6} represents any combinations described in the combination A (hereinafter, referred to as Compound Class SX64).

A compound (1D) wherein Q represents Q2, L represents an oxygen atom, R¹ represents a methyl group, R⁴ represents a hydrogen atom, and a combination of R^{X2}, R^{X3}, R^{X4}, R^{X5}, and R^{X6} represents any combinations described in the combination A (hereinafter, referred to as Compound Class SX65).

A compound (1D) wherein Q represents Q2, L represents an oxygen atom, R¹ represents a chlorine atom, R⁴ represents a hydrogen atom, and a combination of R^{X2}, R^{X3}, R^{X4}, R^{X5}, and R^{X6} represents any combinations described in the combination A (hereinafter, referred to as Compound Class SX66).

A compound (1D) wherein Q represents Q2, L represents NH, R¹ represents a methyl group, R⁴ represents a methyl group and a combination of R^{X2}, R^{X3}, R^{X4}, R^{X5}, and R^{X6} represents any combinations described in the combination A (hereinafter, referred to as Compound Class SX67).

A compound (1D) wherein Q represents Q2, L represents NH, R¹ represents a chlorine atom, R⁴ represents a methyl group and a combination of R^{X2}, R^{x3}, R^{X4}, R^{x5}, and R^{X6} represents any combinations described in the combination A (hereinafter, referred to as Compound Class SX68).

A compound (1D) wherein Q represents Q2, L represents NH, R¹ represents a methyl group, R⁴ represents an ethyl group and a combination of R^{X2}, R^{x3}, R^{X4}, R^{X5}, and R^{x6} represents any combinations described in the combination A (hereinafter, referred to as Compound Class SX69).

A compound (1D) wherein Q represents Q2, L represents NH, R¹ represents a chlorine atom, R⁴ represents an ethyl group and a combination of R^{X2}, R^{x3}, R^{X4}, R^{x5}, and R^{X6} represents any combinations described in the combination A (hereinafter, referred to as Compound Class SX70).

A compound (1D) wherein Q represents Q2, L represents NH, R¹ represents a methyl group, R⁴ represents a hydrogen atom and a combination of R^{x2}, R^{X3}, R^{X4}, R^{x5}, and R^{X6} represents any combinations described in the combination A (hereinafter, referred to as Compound Class SX71).

A compound (1D) wherein Q represents Q2, L represents NH, R¹ represents a chlorine atom, R⁴ represents a hydrogen atom and a combination of R^{x2}, R^{x3}, R^{x4}, R^{x5}, and R^{X6} represents any combinations described in the combination A (hereinafter, referred to as Compound Class SX72).

A compound represented by formula (1E):

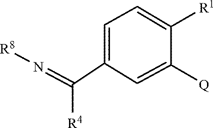

(hereinafter, referred to as “Compound (1E)”),
wherein Q represents Q1, R¹ represents a methyl group, R⁴ represents a methyl group, and R⁸ represents any substituents selected from Group X (hereinafter, referred to as Compound Class SX73).

A compound (1E) wherein Q represents Q1, R¹ represents a chlorine atom, R⁴ represents a methyl group, and R⁸ represents any substituents selected from Group X (hereinafter, referred to as Compound Class SX74).

A compound (1E) wherein Q represents Q1, R¹ represents a methyl group, R⁴ represents a hydrogen atom, and R⁸ represents any substituents selected from Group X (hereinafter, referred to as Compound Class SX75).

A compound (1E) wherein Q represents Q1, R¹ represents a chlorine atom, R⁴ represents a hydrogen atom, and R⁸ represents any substituents selected from Group X (hereinafter, referred to as Compound Class SX76).

A compound (1E) wherein Q represents Q2, L represents an oxygen atom, R¹ represents a methyl group, R⁴ represents a methyl group, and R⁸ represents any substituents selected from Group X (hereinafter, referred to as Compound Class SX77) .

A compound (1E) wherein Q represents Q2, L represents an oxygen atom, R¹ represents a chlorine atom, R⁴ represents a methyl group, and R⁸ represents any substituents selected from Group X (hereinafter, referred to as Compound Class SX78) .

A compound (1E) wherein Q represents Q2, L represents an oxygen atom, R¹ represents a methyl group, R⁴ represents a hydrogen atom, and R⁸ represents any substituents selected from Group X (hereinafter, referred to as Compound Class SX79) .

A compound (1E) wherein Q represents Q2, L represents an oxygen atom, R¹ represents a chlorine atom, R⁴ represents a hydrogen atom, and R⁸ represents any substituents selected from Group X (hereinafter, referred to as Compound Class SX80) .

A compound (1E) wherein Q represents Q2, L represents NH, R¹ represents a methyl group, R⁴ represents a methyl group, and R⁸ represents any substituents selected from Group X (hereinafter, referred to as Compound Class SX81).

A compound (1E) wherein Q represents Q2, L represents NH, R¹ represents a chlorine atom, R⁴ represents a methyl group, and R⁸ represents any substituents selected from Group X (hereinafter, referred to as Compound Class SX82).

A compound (1E) wherein Q represents Q2, L represents NH, R¹ represents a methyl group, R⁴ represents a hydrogen atom, and R⁸ represents any substituents selected from Group X (hereinafter, referred to as Compound Class SX83).

A compound (1E) wherein Q represents Q2, L represents NH, R¹ represents a chlorine atom, R⁴ represents a hydrogen atom, and R⁸ represents any substituents selected from Group X (hereinafter, referred to as Compound Class SX84).

A compound represented by formula (1F):

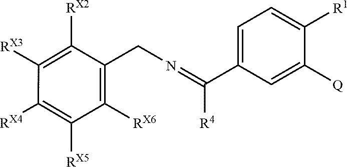

(hereinafter, referred to as “Compound (1F)”),
wherein Q represents Q1, R¹ represents a methyl group, R⁴ represents a methyl group, and a combination of R^{x2}, R^{X3}, R^{x4}, R^{x5}, and R^{x6} represents any combinations described in the combination A (hereinafter, referred to as Compound Class SX85) .

A compound (1F) wherein Q represents Q1, R¹ represents a chlorine atom, R⁴ represents a methyl group, and a combination of R^{X2}, R^{x3}, R^{X4}, R^{x5}, and R^{x6} represents any combinations described in the combination A (hereinafter, referred to as Compound Class SX86).

A compound (1F) wherein Q represents Q1, R¹ represents a methyl group, R⁴ represents a hydrogen atom, and a combination of R^{X2}, R^{X3}, R^{X4}, R^{x5}, and R^{x6} represents any combinations described in the combination A (hereinafter, referred to as Compound Class SX87).

A compound (1F) wherein Q represents Q1, R¹ represents a chlorine atom, R⁴ represents a hydrogen atom, and a combination of R^{X2}, R^{x3}, R^{x4}, R^{X5}, and R^{X6} represents any combinations described in the combination A (hereinafter, referred to as Compound Class SX88).

A compound (1F) wherein Q represents Q2, L represents an oxygen atom, R¹ represents a methyl group, R⁴ represents a methyl group, and a combination of R^{X2}, R^{x3}, R^{x4}, R^{x5}, and R^{X6} represents any combinations described in the combination A (hereinafter, referred to as Compound Class SX89).

A compound (1F) wherein Q represents Q2, L represents an oxygen atom, R¹ represents a chlorine atom, R⁴ represents a methyl group, and a combination of R^{X2}, R^{x3}, R^{x4}, R^{x5}, and R^{X6} represents any combinations described in the combination A (hereinafter, referred to as Compound Class SX90).

A compound (1F) wherein Q represents Q2, L represents an oxygen atom, R¹ represents a methyl group, R⁴ represents a hydrogen atom, and a combination of R^{x2}, R^{X3}, R^{X4}, R^{x5}, and R^{x6} represents any combinations described in the combination A (hereinafter, referred to as Compound Class SX91).

A compound (1F) wherein Q represents Q2, L represents an oxygen atom, R¹ represents a chlorine atom, R⁴ represents a hydrogen atom, and a combination of R^{X2}, R^{x3}, R^{X4}, R^{X5}, and R^{x6} represents any combinations described in the combination A (hereinafter, referred to as Compound Class SX92).

A compound (1F) wherein Q represents Q2, L represents NH, R¹ represents a methyl group, R⁴ represents a methyl group, and a combination of R^{X2}, R^{x3}, R^{x4}, R^{X5}, and R^{X6} represents any combinations described in the combination A (hereinafter, referred to as Compound Class SX93).

A compound (1F) wherein Q represents Q2, L represents NH, R¹ represents a chlorine atom, R⁴ represents a methyl group, and a combination of R^{x2}, R^{x3}, R^{X4}, R^{X5}, and R^{X6} represents any combinations described in the combination A (hereinafter, referred to as Compound Class SX94).

A compound (1F) wherein Q represents Q2, L represents NH, R¹ represents a methyl group, R⁴ represents a hydrogen atom, and a combination of R^{X2}, R^{X3}, R^{X4}, R^{X5}, and R^{X6} represents any combinations described in the combination A (hereinafter, referred to as Compound Class SX95).

A compound (1F) wherein Q represents Q2, L represents NH, R¹ represents a chlorine atom, R⁴ represents a hydrogen atom, and a combination of R^{X2}, R^{x3}, R^{x4}, R^{X5}, and R^{X6} represents any combinations described in the combination A (hereinafter, referred to as Compound Class SX96).

A compound represented by formula (1G):

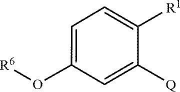

(hereinafter, referred to as “Compound (1G)”),
wherein Q represents Q1, R¹ represents a methyl group, and R⁶ represents any substituents selected from Group Y (hereinafter, referred to as Compound Class SX97).

Group Y: a group consisting of Et,Pr,i— Pr, (CH₂ ) ₃CH₃ , CH₂ CH (CH₃ ) ₂,CH (CH₃ ) CH₂CH₃ , t-Bu,CH₂ c-Pr, (CH₂ ) ₄ CH₃ , (CH₂ ) ₂ CH (CH₃ ) ₂ , CH (CH₃) (CH₂ ) ₂ CH₃ , CH (CH₂ CH₃ ) CH₂ C H₃ ,CH₂ CH (CH₃ ) CH₂ CH₃ ,C(CH₃ ) ₂ CH₂ CH₃ , CH₂ t-Bu, CH_{2 C}-B_{U}, CH_{2 C}-Pen, (CH₂)₂c-Pr, (1-methylcyclopropyl)methyl, (2-methylcyclopropyl)methyl,1-cyclopropylethyl, (CH₂)₃C-Pr, (CH₂)₅ CH₃, (CH₂)₃CH(CH₃)₂, (CH₂ )₂ CH(CH₃)CH₂CH₃,CH₂CH(CH₃) ( CH₂ ) ₂ CH₃ , CH (CH₃ ) (CH₂ ) ₃ CH₃ , CH₂ CH (CH₂ CH₃ ) ₂ , heptyl, CH₂ CH═CH₂ H₂ CH═CHCH₃ , CH₂ CH═C (CH₃ ) ₂ , CH₂ CH═CF₂ , CH₂ CH═CC1₂ , CH₂ CH═CHCH₂ CH ₃ , CH₂ CH═CH (CH₂ ) ₂ CH₃ , CH₂ C (CH₃ ) ═CH₂ , CH₂C (CH₃ ) ═CHCH₃ , CH₂ C (CH₃ ) ═C(CH₃ )₂ , CH₂ C(CH₃)═CHCH₂ CH₃ , CH₂ CF═CH₂,CH₂ CF═CHCH₃,CH₂ CF═C(C H₃ ) ₂ , CH₂ CF═CF₂ , CH₂ CF═CHCH₂ CH₃ , CH₂ CF═CH (CH₂ ) ₂ CH₃ , CH₂ CC1═CH₂ , CH₂CCl═CHCH₃,CH₂CCl═C(CH₃)₂,CH₂CCl═CCl₂,CH₂CCl═CHCH₂CH₃,CH₂ CCl═CH (CH₂ )₂CH₃, (CH₂ )₂CH═CH₂ , (CH₂ ) ₂CH═CHCH₃, (CH₂ )₂ CH═CHCH₂C H₃ , (CH₂ )₂CH═C (CH₃ ) ₂ , (CH₂ ) ₂ C (CH₃ )═CH₂ , (CH₂ ) ₂C (CH₃ ) ═CHCH₃ , (CH ₂ ) ₃ CH═CH₂ , (CH₂ ) ₃C (CH₃ ) ═CH₂ , (CH₂ ) ₄ CH═CH₂ ,CH₂ C═CH, CH₂C═CCH₃ , C H₂C═CCH₂CH₃ , CH₂C═Cc—Pr, (CH2 )₂ C═CH, (CH₂ ) ₂ C═CCH3 , (CH₂ ) ₂ C═CCH₂ CH₃ , (CH₂) ₃ C═CH, (CH₂ ) ₃ C═ CCH₃ , CH₂ C1, CH₂ Br, CH₂ OCH₃ , CH₂ 0CH₂ CH₃ , CH₂CH₂OCH₃ , CH₂ O (CH₂ ) ₂ CH₃ , CHF₂ , CF₃ , CH₂ CF₃ , CH₂ CHF₂ , CH₂ CH₂ F, CH₂ CH₂ CH₂ F, CH₂ CH₂ CHF₂ , CH₂ C H₂CF₃ ,CH₂ CF₂ CF₃ , (CH₂ ) ₂CF₂ CF₃ , CH₂ (CF₂ ) ₂ CF₃ , (CH₂ ) ₂CF (CF₃ ) ₂, (C H₂ ) ₂ (CF₂ ) ₅ CF₃ , CF₃ , CF₂ CHF (CF₃ ) , CF₂CHF (OCF₃ ) , CH₂ CF₂ CF₂ H, c—Pr, and c—Bu.

A compound (1G) wherein Q represents Q1, R¹ represents a chlorine atom, and R⁶ represents any substituents selected from Group Y (hereinafter, referred to as Compound Class SX98).

A compound (1G) wherein Q represents Q1, R¹ represents a fluorine atom, and R⁶ represents any substituents selected from Group Y (hereinafter, referred to as Compound Class SX99).

A compound (1G) wherein Q represents Q1, R¹ represents an ethyl group, and R⁶ represents any substituents selected from Group Y (hereinafter, referred to as Compound Class SX100).

A compound (1G) wherein Q represents Q1, R¹ represents a cyclopropyl group, and R⁶ represents any substituents selected from Group Y (hereinafter, referred to as Compound Class SX101).

A compound (1G) wherein Q represents Q2, L represents an oxygen atom, R¹ represents a methyl group, and R⁶ represents any substituents selected from Group Y (hereinafter, referred to as Compound Class SX102).

A compound (1G) wherein Q represents Q2, L represents an oxygen atom, R¹ represents a chlorine atom, and R⁶ represents any substituents selected from Group Y (hereinafter, referred to as Compound Class SX103).

A compound (1G) wherein Q represents Q2, L represents NH, R¹ represents a methyl group, and R⁶ represents any substituents selected from Group Y (hereinafter, referred to as Compound Class SX104).

A compound (1G) wherein Q represents Q2, L represents NH, R¹ represents a chlorine atom, and R⁶ represents any substituents selected from Group Y (hereinafter, referred to as Compound Class SX105).

A compound represented by formula (1H):

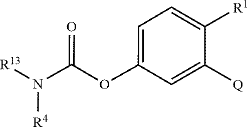

(hereinafter, referred to as “Compound (1H)”) ,
wherein Q represents Q1, R¹ represents a methyl group, R⁴ represents a methyl group, and R¹³ represents any substituents selected from Group X (hereinafter, referred to as Compound Class SX106).

A compound (1H) wherein Q represents Q1, R¹ represents a chlorine atom, R⁴ represents a methyl group, and R¹³ represents any substituents selected from Group X (hereinafter, referred to as Compound Class SX107).

A compound (1H) wherein Q represents Q1, R¹ represents a methyl group, R⁴ represents a hydrogen atom, and R¹³ represents any substituents selected from Group X (hereinafter, referred to as Compound Class SX108).

A compound (1H) wherein Q represents Q1, R¹ represents a chlorine atom, R⁴ represents a hydrogen atom, and R¹³ represents any substituents selected from Group X (hereinafter, referred to as Compound Class SX109).

A compound (1H) wherein Q represents Q2, L represents an oxygen atom, R¹ represents a methyl group, R⁴ represents a methyl group, and R¹³ represents any substituents selected from Group X (hereinafter, referred to as Compound Class SX110) .

A compound (1H) wherein Q represents Q2, L represents an oxygen atom, R¹ represents a chlorine atom, R⁴ represents a methyl group, and R¹³ represents any substituents selected from Group X (hereinafter, referred to as Compound Class SX111) .

A compound (1H) wherein Q represents Q2, L represents an oxygen atom, R¹ represents a methyl group, R⁴ represents a hydrogen atom, and R¹³ represents any substituents selected from Group X (hereinafter, referred to as Compound Class SX112).

A compound (1H) wherein Q represents Q2, L represents an oxygen atom, R¹ represents a chlorine atom, R⁴ represents a hydrogen atom, and R¹³ represents any substituents selected from Group X (hereinafter, referred to as Compound Class SX113) .

A compound (1H) wherein Q represents Q2, L represents NH, R¹ represents a methyl group, R⁴ represents a methyl group, and R¹³ represents any substituents selected from Group X (hereinafter, referred to as Compound Class SX114).

A compound (1H) wherein Q represents Q2, L represents NH, R¹ represents a chlorine atom, R⁴ represents a methyl group, and R¹³ represents any substituents selected from Group X (hereinafter, referred to as Compound Class SX115).

A compound (1H) wherein Q represents Q2, L represents NH, R¹ represents a methyl group, R⁴ represents a hydrogen atom, and R¹³ represents any substituents selected from Group X (hereinafter, referred to as Compound Class SX116).

A compound (1H) wherein Q represents Q2, L represents NH, R¹ represents a chlorine atom, R⁴ represents a hydrogen atom, and R¹³ represents any substituents selected from Group X (hereinafter, referred to as Compound Class SX117).

A compound represented by formula (1I):

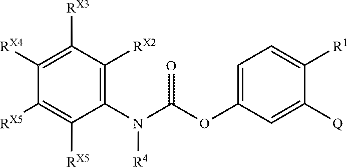

(hereinafter, referred to as “Compound (1I)”) wherein Q represents Q1, R¹ represents a methyl group, R⁴ represents a methyl group, and a combination of R^{x2}, R^{x3}, R^{x4}, R^{x5}, and R^{x6} represents any substituents selected from Group A (hereinafter, referred to as Compound class SX118).

A compound (1I) wherein Q represents Q1, R¹ represents a chlorine atom, R⁴ represents a methyl group, and a combination of R^{x2}, R^{x3}, R^{x4}, R^{x5} , and R^{x6} represents any substituents selected from Group A (hereinafter, referred to as Compound class SX119).

A compound (1I) wherein Q represents Q1, R¹ represents a methyl group, R⁴ represents an ethyl group, and a combination of R^{x2}, R^{x3}, R^{x4}, R^{x5}, and R^{x6} represents any substituents selected from Group A (hereinafter, referred to as Compound class SX120).

A compound (1I) wherein Q represents Q1, R¹ represents a chlorine atom, R⁴ represents an ethyl group, and a combination of R^{x2}, R^{x3}, R^{x4}, R^{x5}, and R^{x6} represents any substituents selected from Group A (hereinafter, referred to as Compound class SX121).

A compound (1I) wherein Q represents Q1, R¹ represents a methyl group, R⁴ represents a hydrogen atom, and a combination of R^{x2}, R^{x3}, R^{x4}, R^{x5}, and R^{x6} represents any substituents selected from Group A (hereinafter, referred to as Compound class SX122).

A compound (1I) wherein Q represents Q1, R¹ represents a chlorine atom, R⁴ represents a hydrogen atom, and a combination of R^{x2}, R^{x3}, R^{x4}, R^{x5}, and R^{x6} represents any substituents selected from Group A (hereinafter, referred to as Compound class SX123).

A compound (1I) wherein Q represents Q2, L represents an oxygen atom, R¹ represents a methyl group, R⁴ represents a methyl group, and a combination of R^{x2}, R^{x3}, R^{x4}, R^{x5}, and R^{x6} represents any substituents selected from Group A (hereinafter, referred to as Compound class SX124).

A compound (1I) wherein Q represents Q2, L represents an oxygen atom, R¹ represents a chlorine atom, R⁴ represents a methyl group, and a combination of R^{x2}, R^{x3}, R^{x4}, R^{x5}, and R^{x6} represents any substituents selected from Group A (hereinafter, referred to as Compound class SX125).

A compound (1I) wherein Q represents Q2, L represents an oxygen atom, R¹ represents a methyl group, R⁴ represents an ethyl group, and a combination of R^{x2}, R^{x3}, R^{x4}, R^{x5}, and R^{x6} represents any substituents selected from Group A (hereinafter, referred to as Compound class SX126).

A compound (1I) wherein Q represents Q2, L represents an oxygen atom, R¹ represents a chlorine atom, R⁴ represents an ethyl group, and a combination of R^{x2}, R^{x3}, R^{x4}, R^{x5}, and R^{x6} represents any substituents selected from Group A (hereinafter, referred to as Compound class SX127).

A compound (1I) wherein Q represents Q2, L represents an oxygen atom, R¹ represents a methyl group, R⁴ represents a hydrogen atom, and a combination of R^{x2}, R^{x3}, R^{x4}, R^{x5}, and R^{x6} represents any substituents selected from Group A (hereinafter, referred to as Compound class SX128).

A compound (1I) wherein Q represents Q2, L represents an oxygen atom, R¹ represents a chlorine atom, R⁴ represents a hydrogen atom, and a combination of R^{x2}, R^{x3}, R^{x4}, R^{x5}, and R^{x6} represents any substituents selected from Group A (hereinafter, referred to as Compound class SX129).

A compound (1I) wherein Q represents Q2, L represents NH, R¹ represents a methyl group, R⁴ represents a methyl group, and a combination of R^{x2}, R^{x3}, R^{x4}, R^{x5}, and R^{x6} represents any substituents selected from Group A (hereinafter, referred to as Compound class SX130).

A compound (1I) wherein Q represents Q2, L represents NH, R¹ represents a chlorine atom, R⁴ represents a methyl group, and a combination of R^{x2}, R^{x3}, R^{x4}, R^{x5}, and R^{x6} represents any substituents selected from Group A (hereinafter, referred to as Compound class SX131).

A compound (1I) wherein Q represents Q2, L represents NH, R¹ represents a methyl group, R⁴ represents an ethyl group, and a combination of R^{x2}, R^{x3}, R^{x4}, R^{x5}, and R^{x6} represents any substituents selected from Group A (hereinafter, referred to as Compound class SX132).

A compound (1I) wherein Q represents Q2, L represents NH, R¹ represents a chlorine atom, R⁴ represents an ethyl group, and a combination of R^{x2}, R^{x3}, R^{x4}, R^{x5}, and R^{x6} represents any substituents selected from Group A (hereinafter, referred to as Compound class SX133).

A compound (1I) wherein Q represents Q2, L represents NH, R¹ represents a methyl group, R⁴ represents a hydrogen atom, and a combination of R^{x2}, R^{x3}, R^{x4}, R^{x5}, and R^{x6} represents any substituents selected from Group A (hereinafter, referred to as Compound class SX134).

A compound (1I) wherein Q represents Q2, L represents NH, R¹ represents a chlorine atom, R⁴ represents a hydrogen atom, and a combination of R^{x2}, R^{x3}, R^{x4}, R^{x5}, and R^{x6} represents any substituents selected from Group A (hereinafter, referred to as Compound class SX135).

A compound represented by formula (1J):

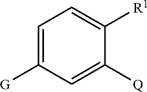

[wherein G represents any one of formulae G1 to formula G35.

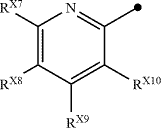

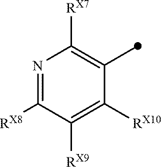

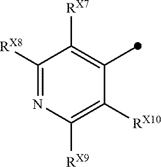

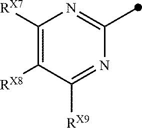

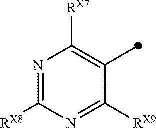

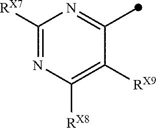

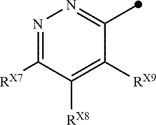

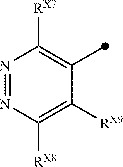

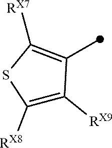

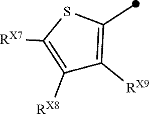

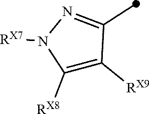

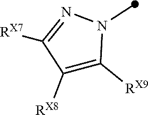

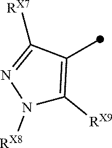

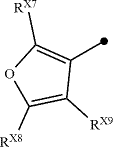

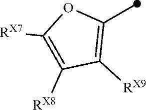

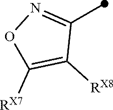

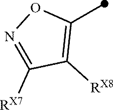

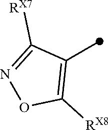

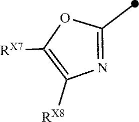

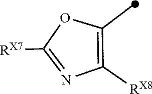

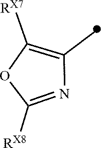

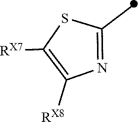

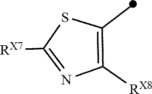

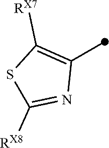

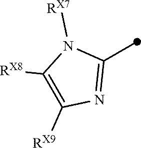

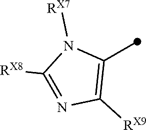

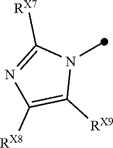

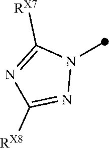

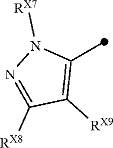

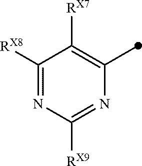

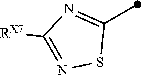

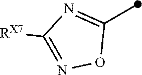

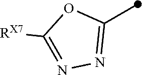

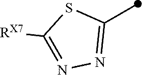

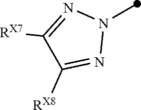

] (hereinafter, referred to as “Compound (1J)”) wherein Q represents Q1, R¹ represents a methyl group, and a combination of structure of G and substituents of R^{x7}, R^{x8}, R^{x9} and R^{x10} applied to the structure of G represents any combination described in Combination B (hereinafter, referred to as Compound class SX136).

Combination B consists of substituent numbers ZB1 to ZB1539.

Substituent numbers ZB1 to ZB1539 represent combinations of structure of G and substituents of R^{X7}, R^{x8}, R^{x9} and R^{x10} applied to the structure of G according to Compound (1J), which hereinafter, is referred to as [Substituent No.; G, R^{x7}, R^{x8}, R^{x9}, R^{x10}] . For example, Substituent No. ZB2 represents a combination wherein G represents G1, R^{x7} represents a methyl group, and R^{x8}, R^{x9}, and R^{x10} represent a hydrogen atom.

### Combination B

[ZB1;G1,H,H,H,H],[ZB2;G1,Me,H,H,H],[ZB3;G1,Et,H,H,H],[ZB4;G 1,Pr,H,H,H] , [ZB5;G1,i-Pr,H,H,H] , [ZB6;G1,c-Pr,H,H,H],[ZB7;G1,Ph,H,H,H] , [ZB8;G1,OMe,H,H,H],[ZB9;G1,OEt, H,H,H] , [ZB10;G1,OPh,H,H,H] , [ZB11;G1,CF₃,H,H,H] , [ZB12;G1,F,H ,H,H],[ZB13;G1,Cl,H,H,H] , [ZB14;G1,Br,H,H,H] , [ZB15;G1,CN,H,H ,H] , [ZB16;G1,H,Me,H,H] , [ZB17;G1,H,Et,H,H] , [ZB18;G1,H,Pr,H,H ] , [ZB19; G1,H,i-Pr,H,H] , [ZB20;G1,H,c-Pr,H,H] , [ZB21;G1,H,Ph,H,H] , [ZB22;G1,H,OMe,H,H] , [ZB23;G1,H,O Et,H,H] , [ZB24;G1,H,OPh,H,H] , [ZB25;G1,H,CF₃,H,H] , [ZB26;G1,H, F,H,H] , [ZB27;G1,H,Cl,H,H] , [ZB28;G1,H,Br,H,H] , [ZB29;G1,H,eN, H,H] , [ZB30;G1,H,H,Me,H] , [ZB31;G1,H,H,Et,H] , [ZB32;G1,H,H,Pr, H] , [ZB33;G1,H,H,i-Pr,H] , [ZB34;G1,H,H,c-Pr,H] , [ZB35;G1,H,H,Ph,H] , [ZB36;G1,H,H,OMe,H] , [ZB37;G1,H,H,O Et,H] , [ZB38;G1,H,H,OPh,H] , [ZB39;G1,H,H,CF₃,H] , [ZB40;G1,H,H, F,H] , [ZB41;G1,H,H,Cl,H] , [ZB42;G1,H,H,Br,H] , [ZB43;G1,H,H,CN, H] , [ZB44;G1,H,H,H,Me] , [ZB45;G1,H,H,H,Et] , [ZB46;G1,H,H,H,Pr] , [ZB47;G1,H,H,H,i-Pr] , [ZB98;G1,H,H,H,c-Pr] , [ZB49;G1,H,H,H,Ph] , [ZB50;G1,H,H,H,OMe] , [ZB51;G1,H,H,H,O Et] , [ZB52;G1,H,H,H,OPh] , [ZB53;G1,H,H,H,CF₃] , [ZB54;G1,H,H,H, F] , [ZB55;G1,H,H,H,Cl] , [ZB56;G1,H,H,H,Br] , [ZB57;G1,H,H,H,CN] , [ZB58;G2,H,H,H,H] , [ZB59;G2,Me,H,H,H] , [ZB60;G2,Et,H,H,H] , [Z B61;G2,Pr,H,H,H] , [ZB62;G2,i-Pr,H,H,H] , [ZB63;G2,c-Pr,H,H,H] , [ZB64;G2,Ph,H,H,H] , [ZB65;G2,OMe,H,H,H] , [ZB66;G2,O Et,H,H,H] , [ZB67;G2,OPh,H,H,H] , [ZB68;G2,CF₃,H,H,H] , [ZB69;G2, F,H,H,H] , [ZB70;G2,Cl,H,H,H] , [ZB71;G2,Br,H,H,H] , [ZB72;G2,CN, H,H,H] , [ZB73;G2,H,Me,H,H] , [ZB74;G2,H,Et,H,H] , [ZB75;G2,H,Pr, H,H] , [ZB76;G2,H,i-Pr,H,H] , [ZB77;G2,H,c-Pr,H,H] , [ZB78;G2,H,Ph,H,H] , [ZB79;G2,H,OMe,H,H] , [ZB80;G2,H,O Et,H,H] , [ZB81;G2,H,OPh,H,H] , [ZB82;G2,H,CF₃,H,H] , [ZB83;G2,H, F,H,H] , [ZB84;G2,H,Cl,H,H] , [ZB85;G2,H,Br,H,H] , [ZB86;G2,H,CN, H,H] , [ZB87;G2,H,H,Me,H] , [ZB88;G2,H,H,Et,H] , [ZB89;G2,H,H,Pr, H] , [ZB90;G2,H,H,i-Pr,H] , [ZB91;G2,H,H,c-Pr,H] , [ZB92;G2,H,H,Ph,H] , [ZB93;G2,H,H,OMe,H] , [ZB94;G2,H,H,O Et,H] , [ZB95:G2,H,H,OPh,H] , [ZB96;G2,H,H,CF₃,H] , [ZB97;G2,H,H, F,H] , [ZB98;G2,H,H,Cl,H] , [ZB99;G2,H,H,Br,H] , [ZB100;G2,H,H,CN ,H] , [ZB101;G2,H,H,H,Me] , [ZB102;G2,H,H,H,Et] , [ZB103;G2,H,H,H ,Pr] , [ZB104;G2,H,H,H,i-Pr] , [ZB105;G2,H,H,H,c-Pr] , [ZB106;G2,H,H,H,Ph] , [ZB107;G2,H,H,H,OMe] , [ZB108;G2,H,H, H,OEt] , [ZB109;G2,H,H,H,OPh] , [ZB110;G2,H,H,H,CF₃] , [ZB111;G2, H,H,H,F] , [ZB112;G2,H,H,H,Cl] , [ZB113;G2,H,H,H,Br] , [ZB114;G2, H,H,H,CN] , [ZB115;G3,H,H,H,H] , [ZB116;G3,Me,H,H,H] , [ZB117;G3, Et,H,H,H] , [ZB118;G3,Pr,H,H,H] , (ZB119;G3,i-Pr,H,H,H] , [ZB120;G3,c-Pr,H,H,H] , [ZB121;G3,Ph,H,H,H] , [ZB122;G3,OMe,H,H,H] , [ZB123;G 3,OEt,H,H,H] , [ZB124;G3,OPh,H,H,H] , [ZB125;G3,CF₃,H,H,H] , [ZB1 26;G3,F,H,H,H] , [ZB127;G3,Cl,H,H,H] , [ZB228;G3,Br,H,H,H] , [ZB1 29;G3,CN,H,H,H] , [ZB130;G3,H,Me,H,H] , [ZB131;G3,H,Et,H,H] , [ ZB 132;G3,H,Pr,H,H] , [ZB133;G3,H,i-Pr,H,H] , [ZB134;G3,H,c-Pr,H,H] , [ZB135;G3,H,Ph,H,H] , [ZB136;G3,H,OMe,H,H] , [ZB137;G3, H,OEt,H,H] , [ZB138:G3,H,OPh,H,H] , [ZB139;G3,H,CF₃,H,H] , [ZB140 ;G3,H,F,H,H] , [ZB141;G3,H,Cl,H,H] , [ZB142;G3,H,Br,H,H] , [ZB143 ;G3,H,CN,H,H] , [ZB144;G3,H,H,Me,H] , [ZB145;G3,H,H,Et,H] , [ZB14 6;G3,H,H,Pr,H] , [ZB147;G3,H,H,i-Pr,H] , [ZB148;G3,H,H,c-Pr,H] , [ZB149;G3,H,H,Ph,H] , [ZB150;G3,H,H,OMe,H] , [ZB151;G3,H, H,OEt,H] , [ZB152;G3,H,H,OPh,H] , [ZB153;G3,H,H,CF₃,H] , [ZB154;G 3,H,H,F,H] , [ZB155;G3,H,H,Cl,H] , [ZB156;G3,H,H,Br,H] , [ZB157;G 3,H,H,eN,H] , [ZB158;G3,H,H,H,Me] , [ZB159;G3,H,H,H,Et] , [ZB160; G3,H,H,H,Pr] , [ZB161;G3,H,H,H,i-Pr] , [ZB162;G3,H,H,H,c-Pr] , [ZB163;G3,H,H,H,Ph] , [ZB164;G3,H,H,H,OMe] , [ZB165;G3,H,H, H,OEt] , [ZB166;G3,H,H,H,OPh] , [ZB167;G3,H,H,H,CF_{3]} , [ZB168;G3, H,H,H,F] ,[ZB169;G3,H,H,H,Cl] , [ZB170;G3,H,H,H,Br] , [ZB171;G3, H,H,H,eN] , [ZB172;G4,H,H,H,-] , [ZB173;G4,Me,H,H,-] , [ZB174;G4, Et,H,H,-] , [ZB175;G4,Pr,H,H,-] , [ZB176;G4,i-Pr,H,H,-] , [ZB177;G4,c-Pr,H,H,-] , [ZB178;G4,Ph,H,H,-] , [ZB179;G4,OMe,H,H,-] , [ZB180;G 4,OEt,H,H,-] , [ZB181;G4,OPh,H,H,-] , [ZB182;G4,CF₃,H,H,-] , [ZB1 83;G4,F,H,H,-] , [ZB184;G4,Cl,H,H,-] , [ZB185;G4,Br,H,H,-] , [ZB1 86;G4,CN,H,H,-] , [ZB187;G4,H,Me,H,-] , [ZB188;G4,H,Et,H,-] , [ZB 189;G4,H,Pr,H,-] , [ZB190;G4,H,i-Pr,H,-] , [ZB191;G4,H,c-Pr,H,-] , [ZB192;G4,H,Ph,H,-] , [ZB193;G4,H,OMe,H,-] , [ZB194;G4, H,OEt,H,-] , [ZB195;G4,H,OPh,H,-j , [ZB196;G4,H,CF₃,H,-] , [ZB197 ;G4,H,F,H,-] , [ZB198:G4,H,C1,H,-] , [ZB199;G4,H,Br,H,-] , [ZB200 ;G4,H,CN,H,-],[ZB201;G4,H,H,Me,-],[ZB202;G4,H,H,Et,-],[ZB20 3;G4,H,H,Pr,-] , [ZB204;G4,H,H,i-Pr,-] , [ZB205;G4,H,H,c-Pr,-] , [ZB206;G4,H,H,Ph-] , [ZB207;G4,H,H,OMe,-] , [ZB208;G4,H, H,OEt,-] , [ZB209;G4,H,H,OPh,-] , [ZB210;G4,H,H,CF₃-] , [ZB211;G 4,H,H,F,-] , [ZB212;G4,H,H,Cl,-] , [ZB213;G4,H,H,Br,-] , [ZB214;G 4,H,H,CN,-] , [ZB215;G5,H,H,H,-] , [ZB216;G5,Me,H,H,-] , [ZB217;G 5,Et,H,H,-] , [ZB218;G5,Pr,H,H,-] , [ZB219;G5,i-Pr,H,H,-] , [ZB220;G5,c-Pr,H,H,-] , [ZB221;G5,Ph,H,H,-] , [ZB222;G5,OMe,H,H,-] , [Z8223;G 5,OEt,H,H,-] , [ZB224;G5,OPh,H,H,-] , [ZB225;G5,CF₃,H,H,-] , [ZB2 26;G5,F,H,H,-] , [ZB227;G5,Cl,H,H,-] , [ZB228;G5,Br,H,H,-] , [ZB2 29:G5,CN,H,H,-] , [ZB230;G5,H,Me,H,-] , [ZB231;G5,H,Et,H,-] , [ZB 232;G5,H,Pr,H,-] , [ZB233;G5,H,i-Pr,H,-] , [ZB234;G5,H,c-Pr,H,-] , [ZB235;G5,H,Ph,H,-] , [ZB236;G5,H,OMe,H,-] , [ZB237;G5, H,OEt,H,-] , [ZB238;G5,H,OPh,H,-] , [ZB239;G5,H,CF₃,H,-] , [ZB240 ;G5,H,F,H,-],[ZB241;G5,H,Cl,H,-],[ZB242;G5,H,Br,H,-],[ZB243 ;G5,H,CN,H,-] , [ZB244;G5,H,H,Me,-] , [ZB245;G5,H,H,Et,-] , [ZB24 6;G5,H,I-i,Pr,-] , [ZB247;G5,H,H,i-Pr,-] , [ZB248;G5,H,H,c-Pr,-] , [ZB249;G5,H,H,Ph,-] , [ZB250;G5,H,H,OMe,-] , [ZB251;G5,H, H,OEt,-] , [ZB252;G5,H,H,OPh,-] , [ZB253;G5,H,H,CF₃,-] , [ZB254;G 5,H,H,F,-] , [ZB255;G5,H,H,Cl,-] , [ZB256;G5,H,H,Br,-] , [ZB257;G 5,H,H,CN,-] , [ZB258;G6,H,H,H,-] , [ZB259;G6,Me,H,H,-] , [ZB260;G 6,Et,H,H,-] , [ZB261;G6,Pr,H,H,-] , [ZB262;G6,i-Pr,H,H,-] , [ZB263;G6,c-Pr,H,H,-] , [ZB254;G6,Ph,H,H,-] , [ZB265;G6,OMe,H,H,-] , [ZB266;G 6,OEt,H,H,-] , [ZB267;G6,OPh,H,H,-] , [ZB268;G6,CF₃,H,H,-] , [ZB2 69;G6,F,H,H,-] , [ZB270;G6,Cl,H,H,-] , [ZB271;G6,Br,H,H,-] , [ZB2 72;G6,CN,H,H,-],[ZB273;G6,H,Me,H,-],[ZB274;G6,H,Et,H,-],[ZB 275;G6,H,Pr,H,-] , [ZB276;G6,H,i-Pr,H,-] , [ZB277;G6,H,c-Pr,H,-] , [ZB278;G6,H,Ph,H,-] , [ZB279:G6,H,OMe,H,-] , [ZB280;G6, H,OEt,H,-] , [ZB281;G6,H,OPh,H,-] , [ZB282;G6,H,CF₃,H,-] , [ZB283 ;G6,H,F,H,-] , [ZB284;G6,H,Cl,H,-] , [ZB285;G6,H,Br,H,-] , [ZB286 ;G6,H,CN,H,-] , [ZB287;G6,H,H,Me,-] , [ZB288;G6,H,H,Et,-] , [ZB28 9;G6,H,H,Pr,-] , [ZB290;G6,H,H,i-Pr,-] , [ZB291;G6,H,H,c-Pr,-] , [ZB292;G6,H,H,Ph,-] , [ZB293;G6,H,H,OMe,-] , [ZB294;G6,H, H,OEt,-] , [ZB295;G6,H,H,OPh,-] , [ZB296;G6,H,H,CF₃,-] , [ZB297;G 6,H,H,F,-] , [ZB298;G6,H,H,Cl,-] , [ZB299;G6,H,H,Br,-] , [ZB300;G 6,H,H,CN,-] , [ZB301;G7,H,H,H,-] , [ZB302;G7,Me,H,H,-] , [ZB303;G 7,Et,H,H,-] , [ZB304;G7,Pr,H,H,-] , [ZB3G5;G7,i-Pr,H,H,-] , [ZB306;G7,c-Pr,H,H,-] , [ZB307;C7,Ph,H,H,-] , [ZB308;G7,OMe,H,H,-] , [ZB309;G 7,OEt,H,H,-] , [ZB310;G7,OPh,H,H,-] , [ZB311;G7,CF₃,H,H,-] , [ZB3 12;G7,F,H,H,-] , [ZB313;G7,Cl,H,H,-] , [ZB314;G7,Br,H,H,-] , [ZB3 15;G7,CN,H,H,-],[ZB316;G7,H,Me,H,-],[ZB317;G7,H,Et,H,-],[ZB 318;G7,H,Pr,H,-] , [ZB319;G7,H,i-Pr,H,-] , [ZB320;G7,H,c-Pr,H,-] , [ZB321;G7,H,Ph,H,-] , [ZB322;G7,H,OMe,H,-] , [ZB323;G7, H,OEt,H,-] , [ZB324;G7,H,OPh,H,-] , [ZB325;G7,H,CF₃,H,-] , [ZB326 ;G7,H,F,H,-] , [ZB327;G7,H,Cl,H,-] , [ZB328;G7,H,Br,H,-] , [ZB329 ;G7,H,CN,H,-] , [ZB330;G7,H,H,Me,-] , [ZB331;G7,H,H,Et-] , [ZB33 2;G7,H,H,Pr,-] , [ZB333;G7,H,H,i-Pr,-] , [ZB334;G7,H,H,c-Pr,-] , [ZB335;G7,H,H,Ph,-] , [ZB336;G7,H,H,OMe,-] , [ZB337;G7,H, H,OEt,-] , [ZB338;G7,H,H,OPh,-] , [ZB339;G7,H,H,CF₃,-] , [ZB340;G 7,H,H,F,-] , [ZB341;G7,H,H,Cl,-] , [ZB342;G7,H,H,Br,-] , [ZB343;G 7,H,H,CN,-] , [ZB344;G8,H,H,H,-] , [ZB345;G8,Me,H,H,-] , [ZB346;G 8,Et,H,H,-] , [ZB347;G8,Pr,H,H,-] , [ZB348;G8,i-Pr,H,H,-] , [ZB349;G8,c-Pr,H,H,-],[ZB350;G8,Ph,H,H,-] , [ZB351;G8,OMe,H,H,-] , [ZB352;G 8,OEt,H,H,-] , [ZB353;G8,OPh,H,H,-] , [ZB354;G8,CF₃,H,H,-] , [ZB3 55;G8,F,H,H,-] , [ZB356;G8,Cl,H,H,-] , [ZB357;G8,Br,H,H,-] , [ZB3 58;G8,CN,H,H,-] , [ZB359;G8,H,Me,H,-] , [ZB360;G8,H,Et,H,-] , (ZB 361;G8,H,Pr,H,-] , [ZB362;G8,H,i-Pr,H,-] , [ZB363;G8,H,c-Pr,H,-], [ZB364;G8,H,Ph,H,-], [ZB365;G8,H,OMe,H,-], [ZB366;G8, H,OEt,H,-] , [ZB367;G8,H,OPh,H,-] , [ZB368;G8,H,CF₃,H,-] , [ZB369 ;G8,H,F,H,-] , [ZB370;G8,H,Cl,H,-] , [ZB371;G8,H,Br,H,-] , [ZB372 ;G8,H,CN,H,-] , [ZB373;G8,H,H,Me,-] , [ZB374;G8,H,H,Et,-] , [ZB37 5;G8,H,H,Pr′,-] , [ZB376;G8,H,H,1-Pr,-] , [ZB377;G8,H,H,c-Pr,-] , [ZB378;G8,H,H,Ph,-] , [ZB379;G8,H,H,OMe,-] , [ZB380;G8,H, H,OEt,-] , [ZB382;G8,H,H,OPh,-] , [ZB382;G8,H,H,CF₃.-] , [ZB383;G 8,H,H,F,-] , [ZB384;G8,H,H,Cl,-] , [ZB385;G8,H,H,Br,-] , [ZB386;G 8,H,H,CN,-] , [ZB387;G9,H,H,H,-] , [ZB388;G9,Me,H,H,-] , [ZB389;G 9,Et,H,H,-] , [ZB390;G9,Pr,H,H,-] , [ZB391;G9,i-Pr,H,H,-] , [ZB392;G9,c-Pr,H,H,-] , [ZB393;G9,Ph,H,H,-] , [ZB394;G9,OMe,H,H,-] , [ZB395;G 9,OEt,H,H,-] , [ZB396;G9,OPh,H,H,-] , [ZB397;G9,CF₃,H,H,-] , [ZB3 98;G9,F,H,H,-] , [ZB399;G9,Cl,H,H,-] , [ZB400;G9,Br,H,H,-] , [ZB4 01;G9,CN,H,H,-] , [ZB402;G9,H,Me,H,-] , [ZB403;G9,H,Et,H,-] , [ZB 404;G9,H,Pr,H,-] , [ZB405;G9,H,i-Pr,H,-] , [ZB406;G9,H,c-Pr,H,-] , [ZB407;G9,H,Ph,H,-] , [ZB408;G9,H,OMe,H,-] , [ZB409;G9, H,OEt,H,-] , [ZB410;G9,H,OPh,H,-] , [ZB411;G9,H,CF₃,H, -] , [ZB412 ;G9,H,F,H,-] , [ZB413;G9,H,Cl,H,-] , [ZB414;G9,H,Br,H,-] , [ZB415 ;G9,H,CN,H,-] , [ZB416;G9,H,H,Me,-] , [ZB417;G9,H,H,Et,-] , [ZB41 8;G9,H,H,Pr,-] , [ZB419;G9,H,H,i-Pr,-] , [ZB420;G9,H,H,c-Pr,-] , [ZB421;G9,H,H,Ph,-] , [ZB422;G9,H,H,OMe,-] , [ZB423;G9,H, H,OEt,-] , [ZB424;G9,H,H,OPh,-] , [ZB425;G9,H,H,CF₃,-] , [ZB426;G 9,H,H,F,-] , [ZB427;G9,H,H,Cl,-] , [ZB428;G9,H,H,Br,-] , [ZB429;G 9,H,H,CN,-] , [ZB430;G10,H,H,H,-] , [ZB431;G10,Me,H,H,-] , [ZB432 ;G10,Et,H,H,-] , [ZB433;G10,Pr,H,H,-] , [ZB434;G10,i-Pr,H,H,-] , [ZB435;G10,c-Pr,H,H,-] , [ZB436;G10,Ph,H,H,-] , [ZB437;G10,OMe,H,H,-], [ZB438 ;G10,OEt,H,H,-] , [ZB439;G10,OPh,H,H,-] , [ZB440;G10,CF₃,H,H,-] , [ZB441;G10,F,H,H,-] , [ZB442;G10,Cl,H,H,-] , [ZB443;G10,Br,H,H ,-] , [ZB444;G10,CN,H,H,-] , [ZB445;G10,H,Me,H,-] , [ZB446;G10,H, Et,H,-] , [ZB447;G10,H,Pr,H,-] , [ZB448;G10,H,i-Pr,H,-] , [ZB449;G10,H,c-Pr,H,-] , [ZB450;G10,H,Ph,H,-] , [ZB451;G10,H,OMe,H,-] , [ZB452;G 10,H,OEt,H,-] , [ZB453;G10,H,OPh,H,-] , [ZB454;G10,H,CF₃,H,-] , [ ZB455;G10,H,F,H,-] , [ZB456;G10,H,Cl,H,-) , [ZB457;G10,H,Br,H,-] , [ZB458;G10,H,CN,H,-] , [ZB459;G10,H,H,Me,-] , [ZB46C;G10,H,H, Et,-] , [ZB461;G10,H,H,Pr,-] , [ZB462;G10,H,H,i-Pr,-] , [ZB463;G10,H,H,c-Pr,-] , [ZB464;G10,H,H,Ph,-] , [ZB465;G10,H,H,OMe,-] , [ZB466;G10 ,H,H,OEt,-] , [ZB467;G10,H,H,OPh,-] , [ZB468;G10,H,H,CF₃,-] , [ZB 469;G10,H,H,F,-] , [ZB470;G10,H,H,Cl,-] , [ZB471;G10,H,H,Br,-] , [ZB472;G10,H,H,CN,-] , [ZB473;G11,H,H,H,-] , [ZB474;G11,H,Me,H, -] , [2B475;G11,H,Et,H,-] , [ZB476;G11,H,Pr,H,-] , [ZB477;G11,H,i-Pr,H,-] , [ZB478;G11,H,c-Pr,H,-] , [ZB479;G11,H,Ph,H,-] , [ZB480;G11,H,OMe,H,-] , [ZB481;G 11,H,OEt,H,-] , [ZB482;G11,H,OPh,H,-] , [ZB483;G11,H,CF₃,H,-] , [ ZB484;G11,HF,H,-] , [ZB485;G11,H,Cl,H,-] , [ZB486;G11,H,Br,H,-] , [ZB487;G11,H,CN,H,-] , [ZB488;G11,H,H,Me,-] , [ZB489;G11,H,H,E t,-] , [ZB490;G11,H,H,Pr,-] , [ZB491;G11,H,H,i-Pr,-] , [ZB492;G11,H,H,c-Pr,-] , [ZB493;G11,H,H,Ph,-] , [ZB494;G11,H,H,OMe,-] , [ZB495;G11 ,H,H,OEt,-] , [ZB496;G11,H,H,OPh,-] , [ZB497;G11,H,H,CF₃,-] , [ZB 498;G11,H,H,F,-] , [ZB499;G11,H,H,C1,-] , [ZB500;G11,H,H,Br,-] , [ZB501;G11,H,H,CN,-] , [ZB502;G11,Me,H,H,-] , [ZB503;G11,Me,Me, H,-] , [ZB504;G11,Me,Et,H,-] , [ZB505;G11,Me,Pr,H,-] , [ZB506;G11 ,Me,i-Pr,H,-] , [ZB507;G11,Me,c-Pr,H,-] , [ZB508;G11,Me,Ph,H,-] , [ZB509;G11,Me,OMe,H,-] , [ZB510 ;G11,Me,OEt,H,-] , [ZB511;G11,Me,OPh,H,-] , [ZB512;G11,Me,CF₃,H ,-] , [ZB513;G11,Me,F,H,-] , [ZB514;G11,Me,Cl,H,-] , [ZB515;G11,M e,Br,H,-] , [ZB516;G11,Me,CN,H,-] , [ZB517;G11,Me,H,Me,-] , [ZB51 8;G11,Me,H,Et,-] , [ZB519;G11,Me,H,Pr,-] , [ZB520;G11,Me,H,i-Pr,-] , [ZB521;G11,Me,H,c-Pr,-] , [ZB522;G11,Me,H,Ph,-] , [ZB523;G11,Me,H,OMe,-] , [ZB524;G 11,Me,H,OEt,-] , [ZB525;G11,Me,H,OPh,-] , [ZB526;G11,Me,H,CF₃,-] , [ZB527;G11,Me,H,F,-] , [ZB528;G11, Me,H,Cl,-] , [ZB529;G11,Me, H,Br,-] , [ZB530;G11,Me,H,CN,-] , [ZB531;G12,H,H,H,-] , [ZB532;G1 2,Me,H,H,-] , [ZB533;G12,Et,H,H,-] , [ZB534;G12,Pr,H,H,-] , [ZB53 5;G12,i-Pr,H,H,-] , [ZB536;G12,c-Pr,H,H,-] , [ZB537;G12,Ph,H,H,-] , [ZB538;G12,OMe,H,H,-] , [ZB539 ;G12,OEt,H,H,-] , [ZB540;G12,OPh,H,H,-] , [ZB541;G12,CF₃,H,H,-] , [ZB542;G12,F,H,H,-] , [ZB543;G12,Cl,H,H,-] , [ZB544;G12,Br,H,H ,-] , [ZB545;G12,CN,H,H,-] , [ZB546;G12,H,Me,H,-] , [ZB547;G12,H, Et,H,-] , [ZB548;G12,H,Pr,H,-] , [ZB549;G12,H,i-Pr,H,-] , [ZB550;G12,H,c-Pr,H,-] , [ZB551;G12,H,Ph,H,-] , [ZB552;G12,H,OMe,H,-] , [ZB553;G 12,H,OEt,H,-] , [ZB554;G12,H,OPh,H,-] , [ZB555;G12,H,CF₃,H,-] , [ ZB556;G12,H,F,H,-] , [ZB557;G12,H,Cl,H,-] , [ZB558;G12,H,Br,H,-] , [ZB559;G12,H,CN,H,-] , [ZB560;G12,H,H,Me,-] , [ZB561;G12,H,H, Et,-] , [ZB562;G12,H,H,Pr,-] , [ZB563;G12,H,H,i-Pr,-] , [ZB564;G12,H,H,c-Pr,-] , [ZB565;G12,H,H,Ph,-] , [ZB566;G12,H,H,OMe,-] , [ZB567;G12 ,H,H,OEt,-] , [ZB568;G12,H,H,OPh,-] , [ZB569;G12,H,H,CF₃,-] , [ZB 570;G12,H,H,F,-] , [ZB571;G12,H,H,C1,-] , [ZB572;G12,H,H,Br,-] , [ZB573;G12,H,H,CN,-] , [ZB574;G13,H,H,H,-] , [ZB575;G13,Me,H,H, -] , [ZB576;G13,Et,H,H,-] , [ZB577;G13,Pr,H,H,-] , [ZB578;G13,i-Pr,H,H,-] , [ZB579;G13,c-Pr,H,H,-] , [ZB580;G13,Ph,H,H,-] , [ZB581;G13,OMe,H,H,-] , [ZB582 ;G13,OEt,H,H,-] , [ZB583;G13,OPh,H,H,-] , [ZB584;G13,CF3,H,H,-] , [ZB585;G13,F,H,H,-] , [ZB586;G13,Cl,H,H,-] , [ZB587;G13,Br,H,H ,-] , [ZB588;G13,CN,H,H,-] , [ZB589;G13,H,H,Me,-] , [ZB590;G13,H, H , Et , -] , [ZB591 ; G13 , H , H , Pr , -] , [ZB592 ; G13 , H , H , i-Pr , -] , [ZB593 ; G13 , H , H , c-Pr , -] , [ZB594 ; G13 , H , H , Ph , -] , [ZB595 ; G13 , H , H , OMe , -] , [ZB596 ; G13 , H , H , OEt , -] , [ZB597 ; G13 , H , H , OPh , -] , [ZB598 ; G13 , H , H , CF₃ , -] , [ZB 599 ; G13 , H , H , F , -] , [ZB600 ; G13 , H , H , Cl , -] , [ZB601 ; G13 , H , H , Br , -] , [ZB602 ; G13 , H , H , CN , -] , [ZB603 ; G13 , H , Me , H , -] , [ZB604 ; G13 , Me , Me , H , -] , [ZB605 ; G13 , Et , Me , H , -] , [ZB606 ; G13 , Pr , Me , H , -] , [ZB607 ; G13 , i-Pr , Me , H , -] , [ZB608 ; G13 , c-Pr , Me , H , -] , [ZB609 ; G13 , Ph , Me , H , -] , [ZB610 ; G13 , OMe , Me , H , -] , [ZB 611 ; G13 , OEt , Me , H , -] , [ZB612 ; G13 , OPh , Me , H , -] , [ZB613 ; G13 , CF₃ , M e , H , -] , [ZB614 ; G13 , F , Me , H , -] , [ZB615 ; G13 , Cl , Me , H , -] , [ZB616 ; G1 3 , Br , Me , H , -] , [ZB617 ; G13 , CN , Me , H , -] , [ZB618 ; G13 , H , Me , Me , -] , [Z B619 ; G13 , H , Me , Et , -] , [ZB620 ; G13 , H , Me , Pr , -] , [ZB621 ; G13 , H , Me , i -Pr , -] , [ZB622 ; G13 , H , Me , c-Pr , -] , [ZB623 ; G13 , H , Me , Ph , -] , [ZB624 ; G13 , H , Me , OMe , -] , [ZB625 ; G 13 , H , Me , OEt , -] , [ZB626 ; G13 , H , Me , OPh , -] , [ZB627 ; G13 , H , Me , CF₃ , -] , [ZB628 ; G13 , H , Me , F , -] , [ZB629 ; G13 , H , Me , Cl , -] , [ZB630 ; G13 , H , M e , Br , -] , [ZB631 ; G13 , H , Me , CN , -] , [ZB632 ; G14 , H , H , H , -] , [ZB633 ; G1 4 , Me , H , H , -] , [ZB634 ; G14 , Et , H , H , -] , [ZB635 ; G14 , Pr , H , H , -] , [ZB63 6 ; G14 , i-Pr , H , H , -] , [ZB637 ; G14 , c-Pr , H , H , -] , [ZB638 ; G14 , Ph , H , H , -] , [ZB639 ; G14 , OMe , H , H , -] , [ZB640 ; G14 , OEt , H , H , -] , [ZB641 ; G14 , OPh , H, H , -] , [ZB642 ; G14 , CF₃ , H , H , -] , [ZB643 ; G14 , F, H , H , -] , [ZB644 ; G14 , Cl , H , H, -] , [ZB645 ; G14 , Br , H , H , -] , [ZB646 ; G14 , CN , H , H , -] , [ZB647 ; G14 , H , Me , H , -] , [ZB648 ; G14 , H , Et , H , -] , [ZB649 ; G14 , H , Pr , H , -] , [ZB650 ; G14 , H , i-Pr , H , -] , [ZB651 ; G14 , H , c-Pr , H , -] , [ZB652 ; G14 , H , Ph , H , -] , [ZB653 ; G14 , H , OMe , H , -] , [ZB654 ; G 14 , H , OEt , H , -] , [ZB655 ; G14 , H , OPh , H , -] , [ZB656 ; G14 , H , CF₃ , H , -] , [ ZB657 ; G14 , H , F , H , -] , [ZB658 ; G14 , H, Cl , H , -] , [ZB659 ; G14 , H , Br , H , -] , [ZB660 ; G14 , H , CN , H , -] , [ZB661 ; G14 , H , H , Me , -] , [ZB662 ; G14 , H , H , Et , -] , [ZB663 ; G14 , H , H , Pr , -] , [ZB664 ; G14 , H , H , i-Pr , -] , [ZB665 ; G14 , H , H , c-Pr , -] , [ZB666 ; G14 , H , H , Ph , -] , [ZB667 ; G14 , H , H , OMe , -] , [ZB668 ; G14 , H , H , OEt , -] , [ZB669 ; G14 , H , H , OPh , -] , [ZB670 ; G14 , H , H , CF₃ , -] , [ZB 671 ; G14 , H , H , F , -] , [ZB672 ; G14 , H , H , Cl , -] , [ZB673 ; G14 , H , H , Br , -] , [ZB674 ; G14 , H , H , CN , -] , [ZB675 ; G15 , H , H , H , -] , [ZB676 ; G15 , Me , H , H , -] , [ZB677 ; G15 , Et , H , H , -] , [ZB678 ; G15 , Pr , H , H , -] , [ZB679 ; G15 , i-Pr , H , H , -] , [ZB680 ; G15 , c-Pr , H , H , -] , [ZB681 ; G15 , Ph , H , H , -] , [ZB682 ; G15 , OMe , H , H, -] , [ZB683 ; G15 , OEt , H , H , -] , [ZB684 ; G15 , OPh , H , H , -] , [ZB685 ; G15 , CF₃ , H , H , -] , [ZB686 ; G15 , F , H , H , -] , [ZB687 ; G15 , Cl , H , H , -] , [ZB688 ; G15 , Br , H , H , -] , [ZB689 ; G15 , CN , H , H , -] , [ZB690 ; G15 , H , Me , H , -] , [ZB691 ; G15 , H , Et , H , -] , [ZB692 ; G15 , H , Pr , H , -] , [ZB693 ; G15 , H , i-Pr , H , -] , [ZB694 ; G15 , H , c-Pr , H , -] , [ZB695 ; G15 , H , Ph , H , -] , [ZB696 ; G15 , H , OMe , H , -] , [ZB697 ; G 15 , H , OEt , H , -] , [ZB698 ; G15 , H , OPh , H , -] , [ZB699 ; G15 , H , CF₃ , H , -] , [ ZB700 ; G15 , H , F , H , -] , [ZB701 ; G15 , H , Cl , H , -] , [ZB702 ; G15 , H , Br , H , -] , [ZB703 ; G15 , H , CN , H , -] , [ZB704 ; G15 , H , H , Me , -] , [ZB705 ; G15 , H , H , Et , -] , [ZB706 ; G15 , H , H , Pr , -] , [ZB707 ; G15 , H , H , i-Pr , -] , [ZB708 ; G15 , H, H , c-Pr , -] , [ZB709 ; G15 , H , H , Ph , -] , [ZB710 ; G15 , H , H , OMe , -] , [ZB711 ; G15 , H , H , OEt , -] , [ZB712 ; G15 , H , H , OPh , -] , [ZB713 ; G15 , H , H , CF₃ , -] , [ZB 714 ; G15 , H , H , F , -] , [ZB715 ; G15 , H, H , Cl , -] , [ZB716 ; G15 , H, H , Br , -] , [ZB717 ; G15 , H , H, CN , -] , [ZB718 ; G16 , H , H , - , -] , [ZB719 ; G16 , Me , H , - , -] , [ZB720 ; G16 , Et , H , - , - ] , [ZB721 ; G16 , Pr , H , - , -] , [ZB722 ; G16 , i-Pr , H , - , -] , [ZB723 ; G16 , c-Pr , H , - , -] , [ZB724 ; G16 , Ph ,H , - , -] , [ZB725 ; G16 , OMe , H , - , -] , [ZB726 ; G16 , OEt , H , - , -] , [ZB727 ; G16 , OPh , H , - , -] , [ZB728 ; G16 , CF₃ , H , - , -] , [ZB729 ; G16 , F , H , - , -] , [ZB730 ; G16 , Cl , H , - , -] , [ZB731 ; G16 , Br , H , -, -] , [ZB732 ; G16 , CN , H, - , -] , [ZB733 ; G16 , H , Me , - , -] , [ZB734 ; G16 , H , Et , - , -] , [ZB735 ; G16 , H , Pr , - , -] , [ZB736 ; G16 , H , i-Pr , - , -] , [ZB737 ; G16 , H , c-Pr , - , -] , [ZB738 ; G16 , H , Ph , - , -] , [ZB739 ; G16 , H , OMe , - , -] , [ZB740 ; G 16 , H , OEt , - , -] , [ZB741 ; G16 , H , OPh , - , -] , [ZB742 ; G16 , H , CF₃ , - , -] , [ ZB743 ; G16 , H , F , - , -] , [ZB744 ; G16 , H , Cl , - , -] , [ZB745 ; G16 , H , Br , - , -] , [ZB746 ; G16 , H , CN , - , -] , [ZB747 ; G17 , H , H , - , -] , [ZB748 ; G17 , Me, H , - , -] , [ZB749 ; G17 , Et , H , - , -] , [ZB750 ; G17 , Pr , H , - , -] , [ZB751 ; G17 , i -Pr , H , - , -] , [ZB752 ; G17 , c-Pr , H , - , -] , [ZB753 ; G17 , Ph , H , - , -] , [ZB754 ; G17 , OMe , H , - , -] , [ZB755 ; G17 , OEt , H , - , -] , [ZB756 ; G17 , OPh , H , - , -] , [ZB757 ; G17 , CF₃ , H , - , - ] , [ZB758 ; G17 , F , H , -] , [ZB759 ; G17 , Cl , H , - , -] , [ZB760 ; G17 , Br , H , -, -] , [ZB761 ; G17 , CN , H , - , -] , [ZB762 ; G17 , H , Me , - , -] , [ZB763 ; G17 , H , Et , - , -] , [ZB764 ; G17 , H , Pr , - , -] , [ZB765 ; G17 , H , i-Pr , - , -] , [ZB766 ; G17 , H , c-Pr , - , - ] , [ZB767 ; G17 , H, Ph , - , -] , [ZB768 ; G17 , H , OMe , - , -] , [ZB769 ; G 17 , H , OEt , - , -] , [ZB770 ; G17 , H , OPh , - , -] , [ZB771 ; G17 , H , CF₃ , - , -] , [ ZB772 ; G17 , H , F , - , -] , [ZB773 ; G17 , H , Cl , - , -] , [ZB774 ; G17 , H , Br , - , -] , [ZB775 ; G17 , H , CN , - , -] , [ZB776 ; G18 , H , H , - , -] , [ZB777 ; G18 , Me , H , - , -] , [ZB778 ; G18 , Et , H , - , -] , [ZB779 ; G18 , Pr , H , - , -] , [ZB780 ; G18 , i -Pr , H, - , -] , [ZB781 ; G18 , c-Pr , H , - , -] , [ZB782 ; G18 , Ph , H , - , -] , [ZB783 ; G18 , OMe , H , - , -] , [ZB784 ; G18 , OEt , H , - , -] , [ZB785 ; G18 , OPh , H , - , -] , [ZB786 ; G18 , CF₃ , H , - , -] , [ZB787 ; G18 , F , H , - , -] , [ZB788 ; G18 , Cl , H , - , -] , [ZB789 ; G18 , Br , H , -, -] , [ZB790 ; G18 , CN , H , - , -] , [ZB791 ; G18 , H , Me , - , -] , [ZB792 ; G18 , H , Et , - , - ] , [ZB793 ; G18 , H , Pr , - , -] , [ZB794 ; G18 , H, i-Pr , - , -] , [ZB795 ; G18 , H , c-Pr , - , -] , [ZB796 ; G18 , H , Ph , - , -] , [ZB797 ; G18 ,H , OMe , - , -] , [ZB798 ; G 18 , H ,OEt , - , -] , [ZB799 ; G18 , H , OPh , - , -] , [ZB800 ; G18 , H, CF₃, - , -] , [ ZB801 ; G18 , H , F , - , -] , [ZB802 ; G18 , H , Cl , - , -] , [ZB803 ; G18 , H , Br , -] , [ZB804 ; G18 , H , CN , - , -] , [ZB805 ; G19 , H , H , - , -] , [ZB806 ; G19 , Me , H , - , -] , [ZB807 ; G19 , Et , H , - , -] , [ZB808 ; G19 , Pr , H , - , -] , [ZB809 ; G19, i -Pr , H , - , -] , [ZB810 ; G19 , c-Pr , H , - , -] , [ZB811 ; G19 , Ph , H , - , -] , [ZB812 ; G19 , OMe , H , - , -] , [ZB813 ; G19 , OEt , H , - , -] , [ZB814 ; G19 , OPh , H, - , -] , [ZB815 ; G19 , CF₃ , H , - , -] , [ZB816 ; G19 , F , H , - , -] , [ZB817 ; G19 , Cl , H , - , -] , [ZB818 ; G19 , Br , H , -, -] , [ZB819 ; G19 , CN , H , - , -] , [ZB820 ; G19 , H , Me , - , -] , [ZB821 ; G19 , H , Et , - , -] , [ZB822 ; G19 , H , Pr , - , -] , [ZB823 ; G19 , H , i-Pr , - , -] , [ZB824 ; G19 , H , c-Pr , - , -] , [ZB825 ; G19 , H , Ph , - , -] , [ZB826 ; G19 , H , OMe , - , -] , [ZB827 ; G 19 , H , OEt , - , -] , [ZB828 ; G19 , H , OPh , - , -] , [ZB829 ; G19 , H, CF₃ , - , -] , [ ZB830 ; G19 , H , F , - , -] , [ZB831 ; G19 , H , Cl , - , -] , [ZB832 ; G19 , H , Br , - , -] , [ZB833 ; G19 , H , CN , - , -] , [ZB834 ; G20 , H , H , - , -] , [ZB835 ; G20 , Me , H , - , -] , [ZB836 ; G20 , Et , H , - , -] , [ZB837 ; G20 , Pr , H , - , -] , [ZB838 ; G20 , i -Pr , H , - , -] , [ZB839 ; G20 , c-Pr , H , - , -] , [ZB840 ; G20 , Ph , H , - , -] , [ZB841 ; G20 , OMe , H , - , -] , [ZB842 ; G20 , OEt , H, - , -] , [ZB843 ; G20 , OPh , H , - , -] , [ZB844 ; G20 , CF₃ , H , - , -] , [ZB845 ; G20 , F , H , - , -] , [ZB846 ; G20 , Cl , H , - , -] , [ZB847 ; G20 , Br , H , -, -] , [ZB848 ; G20 , CN , H , - , -] , [ZB849 ; G20 , H , Me , - , -] , [ZB850 ; G20 , H , Et , - , -] , [ZB851 ; G20 , H , Pr , - , -] , [ZB852 ; G20 , H , i-Pr , - , -] , [ZB853 ; G20 , H , c-Pr , - , -] , [ZB854 ; G20 , H , Ph , - , -] , [ZB855 ; G20 , H , OMe , - , -] , [ZB856 ; G 20 , H , OEt , - , -] , [ZB857 ; G20 , H , OPh , - , -] , [ZB858 ; G20 , H , CF₃, - , -] , [ ZB859 ; G20 , H , F , - , -] , [ZB860 ; G20 , H , Cl , - , -] , [ZB861 ; G20 , H , Br , - , -] , [ZB862 ; G20 , H , CN , - , -] , [ZB863 ; G21 , H , H , - , -] , [ZB864 ; G21 , Me , H , - , -] , [ZB865 ; G21 , Et , H , - , -] , [ZB866 ; G21 , Pr , H , - , -] , [ZB867 ; G21 , i -Pr , H, - , -] , [ZB868 ; G21 , c-Pr , H , - , -] , [ZB869 ; G21 , Ph , H , - , -] , [ZB870 ; G21 , OMe , H , - , -] , [ZB871 ; G21 , OEt , H , - , -] , [ZB872 ; G21 , OPh , H ,- , -] , [ZB873 ; G21 , CF₃ , H , - , -] , [ZB874 ; G21 , F , H , - , -] , [ZB875 ; G21 , Cl , H , - , -] , [ZB876 ; G21 , Br , H , -, -] , [ZB877 ; G21 , CN , H , - , -] , [ZB878 ; G21 , H , Me , - , -] , [ZB879 ; G21 , H , Et , - , -] , [ZB880 ; G21 , H , Pr , - , -] , [ZB881 ; G21 , H , i-Pr , - , -] , [ZB882 ; G21 , H , c-Pr , - , -] , [ZB883 ; G21 , H , Ph , - , -] , [ZB884 ; G21 , H , OMe , - , -] , [ZB885 ; G 21 , H , OEt , - , -] , [ZB886 ; G21 , H , OPh , - , -] , [ZB887 ; G21 , H , CF₃ , - , -] , [ ZB888 ; G21 , H , F , - , -] , [ZB889 ; G21 , H , Cl , - , -] , [ZB890 ; G21 , H , Br , - , -] , [ZB891 ; G21 , H , CN , - , -] , [ZB892 ; G22 , H , H , - , -] , [ZB893 ; G22 , Me , H , - , -] , [ZB894 ; G22 , Et , H , - , -] , [ZB895 ; G22 , Pr , H, - , -] , [ZB896 ; G22 , i -Pr , H , - , -] , [ZB897 ; G22 , c-Pr , H , - , -] , [ZB898 ; G22 , Ph , H, - , -] , [ZB899 ; G22 , OMe , H , - , -] , [ZB900 ; G22 , OEt , H , - , -] , [ZB901 ; G22 , OPh , H , - , -] , [ZB902 ; G22 , CF₃ , H , - , -] , [ZB903 ; G22 , F , H , - , -] , [ZB904 ; G22 , Cl , H , - , -] , [ZB905 ; G22 , Br , H , -, -] , [ZB906 ; G22 , CN , H , - , -] , [ZB907 ; G22 , H , Me , - , -] , [ZB908 ; G22 , H , Et , - , -] , [ZB909 ; G22 , H , Pr , - , -] , [ZB910 ; G22 , H , i-Pr , - , -] , [ZB911 ; G22 , H , c-Pr , - , -] , [ZB912 ; G22 , H , Ph , - , - ] , [ZB913 ; G22 , H , OMe , - , -] , [ZB914 ; G 22 , H , OEt , - , -] , [ZB915 ; G22 , H , OPh , - , -] , [ZB916 ; G22 , H , CF₃ , - , -] , [ ZB917 ; G22 , H , F , - , -] , [ZB918 ; G22 , H , Cl , - , -] , [ZB919 ; G22 , H , Br , - , -] , [ZB920 ; G22 , H , CN , - , -] , [ZB921 ; G23 , H , H , - , -] , [ZB922 ; G23 , Me , H , - , -] , [ZB923 ; G23 , Et , H , - , -] , [ZB924 ; G23 , Pr , H , - , -] , [ZB925 ; G23 , i -Pr , H , - , -] , [ZB926 ; G23 , c-Pr , H , - , -] , [ZB927 ; G23 , Ph , H , - , -] , [ZB928 ; G23 , OMe , H , - , -] , [ZB929 ; G23 , OEt , H , - , -] , [ZB930 ; G23 , OPh , H , - , -] , [ZB931 ; G23 , CF₃ , H , - , -] , [ZB932 ; G23 , F , H , - , -] , [ZB933 ; G23 , Cl , H , - , -] , [ZB934 ; G23 , Br , H , -, -] , [ZB935 ; G23 , CN , H , - , -] , [ZB936 ; G23 , H , Me , - , -] , [ZB937 ; G23 , H , Et , - , -] , [ZB938 ; G23 , H, Pr , - , -] , [ZB939 ; G23 , H , i-Pr , - , -] , [ZB940 ; G23 , H , c-Pr , - , -] , [ZB941 ; G23 , H , Ph , - , -] , [ZB942 ; G23 , H , OMe , - , -] , [ZB943 ; G 23 , H , OEt , - , -] , [ZB944 ; G23 , H , OPh , - , -] , [ZB945 ; G23 , H , CF₃ , - , -] , [ ZB946 ; G23 , H , F , - , -] , [ZB947 ; G23 , H , Cl , - , -] , [ZB948 ; G23 , H , Br , - , -] , [ZB949 ; G23 , H , CN , - , -] , [ZB950 ; G24 , H , H , - , -] , [ZB951 ; G24 , Me , H , - , -] , [ZB952 ; G24 , Et , H , - , -] , [ZB953 ; G24 , Pr , H , - , -] , [ZB954 ; G24 , i -Pr , H , - , -], [ZB955 ; G24 , c-Pr , H , - , -] , [ZB956 ; G24 , Ph , H , - , -] , [ZB957 ; G24 , OMe , H , - , -] , [ZB958 ; G24 , OEt , H , - , -] , [ZB959 ; G24 , OPh , H , - , -] , [ZB960 ; G24 , CF₃ , H , - , -] , [ZB961 ; G24 , F , H , - , -] , [ZB962 ; G24 , Cl , H , - , -] , [ZB963 ; G24 , Br , H , -, -] , [ZB964 ; G24 , CN , H , - , -] , [ZB965 ; G24 , H , Me , - , -] , [ZB966 ; G24 , H , Et , - , -] , [ZB967 ; G24 , H , Pr , - , -] , [ZB968 ; G24 , H , i-Pr , - , -] , [ZB970 ; G24 , H , Ph , - , -] , [ZB971 ; G24 , H , OMe , - , -] , [ZB972 ; G 24 , H , OEt , - , -] , [ZB973 ; G24 , H , OPh , - , -] , [ZB974 ; G24 , H , CF₃ , - , -] , [ ZB975 ; G24 , H , F , - , -] , [ZB976 ; G24 , H , Cl , - , -] , [ZB977 ; G24 , H , Br , - , -] , [ZB978 ; G24 , H , CN , - , -] , [ZB979 ; G25 , H , H , H , -] , [ZB980 ; G25 , H , Me , H , -] , [ZB981 ; G25 , H , Et , H , -] , [ZB982 ; G25 , H , Pr , H , -] , [ZB983 ; G25 , H , i-Pr , H , -] , [ZB984 ; G25 , H , c-Pr , H , -] , [ZB985 ; G25 , H , Ph , H , -] , [ZB986 ; G25 , H , OMe , H , -] , [ZB987 ; G 25 , H , OEt , H , -] , [ZB988 ; G25 , H , OPh , H , -] , [ZB989 ; G25 , H , CF₃ , H , -] , [ ZB990 ; G25 , H , F , H , -] , [ZB991 ; G25 , H , Cl , H , -] , [ZB992 ; G25 , H , Br , H , -] , [ZB993 ; G25 , H , CN , H , -] , [ZB994 ; G25 , H , H , Me , -] , [ZB995 ; G25 , H , H , Et , -] , [ZB996 ; G25 , H , H , Pr , -] , [ZB997 ; G25 , H , H , i-Pr , -] , [ZB998 ; G25 , H , H , c-Pr , -] , [ZB999 ; G25 , H , H , Ph , -] , [ZB1000 ; G25 , H , H , OMe , -] , [ZB1001 ; G 25 , H , H , OEt , -] , [ZB1002 ; G25 , H , H , OPh , -] , [ZB1003 ; G25 , H , H , CF₃ , -] , [ZB1004 ; G25 , H , H , F , -] , [ZB1005 ; G25 , H , H , Cl , -] , [ZB1006 ; G25 , H , H , Br , -] , [ZB1007 ; G25 , H , H , CN , -] , [ZB1008 ; G25 , Me , H , H , -] , [ZB1009 ; G25 , Me , Me , H , -] , [ZB1010 ; G25 , Me , Et , H , -] , [ZB1011 ; G25 , Me , Pr , H , -] , [ZB1012 ; G25 , Me , i-Pr , H , -] , [ZB1013 ; G25 , Me , c-Pr , H , -] , [ZB1014 ; G25 , Me , Ph , H , -] , [ZB1015 ; G25 , Me , OMe , H , -] , [ZB1 016 ; G25 , Me , OEt , H , -] , [ZB1017 ; G25 , Me , OPh , H , -] , [ZB1018 ; G25 , Me , CF₃ , H , -] , [ZB1019 ; G25 , Me , F , H , -] , [ZB1020 ; G25 , Me , Cl , H , -] , [ZB10 21 ; G25 , Me , Br , H , -] , [ZB1022 ; G25 , Me , CN , H , -] , [ZB1023 ; G25 , Me , H , M e , -] , [ZB1024 ; G25 , Me , H , Et , -] , [ZB1025 ; G25 , Me , H , Pr , -] , [ZB1026 ; G25 , Me , H , i-Pr , -] , [ZB1027 ; G25 , Me , H , c-Pr , -] , [ZB1028 ; G25 , Me , H , Ph , -] , [ZB1029 ; G25 , Me , H , OMe , -] , [ZB103 0 ; G25 , Me , H , OEt , -] , [ZB1031 ; G25 , Me , H , OPh , -] , [ZB1032 ; G25 , Me , H , CF₃ , -] , [ZB1033 ; G25 , Me , H , F , -] , [ZB1034 ; G25 , Me , H , Cl , -] , [ZB1035 ; G25 , Me , H , Br , -] , [ZB1036 ; G25 , Me , H , CN , -] , [ZB1037 ; G26 , H , H , H , -] , [ZB1038 ; G26 , H , Me , H , -] , [ZB1039 ; G26 , H , Et , H , -] , [ZB1040 ; G26 , H , Pr , H , -] , [ZB1041 ; G26 , H , i-Pr , H , -] , [ZB1042 ; G26 , H , c-Pr , H , -] , [ZB1043 ; G26 , H , Ph , H , -] , [ZB1044 ; G26 , H , OMe , H , -] , [ZB104 5 ; G26 , H , OEt , H , -] , [ZB1046 ; G26 , H , OPh , H , -] , [ZB1047 ; G26 , H , CF₃ , H , -] , [ZB1048 ; G26 , H , F ,H , -] , [ZB1049 ; G26 , H , Cl , H , -] , [ZB1050 ; G26 , H , Br , H , -] , [ZB1051 ; G26 , H , CN , H , -] , [ZB1052 ; G26 , H , H , Me , -] , [ZB10 53 ; G26 , H , H , Et , -] , [ZB1054 ; G26 , H , H , Pr , -] , [ZB1055 ; G26 , H , H , i-Pr , -] , [ZB1056 ; G26 , H , H , c-Pr , -] , [ZB1057 ; G26 , H , H , Ph , -] , [ZB1058 ; G26 , H , H , OMe , -] , [ZB1059 ; G26 , H , H , OEt , -] , [ZB1060 ; G26 , H , H , OPh , -] , [ZB1061 ; G26 , H , H , CF₃ , -] , [ZB1062 ; G26 , H , H , F, -] , [ZB1063 ; G26 , H , H , Cl , -] , [ZB1064 ; G26 , H , H , Br , -] , [ZB1065 ; G26 , H , H , CN , -] , [ZB1066 ; G26 , Me , H , H , -] , [ZB1067 ; G26 , Me , Me , H , -] , [ZB1068 ; G26 , Me , Et , H , -] , [ZB1069 ; G26 , Me , Pr , H , -] , [ZB1070 ; G26 , Me , i-Pr , H , -] , [ZB1071 ; G26 , Me , c-Pr , H , -] , [ZB1072 ; G26 , Me , Ph , H , -] , [ZB1073 ; G26 , Me , OMe , H , -] , [ZB1 074 ; G26 , Me , OEt , H , -] , [ZB1075 ; G26 , Me , OPh , H , -] , [ZB1076 ; G26 , Me , CF₃ , H , -] , [ZB1077 ; G26 , Me , F , H , -] , [ZB1078 ; G26 , Me , Cl , H , -] , [ZB10 79 ; G26 , Me , Br , H , -] , [ZB1080 ; G26 , Me , CN , H , -] , [ZB1081 ; G26 , Me , H , M e , -] , [ZB1082 ; G26 , Me , H , Et , -] , [ZB1083 ; G26 , Me , H , Pr , -] , [ZB1084 ; G26 , Me , H , i-Pr , -] , [ZB1085 ; G26 , Me , H , c-Pr , -] , [ZB1086 ; G26 , Me , H, Ph , -] , [ZB1087 ; G26 , Me , H , OMe , -] , [ZB108 8 ; G26 , Me , H , OEt , -] , [ZB1089 ; G26 , Me, H , OPh , -] , [ZB1090 ; G26 , Me , H , CF₃ , -] , [ZB1091 ; G26 , Me , H , F , -] , [ZB1092 ; G26 , Me , H , Cl , -] , [ZB1093 ; G26 , Me , H , Br , -] , [ZB1094 ; G26 , Me , H , CN , -] , [ZB1095 ; G27 , H , H , H , -] , [ZB1096 ; G27 , Me , H , H , -] , [ZB1097 ; G27 , Et , H , H , -] , [ZB1098 ; G27 , Pr , H , H , -] , [ZB1099 ; G27 , i-Pr , H , H , -] , [ZB1100 ; G27 , c-Pr , H , H , -] , [ZB1101 ; G27 , Ph ,H , H , -] , [ZB1102 ; G27 , OMe , H , H , -] , [ZB1 103 ; G27 , OEt , H , H , -] , [ZB1104 ; G27 , OPh , H , H , -] , [ZB1105 ; G27 , CF₃ , H , H , -] , [ZB1106 ; G27 , F , H , H , -] , [ZB1107 ; G27 , Cl , H , H , -] , [ZB1108 ; G2 7 , Br , H , H , -] , [ZB1109 ; G27 , CN , H , H , -] , [ZB1110 ; G27 , H , Me , H , -] , [ZB 1111 ; G27 , H , Et , H , -] , [ZB1112 ; G27 , H , Pr , H , -] , [ZB1113 ; G27 , H , i-Pr , H , -] , [ZB1114 ; G27 , H , c-Pr , H , -] , [ZB1115 ; G27 , H , Ph , H , -] , [ZB1116 ; G27 , H , OMe , H , -] , [ZB111 7 ; G27 , H , OEt , H , -] , [ZB1118 ; G27 , H , OPh , H , -] , [ZB1119 ; G27 , H , CF₃ , H , -] , [ZB1120 ; G27 , H , F , H , -] , [ZB1121 ; G27 , H , Cl , H , -] , [ZB1122 ; G27 , H , Br , H , -] , [ZB1123 ; G27 , H , CN , H , -] , [ZB1124 ; G27 , H , H , Me , -] , [ZB1125 ; G27 , H , H , Et , -] , [ZB1126 ; G27 , H , H , Pr , -] , [ZB1127 ; G27 , H , H , i-Pr , -] , [ZB1128 ; G27 , H , H , c-Pr , -] , [ZB1129 ; G27 , H , H , Ph , -] , [ZB1130 ; G27 , H , H , OMe , -] , [ZB1131 ; G27 , H , H , OEt , -] , [ZB1132 ; G27 , H , H ,OPh , -] , [ZB1133 ; G27 , H , H , CF₃ , -] , [ZB1134 ; G27 , H , H , F , -] , [ZB1135 ; G27 , H , H , Cl , -] , [ZB1136 ; G27 , H , H , Br , -] , [ZB1137 ; G27 , H , H , CN , -] , [ZB1138 ; G28 , H , H , - , -] , [ZB1139 ; G28 , Me , H , - , -] , [ZB1140 ; G28 , Cl , H , - , -] , [ZB1141 ; G28 , CF₃, H , - , -] , [ZB1142 ; G28 , H , Me , - , -] , [ZB1143 ; G28 , H , Cl , - , -] , [ZB1144 ; G28 , H , C F₃ , - , -] , [ZB1145 ; G28 , H , SH , - , -] , [ZB1146 ; G29 , H , H , H , -] , [ZB1147 ; G29 , H, Me , H , -] , [ZB1148 ; G29 , H , Et , H , -] , [ZB1149 ; G29 , H , Pr , H , -] , [ ZB1150 ; G29 , H , i-Pr , H , -] , [ZB1151 ; G29 , H , c-Pr , H , -] , [ZB1152 ; G29 , H , Ph , H , -] , [ZB1153 ; G29 , H , OMe , H , -] , [ZB115 4 ; G29 , H , OEt , H , -] , [ZB1155 ; G29 , H , OPh , H , -] , [ZB1156 ; G29 , H , CF₃ , H , -] , [ZB1157 ; G29 , H , F , H , -] , [ZB1158 ; G29 , H , Cl , H , -] , [ZB1159 ; G29 , H , Br , H , -] , [ZB1160 ; G29 , H , CN , H , -] , [ZB1161 ; G29 , H ,H , Me , -] , [ZB11 62 ; G29 , H , H , Et , -] , [ZB1163 ; G29 , H , H , Pr , -] , [ZB1164 ; G29 , H , H , i-Pr , -] , [ZB1165 ; G29 , H , H , c-Pr , -] , [ZB1166 ; G29 , H , H, Ph , -] , [ZB1167 ; G29 , H , H , OMe , -] , [ZB1168 ; G29 , H, H , OE t , -] , [ZB1169 ; G29 , H , H , OPh , -] , [ZB1170 ; G29 , H , H , CF₃ , -] , [ZB1171 ; G29 , H , H ,F , -] , [ZB1172 ; G29 , H , H , Cl , -] , [ZB1173 ; G29 , H , H , Br , -] , [ZB1174 ; G29 , H , H , CN , -] , [ZB1175 ; G29 , Me , H , H , -] , [ZB1176 ; G29 , Me , Me , H , -] , [ZB1177 ; G29 , Me , Et , H , -] , [ZB1178 ; G29 , Me , Pr , H , -] , [ZB1179 ; G29 , Me , i-Pr , H , -] , [ZB1180 ; G29 , Me , c-Pr , H , -] , [ZB1181 ; G29 , Me , Ph , H , -] , [ZB1182 ; G29 , Me , OMe, H , -] , [ZB1 183 ; G29 , Me , OEt , H , -] , [ZB1184 ; G29 , Me , OPh , H , -] , [ZB1185 ; G29 , Me , CF₃ , H , -] , [ZB1186 ; G29 , Me , F , H , -] , [ZB1187 ; G29 , Me , Cl , H , -] , [ZB11 88 ; G29 , Me , Br , H , -] , [ZB1189 ; G29 , Me , CN , H , -] , [ZB1190 ; G29 , Me , H , M e , -] , [ZB1191 ; G29 , Me , H , Et , -] , [ZB1192 ; G29 , Me , H , Pr , -] , [ZB1193 ; G29 , Me , H , i-Pr , -] , [ZB1194 ; G29 , Me , H , c-Pr , -] , [ZB1195 ; G29 , Me , H , Ph , -] , [ZB1196 ; G29 , Me , H , OMe , -] , [ZB119 7 ; G29 , Me , H , OEt , -] , [ZB1198 ; G29 , Me , H , OPh , -] , [ZB1199 ; G29 , Me , H , CF₃ , -] , [ZB1200 ; G29 , Me , H , F, -] , [ZB1201 ; G29 , Me , H , Cl , -] , [ZB1202 ; G29 , Me , H , Br , -] , [ZB1203 ; G29 , Me , H , CN , -] , [ZB1204 ; G30 , H , H , H , -] , [ZB1205 ; G30 , Me , H , H , -] , [ZB1206 ; G30 , Et , H , H , -] , [ZB1207 ; G30 , Pr , H , H , -] , [ZB1208 ; G30 , i-Pr , H , H , -] , [ZB1209 ; G30 , c-Pr , H , H , -] , [ZB1210 ; G30 , Ph , H , H , -] , [ZB1211 ; G30 , OMe , H , H , -] , [ZB1 212 ; G30 , OEt , H , H , -] , [ZB1213 ; G30 , OPh , H ,H , -] , [ZB1214 ; G30 , CF₃ , H , H , -] , [ZB1215 ; G30 , F , H , H , -] , [ZB1216 ; G30 , C1 ,H , H , -] , [ZB1217 ; G3 0 , Br , H , H , -] , [ZB1218 ; G30 , CN , H , H , -] , [ZB1219 ; G30 , H , Me , H , -] , [ZB 1220 ; G30 , H, Et , H , -] , [ZB1221 ; G30 , H , Pr , H , -] , [ZB1222 ; G30 , H , i-Pr , H , -] , [ZB1223 ; G30 , H , c-Pr , H , -] , [ZB1224 ; G30 , H , Ph , H , -] , [ZB1225 ; G30 , H , OMe , H , -] , [ZB122 6 ; G30 , H , OEt , H , -] , [ZB1227 ; G30 , H , OPh , H , -] , [ZB1228 ; G30 , H , CF₃ , H , -] , [ZB1229 ; G30 , H , F , H , -] , [ZB1230 ; G30 , H , C1 , H , -] , [ZB1231 ; G30 , H , Br ,H , -] , [ZB1232 ; G30 , H , CN , H , -] , [ZB1233 ; G30 , H , H , Me , -] , [ZB12 34 ; G30 , H , H , Et , -] , [ZB1235 ; G30 , H , H , Pr , -] , [ZB1236 ; G30 , H , H ,i-Pr , -] , [ZB1237 ; G30 , H , H , c-Pr , -] , [ZB1238 ; G30 , H , H , Ph , -] , [ZB1239 ; G30 , H , H , OMe , -] , [ZB1240 ; G30 , H , H , OEt , -] , [ZB1241 ; G30 , H , H , OPh , -] , [ZB1242 ; G30 , H , H , CF₃ , -] , [ZB1243 ; G30 , H , H , F , -] , [ZB1244 ; G30 , H , H , Cl , -] , [ZB1245 ; G30 , H , H , Br , -] , [ZB1246 ; G30 , H , H , CN , -] , [ZB1247 ; G30 , H , Oi-Pr , H , -] , [ZB1248 ; G30 , H, Oc-Pr , H , -] , [ZB1249 ; G30 , H , Ot-Bu , H , -] , [ZB1250 ; G30 , H , OCF₃ , H , -] , [ZB1251 ; G30 , H , OCHF₂ , H , -] , [Z B1252 ; G30 , H , OPr , H , -] , [ZB1253 ; G30 , H , OCH₂Ph , H , -] , [ZB1253 ; G30 , H , OCH₂-i-Pr , H , -] , [ZB1253 ; G30 , H , OCH₂-c-Pr , H , -] , [ZB1253 ; G30 , H , OCH₂-t-Bu , H , -] , [ZB1254 ; G31 , H ,- , - , -] , [ZB1255 ; G31 , Me , - , - , -] , [ZB1256 ; G31 , Et , - , - , -] , [ZB1257 ; G31 , Pr , - , - , -] , [ZB1258 ; G31 , i-Pr , - , - , -] , [ZB1259 ; G31 , c-Pr , - , - , -] , [ZB1260 ; G31 , Ph , - , - , -] , [ZB1261 ; G31 , OMe , - , - , -] , [ZB1 262 ; G31 , OEt , - , - , -] , [ZB1263 ; G31 , OPh , - , - , -] , [ZB1264 ; G31 , CF₃ , -, - , -] , [ZB1265 ; G31 , F , - , - , -] , [ZB1266 ; G31 , C1 , - , - , -] , [ZB1267 ; G3 1 , Br , - , - , -] , [ZB1268 ; G31 , CN , - , - , -] , [ZB1269 ; G31 , t-Bu , - , - , -] , [ZB1270 ; G32 , H , - , - , -] , [ZB1271 ; G32 , Me , - , - , -] , [ZB127 2 ; G32 , Et , - , - , -] , [ZB1273 ; G32 , Pr , - , - , -] , [ZB1274 ; G32 , i-Pr , - , - , -] , [ZB1275 ; G32 , c-Pr , - , - , -] , [ZB1276 ; G32 , Ph, - , - , -] , [ZB1277 ; G32 , OMe , - , - , -] , [ZB1 278 ; G32 , OEt , - , - , -] , [ZB1279 ; G32 , OPh , - , - , -] , [ZB1280 ; G32 , CF₃ , -, - , -] , [ZB1281 ; G32 ,F , - , - , -] , [ZB1282 ; G32 , Cl , - , - , -] , [ZB1283 ; G3 2 , Br , - , - , -] , [ZB1284 ; G32 , CN , - , - , -] , [ZB1285 ; G32 , t-Bu , - , - , -] , [ZB1286 ; G33 , H , - , - , -] , [ZB1287 ; G33 , Me , - , - , -] , [ZB128 8 ; G33 , Et , - , - , -] , [ZB1289 ; G33 , Pr , - , - , -] , [ZB129G ; G33 , i-Pr , - , - , -] , [ZB1291 ; G33 , c-Pr , - , - , -] , [ZB1292 ; G33 , Ph , - , - , -] , [ZB1293 ; G33 , OMe , - , - , -] , [ZB1 294 ; G33 , OEt , - , - , -] , [ZB1295 ; G33 , OPh , - , - , -] , [2B1296 ; G33 , CF₃ , -, - , -] , [ZB1297 ; G33 , F , - , - , -] , [ZB1298 ; G33 , Cl , - , - , -] , [ZB1299 ; G3 3 , Br , - , - , -] , [ZB1300 ; G33 , CN , - , - , -] , [ZB1301 ; G33 , t-Bu , - , - , -] , [ZB1302 ; G34 , H , - , - , -] , [ZB1303 ; G34 , Me , - , - , -] , [ZB130 4 ; G34 , Et , - , - , -] , [ZB1305 ; G34 , Pr , - , - , -] , [ZB1306 ; G34 , i-Pr , - , - , -] , [ZB1307 ; G34 , c-Pr , - , - , -] , [ZB1308 ; G34 , Ph , - , - , -] , [ZB1309 ; G34 , OMe , - , - , -] , [ZB1 310 ; G34 , OEt , - , - , -] , [ZB1311 ; G34 , OPh , - , - , -] , [ZB1312 ; G34 , CF₃ , -, - , -] , [ZB1313 ; G34 , F , - , - , -] , [ZB1314 ; G34 , Cl , - , - , -] , [ZB1315 ; G3 4 , Br , - , - , -] , [ZB1316 ; G34 , CN , - , - , -] , [ZB1317 ; G34 , t-Bu , - , - , -] , [ZB1318 ; G12 , t-Bu , H , H , -] , [ZB1319 ; G12 , CH(Me)Et , H , H , -] , [ZB1320 ; G12 , CH(Et)₂ , H , H , -] , [ZB1321 ; G12 , CH₂(i-Pr) , H , H , -] , [ZB1322 ; G12 , C(Me)=CH(Me) , H , H , -] , [ZB1323 ; G12 , C(Me )=CH₂ , H , H , -] , [ZB1324 ; G12 , CH=CH(Me) , H , H , -] , [ZB1325 ; G12 , CH=CH ₂ , H , H , -] , [ZB1326 ; G12 , CC(c-Pr) , H , H , -] , [ZB1327 ; G12 , CC(Me) , H , H , -] , [ZB1328 ; G12 , CHF₂ , H , H , -] , [ZB1329 ; G12 , CF₂Me , H , H , -] , [ZB1330 ; G12 , CF₂CF₃ , H , H , -] , [ZB1331 ; G12 , C(CF₃)CH₂ , H , H , -] , [ZB1332 ; G12 , CF₂Cl , H , H , -] , [ZB1333 ; G12 , C H(Me)CF₃ , H , H , -] , [ZB1334 ; G12 , c-Bu , H , H , -] , [ZB1335 ; G12 , c-Pen , H , H , -] , [ZB1336 ; G12 , c-Hex , H , H , -] , [ZB1337 ; G12 , 1-Me-(c-Pr) , H , H , -] , [ZB1338 ; G12 , 1-F-(c-Pr) , H , H , -] , [ZB1339 ; G12 , 2 , 2-F₂-(_{C}-Pr) , H , H , -] , [ZB1340 ; G12 , I , H , H , -] , [ZB1341 ; G12 , CH₂CN , H , H , -] , [Z B1342 ; G12 , CH₂OMe , H , H , -] , [ZB1343 ; G12 , CH₂SMe , H , H , -] , [ZB1344 ; G 12 , c-hexen-1-yl , H , H , -] , [ZB1345 ; G12 , OPr , H , H , -] , [ZB1346 ; G12 , O(i-Pr) , H , H , -] , [ZB1347 ; G12 , OCHF₂ , H , H , -] , [ZB1348 ; G12 , SCF₃ , H , H , -] , [ZB1349 ; G12 , C(Me)=NOMe , H , H , -] , [ZB1350 ; G12 , H , t-Bu , H , -] , [ZB1351 ; G12 , H , c-Bu , H , -] , [ZB1352 ; G12 , H , c-Pen , H , -] , [ZB1353 ; G12 , H , CCH , H , -] , [ZB1354 ; G12 , H , CH₂OMe , H , -] , [ ZB1355 ; G12 , H , I , H , -] , [ZB1356 ; G12 , H , S(i-Pr) , H , -] , [ZB1357 ; G12 , Me , Me , H , -] , [ZB1358 ; G12 , Pr , Me , H , -] , [ZB1 359 ; G12 , i-Pr , Me , H , -] , [ZB1360 ; G12 , CHF₂ , Me , H , -] , [ZB1361 ; G12 , CF₃ , Me , H , -] , [ZB1362 ; G12 , c-Pr , Me , H , -] , [ZB1363 ; G12 , c-Hex , Me , H , -] , [ZB1364 ; G12 , OMe , Me , H , -] , [ZB1365 ; G12 , O(i-Pr) , Me , H , -] , [ZB1366 ; G12 , F , Me , H , -] , [ZB1367 ; G12 , Cl , Me , H , -] , [Z B1368 ; G12 , Br , Me , H , -] , [ZB1369 ; G12 , i-Pr , F , H , -] , [ZB1370 ; G12 , Me , F , H , -] , [ZB1371 ; G12 , Pr , F , H , -] , [ZB13 72 ; G12 , CHF₂ , F, H , -] , [ZB1373 ; G12 , CF₃ , F , H , -] , [ZB1374 ; G12 , c-Pr , F , H , -] , [ZB1375 ; G12 , c-Hex , F , H , -] , [ZB1376 ; G12 , OMe , F , H , -] , [ZB1377 ; G12, O(i-Pr) , F , H , -] , [ZB1378 ; G12 , F , F , H , -] , [ZB1379 ; G12 , Cl , F , H , -] , [ZB13 80 ; G12 , Br , F , H , -] , [ZB1381 ; G12 , Pr , Cl , H , -] , [ZB1382 ; G12 , i-Pr , Cl , H , -] , [ZB1383 ; G12 , c-Hex , Cl , H , -] , [ZB1384 ; G12 , Cl , Cl , H , -] , [ZB1385 ; G12 , O(i-Pr) , Cl , H , -] , [ZB1386 ; G12 , Me , Cl , H , -] , [ZB1387 ; G12 , CHF₂ , Cl , H , -] , [ZB1388 ; G12 , CF₃ , Cl , H , -] , [ZB1389 ; G12 , c-Pr , Cl , H , -] , [ZB1390 ; G12 , OMe , Cl , H , -] , [ZB1391 ; G12 , F , Cl , H , -] , [Z B1392 ; G12 , Br , Cl , H , -] , [ZB1393 ; G12 , Me , Br , H , -] , [ZB1394 ; G12 , c-Hex , Br , H , -] , [ZB1395 ; G12 , Pr , Br ,H , -] , [ZB1396 ; G12 , i-Pr , Br , H , -] , [ ZB1397 ; G12, CHF₂, Br , H , -] , [ ZB1398 ; G12 , CF₃ , Br , H , -] , [ZB1399 ; G12 , c-Pr,Br,H,-],[ZB1400;G12,OMe,Br,H,-],[ZB1401;G12,O(i-Pr),Br,H,-],[ZB1402;G12,F,Br,H,-],[ZB1403;G12,Cl,Br,H,-],[Z B1404;G12,Br,Br,H,-],[ZB1405;G22,H,Bu,-,-],[ZB1406;G22,H,CH ₂(i-Pr) ,-,-], [ZB1407;G22,H,c-Bu,-,-], [ZB1408;G22,H,c-Pen,-,-],[ZB1409;G22,H,c-Hex,-,-],[ZB1410;G22,H,c-hexen-1-yl, -, -], [ZB1411; G22 , H, CHF₂, -, - ] , [ZB14 12 ; G22 , H, CF₂CF₃, -, -] ,[Z B1413; G22, H, CH=CH₂, -, -] , [ZB1414; G22, H, C (Me) =CH₂, -, -] , [ZB141 5;G22,H,CC(c-Pr),-,-], [ZB1415;G22,H,CC(i-Pr),-,-],[ZB1417;G22,H,CC(Me),-,-],[ZB1418;G22,H,CH=NOMe,-, -], [ZB1419;G22,H,C(Me)=NOMe,-,-],[ZB1420;G22,H,CH₂O(i-Pr) ,- ,-], [ZB1421;G22,H,CH₂OEt, -, -] , [ZB1422;G22,H,CH₂OPh, -, -],[ZB1423;G22,Me,H,-,-],[ZB1424;G22,Et,H,-,-],[ZB1425;G22,P r,H,-,-], [ZB1426;G22,Bu,H,-,-], [ZB1427;G22,CH₂(i-Pr) , H, -, -] , [ 2B1428; G22, c-Bu, H, -, -] , [ ZB1429; G22, c-Pen,H,-,-],[ZB1430;G22,c-Hex,H,-,-],[ZB1431;G22,c-hexen-1-yl, H, - , - ] , [ZB1432;G22, CHF₂, H, -, -] , [ZB1433;G22, CF₂CF₃, H, -, -] , [ZB1434;G22,CN,H,-,-], [ZB1435;G22,OMe,H,-,-], [ZB1436;G22,CH =CH₂,H,-,-], [ZB1437;G22,C(Me)=CH₂, H,-,-] , [ZB1438;G22,CC(c-Pr),H,-,-],[ZB1439;G22,CC(i-Pr) , H,-,-] , [ZB1440;G22, CC (Me) , H, -, -] , [ZB1441;G22, CH=NOMe, H, -,-],[ZB1442;G22, C(Me)=NOMe,H,-,-],[ZB1443;G22,CH₂O(i-Pr),H,-,-], [ZB1444;G22, CH₂OEt, H, -,-], [ZB1445;G22,CH₂OPh, H,-,-],[ZB1446;G22,Me,Me,-,-],[ZB1447;G22,Me,Et,-,-],[ZB1448;G 22,Me,i-Pr,-,-],[ZB1449;G22,Me,c-Pr,-,-],[ZB1450;G22,Me,c-Hex, -, -] , [ZB1451;G22,Me,CHF₂, - , - ] , [ ZB1452 ; G22, Me,CF₃, -, -], [ ZB1453;G22,Me,F,-,-], [ZB1454;G22,Me,Cl,-,-], [ZB1455;G22,Me, Br,-,-], [ZB1456;G22,Cl,Me,-,-], [ZB1457;G22,Cl,Et,-,-], [ZB14 58;G22,Cl,i-Pr,-,-],[ZB1459;G22,Cl,c-Pr,-,-],[ZB1460;G22,Cl,c-Hex, - , - ] , [ZB1461;G22,Cl,CHF₂, -, -] , [ ZB1462 ; G22 , Cl, CF₃, -, -] , [ ZB1463;G22, Cl,F, -, -] , [ZB1464; G22, Cl, Cl, -, -] , [ZB1465; G22, Cl, Br, -, -] , [ZB1466; G22, F, Me, -, -] , [ZB1467; G22, F, Et, -, -] , [ZB1468 ;G22, F, i-Pr, -, -], [ZB1469; G22, F, c-Pr, -, -] , [ZB1470; G22, F′, c-Hex,-,-], [ZB1471;G22,F,CHF₂, -, -], [ZB1472;G22, F, CF₃, -, -], [ZB 1473; G22, F, F, -, -] , [ZB1474;G22, F, Cl, -, -] , [ZB 1475; G22, F, Br, -, -], [ZB1476;G35,Me,H,-,-], [ZB1477;G35,Et,H,-,-], [ZB1478;G35, Pr,H,-,-] , [ZB1479; G35, Bu, H, -, -] , [ZB1480;G35, i-Pr, H, -, -], [ZB1481;G35, C(Me)=CH₂, H, -, -], [ZB1482;G35, CHF₂, H,-, -], [ZB1483;G35, CF₃, H, -, -; , [ZB1484; G35, c-Pr, H, -, -] , [ZB1485;G35, c-Pen, H, -, -] , [ZB1486; G35, c-Hex,H,-,-],[ZB1487;G35,C(Me)=NOH,H,-,-], [ZB1488;G35,C(Me)=N OEt,H,-,-] , [ZB1489;G35,F,H,-,-],[ZB1490;G35,Cl,H,,[ZB14 91; G35, Br, H, -, -] , [ZB1492; G35, Me, C1, -, -] , [ZB14193; G35, Et, C1, -,-],[ZB1494;G35,Pr,Ci,-,-],[ZB1495;G35,Bu,Cl,-,-],[ZB1496;G 35, i-Pr, Cl, [ZB1497;G35,C(Me)=CH₂, Cl, [ZB1498;G35, CHF₂, C 1,-,-], [ZB1499; G35, CF₃, Cl, -, - ] , [ ZB1500 ; G35, c-Pr,Cl,-,-], [ZB1501;G35,c-Pen,Cl,-,-],[ZB1502;G35,c-Hex, C1, -, -] , [ ZB1503; C35, C(Me)=NOH, Cl, -, -] , [ ZB1504; G35, C(Me) =NOEt, Cl, -, -] , [ZB1505; G35, F, C1, -, -] , [ZB1506; G35, C1, C1, -, -] , [ZB1507;G35,Br,Cl,-,-], [ZB1508;G35,Me,Br,-,-], [ZB1509;G35,E t, Br, -, -] , [ZB1510;G35, Pr, Br, -, -] , [ZB1511; G35, Bu, Br, -, -] , [ZB 1512;G35,i-Pr, Br, - , - ] , [ZB1513;G35, C (Me) =CH₂, Br, -, -] , [ZB1514 ; G35, CHF₂, B r,-,-], [ZB1515; G35, CF₃, Br,-,-] [ZB1516;G35,c-Pr,Br,-,-], [ZB1517;G35,c-Pen,Br,-,-], [ZB1518;G35,c-Hex, Br, -, -] , [ZB1519; G35, C(Me)=NOH, Br, -, -] , [ ZB1520;G35, C(Me) =NOEt, Br, -, -] , [ZB1521; G35, F, Br, -, -] , [ZB1522; G35, Cl, Br, -, -] , [ZB1523; G35, Br, Br, -, -) , [ZB1524; G35, Me, Me, -, -] , [ZB1525; G35, E t,Me,-,-], [ZB1526;G35,Pr,Me,-,-], [ZB1527;G35,Bu,Me,-,-] , [ZB 1528;G35,i-Pr, Me, -,- , [ ZB1529; G35, C(Me) =CH₂, Me, ZB1530; G35, CHF₂, M e, -, -] , [ZB1531; G35, CF₃, Me, -, -] , [ZB1532; G35, c-Pr,Me,-,-],[ZB1533;G35,c-Pen,Me,-,-],[ZB1534;G35,c-Hex, Me, -, -] , [ ZB1535; G35, C (Me)=NOH, Me, -, -] , [ ZB1536; G35, C(Me ) =NOEt,Me, -, -], [ZB1537;G35,F,Me, -, -], [ZB1538;G35,Cl,Me, -, -], [ZB1539; G35, Br, Me, -, -]

A compound (1J) wherein Q represents Q1, R¹ represents a chlorine atom, and a combination of structure of G and substituents of R^{x7}, R^{x8}, R^{x9} and R^{x10} applied to the structure of G represents any combinations described in Combination B (hereinafter, referred to as Compound class SX137).

A compound (1J) wherein Q represents Q1, R¹ represents a fluorine atom, and a combination of structure of G and substituents of R^{X7,} R^{x8}, R^{x9} and R^{x10} applied to the structure of G represents any combinations described in Combination B (hereinafter, referred to as Compound class SX138).

A compound (1J) wherein Q represents Q1, R¹ represents an ethyl group, and a combination of structure of G and substituents of R^{x7}, R^{x8}, R^{x9} and R^{x10} applied to the structure of G represents any combinations described in Combination B (hereinafter, referred to as Compound class SX139).

A compound (1J) wherein Q represents Q1, R¹ represents a cyclopropyl group, and a combination of structure of G and substituents of R^{x7}, R^{x8}, R^{x9} and R^{X10} applied to the structure of G represents any combinations described in Combination B (hereinafter, referred to as Compound class SX140).

A compound (1J) wherein Q represents Q2, L represents an oxygen atom, R¹ represents a methyl group, and a combination of structure of G and substituents of R^{x7}, R^{,X8} R^{X9} and R^{x10} applied to the structure of G represents any combinations described in Combination B (hereinafter, referred to as Compound class SX141).

A compound (1J) wherein Q represents Q2, L represents an oxygen atom, R¹ represents a chlorine atom, and a combination of structure of G and substituents of R^{x7}, R^{x8}, R^{X9} and R^{X10} applied to the structure of G represents any combinations described in Combination B (hereinafter, referred to as Compound class SX142).

A compound (1J) wherein Q represents Q2, L represents NH, R¹ represents a methyl group, and a combination of structure of G and substituents of R^{x7}, R^{x8}, R^{X9} and R^{X10} applied to the structure of G represents any combinations described in Combination B (hereinafter, referred to as Compound class SX143).

A compound (1J) wherein Q represents Q2, L represents NH, R¹ represents a chlorine atom, and a combination of structure of G and substituents of R^{x7}, R^{x8}, R^{X9} and R^{X10} applied to the structure of G represents any combinations described in Combination B (hereinafter, referred to as Compound class SX144).

A compound represented by formula (1K):

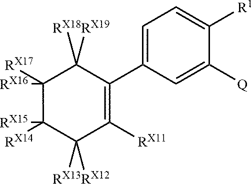

(hereinafter, referred to as “Compound (1K)”),
wherein Q represents Q1, R¹ represents a methyl group, and a combination of R^{X11}, R^{X12,} R^{X13}, R^{X14}, R^{x15}_{,} R^{x16}_{,} R^{X17}, R^{X18}, and R^{X19} represent any combinations described in Combination C (hereinafter, referred to as Compound Class SX145).

Combination C consists of substituent numbers ZC1 to ZC30.

Substituent numbers ZC1 to ZC30 represent combinations of R^{X11}, R^{X12}, R^{X13}, R^{X14}, R^{X15}, R^{X16}, R^{X17}, R^{X18}, and R^{X19} in ₜₕₑ compound (1K), which hereinafter, is referred to as [Substituent No.; R^{X11}, R^{X12,} R^{X13}, R^{X14}, R^{X15}, R^{X16}, R^{X17}, R^{X18}, R^{X19}]. For example, Substituent No. ZC2 represents a combination wherein R^{X11} represents a methyl group, and R^{X12,} R^{X13}, R^{X14}, R^{x15}_{,} R^{X16}, R^{X17}, R^{X18}, and R^{X19} represent a hydrogen atom.

### Combination C

[ZC2;H,H,H,H,H,H,H,H,H], [ZC2;Me,H,H,H,H,H,H,H,H],[ZC3;F,H,H , H, H, H, H, H H] , [ZC4; Cl, H, H, H, H, H, H, H, H] , [ZC5; H, F, F, H, H, H, H, H ,H], [ZC6;Me,F,F,H,H,H,H,H,H], [ZC7;F,F,F,H,H,H,H,H,H], [ZC8;C 1, F, F, H, H, H, H, H, H] , [ZC9; H, H, H, H, H, H, H, F, F] , [ZC10; Me, H, H, H, H , H, H, F, F] , [ZC11; F, H, H, H, H, H, H, F, F] , [ZC12 ; Cl, H, H, H, H, H, H, F, F ] , [ZC13; H, C1, C1, H, H, H, H, H, H] , [ ZC14 ; Me, Cl, Ci, H, H, H, H, H, H] , [ Z C15;F,Cl,Cl,H,H,H,H,H,H], [ZC16;Cl,Cl,Cl,H,H,H,H,H,H] , [ZC17; H, H, H, H, H, H, H, Cl, Cl] , [ZC18 ; Me, H, H, H, H, H, H, Cl, Cl] , [ZC19; F, H, H, H, H, H, H, Cl, Cl] , [ZC2O;C1, H, H, H, H, H, H, Cl, Cl] , [ZC21;H, H, H, F, F,H,H,H,H],[ZC22 ; H, H, H, Me, Me, H, H, H, H],[ZC23;H,H,H,Cl,Cl,H,H , H, H] , [ZC24 ; H, H, H, i-Pr,H,H,H,H,H] , [ZC2 5 ; H, H, H, CHF₂, H, H, H, H, H ] , [ZC26; H, H, H, CF₃, H , H, H, H, H] , [ZC27; H, H, H, CN, H, H, H, H, H] , [ZC28; H, H, H, OH, H, H, H, H, H ] , [ZC29; H, H, H, OMe, H, H, H, H, [ZC30; H, H, H, OCHF₂, H, H, H, H, H

A compound (1K) wherein Q represents Q1, R¹ represents a chlorine atom, and a combination of R^{X11}, R^{X12,} R^{X13}, R^{X14}, R^{X15}, R^{X16}, R^{X17}, R^{X18}, and R^{X19} represents any combinations described in the Combination C (hereinafter, referred to as Compound Class SX146).

A compound (1K) wherein Q represents Q2, L represents an oxygen atom, R¹ represents a methyl group, and a combination of R^{X11}, R^{X12}, R^{X13}, R^{X14}, R^{X15}, R^{X16}, R^{X17}, R^{X18}, and R^{X19} represents any combinations described in the Combination C (hereinafter, referred to as Compound Class SX147).

A compound (1K) wherein Q represents Q2, L represents an oxygen atom, R¹ represents a chlorine atom, and a combination of R^{X11}, R^{X12}, R^{X13}, R^{X14}, R^{X15}, R^{X16}, R^{X17}, R^{X18}, and R^{X19} represents any combinations described in the Combination C (hereinafter, referred to as Compound Class SX148).

A compound (1K) wherein Q represents Q2, L represents NH, R¹ represents a methyl group, and a combination of R^{X11}, R^{X12,} R^{X13}, R^{X14}, R^{x15}_{,} R^{X16}, R^{X17}, R^{X18}, and R^{X19} represents any combinations described in the Combination C (hereinafter, referred to as Compound Class SX149).

A compound (1K) wherein Q represents Q2, L represents NH, R¹ represents a chlorine atom, and a combination of R^{X11}, R^{X12}, R^{X13}, R^{X14}, R^{X15}, R^{X16}, R^{X17}, R^{X18}, and R^{X19} represents any combinations described in the Combination C (hereinafter, referred to as Compound Class SX150).

A compound represented by formula (1L):

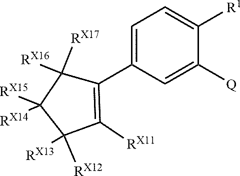

(hereinafter, referred to as “Compound (1L)”),
wherein Q represents Q1, R¹ represents a methyl group, and a combination of R^{X11}, R^{X12}, R^{X13}, R^{X14}, R^{X15}, R^{X16}and R^{X17} represent any combinations described in Combination E (hereinafter, referred to as Compound Class SX151).

Combination E consists of substituent numbers ZE1 to ZE20.

Substituent numbers ZE1 to ZE20 represent combinations of R^{X11}, R^{X12}, R^{X13}, R^{X14}, R^{X15}, R^{X16}, and R^{X17} in the compound (1L), which hereinafter, is referred to as [Substituent No.; R^{X11}, R^{X12,} R^{X13}, R^{X14}, R^{X15}, R^{X16}, R^{X17}]. For example, Substituent No. ZE2 represents a combination wherein R^{X11} represents a methyl group, and R^{X12}, R^{X13}, R^{X14}, R^{X15}, R^{X16}, and R^{X17} represent a hydrogen atom.

### Combination E

[ZE1; H, H, H, H, H, H, H] , [ ZE2;Me, H, H, H, H, H, H] , [ZE3; F, H, H, H, H, H, H ] , [ZE4; Cl, H, H, H, H, H, H] , [ZE5;H, F, F, H, H, H, H] , [ZE6;Me, F, F, H, H, H,H], [ZE7;F, F, F, H, H, H, H] , [ZE8 ; Cl, F, F, H, H, H, H] , [ZE9; H, H, H, H, H, F, F] , [ZE10;Me,H,H,H,H,F, F] , [ZE11; F, H, H, H, H, F, F] , [ZE12;C1, H,H,H,H,F,F],[ZE13;H,Cl,Cl,H,H,H,H],[ZE14;Me,Cl,Cl,H,H,H,H] ,[ZE15;F,Cl,Cl,H,H,H,H],[ZE16;Cl,Cl,Cl,H,H,H,H],[ZE17;H,H,H ,H,H,Cl,Cl],[ZE18;Me,H,H,H,H,Cl,Cl],[ZE19;F,H,H,H,H,Cl,Cl] , [ZE20;Cl,H,H,H,H,Cl,Cl]

A compound (1L) wherein Q represents Q2, L represents NH, R¹ represents a chlorine atom, and a combination of R^{X11}, R^{X12,} R^{X13}, R^{X14}, R^{X15}, R^{X16}, and R^{X17} represents any combinations described in the Combination E (hereinafter, referred to as Compound Class SX152).

A compound (1L) wherein Q represents Q2, L represents an oxygen atom, R¹ represents a methyl group, and a combination of R^{X11}, R^{X12}, R^{X13}, R^{X14}, R^{X15}, R^{X16}, and R^{X17} represents any combinations described in the Combination E (hereinafter, referred to as Compound Class SX153).

A compound (1L) wherein Q represents Q2, L represents an oxygen atom, R¹ represents a chlorine atom, and a combination of R^{X11}, R^{X12,} R^{X13}, R^{X14}, R^{X15}, R^{X16}, and R^{X17} represents any combinations described in the Combination E (hereinafter, referred to as Compound Class SX154).

A compound (1L) wherein Q represents Q2, L represents NH, R¹ represents a methyl group, and a combination of R^{X11}, R^{X12}, R^{X13}, R^{X14}, R^{X15}, R^{X16}, and R^{X17} represents any combinations described in the Combination E (hereinafter, referred to as Compound Class SX155).

A compound (1L) wherein Q represents Q2, L represents NH, R¹ represents a chlorine atom, and a combination of R^{X11}, R^{X12}, R^{X13}, R^{X14}, R^{X15}, R^{X16}, and R^{X17} represents any combinations described in the Combination E (hereinafter, referred to as Compound Class SX156).

A compound represented by formula (1M):

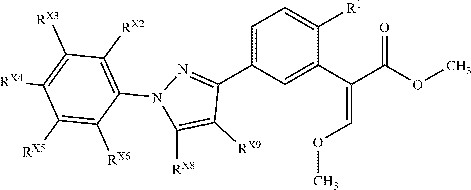

(hereinafter, referred to as “Compound (1M)”),
wherein R¹ represents a methyl group, R^{X8} represents a hydrogen atom, R^{X9} represents a hydrogen atom, and a combination of R^{X2}, R^{X3}, R^{X4}, R^{X5}, and R^{X6} represents any combinations described in Combination A
(hereinafter, referred to as Compound Class SX157).

A compound (1M) wherein R¹ represents a methyl group, R^{X8} represents a methyl group, R^{X9} represents a hydrogen atom, and a combination of R^{X2}, R^{X3}, R^{X4}, R^{X5}, and R^{X6} represents any combinations described in Combination A (hereinafter, referred to as Compound Class SX158).

A compound (1M) wherein R¹ represents a methyl group, R^{X8} represents a hydrogen atom, R^{X9} represents a methyl group, and a combination of R^{X2}, R^{X3}, R^{X4}, R^{X5}, and R^{X6} represents any combinations described in Combination A (hereinafter, referred to as Compound Class SX159).

A compound (1M) wherein R¹ represents a methyl group, R^{X8} represents a fluorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX160).

A compound (1M) wherein R¹ represents a methyl group, R^{X8} represents a hydrogen atom, R^{X9} represents a fluorine atom, and a combination of R^{X2}, R^{X3}, R^{X4}, R^{X5}, and R^{X6} represents any combinations described in Combination A (hereinafter, referred to as Compound Class SX161).

A compound (1M) wherein R¹ represents a methyl group, R^{X8} represents a chlorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX162).

A compound (1M) wherein R¹ represents a methyl group, R^{X8} represents a hydrogen atom, R^{X9} represents a chlorine atom, and a combination of R^{X2}, R^{X3}, R^{X4}, R^{X5}, and R^{X6} represents any combinations described in Combination A (hereinafter, referred to as Compound Class SX163).

A compound (1M) wherein R¹ represents a chlorine atom, R^{X8} represents a hydrogen atom, R^{X9} represents a hydrogen atom, and a combination of R^{X2}, R^{x3}, R^{X4}, R^{X5}, and R^{X6} represents any combinations described in Combination A (hereinafter, referred to as Compound Class SX164).

A compound (1M) wherein R¹ represents a chlorine atom, R^{X8} represents a methyl group, R^{X9} represents a hydrogen atom, and a combination of R^{X2}, R^{X3}, R^{X4}, R^{X5}, and R^{X6} represents any combinations described in Combination A (hereinafter, referred to as Compound Class SX165).

A compound (1M) wherein R¹ represents a chlorine atom, R^{X8} represents a hydrogen atom, R^{X9} represents a methyl group, and a combination of R^{X2}, R^{X3}, R^{X4}, R^{X5}, and R^{X6} represents any combinations described in Combination A (hereinafter, referred to as Compound Class SX166).

A compound (1M) wherein R¹ represents a chlorine atom, R^{X8} represents a fluorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX167).

A compound (1M) wherein R¹ represents a chlorine atom, R^{X8}represents a chlorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX168).

A compound (1M) wherein R¹ represents a chlorine atom, R^{X8} represents a chlorine atom, R^{X9} represents a hydrogen atom, and a combination of R^{X2}, R^{Xx3}, R^{X4}, R^{X5}, and R^{X6} represents any combinations described in Combination A (hereinafter, referred to as Compound Class SX169).

A compound (1M) wherein R¹ represents a chlorine atom, R^{X8} represents a hydrogen atom, R^{X9} represents a chlorine atom, and a combination of R^{X2}, R^{X3}, R^{X4}, R^{X5}, and R^{X6} represents any combinations described in Combination A (hereinafter, referred to as Compound Class SX170).

A compound represented by formula (1N):

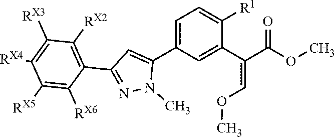

(hereinafter, referred to as “Compound (1N)”),
wherein R¹ represents a methyl group, R^{X9} represents a hydrogen atom, and a combination of R^{X2}, R^{X3}, R^{X4}, R^{X5}, and R^{X6} represents any combinations described in Combination A (hereinafter, referred to as Compound Class SX171).

A compound (1N) wherein R¹ represents a hydrogen atom, R^{X9} represents a methyl group, and a combination of R^{X2}, R^{X3}, R^{X4}, R^{X5} , and R^{X6} represents any combinations described in Combination A (hereinafter, referred to as Compound Class SX172) .

A compound (1N) wherein R¹ represents a chlorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX173) .

A compound (1N) wherein R¹ represents a chlorine atom, R^{X9} represents a methyl group, and a combination of R^{X2}, R^{X3}, R^{X4}, R^{X5}, and R^{X6} represents any combinations described in Combination A (hereinafter, referred to as Compound Class SX174).

A compound represented by formula (1O):

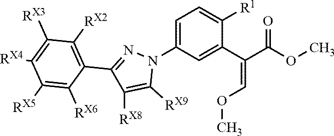

(hereinafter, referred to as “Compound (1O)”),
wherein R¹ represents a methyl group, R^{X8} represents a hydrogen atom, R^{X9} represents a hydrogen atom, and a combination of R^{X2}, R^{X3}, R^{X4}, R^{X5}, and R^{X6} represents any combinations described in Combination A
(hereinafter, referred to as Compound Class SX175).

A compound (1O) wherein R¹ represents a methyl group, R^{X8} represents a fluorine atom, R^{X9} represents a hydrogen atom, and a combination of R^{X2}, R^{X3},R^{X4}, R^{X5}, and R^{X6} represents any combinations described in Combination A (hereinafter, referred to as Compound Class SX176).

A compound (10) wherein R¹ represents a methyl group, R^{X8} represents a chlorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX177).

A compound (10) wherein R¹ represents a methyl group, R^{X8} represents a methyl group, R^{X9} represents a hydrogen atom, and a combination of R^{X2}, R^{X3}, R^{X4}, R^{X5}, and R^{X6} represents any combinations described in Combination A (hereinafter, referred to as Compound Class SX178).

A compound (10) wherein R¹ represents a chlorine atom, R^{X8} represents a hydrogen atom, R^{X9} represents a hydrogen atom, and a combination of R^{X2}, R^{X3},R^{X4}, R^{X5}, and R^{X6} represents any combinations described in Combination A (hereinafter, referred to as Compound Class SX179).

A compound (10) wherein R¹ represents a chlorine atom, R^{X8} represents a fluorine atom, R^{X9} represents a hydrogen atom, and a combination of R^{X2}, R^{X3},R^{X4}, R^{X5}, and R^{X6} represents any combinations described in Combination A (hereinafter, referred to as Compound Class SX180).

A compound (10) wherein R¹ represents a chlorine atom, R^{X8} represents a chlorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX181).

A compound (10) wherein R¹ represents a chlorine atom, R^{X8} represents a methyl group, R^{X9} represents a hydrogen atom, and a combination of R^{X2}, R^{x3}, R^{X4}, R^{X5}, and R^{X6} represents any combinations described in Combination A (hereinafter, referred to as Compound Class SX182).

A compound represented by formula (1P):

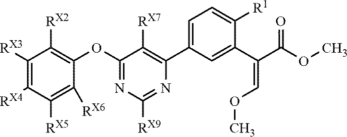

(hereinafter, referred to as “Compound (1P)”),
wherein R¹ represents a methyl group, R^{X7} represents a hydrogen atom, R^{X9} represents a hydrogen atom, and a combination of R^{X2}, R^{X3},R^{X4}, R^{X5}, and R^{X6} represents any combinations described in Combination A
(hereinafter, referred to as Compound Class SX183).

A compound (1P) wherein R¹ represents a methyl group, R^{X7} represents a fluorine atom, R^{X9} represents a hydrogen atom, and a combination of R^{X2}, R^{X3}, R^{X4}, R^{X3}, and R^{X6} represents any combinations described in Combination A (hereinafter, referred to as Compound Class SX184).

A compound (1P) wherein R¹ represents a methyl group, R^{X7} represents a chlorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX185).

A compound (1P) wherein R¹ represents a methyl group, R^{X7} represents a hydrogen atom, R^{X9} represents a fluorine atom, and a combination of R^{X2}, R^{X3},R^{X4}, R^{X5}, and R^{X6} represents any combinations described in Combination A (hereinafter, referred to as Compound Class SX186).

A compound (1P) wherein R¹ represents a chlorine atom, R^{X7} represents a hydrogen atom, R^{X9} represents a hydrogen atom, and a combination of R^{X2}, R^{X3},R^{X4}, R^{X5}, and R^{X6} represents any combinations described in Combination A (hereinafter, referred to as Compound Class SX187).

A compound (1P) wherein R¹ represents a chlorine atom, R^{X7} represents a fluorine atom, R^{X9} represents a hydrogen atom, and a combination of R^{X2}, R^{X3}, R^{X4}, R^{X5}, and R^{X6} represents any combinations described in Combination A (hereinafter, referred to as Compound Class SX188).

A compound (1P) wherein R¹ represents a chlorine atom, R^{X7} represents a chlorine atom, R^{X9} represents a hydrogen atom, and a combination of R^{X2}, R^{X3},R^{X4}, R^{X5}, and R^{X6} represents any combinations described in Combination A (hereinafter, referred to as Compound Class SX189).

A compound (1P) wherein R¹ represents a chlorine atom, R^{X7} represents a hydrogen atom, R^{X9} represents a fluorine atom, and a combination of R^{X2}, R^{X3}, R^{X4}, R^{X5}, and R^{X6} represents any combinations described in Combination A (hereinafter, referred to as Compound Class SX190).

A compound represented by formula (1Q):

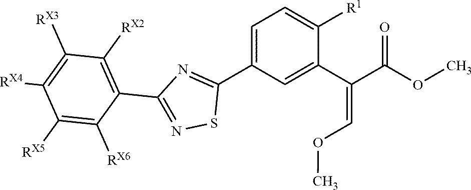

(hereinafter, referred to as “Compound (1Q)”),
wherein R¹ represents a methyl group, and a combination of R^{X2}, R^{X3}, R^{X4}, R^{X5}, and R^{X6} represents any combinations described in Combination A
(hereinafter, referred to as Compound Class SX191).

A compound (1Q) wherein R¹ represents a chlorine atom, and a combination of R^{X2}, R^{X3}, R^{X4}, R^{X5}, and R^{X6} represents any combinations described in Combination A (hereinafter, referred to as Compound Class SX192).

A compound represented by formula (1R):

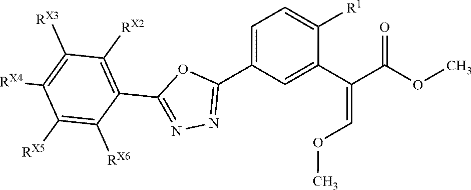

(hereinafter, referred to as “Compound (1R)”),
wherein R¹ represents a methyl group, and a combination of R^{X2}, R^{X3},R^{X4}, R^{X5}, and R^{X6} represents any combinations described in Combination A
(hereinafter, referred to as Compound Class SX193).

A compound (1R) wherein R¹ represents a chlorine atom, and a combination of R^{X2}, R^{X3},R^{X4}, R^{X5}, and R^{X6} represents any combinations described in Combination A (hereinafter, referred to as Compound Class SX194).

A compound represented by formula (1S):

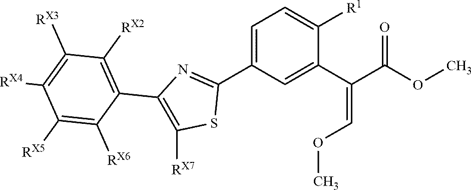

(hereinafter, referred to as “Compound (1S)”),
wherein R¹ represents a methyl group, R^{X7} represents a hydrogen atom, and a combination of R^{X2}, R^{X3},R^{X4}, R^{X5}, and R^{X6} represents any combinations described in Combination A
(hereinafter, referred to as Compound Class SX195).

A compound (1S) wherein R¹ represents a chlorine atom, R^{X7} represents a hydrogen atom, and a combination of R^{X2}, R^{X3}, R^{X4}, R^{X5}, and R^{X6} represents any combinations described in Combination A (hereinafter, referred to as Compound Class SX196) .

A compound (1S) wherein R¹ represents a methyl group, R^{X7} represents a fluorine atom, and a combination of R^{X2}, R^{X3}, R^{X4}, R^{X5}, and R^{X6} represents any combinations described in Combination A (hereinafter, referred to as Compound Class SX197).

A compound (1S) wherein R¹ represents a chlorine atom, R^{X7} represents a fluorine atom, and a combination of R^{X2}, R^{X3}, R^{X4}, R^{X5}, and R^{X6} represents any combinations described in Combination A (hereinafter, referred to as Compound Class SX198).

A compound represented by formula (1T):

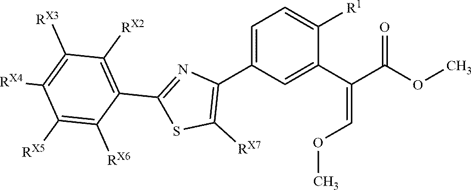

(hereinafter, referred to as “Compound (1T)”),
wherein R¹ represents a methyl group, R^{X7} represents a hydrogen atom, and a combination of R^{X2}, R^{X3},R^{X4}, R^{X5}, and R^{X6} represents any combinations described in Combination A
(hereinafter, referred to as Compound Class SX199).

A compound (1T) wherein R¹ represents a chlorine atom, R^{X7} represents a hydrogen atom, and a combination of R^{X2}, R^{X3}, R^{X4}, R^{X5}, and R^{X6} represents any combinations described in Combination A (hereinafter, referred to as Compound Class SX200).

A compound (1T) wherein R¹ represents a methyl group, R^{X7} represents a fluorine atom, and a combination of R^{X2}, R^{X3}, R^{X4}, R^{X5}, and R^{X6} represents any combinations described in Combination A (hereinafter, referred to as Compound Class SX201) .

A compound (1T) wherein R¹ represents a chlorine atom, R^{X7} represents a fluorine atom, and a combination of R^{X2}, R^{X3}, R^{X4}, R^{X5}, and R^{X6} represents any combinations described in Combination A (hereinafter, referred to as Compound Class SX202) .

A compound represented by formula (1U):

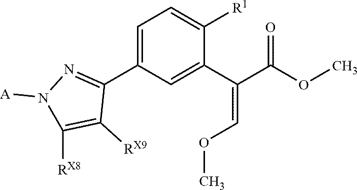

(hereinafter, referred to as “Compound (1U)”),
wherein A represents any one of formula AA1 to formula AA4.

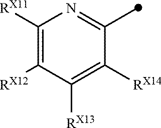

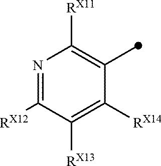

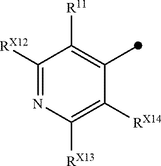

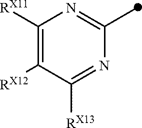

R¹ represents a methyl group, R^{X8} represents a hydrogen atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX203).

Combination D consists of substituent numbers ZD1 to ZD334.

Substituent numbers ZD1 to ZD334 represent combinations of R^{X11}, R^{X12}, R^{X13}, and R^{X14} in the compound (1U), a compound represented by formula (1V), a compound represented by formula (1W), a compound represented by formula (1X), a compound represented by formula (1Y), a compound represented by formula (1Z), a compound represented by formula (2A), which hereinafter, is referred to as [Substituent No.; A, R^{X11}, R^{X12}, R^{X13}, R^{X14}] . For example, Substituent No. ZD2 represents a combination wherein A represents AA1, R^{X11} represents a methyl group, and R^{X12}, R^{X13}, and R^{X14} represent a hydrogen atom.]

### Combination D

[ZD1 ; AA1 , H , H , H , H] , [ZD2 ; AA1 , Me , H , H , H] , [ZD3 ; AA1 , Et , H , H , H] , [ZD 4 ; AA1 , Pr , H , H , H] , [ZD5 ; AA1 , i-Pr , H , H , H] , [ZD6 ; AA1 , c-Pr , H , H , H] ,[ZD7 ; AA1 , Ph , H , H , H] ,[ZD8 ; AA1 , OMe , H , H , H] , [ZD9 ; AA1 , O Et , H , H , H] , [ZD10 ; AA1 , OPh , H , H , H] , [ZD11 ; AA1 , CF₃ , H , H , H] , [ZD12 ; A A1 , F , H , H , H] ,[ZD13 ; AA1 , Cl , H , H , H] , [ZD14 ; AA1 , Br , H , H , H] , [ZD15 ; A A1 , CN , H , H , H] , [ZD16 ; AA1 , H , Me , H , H] , [ZD17 ; AA1 , H , Et , H , H] ,[ZD18 ; AA1 , H , Pr , H , H] , [ZD19 ; AA1 , H , i-Pr , H , H] , [ZD20 ; AA1 , H ,c-Pr , H , H] , [ZD21 ; AA1 , H , Ph , H , H] , [ZD22 ; AA1 , H , OMe , H , H] , [ZD23 ; AA1 , H , OEt , H , H] , [ZD24 ; AA1 , H , OPh , H , 9 H] , [ZD25;AA1,H,CF₃,H,H] , [ZD26 ; AA1 , H , F , H , H] , [ZD27 ; AA1 , H , C1 , H , H] , [ZD28 ; AA1 , H , Br , H , H] , [ZD29 ; AA1 , H , CN , H , H] , [ZD30 ; AA1 , H , H , Me , H] , [ZD31 ; AA1 , H , H , Et , H] , [ZD32 ; AA1 , H , H , Pr , H] , [ZD33 ; AA1 , H , H , i-Pr , H] , [ZD34 ; AA1 , H , H , c-Pr , H] , [ZD35 ; AA1 , H , H , Ph , H] , [ZD36 ; AA1 , H , H , OMe , H] , [ZD37 ; AA1 , H , H , OEt , H] , [ZD38 ; AA1 , H , H , OPh , H] , [ZD39 ; AA1 , H , H , CF₃ , H] , [ZD40 ; AA 1 , H , H , F , H] , [ZD41 ; AA1 , H , H , Cl , H] , [ZD42 ; AA1 , H , H , Br , H] , [ZD43 ; AA 1 , H , H , CN ,H] , [ZD44 ; AA1 , H , H ,H , Me] , [ZD45 ; AA1 , H , H , H , Et] , [ZD46 ; A A1 , H , H , H , Pr] , [ZD47 ; AA1 , H , H , H , i-Pr] , [ZD48 ; AA1 , H , H , H, c-Pr] , [ZD49 ; AA1 , H , H , H , Ph] , [ZD50 ; AA1 , H , H , H , OMe] , [ZD51 ; AA1 , H , H , H , OEt] , [ZD52 ; AA1 , H, H , H , OPh] , [ZD53 ; AA1 , H , H, H , CF₃] , [ZD54 ; AA1 , H , H , H , F] , [ZD55 ; AA1 , H , H , H ,Cl] , [ZD56 ; AA1 , H , H , H , Br] , [ZD57 ; AA1 , H , H , H , CN] , [ZD58 ; AA1 , Me , Me , H ,H] , [ZD59 ; AA1 , Me , Et , H , H] , [ZD60 ; A A1 , Me , OMe, H , H] , [ZD61 ; AA1 , Me , OEt , H , H] , [ZD62 ; AA1 , Me , SMe , H , H] , [ZD63 ; AA1 , Me , F , H , H] , [ZD64 ; AA1 , Me , Cl , H , H] , [ZD65 ; AA1 , Me , CN , H , H] , [ZD66 ; AA1 , F ,Me , H , H] , [ZD67 ; AA1 , F , Et , H ,H] , [ZD68 ; AA1 , F , OMe , H , H] , [ZD69 ; AA1 , F , OEt , H , H] , [ZD70 ; AA1 , F , SMe , H , H] , [ZD71 ; AA1 , F , F , H , H] , [ZD72 ; AA1 , F, Cl , H, H] , [ZD73 ; AA1 , F , CN , H , H] , [ZD74 ; AA1 , Cl , Me , H , H] , [ZD75 ; AA1 , Cl , Et , H , H] , [ZD76 ; AA1 , Cl , OMe , H , H] , [ZD77 ; A A1 , Cl , OEt , H , H] , [ZD78 ; AA1 , Cl , SMe , H , H] , [ZD79 ; AA1 , Cl , F , H , H] , [Z D80 ; AA1 , Cl , Cl , H , H] , [ZD81 ; AA1 , Cl , CN , H , H] , [ZD82 ; AA1 , OMe , Me , H , H] , [ZD83 ; AA1 , OMe , Et , H , H] , [ZD84 ; AA1 , OMe , OMe , H ,H] , [ZD85 ; AA1 , O Me , OEt , H , H] , [ZD86 ; AA1 , OMe , SMe , H , H] , [ZD87 ; AA1 , OMe , F , H , H] , [ZD 88 ; AA1 , OMe , Cl , H , H] , [ZD89 ; AA1 , OMe , CN , H , H] , [ZD90 ; AA1 , SMe , Me , H , H] , [ZD91 ; AA1 , SMe , Et , H , H] , [ZD92 ; AA1 , SMe , OMe , H , H] , [ZD93 ; AA1 , SMe , OEt , H , H] , [ZD94 ; AA1 , SMe , SMe , H , H] , [ZD95 ; AA1 , SMe , F, H , H] , [Z D96 ; AA1, SMe , Cl , H , H] , [ZD97 ; AA1 , SMe , CN , H , H] , [ZD98 ; AA2 , H , H , H , H ] , [ZD99 ; AA2 , Me , H , H , H] , [ZD100 ; AA2 , Et , H , H , H] , [ZD101 ; AA2 , Pr , H , H , H] , [ZD102 ; AA2 , i-Pr , H , H , H] , [ZD103 ; AA2 , c-Pr , H , H ,H] , [ZD104 ; AA2 , Ph , H , H , H] , [ZD105 ; AA2 , OMe , H , H, H] , [ZD106 ; AA2 , OEt , H , H , H] , [ZD107 ; AA2 , OPh , H , H , H] , [ZD108 ; AA2 , CF₃ , H , H , H] , [ZD109 ; AA2 , F , H , H , H] , [ZD110 ; AA2 , Cl ,H , H , H] , [ZD111 ; AA2 , Br , H , H , H] , [ZD112 ; AA2 , CN , H , H , H] , [ZD113 ; AA2 , H , Me , H , H ] , [ZD114 ; AA2 , H , Et , H , H] , [ZD115 ; AA2 , H , Pr , H ,H] , [ZD116 ; AA2 , H , i-Pr , H , H] , [ZD117 ; AA2 , H , c-Pr , H , H] , [ZD118 ; AA2 , H , Ph , H , H ] , [ZD119 ; AA2 , H , OMe , H , H] , [ZD120 ; A A2 , H , OEt , H , H] , [ZD121 ; AA2 , H , OPh , H , H] , [ZD122 ; AA2 , H , CF₃, H , H] , [ ZD123 ; AA2 , H , F , H , H] , [ZD124 ; AA2 , H , Cl , H, H] , [ZD125 ; AA2 , H , Br , H , H ] , [ZD126 ; AA2 , H , CN , H , H] , [ZD127 ; AA2 , H , H , Me , H] , [ZD128 ; AA2 , H , H , Et , H] , [ZD129 ; AA2 , H , H , Pr , H] , [ZD130 ; AA2 , H , H , i-Pr , H] , [ZD131 ; AA2 , H , H , c-Pr , H] , [ZD132 ; AA2 , H , H , Ph , H] , [ZD133 ; AA2 , H , H , OMe , H] , [ZD134 ; AA2 , H , H , OEt , H] , [ZD135 ; AA2 , H , H , OPh , H] , [ZD136 ; AA2 , H , H , CF₃ , H] , [ZD 137 ; AA2 , H , H , F , H] , [ZD138 ; AA2 , H , H , Cl , H] , [ZD139 ; AA2 , H , H , Br , H] , [ZD140 ; AA2 , H , H , CN , H] , [ZD141 ; AA2 , H , H , H , Me] , [ZD142 ; AA2 , H , H , H , Et] , [ZD143 ; AA2 , H , H , H , Pr] , [ZD144 ; AA2 , H, H , H , i-Pr] , [ZD145 ; AA2 , H , H , H , c-Pr] , [ZD146 ; AA2 , H , H , H , Ph] , [ZD147 ; AA2 , H , H , H , OMe] , [ZD148 ; AA2 , H , H , H , OEt] , [ZD149 ; AA2 , H , H , H , OPh] , [ZD150 ; AA2 , H , H , H , CF₃] , [ZD15 1 ; AA2 , H ,H , H , F] , [ZD152 ; AA2 , H , H , H , Cl] , [ZD153 ; AA2 , H , H , H , Br] , [Z D154 ; AA2 , H , H , H , CN] , [ZD155 ; AA2 , Me , Me , H , H] , [ZD156 ; AA2 , Me , Et , H , H] , [ZD157 ; AA2 , Me , OMe , H , H] , [ZD158 ; AA2 , Me , OEt , H , H] , [ZD159 ; AA 2 , Me , SMe , H , H] , [ZD160 ; AA2 , Me , F, H , H] , [ZD161 ; AA2 , Me , Cl , H , H] , [Z D162 ; AA2 , Me , CN , H , H] , [ZD163 ; AA2 , F , Me , H , H ] , [ZD164 ; AA2 , F , Et , H , H] , [ZD165 ; AA2 , F , OMe , H , H ] , [ZD166 ; AA2 , F , OEt ,H , H] , [ZD167 ; AA 2 , F , SMe , H , H] , [ZD168 ; AA2 , F , F , H ,H ] , [ZD169 ; AA2 , F , Cl , H , H] , [ZD170 ; A A2 , F ,CN , H , H] , [ZD171 ; AA2 , Cl , Me , H , H] , [ZD172 ; AA2 , Cl , Et , H , H] , [Z D173 ; AA2 , Cl , OMe , H , H] , [ZD174 ; AA2 , Cl , OEt , H , H] , [ZD175 ; AA2 , Cl , S Me , H , H] , [ZD176 ; AA2 , Cl , F, H , H] , [ZD177 ; AA2 , Cl , Cl , H , H] , [ZD178 ; A A2 , Cl , CN , H , H] , [ZD179 ; AA2 , OMe , Me , H , H] , [ZD180 ; AA2 , OMe , Et , H , H] , [ZD181 ; AA2 , OMe , OMe , H , H] , [ZD182 ; AA2 , OMe , OEt , H , H] , [ZD183 ; AA2 , OMe , SMe , H , H] , [ZD184 ; AA2 , OMe , F , H , H] , [ZD185 ; AA2 , OMe , Cl , H , H] , [ZD186 ; AA2 , OMe , CN , H , H] , [ZD187 ; AA2 , SMe , Me , H , H] , [ZD188 ; AA2 , SM e , Et , H , H] , [ZD189 ; AA2 , SMe , OMe , H , H] , [ZD190 ; AA2 , SMe , OEt , H , H] ,[ ZD191 ; AA2 , SMe , SMe , H , H] , [ZD192 ; AA2 , SMe , F , H , H] , [ZD193 ; AA2 , SMe , Cl , H , H] , [ZD194 ; AA2 , SMe , CN , H , H] , [ZD195 ; AA3 , H , H , H , H] , [ZD196 ; AA3 , Me , H , H , H] , [ZD197 ; AA3 , Et , H , H, H] , [ZD198 ; AA3 , Pr , H, H , H] , [ZD 199 ; AA3 , i-Pr , H , H , H] , [ZD200 ; AA3 , c-Pr , H , H , H] , [ZD201 ; AA3 , Ph , H , H , H] , [ZD202 ; AA3 , OMe , H , H , H] , [ZD203 ; AA3 , OEt , H , H , H] , [ZD204 ; AA3 , OPh , H , H , H] , [ZD205 ; AA3 , CF₃ , H , H , H] , [ZD206 ; AA3 , F , H , H , H] , [ZD207 ; AA3 , Cl , H , H , H] , [ZD208 ; AA3 , Br , H , H , H] , [ZD209 ; AA3 , CN , H , H , H] , [ZD210 ; AA3 , H , Me , H , H] , [ZD211 ; AA3 , H , Et , H , H] , [ZD212 ; AA3 , H , Pr , H , H] , [ZD213 ; AA3 , H , i-Pr , H , H] , [ZD214 ; AA3 , H , c-Pr , H , H] , [ZD215 ; AA3 , H , Ph , H , H] , [ZD216 ; AA3 , H , OMe , H , H] , [ZD217 ; A A3 , H , OEt , H , H] , [ZD218 ; AA3 , H , OPh , H , H] , [ZD219 ; AA3 , H , CF₃ , H , H] , [ ZD220 : AA3 , H , F , H , H] , [ZD221 ; AA3 , H , Cl , H , H] , [ZD222 ; AA3 , H , Br , H , H ] , [ZD223 ; AA3 , H , CN , H , H] , [ZD224 ; AA3 , H , H , Me , H] , [ZD225 ; AA3 , H , H , Et , H] , [ZD226 ; AA3 , H , H , Pr , H] , [ZD227 ; AA3 , H , H , i-Pr , H] , [ZD228 ; AA3 , H , H , c-Pr , H] , [ZD229 ; AA3 , H , H , Ph , H] , [ZD230 ; AA3 , H , H , OMe , H] , [ZD231 ; AA3 , H , H , OEt , H] , [ZD232 ; AA3 , H , H , OPh , H] , [ZD233 ; AA3 , H , H , CF₃ , H] , [ZD 234 ; AA3 , H , H , F , H ] , [ZD235 ; AA3, H , H , Cl , H] , [ZD236 ; AA3, H , H , Br , H] , [ZD237 ; AA3 , H , H, CN , H] , [ZD238 ; AA3 , H , H , H , Me] , [ZD239 ; AA3 , H , H , H , Et] , [ZD240 ; AA3 , H , H , H , Pr] , [ZD241 ; AA3 , H , H , H ,i-Pr] , [ZD242 ; AA3 , H , H, H , c-Pr] , [ZD243 ; AA3 , H , H , H , Ph] , [ZD244 ; AA3 , H , H ,H , OMe] , [ZD245 ; AA3 , H , H , H, OEt] , [ZD246 ; AA3 , H , H, H , OPh] , [ZD247 ; AA3 , H , H , H , CF₃] , [ZD24 8 ; AA3 , H , H , H , F] , [ZD249 ; AA3 , H , H , H , Cl] , [ZD250 ; AA3 , H , H , H ,Br] , [Z D251 ; AA3 , H , H , H , CN] , [ZD252 ; AA3 , Me , Me , H , H] , [ZD253 ; AA3 , Me , Et , H , H] , [ZD254 ; AA3 , Me , OMe , H , H] , [ZD255 ; AA3 , Me, OEt , H , H] , [ZD256 ; AA 3 , Me , SMe , H , H] , [ZD257 ; AA3 , Me , F , H , H] , [ZD258 ; AA3 , Me , Cl , H , H] , [Z D259 ; AA3 , Me , CN , H , H] , [ZD260 ; AA3 , F, Me , H , H] , [ZD261 ; AA3 , F , Et , H , H] , [ZD262 ; AA3 , F , OMe , H , H] , [ZD263 ; AA3, F , OEt , H , H] , [ZD264 ; AA3 , F , SMe , H , H] , [ZD265 ; AA3 , F , F , H , H] , [ZD266 ; AA3 , F , Cl , H , H] , [ZD267 ; A A3 , F , CN , H , H] , [ZD268 ; AA3 , Cl , Me , H , H] , [ZD269 ; AA3 , Cl , Et , H , H] , [Z D270 ; AA3 , Cl , OMe , H , H] , [ZD271 ; AA3 , Cl , OEt , H , H] , [ZD272 ; AA3 , C1 , S Me , H , H] , [ZD273 ; AA3, Cl , F , H , H] , [ZD274 ; AA3 , Cl , Cl , H , H] , [ZD275 ; A A3 , Cl , CN , H , H] , [ZD276 ; AA3 , OMe , Me , H , H] , [ZD277 ; AA3 , OMe , Et , H , H] , [ZD278 ; AA3 , OMe , OMe , H , H] , [ZD279 ; AA3 , OMe , OEt , H , H] , [ZD280 ; AA3 , OMe , SMe , H , H] , [ZD281 ; AA3 , OMe , F , H , H] , [ZD282 ; AA3 , OMe , Cl , H , H] , [ZD283 ; AA3 , OMe , CN , H , H] , [ZD284 ; AA3 , SMe , Me , H , H] , [ZD285 ; AA3 , SM e , Et , H , H] , [ZD286 ; AA3 , SMe , OMe , H , H] , [ZD287 ; AA3 , SMe , OEt , H , H] , [ ZD288 ; AA3 , SMe , SMe , H , H] , [ZD289 ; AA3 , SMe , F , H , H] , [ZD290 ; AA3 , SMe , Cl , H , H] , [ZD291 ; AA3 , SMe , CN , H , H] , [ZD292 ; AA4 , H , H , H , -] , [ZD293 ; AA4 , Me , H , H , -] , [ZD294 ; AA4 , Et , H , H , -] , [ZD295 ; AA4 , Pr , H , H , -] , [ZD 296 ; AA4 , i-Pr , H , H , -] , [ZD297 ; AA4 , c-Pr , H , H , -] , [ZD298 ; AA4 , Ph , H , H , -] , [ZD299 ; AA4 , OMe , H , H , -] , [ZD300 ; AA4 , OEt , H , H , -] , [ZD301 ; AA4 , OPh , H , H , -] , [ZD302 ; AA4 , CF₃ , H , H , -] , [ZD303 ; AA4 , F , H , H , -] , [ZD304 ; AA4 , Cl , H , H , -] , [ZD305 ; AA4 , Br , H , H , -] , [ZD306 ; AA4 , CN , H , H , -] , [ZD307 ; AA4 , H , Me , H, -] , [ZD308 ; AA4 , H , Et , H , -] , [ZD309 ; AA4 , H , Pr , H , -] , [ZD310 ; AA4 , H , i-Pr , H , -] , [ZD311 ; AA4 , H , c-Pr , H , -] , [ZD312 ; AA4 , H , Ph , H , -] , [ZD313 ; AA4 , H , OMe , H , -] , [ZD314 ; A A4 , H , OEt , H, -] , [ZD315 ; AA4 , H , OPh , H , -] , [ZD316 ; AA4 , H , CF₃ , H , -] , [ ZD317;AA4,H,F,H,-], [ZD318;AA4,H,Cl,H,-], [ZD319;AA4,H,Br,H,-],[ZD320;AA4,H,CN,H,-],[ZD321;AA4,H,H,Me,-],[ZD322;AA4,H,H, Et,-],[ZD323;AA4,H,H,Pr,-],[ZD324;AA4,H,H,i-Pr,-],[ZD325;AA4,H,H,c-Pr,-],[ZD326;AA4,H,H,Ph,-],[ZD327;AA4,H,H,OMe,-],[ZD328;AA4 ,H,H,OEt,-],[ZD329;AA4,H,H,OPh,-],[ZD330;AA4,H,H,CF₃-],[ZD 331;AA4,H,H,F,-],[ZD332;AA4,H,H,Cl,-],[ZD333;AA4,H,H,Br,-], [ZD334;AA4,H,H,CN,-]

A compound (1U) wherein R¹ represents a methyl group, R^{X8} represents a fluorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX204).

A compound (1U) wherein R¹ represents a methyl group, R^{X8} represents a hydrogen atom, R^{X9} represents a fluorine atom, and a combination of structure of A and substituents of R^{X11}, R^{X12}, R^{X13} and R^{X14} applied to the structure of A represents any combinations described in Combination D (hereinafter, referred to as Compound class SX205).

A compound (1U) wherein R¹ represents a chlorine atom, R^{X8} represents a hydrogen atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX206).

A compound (1U) wherein R¹ represents a chlorine atom, R^{X8} represents a fluorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX207).

A compound (1U) wherein R¹ represents a chlorine atom, R^{X8} represents a hydrogen atom, R^{X9} represents a fluorine atom, and a combination of structure of A and substituents of R^{X11}, R^{X12}, R^{X13} and R^{X14} applied to the structure of A represents any combinations described in Combination D (hereinafter, referred to as Compound class SX208).

A compound represented by formula (1V):

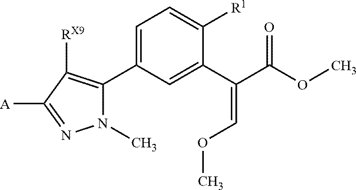

(hereinafter, referred to as “Compound (1V)”),
wherein A represents any one of formula AA1 to formula AA4.

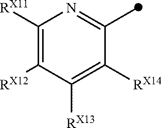

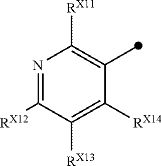

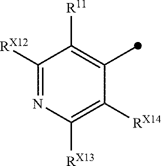

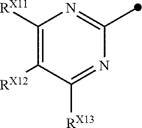

R¹ represents a methyl group, R^{X8} represents a hydrogen atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX209).

A compound (1V) wherein R¹ represents a methyl group, R^{X9} represents a methyl group, and a combination of structure of A and substituents of R^{X11}, R^{X12}, R^{X13} and R^{X14} applied to the structure of A represents any combinations described in Combination D (hereinafter, referred to as Compound class SX210) .

A compound (1V) wherein R¹ represents a chlorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX211).

A compound (1V) wherein R¹ represents a chlorine atom, R^{X9} represents a methyl group, and a combination of structure of A and substituents of R^{X11}, R^{X12}, R^{X13} and R^{X14} applied to the structure of A represents any combinations described in Combination D (hereinafter, referred to as Compound class SX212).

A compound represented by formula (1W):

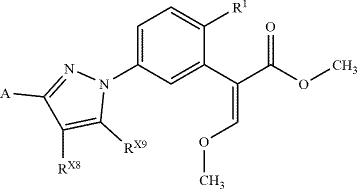

(hereinafter, referred to as “Compound (1W)”), wherein
A represents any one of formula AA1 to formula AA4,
R¹ represents a methyl group, R^{X8} represents a hydrogen atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX213).

A compound (1W) wherein R¹ represents a methyl group, R^{X8} represents a fluorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX214).

A compound (1W) wherein R¹ represents a methyl group, R^{X8} represents a chlorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX215).

A compound (1W) wherein R¹ represents a methyl group, R^{X8} represents a methyl group, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX216).

A compound (1W) wherein R¹ represents a chlorine atom, R^{X8} represents a hydrogen atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX217).

A compound (1W) wherein R¹ represents a chlorine atom, R^{X8} represents a fluorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX218).

A compound (1W) wherein R¹ represents a chlorine atom, R^{X8} represents a chlorine atom, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX219).

A compound (1W) wherein R¹ represents a chlorine atom, R^{X8} represents a methyl group, R^{X9} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX220).

A compound represented by formula (1X):

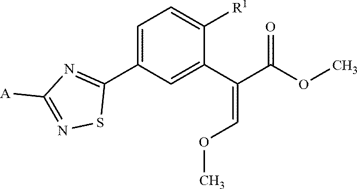

(hereinafter, referred to as “Compound (1X)”), wherein
A represents any one of formula AA1 to formula AA4,
R¹ represents a methyl group, and a combination of structure of A and substituents of R^{X11}, R^{X12}, R^{X13} and R^{X14} applied to the structure of A represents any combinations described in Combination D (hereinafter, referred to as Compound class SX221).

A compound (1X) wherein R¹ represents a chlorine atom, and substituents of R^{X11}, R^{X12}, R^{X13} and R^{X14} applied to the structure of A represents any combinations described in Combination D (hereinafter, referred to as Compound class SX222).

A compound represented by formula (1Y):

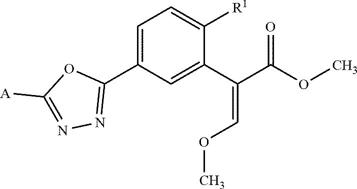

(hereinafter, referred to as “Compound (1Y)”), wherein
A represents any one of formula AA1 to formula AA4,
R¹ represents a methyl group, and a combination of structure of A and substituents of R^{X11}, R^{X12}, R^{X13} and R^{X14} applied to the structure of A represents any combinations described in Combination D (hereinafter, referred to as Compound class SX223).

A compound (1Y) wherein R¹ represents a chlorine atom, and substituents of R^{X11}, R^{X12}, R^{X13} and R^{X14} applied to the structure of A represents any combinations described in Combination D (hereinafter, referred to as Compound class SX224).

A compound represented by formula (1Z):

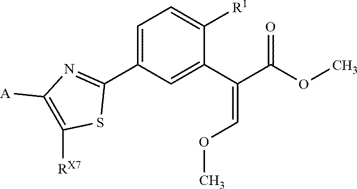

(hereinafter, referred to as “Compound (1Z)”), wherein
A represents any one of formula AA1 to formula AA4,
R¹ represents a methyl group, R^{X7} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX225).

A compound (1Z) wherein R¹ represents a chlorine atom, R^{X7} represents a hydrogen atom, and substituents of R^{X11}, R^{X12}, R^{X13} and R^{X14} applied to the structure of A represents any combinations described in Combination D (hereinafter, referred to as Compound class SX226).

A compound (1Z) wherein R¹ represents a methyl group, R^{X7} represents a fluorine atom, and substituents of R^{X11}, R^{X12}, R^{X13} and R^{X14} applied to the structure of A represents any combinations described in Combination D (hereinafter, referred to as Compound class SX227).

A compound (1Z) wherein R¹ represents a chlorine atom, R^{X7} represents a fluorine atom, and substituents of R^{X11}, R^{X12}, R^{X13} and R^{X14} applied to the structure of A represents any combinations described in Combination D (hereinafter, referred to as Compound class SX228).

A compound represented by formula (2A):

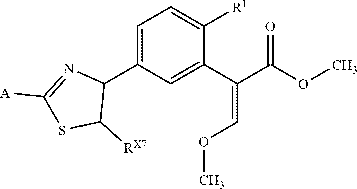

(hereinafter, referred to as “Compound (2A)”), wherein
A represents any one of formula AA1 to formula AA4,
R¹ represents a methyl group, R^{X7} represents a hydrogen atom, and a combination of structure of A and substituents of R^{X11}, R^{X12}, R^{X13} and R^{X14} applied to the structure of A represents any combinations described in Combination D (hereinafter, referred to as Compound class SX229).

A compound (2A) wherein R¹ represents a chlorine atom, R^{X7} represents a hydrogen atom, and substituents of R^{X11}, R^{X12}, R^{X13} and R^{X14} applied to the structure of A represents any combinations described in Combination D (hereinafter, referred to as Compound class SX230).

A compound (2A) wherein R¹ represents a methyl group, R^{X7} represents a fluorine atom, and substituents of R^{X11}, R^{X12}, R^{X13} and R^{X14} applied to the structure of A represents any combinations described in Combination D (hereinafter, referred to as Compound class SX231).

A compound (2A) wherein R¹ represents a chlorine atom, R^{X7} represents a fluorine atom, and substituents of R^{X11}, R^{X12}, R^{X13} and R^{X14} applied to the structure of A represents any combinations described in Combination D (hereinafter, referred to as Compound class SX232).

A compound represented by formula (2B):

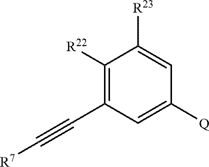

(hereinafter, referred to as “Compound (2B)”), wherein

Q represents Q1, R²² represents a methyl group, R²³ represents a hydrogen atom, and R⁷ represents any substituents selected from Group X (hereinafter, referred to as Compound class SX233).

A compound (2B) wherein Q represents Q1, R²² represents a hydrogen atom, R²³ represents a methyl group, and R⁷ represents any substituents selected from Group X (hereinafter, referred to as Compound class SX234).

A compound (2B) wherein Q represents Q1, R²² represents a chlorine atom, R²³ represents a hydrogen atom, and R⁷ represents any substituents selected from Group X (hereinafter, referred to as Compound class SX235).

A compound (2B) wherein Q represents Q1, R²² represents a hydrogen atom, R²³ represents a chlorine atom, and R⁷ represents any substituents selected from Group X (hereinafter, referred to as Compound class SX236).

A compound (2B) wherein Q represents Q2, L represents an oxygen atom, R²² represents a methyl group, R²³ represents a hydrogen atom, and R⁷ represents any substituents selected from Group X (hereinafter, referred to as Compound class SX237).

A compound (2B) wherein Q represents Q2, L represents an oxygen atom, R²² represents a hydrogen atom, R²³ represents a methyl group, and R⁷ represents any substituents selected from Group X (hereinafter, referred to as Compound class SX238).

A compound (2B) wherein Q represents Q2, L represents an oxygen atom, R²² represents a chlorine atom, R²³ represents a hydrogen atom, and R⁷ represents any substituents selected from Group X (hereinafter, referred to as Compound class SX239).

A compound (2B) wherein Q represents Q2, L represents an oxygen atom, R²² represents a hydrogen atom, R²³ represents a chlorine atom, and R⁷ represents any substituents selected from Group X (hereinafter, referred to as Compound class SX240) .

A compound (2B) wherein Q represents Q2, L represents NH, R²² represents a methyl group, R²³ represents a hydrogen atom, and R⁷ represents any substituents selected from Group X (hereinafter, referred to as Compound class SX241).

A compound (2B) wherein Q represents Q2, L represents NH, R²² represents a hydrogen atom, R²³ represents a methyl group, and R⁷ represents any substituents selected from Group X (hereinafter, referred to as Compound class SX242).

A compound (2B) wherein Q represents Q2, L represents NH, R²² represents a chlorine atom, R²³ represents a hydrogen atom, and R⁷ represents any substituents selected from Group X (hereinafter, referred to as Compound class SX243).

A compound (2B) wherein Q represents Q2, L represents NH, R²² represents a hydrogen atom, R²³ represents a chlorine atom, and R⁷ represents any substituents selected from Group X (hereinafter, referred to as Compound class SX244).

A compound represented by formula (2C):

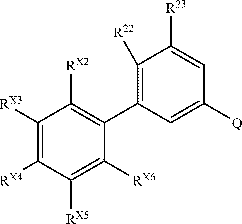

(hereinafter, referred to as “Compound (2C)”), wherein

Q represents Q1, R²² represents a methyl group, R²³ represents a hydrogen atom, and a combination of R^{X2}, R^{X3}, R^{X4}, R^{X5}, and R^{X6} represents any combinations selected from Combination A

(hereinafter, referred to as Compound class SX245).

A compound (2C) wherein Q represents Q1, R²² represents a hydrogen atom, R²³ represents a methyl group, and a combination of R^{X2}, R^{X3}, R^{X4}, R^{X5}, and R^{X6} represents any combinations selected from Combination A (hereinafter, referred to as Compound class SX246).

A compound (2C) wherein Q represents Q1, R²² represents a chlorine atom, R²³ represents a hydrogen atom, and a combination of R^{X2}, R^{X3}, R^{X4}, R^{X5}, and R^{X6} represents any combinations selected from Combination A (hereinafter, referred to as Compound class SX247).

A compound (2C) wherein Q represents Q1, R²² represents a hydrogen atom, R²³ represents a chlorine atom, and a combination of R^{X2}, R^{X3}, R^{X4}, R^{X5}, and R^{X6} represents any combinations selected from Combination A (hereinafter, referred to as Compound class SX248).

A compound (2C) wherein Q represents Q2, L represents an oxygen atom, R²² represents a methyl group, R²³ represents a hydrogen atom, and a combination of R^{X2}, R^{X3}, R^{X4}, R^{X5}, and R^{X6} represents any combinations selected from Combination A (hereinafter, referred to as Compound class SX249).

A compound (2C) wherein Q represents Q2, L represents an oxygen atom, R²² represents a hydrogen atom, R²³ represents a methyl group, and a combination of R^{X2}, R^{X3}, R^{X4}, R^{X5}, and R^{X6} represents any combinations selected from Combination A (hereinafter, referred to as Compound class SX250).

A compound (2C) wherein Q represents Q2, L represents an oxygen atom, R²² represents a chlorine atom, R²³ represents a hydrogen atom, and a combination of R^{X2}, R^{X3}, R^{X4}, R^{X5}, and R^{X6} represents any combinations selected from Combination A (hereinafter, referred to as Compound class SX251).

A compound (2C) wherein Q represents Q2, L represents an oxygen atom, R²² represents a hydrogen atom, R²³ represents a chlorine atom, and a combination of R^{X2}, R^{X3}, R^{X4}, R^{X5}, and R^{X6} represents any combinations selected from Combination A (hereinafter, referred to as Compound class SX252).

A compound (2C) wherein Q represents Q2, L represents an oxygen atom, R²² represents a methyl group, R²³ represents a hydrogen atom, and a combination of R^{X2}, R^{X3}, R^{X4}, R^{X5}, and R^{X6} represents any combinations selected from Combination A (hereinafter, referred to as Compound class SX253).

A compound (2C) wherein Q represents Q2, L represents NH, R²² represents a hydrogen atom, R²³ represents a methyl group, and a combination of R^{X2}, R^{X3}, R^{X4}, R^{X5}, and R^{X6} represents any combinations selected from Combination A (hereinafter, referred to as Compound class SX254).

A compound (2C) wherein Q represents Q2, L represents NH, R²² represents a chlorine atom, R²³ represents a hydrogen atom, and a combination of R^{X2}, R^{X3}, R^{X4}, R^{X5}, and R^{X6} represents any combinations selected from Combination A (hereinafter, referred to as Compound class SX255).

A compound (2C) wherein Q represents Q2, L represents NH, R²² represents a hydrogen atom, R²³ represents a chlorine atom, and a combination of R^{X2}, R^{X3}, R^{X4}, R^{X5}, and R^{X6} represents any combinations selected from Combination A (hereinafter, referred to as Compound class SX256).

A compound represented by formula (2D):

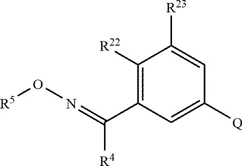

(hereinafter, referred to as “Compound (2D)”), wherein

Q represents Q1, R²² represents a methyl group, R²³ represents a hydrogen atom, R⁴ represents a methyl group, and R⁵ represents any substituents selected from Group X (hereinafter, referred to as Compound class SX247).

A compound (2D) wherein Q represents Q1, R²² represents a hydrogen atom, R²³ represents a methyl group, R⁴ represents a methyl group, and R⁵ represents any substituents selected from Group X (hereinafter, referred to as Compound class SX258).

A compound (2D) wherein Q represents Q1, R²² represents a chlorine atom, R²³ represents a hydrogen atom, R⁴ represents a methyl group, and R⁵ represents any substituents selected from Group X (hereinafter, referred to as Compound class SX259).

A compound (2D) wherein Q represents Q1, R²² represents a hydrogen atom, R²³ represents a chlorine atom, R⁴ represents a methyl group, and R⁵ represents any substituents selected from Group X (hereinafter, referred to as Compound class SX260).

A compound (2D) wherein Q represents Q2, L represents an oxygen atom, R²² represents a methyl group, R²³ represents a hydrogen atom, R⁴ represents a methyl group, and R⁵ represents any substituents selected from Group X (hereinafter, referred to as Compound class SX261).

A compound (2D) wherein Q represents Q2, L represents an oxygen atom, R²² represents a hydrogen atom, R²³ represents a methyl group, R⁴ represents a methyl group, and R⁵ represents any substituents selected from Group X (hereinafter, referred to as Compound class SX262).

A compound (2D) wherein Q represents Q2, L represents an oxygen atom, R²² represents a chlorine atom, R²³ represents a hydrogen atom, R⁴ represents a methyl group, and R⁵ represents any substituents selected from Group X (hereinafter, referred to as Compound class SX263).

A compound (2D) wherein Q represents Q2, L represents an oxygen atom, R²² represents a hydrogen atom, R²³ represents a chlorine atom, R⁴ represents a methyl group, and R⁵ represents any substituents selected from Group X (hereinafter, referred to as Compound class SX264).

A compound (2D) wherein Q represents Q2, L represents NH, R²² represents a methyl group, R²³ represents a hydrogen atom, R⁴ represents a methyl group, and R⁵ represents any substituents selected from Group X (hereinafter, referred to as Compound class SX265).

A compound (2D) wherein Q represents Q2, L represents NH, R²² represents a hydrogen atom, R²³ represents a methyl group, R⁴ represents a methyl group, and R⁵ represents any substituents selected from Group X (hereinafter, referred to as Compound class SX266).

A compound (2D) wherein Q represents Q2, L represents NH, R²² represents a chlorine atom, R²³ represents a hydrogen atom, R⁴ represents a methyl group, and R⁵ represents any substituents selected from Group X (hereinafter, referred to as Compound class SX267).

A compound (2D) wherein Q represents Q2, L represents NH, R²² represents a hydrogen atom, R²³ represents a chlorine atom, R⁴ represents a methyl group, and R⁵ represents any substituents selected from Group X (hereinafter, referred to as Compound class SX268).

A compound (2D) wherein Q represents Q2, L represents NH, R²² represents a methyl group, R²³ represents a hydrogen atom, R⁴ represents a hydrogen atom and R⁵ represents any substituents selected from Group X (hereinafter, referred to as Compound class SX269).

A compound (2D) wherein Q represents Q1, R²² represents a hydrogen atom, R²³ represents a methyl group, R⁴ represents a hydrogen atom and R⁵ represents any substituents selected from Group X (hereinafter, referred to as Compound class SX270).

A compound (2D) wherein Q represents Q1, R²² represents a chlorine atom, R²³ represents a hydrogen atom, R⁴ represents a hydrogen atom and R⁵ represents any substituents selected from Group X (hereinafter, referred to as Compound class SX271).

A compound (2D) wherein Q represents Q1, R²² represents a hydrogen atom, R²³ represents a chlorine atom, R⁴ represents a hydrogen atom and R⁵ represents any substituents selected from Group X (hereinafter, referred to as Compound class SX272).

A compound (2D) wherein Q represents Q2, L represents an oxygen atom, R²² represents a methyl group, R²³ represents a hydrogen atom, R⁴ represents a hydrogen atom and R⁵ represents any substituents selected from Group X (hereinafter, referred to as Compound class SX273).

A compound (2D) wherein Q represents Q2, L represents an oxygen atom, R²² represents a hydrogen atom, R²³ represents a methyl group, R⁴ represents a hydrogen atom and R⁵ represents any substituents selected from Group X (hereinafter, referred to as Compound class SX274).

A compound (2D) wherein Q represents Q2, L represents an oxygen atom, R²² represents a chlorine atom, R²³ represents a hydrogen atom, R⁴ represents a hydrogen atom and R⁵ represents any substituents selected from Group X (hereinafter, referred to as Compound class SX275).

A compound (2D) wherein Q represents Q2, L represents an oxygen atom, R²² represents a hydrogen atom, R²³ represents a chlorine atom, R⁴ represents a hydrogen atom and R⁵ represents any substituents selected from Group X (hereinafter, referred to as Compound class SX276).

A compound (2D) wherein Q represents Q2, L represents NH, R²² represents a methyl group, R²³ represents a hydrogen atom, R⁴ represents a hydrogen atom and R⁵ represents any substituents selected from Group X (hereinafter, referred to as Compound class SX277).

A compound (2D) wherein Q represents Q2, L represents NH, R²² represents a hydrogen atom, R²³ represents a methyl group, R⁴ represents a hydrogen atom and R⁵ represents any substituents selected from Group X (hereinafter, referred to as Compound class SX278).

A compound (2D) wherein Q represents Q2, L represents NH, R²² represents a chlorine atom, R²³ represents a hydrogen atom, R⁴ represents a hydrogen atom and R⁵ represents any substituents selected from Group X (hereinafter, referred to as Compound class SX279).

A compound (2D) wherein Q represents Q2, L represents NH, R²² represents a hydrogen atom, R²³ represents a chlorine atom, R⁴ represents a hydrogen atom and R⁵ represents any substituents selected from Group X (hereinafter, referred to as Compound class SX280).

A compound represented by formula (2E):

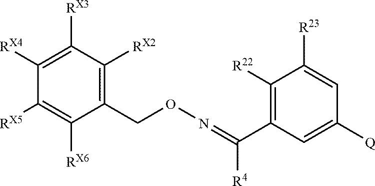

(hereinafter, referred to as “Compound (2E)”), wherein

Q represents Q1, R²² represents a methyl group, R²³ represents a hydrogen atom, R⁴ represents a methyl group, and a combination of R^{X2}, R^{X3}, R^{X4}, R^{X5}, and R^{X5} represents any combinations described in Combination A (hereinafter, referred to as Compound class SX281).

A compound (2E) wherein Q represents Q1, R²² represents a hydrogen atom, R²³ represents a hydrogen atom, R²³ represents a methyl group, R⁴ represents a methyl group, and a combination of R^{X2}, R^{X3}, R^{X4}, R^{X5}, and R^{X5} represents any combinations described in Combination A (hereinafter, referred to as Compound class SX282).

A compound (2E) wherein R²² represents a chlorine atom, Q represents Q1, R²³ represents a hydrogen atom, R⁴ represents a methyl group, and a combination of R^{X2}, R^{X3}, R^{X4}, R^{X5}, and R^{X6} represents any combinations described in Combination A (hereinafter, referred to as Compound class SX283).

A compound (2E) wherein R²² represents a hydrogen atom, Q represents Q1, R²³ represents a chlorine atom, R⁴ represents a methyl group, and a combination of R^{X2}, R^{X3}, R^{X4}, R^{X5}, and R^{X6} represents any combinations described in Combination A (hereinafter, referred to as Compound class SX284).

A compound (2E) wherein Q represents Q1, R²² represents a methyl group, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX285).

A compound (2E) wherein Q represents Q1, R²² represents a hydrogen atom, R²³ represents a methyl group, R⁴ represents a hydrogen atom, and a combination of R^{X2}, R^{X3}, R^{X4}, R^{X5}, and R^{X6} represents any combinations described in Combination A (hereinafter, referred to as Compound class SX286).

A compound (2E) wherein Q represents Q1, R²² represents a chlorine atom, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX287).

A compound (2E) wherein Q represents Q1, R²² represents a hydrogen atom, R²³ represents a chlorine atom, R⁴ represents a hydrogen atom, and a combination of R^{X2}, R^{X3}, R^{X4}, R^{X5}, and R^{X6} represents any combinations described in Combination A (hereinafter, referred to as Compound class SX288).

A compound (2E) wherein Q represents Q2, L represents an oxygen atom, R²² represents a methyl group, R²³ represents a hydrogen atom, R⁴ represents a methyl group, and a combination of R^{X2}, R^{X3}, R^{X4}, R^{X5}, and R^{X6} represents any combinations described in Combination A (hereinafter, referred to as Compound class SX289).

A compound (2E) wherein Q represents Q2, L represents an oxygen atom, R²² represents a hydrogen atom, R²³ represents a methyl group, R⁴ represents a methyl group, and a combination of R^{X2}, R^{X3}, R^{X4}, R^{X5}, and R^{X6} represents any combinations described in Combination A (hereinafter, referred to as Compound class SX290).

A compound (2E) wherein R²² represents a chlorine atom, Q represents Q2, L represents an oxygen atom, R²³ represents a hydrogen atom, R⁴ represents a methyl group, and a combination of R^{X2}, R^{X3}, R^{X4}, R^{X5}, and R^{X6} represents any combinations described in Combination A (hereinafter, referred to as Compound class SX291).

A compound (2E) wherein R²² represents a hydrogen atom, Q represents Q2, L represents an oxygen atom, R²³ represents a chlorine atom, R⁴ represents a methyl group, and a combination of R^{X2}, R^{X3}, R^{X4}, R^{X5}, and R^{X6} represents any combinations described in Combination A (hereinafter, referred to as Compound class SX292).

A compound (2E) wherein Q represents Q2, L represents an oxygen atom, R²² represents a methyl group, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX293).

A compound (2E) wherein Q represents Q2, L represents an oxygen atom, R²² represents a hydrogen atom, R²³ represents a methyl group, R⁴ represents a hydrogen atom, and a combination of R^{X2}, R^{X3}, R^{X4}, R^{X5}, and R^{X6} represents any combinations described in Combination A (hereinafter, referred to as Compound class SX294).

A compound (2E) wherein Q represents Q2, L represents an oxygen atom, R²² represents a chlorine atom, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX295).

A compound (2E) wherein Q represents Q2, L represents an oxygen atom, R²² represents a hydrogen atom, R²³ represents a chlorine atom, R⁴ represents a hydrogen atom, and a combination of R^{X2}, R^{X3}, R^{X4}, R^{X5}, and R^{X6} represents any combinations described in Combination A (hereinafter, referred to as Compound class SX296).

A compound (2E) wherein Q represents Q2, L represents NH, R²² represents a methyl group, R²³ represents a hydrogen atom, R⁴ represents a methyl group, and a combination of R^{X2}, R^{X3}, R^{X4}, R^{X5}, and R^{X6} represents any combinations described in Combination A (hereinafter, referred to as Compound class SX297).

A compound (2E) wherein Q represents Q2, L represents NH, R²² represents a hydrogen atom, R²³ represents a methyl group, R⁴ represents a methyl group, and a combination of R^{X2}, R^{X3}, R^{X4}, R^{X5}, and R^{X6} represents any combinations described in Combination A (hereinafter, referred to as Compound class SX298).

A compound (2E) wherein R²² represents a chlorine atom, Q represents Q2, L represents NH, R²³ represents a hydrogen atom, R⁴ represents a methyl group, and a combination of R^{X2}, R^{X3}, R^{X4}, R^{X5}, and R^{X6} represents any combinations described in Combination A (hereinafter, referred to as Compound class SX299) .

A compound (2E) wherein R²² represents a hydrogen atom, Q represents Q2, L represents NH, R²³ represents a chlorine atom, R⁴ represents a methyl group, and a combination of R^{X2}, R^{X3}, R^{X4}, R^{X5}, and R^{X6} represents any combinations described in Combination A (hereinafter, referred to as Compound class SX300) .

A compound (2E) wherein Q represents Q2, L represents NH, R²² represents a methyl group, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX301).

A compound (2E) wherein Q represents Q2, L represents NH, R²² represents a methyl group, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX302).

A compound (2E) wherein Q represents Q2, L represents NH, R²² represents a chlorine atom, R²³ represents a hydrogen atom, R⁴ represents a hydrogen atom, and a combination of R^{X2}, R^{X3}, R^{X4}, R^{X5}, and R^{X6} represents any combinations described in Combination A (hereinafter, referred to as Compound class SX303).

A compound (2E) wherein Q represents Q2, L represents NH, R²² represents a hydrogen atom, R²³ represents a chlorine atom, R⁴ represents a hydrogen atom, and a combination of R^{X2}, R^{X3}, R^{X4}, R^{X5}, and R^{X6} represents any combinations described in Combination A (hereinafter, referred to as Compound class SX304).

A compound represented by formula (2F):

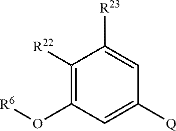

(hereinafter, referred to as “Compound (2F)”), wherein

Q represents Q1, R²² represents a methyl group, R²³ represents a hydrogen atom, and R⁶ represents any substituents selected from Group Y (hereinafter, referred to as Compound class SX305).

A compound (2F) wherein Q represents Q1, R²² represents a chlorine atom, R²³ represents a hydrogen atom, and R⁶ represents any substituents selected from Group Y (hereinafter, referred to as Compound class SX306).

A compound (2F) wherein Q represents Q1, R²² represents a hydrogen atom, R²³ represents a methyl group, and R⁶ represents any substituents selected from Group Y (hereinafter, referred to as Compound class SX307).

A compound (2F) wherein Q represents Q1, R²² represents a hydrogen atom, R²³ represents a chlorine atom, and R⁶ represents any substituents selected from Group Y (hereinafter, referred to as Compound class SX308).

A compound (2F) wherein Q represents Q2, L represents an oxygen atom, R²² represents a methyl group, R²³ represents a hydrogen atom, and R⁶ represents any substituents selected from Group Y (hereinafter, referred to as Compound class SX309) .

A compound (2F) wherein Q represents Q2, L represents an oxygen atom, R²² represents a chlorine atom, R²³ represents a hydrogen atom, and R⁶ represents any substituents selected from Group Y (hereinafter, referred to as Compound class SX310) .

A compound (2F) wherein Q represents Q2, L represents an oxygen atom, R²² represents a hydrogen atom, R²³ represents a methyl group, and R⁶ represents any substituents selected from Group Y (hereinafter, referred to as Compound class SX311).

A compound (2F) wherein Q represents Q2, L represents an oxygen atom, R²² represents a hydrogen atom, R²³ represents a chlorine atom, and R⁶ represents any substituents selected from Group Y (hereinafter, referred to as Compound class SX312) .

A compound (2F) wherein Q represents Q2, L represents NH, R²² represents a methyl group, R²³ represents a hydrogen atom, and R⁶ represents any substituents selected from Group Y (hereinafter, referred to as Compound class SX313).

A compound (2F) wherein Q represents Q2, L represents NH, R²² represents a chlorine atom, R²³ represents a hydrogen atom, and R⁶ represents any substituents selected from Group Y (hereinafter, referred to as Compound class SX314).

A compound (2F) wherein Q represents Q2, L represents NH, R²² represents a hydrogen atom, R²³ represents a methyl group, and R⁶ represents any substituents selected from Group Y (hereinafter, referred to as Compound class SX315).

A compound (2F) wherein Q represents Q2, L represents NH, R²² represents a hydrogen atom, R²³ represents a chlorine atom, and R⁶ represents any substituents selected from Group Y (hereinafter, referred to as Compound class SX316).

Next, the formulation examples of the compounds of the present invention are shown below. In the formulation examples, the “parts” represents “part by weight” unless otherwise specified. The compound S of the present invention represents the compounds described in the Compound Classes SX1 to SX316.

Formulation Example 1

Fifty parts of any one of the compound S of the present invention, 3 parts of calcium lignin sulfonate, 2 parts of Magnesium lauryl sulfate, and 45 parts of synthetic hydrated silicon dioxide are well mixed-grinding to obtain a formulation.

### Formulation Example 2

Twenty (20) parts of any one of the compound S of the present invention, 1.5 parts of sorbitan trioleate are mixed with 28.5 parts of an aqueous solution containing 2 parts of polyvinyl alcohol, and the mixture is then finely-ground by a wet grinding method. To the mixture is then added 40 parts of an aqueous solution containing 0.05 parts of xanthan gum and 0.1 parts of magnesium aluminum silicate, and 10 parts of propylene glycol is further added thereto. The mixture is stirred to obtain a formulation.

### Formulation Example 3

Two(2) parts of any one of the compound S of the present invention, 88 parts of kaolin clay and 10 parts of talc are mixed-grinding to obtain a formulation.

### Formulation Example 4

Five (5) parts of any one of the compound S of the present invention, 14 parts of polyoxyethylene styryl phenyl ether, 6 parts of calcium dodecylbenzene sulfonate and 75 parts of xylene are well mixed to obtain a formulation.

### Formulation Example 5

Two(2) parts of any one of the compound S of the present invention, 1 part of synthetic hydrated silicon dioxide, 2 parts of calcium lignin sulfonate, 30 parts of bentonite and 65 parts of kaolin clay are mixed-grinding, and thereto is added water, and the mixture is well kneaded and is then granulated and dried to obtain a formulation.

### Formulation Example 6

Twenty (20) parts of the present compound S, 35 parts of a mixture of white carbon and ammonium polyoxyethylene alkyl ether sulfate (weight ratio is 1:1) and appropriate amount of water is mixed to make the total amount thereof 100 parts, and the mixture is then finely-ground with a grinder to obtain a formulation.

Next, Test Examples are described.

The untreated groups in Test Example 1 to Test Exmaple 8 represent tested groups in which the same conditions as those of each of the Test Examples were conducted except that DMSO was dispensed in the place of a DMSO diluted solution comprising the compound of the present invention. Also the untreated groups in Test Exmaple 9 to Test Exmaple 20 represent tested groups in which an aqueous diluted solution of a formulation comprising the compound of the present invention is not applied. The untreated groups in Test Example 21 and Test Example 25 represent tested groups in which the same conditions as those of each of the Test Examples were conducted except that no test compounds are used.

### Test Example 1: Control Test Against Wheat Septoria Leaf Blotch Fungus (Septoria Tritici)

The compound of the present invention, that is, compound No. 1-2, 1-3, 1-4, 1-5, 1-6, 1-7, 2-1, 2-2, 2-3, 2-4, 2-5, 2-6, 2-7, 2-8, 2-9, 2-10, 2-11, 2-12, 2-13, 2-14, 2-15, 2-16, 2-17, 2-18, 2-19, 3-1, 3-2, 3-3, 3-4, 3-5, 3-6, 3-7, 3-8, 3-9, 4-1, 4-2, 4-3, 4-4, 4-5, 4-6, 4-7, 4-8, 5-1, 5-2, 5-3, 5-4, 5-5, 5-7, 5-8, 5-10, 5-11, 5-12, 5-13, 5-14, 5-15, 5-16, 5-18, 5-19, 6-1, 6-2, 6-3, 6-5, 6-6, 6-7, 6-8, 7-1, 8-1, 8-2, 8-3, 8-4, 8-5, 8-6, 8-7, 9-1, 9-2, 9-3, 9-4, 9-5, 10-1, 10-2, 10-3, 1-1, 1-8, 1-13, 2-25, 2-26, 2-27, 2-32, 2-33, 2-34, 2-35, 2-36, 2-37, 2-39, 2-41, 2-42, 8-8, 8-9, 8-10, 8-11, 8-12, 8-13, 8-14, 8-15, 8-16, 8-17, 8-18, 8-19, 8-20, 8-21, 9-6, or 10-5 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which conidia of Septoria tritici were inoculated in advance. This plate was cultured at 18° C. for 5 days, thereby allowing Septoria tritici to undergo proliferation, and the absorbance at 550 nm of each well of the titer plate was then measured to determine a degree of growth of Septoria tritici. As a result, every of the growth in the well in treated groups treated with each of the compounds of the present invention showed 50% or less compared to the growth in an untreated well.

### Test Example 2: Control Test Against Cucurbitaceae Phytophthora Rot Fungus (Phytophthora Capsici)

The compound of the present invention, that is, compound No. 1-2, 1-3, 2-6, 2-11, 3-1, 3-4, 6-3, 8-4, 8-12, 8-21, 9-1, or 10-5 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which spores of Phytophthora capsici were inoculated in advance. This plate was cultured at 27° C. for 3 days, thereby allowing Phytophthora capsici to undergo proliferation, and the absorbance at 550 nm of each well of the titer plate was then measured to determine a degree of growth of the Phytophthora capsici. As a result, every of the growth in the well in treated groups treated with each of the compounds of the present invention showed 50% or less compared to the growth in an untreated well.

### Test Example 3: Control Test Against Seedling Blight Fungus (Pythium Ultimum)

The compound of the present invention, that is, compound No. 1-2, 1-3, 2-3, 2-7, 2-8, 2-9, 2-10, 2-13, 2-15, 2-17, 3-1, 3-4, 6-3, 8-1, 8-4, 9-1, 1-1, 2-25, 2-26, 2-33, 8-9, 8-11, 8-12, 8-14, 8-17, 8-18, or 10-5 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which spores of Pythium ultimum were inoculated in advance. This plate was cultured at 23° C. for 5 days, thereby allowing Pythium ultimum to undergo proliferation, and the absorbance at 550 nm of each well of the titer plate was then measured to determine a degree of growth of the Pythium ultimum. As a result, every of the growth in the well in treated groups treated with each of the compounds of the present invention showed 50% or less compared to the growth in an untreated well.

### Test Example 4: Control Test Against Corn Smut Fungus (Ustilago Maydis)

The compound of the present invention, that is, compound No. 1-2, 2-3, 2-4, 2-5, 2-6, 2-7, 2-8, 2-9, 2-10, 2-11, 2-12, 2-13, 2-14, 2-15, 2-17, 3-1, 3-4, 8-1, 1-1, 1-8, 2-25, 2-26, 2-27, 2-33, 2-34, 2-35, 2-36, 2-37, 2-39, 2-41, 2-42, 8-8, 8-9, 8-11, 8-12, 8-13, 8-14, 8-16, 8-17, 8-18, 10-5, or 2-32 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which conidia of Ustilago maydis were inoculated in advance. This plate was cultured at 18° C. for 4 days, thereby allowing Ustilago maydis to undergo proliferation, and the absorbance at 550 nm of each well of the titer plate was then measured to determine a degree of growth of the Ustilago maydis. As a result, every of the growth in the well in treated groups treated with each of the compounds of the present invention showed 50% or less compared to the growth in an untreated well.

### Test Example 5: Control Test Against Barley Scald Fungus (Rhynchosporium Secalis)

The compound of the present invention, that is, compound No. 1-2, 1-3, 2-2, 2-3, 2-4, 2-5, 2-6, 2-7, 2-8, 2-9, 2-10, 2-11, 2-12, 2-13, 2-14, 2-15, 2-16, 2-17, 3-1, 3-4, 3-6, 3-7, 6-2, 6-3, 6-4, 6-5, 7-1, 8-1, 8-2, 8-3, 8-4, 9-1, 1-1, 1-8, 1-9, 1-10, 1-11, 1-12, 1-13, 2-25, 2-26, 2-27, 2-32, 2-33, 2-34, 2-35, 2-36, 2-37, 2-38, 2-39, 2-40, 2-41, 2-42, 8-8, 8-9, 8-10, 8-11, 8-12, 8-13, 8-14, 8-15, 8-16, 8-17, 8-18, 8-19, 8-20, 8-21, 9-6, 10-5, or 11-3 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which conidia of Rhynchosporium secalis were inoculated in advance. This plate was cultured at 18° C. for 7 days, thereby allowing Rhynchosporium secalis to undergo proliferation, and the absorbance at 550 nm of each well of the titer plate was then measured to determine a degree of growth of the Rhynchosporium secalis. As a result, every of the growth in the well in treated groups treated with each of the compounds of the present invention showed 50% or less compared to the growth in an untreated well.

### Test Example 6: Control Test Against Cucumber Botrytis Rot Fungus (Botrytis Cinerea)

The compound of the present invention, that is, compound No. 1-2, 1-3, 2-2, 2-3, 2-4, 2-5, 2-6, 2-7, 2-8, 2-9, 2-10, 2-11, 2-12, 2-13, 2-14, 2-15, 2-16, 2-17, 3-1, 3-4, 3-6, 3-7, 6-2, 6-3, 6-4, 6-5, 7-1, 8-1, 8-2, 8-3, 8-4, 9-1, 1-1, 1-8, 1-9, 1-10, 1-12, 1-13, 2-25, 2-26, 2-27, 2-32, 2-33, 2-34, 2-35, 2-36, 2-37, 2-39, 2-40, 2-41, 2-42, 8-8, 8-9, 8-10, 8-11, 8-12, 8-13, 8-14, 8-15, 8-16, 8-17, 8-18, 8-19, 8-20, 8-21, 9-6, 10-5, or 11-3 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which conidia of Botrytis cinerea were inoculated in advance. This plate was cultured at 18° C. for 4 days, thereby allowing Botrytis cinerea to undergo proliferation, and the absorbance at 550 nm of each well of the titer plate was then measured to determine a degree of growth of the Botrytis cinerea. As a result, every of the growth in the well in treated groups treated with each of the compounds of the present invention showed 50% or less compared to the growth in an untreated well.

### Test Example 7: Control Test Against Peach Scab Fungus (Cladosporium Carpophilum)

The compound of the present invention, that is, compound No. 1-2, 1-3, 2-2, 2-3, 2-4, 2-5, 2-6, 2-7, 2-8, 2-9, 2-10, 2-11, 2-12, 2-13, 2-14, 2-15, 2-16, 2-17, 3-1, 3-4, 3-6, 3-7, 6-2, 6-3, 6-5, 7-1, 8-1, 8-2, 8-3, 8-4, 9-1, 1-1, 1-8, 1-10, 1-11, 1-12, 1-13, 2-25, 2-26, 2-27, 2-32, 2-33, 2-34, 2-35, 2-36, 2-37, 2-39, 2-40, 2-41, 2-42, 8-8, 8-9, 8-10, 8-11, 8-12, 8-13, 8-14, 8-15, 8-16, 8-17, 8-18, 8-19, 8-20, 8-21, 9-6, or 10-5 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which conidia of Cladosporium carpophilum were inoculated in advance. This plate was cultured at 18° C. for 5 days, thereby allowing Cladosporium carpophilum to undergo proliferation, and the absorbance at 550 nm of each well of the titer plate was then measured to determine a degree of growth of the Cladosporium carpophilum. As a result, every of the growth in the well in treated groups treated with each of the compounds of the present invention showed 50% or less compared to the growth in an untreated well.

### Test Example 8: Control Test Against Rice Brown Spot Fungus (Cochliobolus Miyabeanus)

The compound of the present invention, that is, compound No. 1-2, 1-3, 2-2, 2-3, 2-4, 2-5, 2-6, 2-7, 2-8, 2-9, 2-10, 2-11, 2-12, 2-13, 2-14, 2-15, 2-16, 2-17, 3-1, 3-4, 3-6, 3-7, 6-1, 6-2, 6-3, 6-4, 6-5, 7-1, 8-1, 8-2, 8-3, 8-4, 9-1, 1-1, 1-8, 1-9, 1-10, 1-11, 1-12, 1-13, 2-25, 2-26, 2-27, 2-32, 2-33, 2-34, 2-35, 2-36, 2-37, 2-38, 2-39, 2-40, 2-41, 2-42, 8-8, 8-9, 8-10, 8-11, 8-12, 8-13, 8-14, 8-15, 8-16, 8-17, 8-18, 8-19, 8-20, 8-21, 9-6, 10-5, or 11-3 was diluted with DMSO so as to contain 150 ppm, and 1 µL of the dilution solutions were dispensed into titer plate (96 well), and thereafter, thereto was then dispensed 150 µL of a potato dextrose broth (PDB broth) to which conidia of Cochliobolus miyabeanus were inoculated in advance. This plate was cultured at 23° C. for 3 days, thereby allowing Cochliobolus miyabeanus to undergo proliferation, and the absorbance at 550 nm of each well of the titer plate was then measured to determine a degree of growth of the Cochliobolus miyabeanus. As a result, every of the growth in the well in treated groups treated with each of the compounds of the present invention showed 50% or less compared to the growth in an untreated well.

### Test Example 9: Control Test Against Soybean Rust (Phakopsora Pachyrhizi)

Soybean leaf (cv; Kurosengoku) was punched out to 1 cm diameter to prepare a leaf disk. Each 1 mL of an agar medium (agar concentration 1.2 %) was dispensed in each well of 24 well microplate. A piece of the leaf disk was placed on agar medium on each well. To a mixture of 0.5 µL of Sorpol (registered trademark) 1200 KX, 4.5 µL of DMSO, and 5 µL of xylene was added 20 µL of a solution containing 10000 ppm of the test compound in DMSO. The resulting mixture was diluted with ion exchange water to prepare a mixture containing a predetermined concentration of the test compound. The resulting mixture was sprayed in 10 µL per one leaf disk. After 1 day, an aqueous suspension of conidia of Phakopsora pachyrhizi having an amino acid substitution of F129L on mitochondrial cytochrome b protein (1.0 × 10⁵ /mL) was inoculated onto the leaf disks. After the inoculation, the microplate was placed in a growth chamber (light on for 6 hours, light off for 18 hours, 23° C. temperature, 60 % humidity). After 1 day, the leaf disks were air-dried to disappear water droplets on the surface of the leaf disk, and the microplate was placed again in the growth chamber for 12 days. Thereafter, a lesion area of soybean rust disease was assessed. As a result, lesion areas in the leaf disk treated with any one of the compound of the present invention, that is, compound No. 1-2, 1-3, 2-1, 2-3, 2-4, 2-5, 2-6, 2-7, 2-8, 2-10, 2-11, 2-12, 2-13, 2-14, 2-15, 2-16, 2-17, 2-18, 3-1, 3-4, 3-6, 3-7, 3-8, 4-1, 4-2, 4-3, 4-4, 4-5, 4-6, 5-5, 5-8, 5-10, 5-11, 6-1, 6-2, 6-3, 6-4, 6-5, 6-6, 6-7, 6-8, 7-1, 8-1, 8-2, 8-3, 8-4, 8-6, 8-7, 9-1, 9-2, 9-3, 9-4, 9-5, 10-1, 10-2, 10-3, 1-1, 1-8, 1-10, 1-11, 1-13, 2-25, 2-26, 2-27, 2-32, 2-33, 2-34, 2-35, 2-37, 2-38, 2-39, 2-42, 2-43, 8-8, 8-9, 8-10, 8-11, 8-12, 8-13, 8-14, 8-15, 8-16, 8-17, 8-18, 8-19, 8-20, 8-21, or 10-5 as a tested compound at a prescribed concentration of 50 ppm showed 30% or more compared to the lesion areas in an untreated leaf disk.

### Test Example 10: Control Test Against Rice Blast (Pyricularia Oryzae)

Each of plastic pots was filled with soil and thereto rice (cv; HINOHIKARI) seeds were sown and the plants were grown in a greenhouse for 20 days. Thereafter, the compound of the present invention, that is, compound No. 2-9, or 2-15, each of which was made to a formulation according to the similar method to that of Formulation Example 6, was mixed with water so as to be 200 ppm. The mixtures were sprayed to foliar parts so as to adhere adequately on the leaves of the above-mentioned rice. After spraying the mixtures, the rices were air-dried and were placed at 24° C. during daytime and 20° C. during nighttime under a high humidity for 6 to 7 days while the above-mentioned spraying-treated rice were contacted rice seedlings (cv; Hinohikari) infected by Pyricularia oryzae, and a lesion area was observed. As a result, every of the lesion areas in rice treated with each of the compounds of the present invention showed 30% or less compared to the lesion are in an untreated rice.

### Test Example 11: Control Test Against Barley Net Blotch (Pyrenophora Teres)

Each of plastic pots was filled with soil and thereto barley (cv; NISHINOHOSHI) seeds were sown and the barleys were grown in a greenhouse for 7 days. Thereafter, the compound of the present invention, that is, compound No. 1-3, 2-3, 2-4, 2-5, 2-6, 2-7, 2-8, 2-9, 2-10, 2-11, 2-12, 2-13, 2-14, 2-15, 2-16, 2-17, 3-1, 3-4, 3-6, 3-7, 6-1, 6-2, 6-5, 7-1, 8-1, 8-2, 8-3, 9-1, 2-25, 2-26, 2-27, 2-32, 2-37, 2-39, 8-8, 8-9, 8-18, 8-19, 8-20, 10-5, 11-1, 11-2, or 11-3, each of which was made to a formulation according to the similar method to that of Formulation Example 6, was mixed with water so as to be a prescribed concentration (200 ppm). The resulting mixtures were sprayed to foliar parts so as to adhere adequately on the leaves of the above-mentioned barley. After spraying the mixtures, the barleys were air-dried and after 1 day, an aqueous suspension of the conidia of Pyrenophora teres was spraying-inoculated. After the inoculation, the barleys were placed at 23° C. during daytime and 20° C. during nighttime under a high humidity for 3 day and then cultivated in a greenhouse for 7 days, and a lesion area was observed. As a result, every of the lesion areas in barleys treated with each of the compounds of the present invention showed 30% or less compared to the lesion area in an untreated barley.

### Test Example 12: Control Test Against Wheat Brown Rust (Puccinia Recondita)

Each of plastic pots was filled with soil and thereto wheat (cv; SHIROGANE) seeds were sown and the wheats were grown in a greenhouse for 9 days. The compound of the present invention, that is, compound No. 1-2, 2-3, 2-4, 2-5, 2-6, 2-7, 2-8, 2-10, 2-11, 2-12, 2-13, 2-14, 2-15, 2-16, 2-17, 3-1, 3-4, 3-6, 3-7, 6-1, 6-2, 6-3, 7-1, 8-1, 8-2, 8-3, 8-6, 9-1, 1-1, 2-27, 2-36, 2-37, 8-8, 8-9, 8-11, 8-12, 8-13, 8-14, 8-15, 8-16, 8-17, 8-18, 8-21, 9-6, or 10-5, each of which was made to a formulation according to the similar method to that of Formulation Example 6, was mixed with water so as to be 200 ppm, and the mixtures were sprayed to foliar parts so as to adhere adequately on the leaves of the above-mentioned wheat. After spraying the mixtures, the wheats were air-dried and were then cultivated at 20° C. under lighting for 5 to 7 days. The conidia of Puccinia recondita were sprinkling-inoculated. After the inoculation, the wheats were placed under a dark and humid condition at 23° C. for 1 day and were then cultivated at 20° C. under lighting for 8 days, and a lesion area was observed. As a result, every of the lesion areas in wheats treated with each of the compounds of the present invention showed 30% or less compared to the lesion area in an untreated wheat.

### Test Example 13: Control Test Against Septoria Leaf Blotch (Septoria Tritici)

Each of plastic pots was filled with soil and thereto wheat (cv; Apogee) seeds were sown and the wheats were grown in a greenhouse for 10 days. Thereafter, the compound of the present invention, that is, compound No. 1-2, 1-3, 2-2, 2-3, 2-4, 2-5, 2-7, 2-8, 2-9, 2-10, 2-11, 2-12, 2-14, 2-15, 2-16, 2-17, 3-1, 3-4, 3-6, 3-7, 6-1, 6-3, 6-5, 7-1, 8-1, 8-2, 8-3, 8-4, 8-6, 8-7, 9-1, 1-11, 1-13, 2-25, 2-26, 2-27, 2-32, 2-33, 2-34, 8-8, 8-9, 8-11, 8-12, 8-13, 8-14, 8-15, 8-16, 8-17, 8-18, 8-19, 8-21, 9-6, 10-5, 10-6, 11-1, or 11-2, each of which was made to a formulation according to the similar method to that of Formulation Example 6, was mixed with water so as to be 200 ppm. The mixtures were sprayed to foliar parts so as to adhere adequately on the leaves of the above-mentioned wheat. After spraying the mixtures, the wheats were air-dried and after 4 days, an aqueous suspension of the conidia of Septoria tritici was spraying-inoculated. After the inoculation, the wheats were placed at 18° C. under a high humidity for 3 days and then under lighting for 14 to 18 days, and a lesion area was observed. As a result, every of the lesion areas in wheats treated with each of the compounds of the present invention showed 30% or less compared to the lesion area in an untreated wheat.

### Test Example 14: Control Test Against Septoria Leaf Blotch (Septoria Tritici)

Each of plastic pots was filled with soil and thereto wheat (cv; Apogee) seeds were sown and the wheats were grown in a greenhouse for 10 days, and an aqueous suspension containing the conidia of Septoria tritici was spraying-inoculated. After the inoculation, the wheats were placed at 18° C. under a high humidity for 3 days, and thereafter, the compound of the present invention, that is, compound No. 1-2, 1-3, 2-3, 2-4, 2-5, 2-7, 2-8, 2-10, 2-11, 2-12, 2-13, 2-14, 2-15, 2-16, 2-17, 3-1, 3-4, 3-6, 3-7, 6-1, 6-5, 8-1, 8-2, 8-3, 8-4, 8-6, 8-7, 9-1, 2-25, 2-26, 2-27, 2-32, 2-33, 2-34, 2-36, 8-8, 8-9, 8-10, 8-11, 8-12, 8-13, 8-14, 8-15, 8-16, 8-17, 8-18, 8-19, 8-20, 8-21, 9-6, 10-5, or 11-1, each of which was made to a formulation according to the similar method to that of Formulation Example 6, was mixed with water so as to be 200 ppm. The mixtures were sprayed to foliar parts so as to adhere adequately on the leaves of the above-mentioned wheat. After spraying the mixtures, the wheats were air-dried and were placed under lighting for 14 to 18 days, and a lesion area was observed. As a result, every of the lesion areas in wheats treated with each of the compounds of the present invention showed 30% or less compared to the lesion area in an untreated wheat.

### Test Example 15: Control Test Against Tomato Late Blight (Phytophthora Infestans)

Each of plastic pots was filled with soils and thereto tomato (cv; PATIO) seeds were sown and the tomatoes were grown in a greenhouse for 20 days. Thereafter, the compound of the present invention, that is, compound No. 1-3, 1-13, 2-4, 2-7, 2-8, 2-10, 2-12, 2-13, 2-16, 3-1, 3-4, 6-2, 8-9, 9-1, or 11-3, each of which was made to a formulation according to the similar method to that of Formulation Example 6, was mixed with water so as to be 200 ppm. The mixtures were sprayed to foliar parts so as to adhere adequately on the leaves of the above-mentioned tomato. After spraying the mixtures, the tomatoes were air-dried and after 1 day, an aqueous suspension of the spores of Phytophthora infestans were spraying-inoculated. After the inoculation, the tomatoes were placed in a greenhouse of 23° C. during daytime and 20° C. during nighttime under a high humidity for 1 day, and a lesion area was observed. As a result, every of the lesion areas in tomatoes treated with each of the compounds of the present invention showed 30% or less compared to the lesion area in an untreated tomato.

### Test Example 16: Control Test Against Soybean Rust (Phakopsora Pachyrhizi)

Each of plastic pots was filled with soil and thereto soybean (cv: Kurosengoku) seeds were sown and the soybeans were grown in a greenhouse for 10 to 14 days. Thereafter, the compound of the present invention, that is, compound No. 1-2, 1-3, 2-3, 2-4, 2-5, 2-6, 2-7, 2-8, 2-10, 2-11, 2-12, 2-13, 2-14, 2-15, 2-16, 2-17, 3-1, 3-4, 3-6, 3-7, 6-1, 6-2, 6-3, 6-4, 8-1, 8-2, 8-3, 8-4, 8-6, 8-7, 9-1, 1-1, 1-13, 2-25, 2-26, 2-27, 2-32, 2-33, 2-34, 2-35, 2-39, 2-40, 8-8, 8-9, 8-10, 8-11, 8-12, 8-13, 8-14, 8-15, 8-16, 8-17, 8-18, 8-19, 8-20, 8-21, 9-6, or 10-5, each of which was made to a formulation according to the similar method to that of Formulation Example 6, was mixed with water so as to be 200 ppm. The resulting mixtures were sprayed to foliar parts so as to adhere adequately on the leaves of the above-mentioned soybean. After spraying the mixtures, the soybeans were air-dried and after 2 to 5 days, an aqueous suspension of the conidia of Phakopsora pachyrhizi was spraying-inoculated. After the inoculation, the soybeans were placed in a greenhouse of 23° C. during daytime and 20° C. during nighttime under a high humidity for 1 to 2 days, and were then cultivated in the greenhouse for 12 days, and a lesion area was observed. As a result, every of the lesion areas in soybean treated with each of the compounds of the present invention showed 30% or less compared to the lesion area in an untreated soybean.

### Test Example 17: Control Test Against Soybean Rust (Phakopsora Pachyrhizi)

Each of plastic pots was filled with soil and thereto soybean (cv: Kurosengoku) seeds were sown and the soybeans were grown in a greenhouse for 10 days, and an aqueous suspension containing the conidia of Phakopsora pachyrhizi was spraying-inoculated. After the inoculation, the soybeans were placed in a greenhouse of 23° C. during daytime and 20° C. during nighttime under a high humidity for 1 day, and were then cultivated in the greenhouse for 1 to 2 days, and thereafter, the compound of the present invention, that is, compound No. 1-3, 2-3, 2-4, 2-5, 2-7, 2-8, 2-10, 2-11, 2-12, 2-13, 2-14, 2-15, 2-17, 3-1, 3-4, 3-6, 3-7, 6-1, 6-3, 6-4, 8-1, 8-3, 8-4, 8-6, 8-7, 9-1, 1-13, 2-25, 2-26, 2-27, 2-32, 2-33, 2-34, 2-35, 8-8, 8-9, 8-10, 8-11, 8-12, 8-13, 8-14, 8-15, 8-16, 8-17, 8-19, 8-21, or 10-5, each of which was made to a formulation according to the similar method to that of Formulation Example 6, was mixed with water so as to be 200 ppm, and the resulting mixtures were sprayed to foliar parts so as to adhere adequately on the leaves of the above-mentioned soybean. After spraying the mixtures, the soybeans were air-dried and cultivated in a greenhouse for 8 days, and a lesion area was then observed. As a result, every of the lesion areas in soybean treated with each of the compounds of the present invention showed 30% or less compared to the lesion area in an untreated soybean.

### Test Example 18: Control Test Against Soybean Leaf Spot (Cercospora Sojina)

Each of plastic pots was filled with soil and thereto soybean (cv: Tachinagawa) seeds were sown and the soybeans were grown in a greenhouse for 13 days. Thereafter, the compound of the present invention, that is, compound No. 2-3, 2-4, 2-5, 2-6, 2-7, 2-8, 2-9, 2-10, 2-12, 2-13, 2-15, 2-16, 2-17, 3-1, 3-4, 3-6, 3-7, 6-2, 6-5, 7-1, 8-1, 9-1, 1-13, 2-27, 8-10, 8-11, 8-12, 8-13, 8-14, 8-15, 8-16, 8-17, 8-18, 8-19, 8-20, 8-21, 9-6, or 10-5, each of which was made to a formulation according to the similar method to that of Formulation Example 6, was mixed with water so as to be 200 ppm. The resulting mixtures were sprayed to foliar parts so as to adhere adequately on the leaves of the above-mentioned soybean. After spraying the mixtures, the soybeans were air-dried and after 1 day, an aqueous suspension of the conidia of Cercospora sojina was spraying-inoculated. After the inoculation, the soybeans were placed in a greenhouse of 23° C. during daytime and 20° C. during nighttime under a high humidity for 3 days, and were then cultivated in the greenhouse for 16 days, and a lesion area was observed. As a result, every of the lesion areas in soybean treated with each of the compounds of the present invention showed 30% or less compared to the lesion area in an untreated soybean.

### Test Example 19: Control Test Against Tomato Early Blight (Alternaria Solani)

Each of plastic pots was filled with soils and thereto tomato (cv; PATIO) seeds were sown and the tomatoes were grown in a greenhouse for 20 days. Thereafter, the compound of the present invention, that is, compound No. 2-2, 2-4, 2-5, 2-8, 2-13, 2-15, 2-16, 2-17, 3-1, 3-4, 3-6, 6-2, 6-4, 6-5, 7-1, 1-11, 1-13, 2-36, 2-37, or 2-40, each of which was made to a formulation according to the similar method to that of Formulation Example 6, was mixed with water so as to be 200 ppm. The resulting mixtures were sprayed to foliar parts so as to adhere adequately on the leaves of the above-mentioned tomato. After spraying the mixtures, the tomatoes were air-dried and after 1 day, an aqueous suspension of the conidia of Alternaria solani were spraying-inoculated. After the inoculation, the tomatoes were placed at 18° C. under a high humidity for 6 days, and a lesion area was observed. As a result, every of the lesion areas in tomatoes treated with each of the compounds of the present invention showed 30% or less compared to the lesion area in an untreated tomato.

### Test Example 20: Control Test Against Kidney Bean Stem Rot (Sclerotinia Sclerotiorum)

Each of plastic pots was filled with soil and thereto Kidney bean (cv; NAGAUZURA SAITO) seeds were sown and the kidney beans were grown in a greenhouse for 8 days. Thereafter, the compound of the present invention, that is, compound No. 1-2, 1-3, 2-3, 2-4, 2-5, 2-7, 2-8, 2-9, 2-10, 2-11, 2-13, 2-15, 3-1, 3-4, 6-3, 8-1, 8-2, 8-3, 8-6, 8-7, 2-27, 8-8, 8-9, 8-10, 8-12, 8-13, 8-14, 8-18, 8-21, 9-6, or 10-5, each of which was made to a formulation according to the similar method to that of Formulation Example 6, was mixed with water so as to be 200 ppm. The resulting mixtures were sprayed to the foliar parts so as to adhere adequately on the leaves of the above-mentioned kidney bean. After spraying the mixtures, the kidney beans were air-dried and a PDA medium containing hyphae of Sclerotinia sclerotiorum was placed on the leaves of the kidney bean. After the inoculation, all kidney beans were placed under a high humidity during only night and after 4 days, a lesion area was observed. As a result, every of the lesion areas in kidney beans treated with each of the compounds of the present invention showed 30% or less compared to the lesion area in an untreated kidney beans.

### Test Method 21: Tests Against Cotton Aphids

The test compounds is made to a formulation according to a similar method to that described in the Formulation Example 6, and thereto is added water containing 0.03 v/v % of a spreader to prepare a diluted solution containing a prescribed concentration of the test compound.

Cucumber (Cucumis sativus) seedling (on the developmental stage of the second true leaf) is planted in a container and approximately 30 cotton aphids (Aphis gossypii) (all stages of life) are released onto the leaves of the cucumber. After 1 day, the diluted solutions are sprayed into the seedling in a ratio of 10 mL/seedling. Further, after 5 days, the number of the surviving insects is examined and the controlling value is calculated by the following equation.

$$\text{Controlling} \text{value} \left(\%\right) = \left\{1 - \left(\text{Cb} \times \text{Tai}\right) / \left(\text{Cai} \times \text{Tb}\right)\right\} \times 100$$

wherein the symbols in the formula represent the following descriptions.
Cb: Number of the test insects in untreated group;
Cai: Number of the surviving insects at the time of the investigation in untreated group;
Tb: Number of the test insects in treated group;
Tai: Number of the surviving insects at the time of the investigation in treated group;

### Test Example 21

The test was conducted by making the prescribed concentration 500 ppm and using the below-mentioned compounds of the present invention as a test compound according to the test method 21. As a result of the test, the below-mentioned compounds of the present invention showed 90 % or greater as the controlling value.

Compound of the present invention: 1-2, 8-3, 8-4, 9-1, and 2-41

### Test Method 22: Tests Against Brown Planthoppers

The test compounds are made to a formulation according to a similar method to that described in the Formulation Example 6, and thereto is added water containing 0.03 v/v % of a spreader to prepare a diluted solution containing a prescribed concentration of the test compound.

Rice (Oryza sativa) seedling (on the developmental stage of the second true leaf) is planted in a container, and the diluted solutions are sprayed into the seedling in a ratio of 10 mL/seedling. Thereafter, 20 3rd instar larvae of brown planthoppers (Nilaparvata lugens) are released onto the rice leaves. After 6 days, the morality is calculated by the following equation.

$$\begin{matrix}\text{Morality} \left(\text{\%}\right) = \left\{\text{1} - \text{the} \text{number} \text{of} \text{the} \text{surviving}\right) \\ \text{insects} / \left(20\right\} \times 100\end{matrix}$$

### Test Example 22

The test was conducted by making the prescribed concentration 500 ppm and using the below-mentioned compounds of the present invention as a test compound according to the test method 22. As a result of the test, the below-mentioned compound of the present invention showed 90% or greater as the controlling value.

Compound of the present invention: 1-2, and 8-8

### Test Method 23: Tests Against Cotton Worm

Test compounds are made to a formulation according to a similar method to that described in the Formulation example 6, and thereto is added water containing 0.03 v/v% of Shindain (registered trademark) to prepare a diluted solution containing a prescribed concentration of the test compound.

Cabbage (Brassicae oleracea) seedling (on the developmental stage of the second to third true leaf) is planted in a cup, and the diluted solutions are sprayed into the seedling at a ratio of 20 mL/seedling. Thereafter, the stem and leaf thereof is cut out and then is installed into a cup that is covered with filter paper on the bed of the cup. Five (5) cotton worm (Spodoptera litura) at the second instar larval stage are released into the cup. After 5 days, the number of the surviving insects is counted, and the mortality of insects is calculated by tne following equation.

$$\begin{matrix}\text{Mortality} \left(\text{\%}\right) \text{=} \left(1 - \text{Number} \text{of} \text{surviving} \text{insects/5}\right) \times \\ 100\end{matrix}$$

### Test Example 23

The test was conducted by making the prescribed concentration 500 ppm and using the below-mentioned compounds of the present invention as a test compound according to the test method 23. As a result of the test, the below-mentioned compound of the present invention showed 80 % or greater as the mortality of insects.

Compound of the present invention: 1-2, and 8-4

### Test Method 24: Tests Against Diamondback Moth

Test compounds are made to a formulation according to a similar method to that described in the Formulation example 6, and thereto is added water containing 0.03 v/v% of Shindain (registered trademark) to prepare a diluted solution containing a prescribed concentration of the test compound.

Cabbage (Brassicae oleracea) seedling (on the developmental stage of the second to third true leaf) is planted in a cup, and the diluted solutions are sprayed into the seedling at a ratio of 20 mL/seedling. Thereafter, the stem and leaf thereof is cut out and then is installed into a cup that is covered with filter paper on the bed of the cup. Five (5) diamondback moth (Plutella xylostella) at the second instar larval stage are released into the cup. After 5 days, the number of the surviving insects is counted, and the mortality of insects is calculated by the following equation.

$$\begin{matrix}\text{Morality} \left(\text{\%}\right) \text{=} \left(1 - \text{Number} \text{of} \text{surviving} \text{insects} / 5\right) \times \\ 100\end{matrix}$$

### Test Example 24

The test was conducted by making the prescribed concentration 500 ppm and using the below-mentioned compounds of the present invention as a test compound according to the test method 24. As a result of the test, the below-mentioned compounds of the present invention showed 80% or greater as the mortality of insects.

Compound of the present invention: 1-2, 2-6, 8-3, 8-4, 1-1, 2-32, 8-8, and 8-9

### Test Method 25: Tests Against Common Red Spider Mite

The test compounds is made to a formulation according to a similar method to that described in the Formulation Example 6, and thereto is added water containing 0.03 v/v % of a spreader to prepare a diluted solution containing a prescribed concentration of the test compound.

Kidney bean (Phaseolus vulgaris) seedling (on the developmental stage of the first true leaf) is planted in a container and approximately 40 adult female of common red spider mites (Tetranychus urticae) are released onto the leaves of the kidney bean. After 1 day, the diluted solutions are sprayed into the seedling in a ratio of 10 mL/seedling. Further, after 13 days, the number of the surviving insects is examined and the controlling value is calculated by the following equation.

$$\text{Controlling} \text{value} \left(\%\right) = \left\{1 - \left(\text{Cb} \times \text{Tai}\right) / \left(\text{Cai} \times \text{Tb}\right)\right\} \times 100$$

wherein the symbols in the formula represent the following descriptions.
Cb: Number of the test insects in untreated group;
Cai: Number of the surviving insects at the time of the investigation in untreated group;
Tb: Number of the test insects in treated group;
Tai: Number of the surviving insects at the time of the investigation in treated group;
Here the “untreated group” represents a group where the similar treatment procedure to that of the treated group except not using the test compound is done.

### Test Example 25

The test was conducted by making the prescribed concentration 500 ppm and using the below-mentioned compounds of the present invention as a test compound according to the test method 25. As a result of the test, the below-mentioned compounds of the present invention showed 90% or greater as the controlling value.

Compound of the present invention: 1-2, 2-6, 8-2, 8-4, 9-1, 1-1, 1-8, 1-11, 1-12, 2-32, 2-33, 2-34, 2-41, and 8-21

### Industrial Applicability

The compound of the present invention has efficacies for controlling pests and can be used to control pests.

1. A compound represented by formula (I): 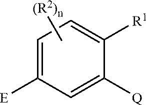
[wherein
Q represents a group represented by Q1, or a group represented by Q2 (where ● represents a binding site to a benzene ring), 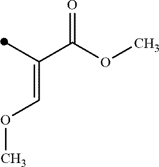 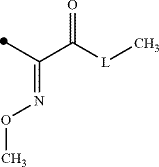
E represents a C5-C6 cycloalkenyl group which may be optionally substituted with one or more substituents selected from Group D, a C6-C10 aryl group, a five- to ten-membered aromatic heterocyclic group {the C6-C10 aryl group and the five- to ten-membered aromatic heterocyclic group may be optionally substituted by one or more substituents selected from Group A }, R⁷—C≡C—, R⁵—O—N═C(R⁴)—, R⁸—N═C(R⁴)—, or R⁶O—,
L represents an oxygen atom or NH,
a combination of R¹ and n represents
a combination where R¹ represents a hydrogen atom, and n is 1; or
a combination where R¹ represents a C1-C3 chain hydrocarbon group which may be optionally substituted with one or more halogen atoms, a methoxy group, a cyclopropyl group, or a halogen atom, and n is 0,
R² represents a methyl group, a cyclopropyl group, or a halogen atom,
R⁴ represents a C1-C3 chain hydrocarbon group which may be optionally substituted with one or more halogen atoms, or a hydrogen atom,
R⁵ represents a C1-C6 chain hydrocarbon group which may be optionally substituted with one or more substituents selected from Group F, a C3-C6 cycloalkyl group which may be optionally substituted with one or more substituents selected from Group D, a phenyl group, a five- to six- membered aromatic heterocyclic group {the phenyl group and the five- to six- membered aromatic heterocyclic group may be optionally substituted with one or more substituents selected from Group C}, or R¹⁸—CH₂—,
R⁷ represents a C1-C6 chain hydrocarbon group which may be optionally substituted with one or more substituents selected from Group F, a C3-C6 cycloalkyl group which may be optionally substituted with one or more substituents selected from Group D, or a five- to six- membered aromatic heterocyclic group {the five- to six- membered aromatic heterocyclic group may be optionally substituted with one or more substituents selected from Group C},
R⁶ represents a methyl group which is substituted with one or more substituents selected from Group F, a C2-C6 chain hydrocarbon group which may be optionally substituted with one or more substituents selected from Group F, a C3-C4 cycloalkyl group which may be optionally substituted with one or more substituents selected from Group D, or R¹³R⁴NC(O)—,
R⁸ and R¹³ are identical to or different from each other and represent a C1-C6 chain hydrocarbon group which may be optionally substituted with one or more substituents selected from Group F, a C3-C6 cycloalkyl group which may be optionally substituted with one or more substituents selected from Group D, a phenyl group, a five- to six- membered aromatic heterocyclic group {the phenyl group and the five- to six- membered aromatic heterocyclic group may be optionally substituted with one or more substituents selected from Group C}, or R¹⁰—(CH₂)ₘ—,
m is 1 or 2,
R¹⁰ and R¹⁸ are identical to or different from each other and represent a phenyl group, or a five- to six- membered aromatic heterocyclic group {the phenyl group and the five- to six- membered aromatic heterocyclic group may be optionally substituted with one or more substituents selected from Group C},
Group A is a group consisting of a C1-C6 chain hydrocarbon group which may be optionally substituted with one or more substituents selected from Group F, a C3-C6 cycloalkyl group which may be optionally substituted with one or more substituents selected from Group D, a C1-C6 alkylthio group which may be optionally substituted with one or more substituents selected from Group F, OR¹⁴, C(O)R¹¹, C(O)OR¹¹, NR¹¹R⁹, C(R⁹)═NOR¹¹, a phenyl group, a five- to six- membered aromatic heterocyclic group {the phenyl group, and the five- to six- membered aromatic heterocyclic group may be optionally substituted with one or more substituents selected from Group E}, a halogen atom, a cyano group, and a nitro group,
R⁹ represents a C1-C3 chain hydrocarbon group which may be optionally substituted with one or more halogen atoms, or a hydrogen atom,
R¹¹ and R¹⁴ are identical to or different from each other and represent a C1-C6 chain hydrocarbon group which may be optionally substituted with one or more substituents selected from Group F, a C3-C6 cycloalkyl group which may be optionally substituted with one or more substituents selected from Group D, a phenyl group, or a five- to six- membered aromatic heterocyclic group {the phenyl group and the five- to six- membered aromatic heterocyclic group may be optionally substituted with one or more substituents selected from Group C},
Group C is a group consisting of a C1-C3 chain hydrocarbon group, a C3-C4 cycloalkyl group, a C1-C3 alkoxy group, and a C1-C3 alkylthio group {the C1-C3 chain hydrocarbon group, the C3-C4 cycloalkyl group, the C1-C3 alkoxy group, and the C1-C3 alkylthio group may be optionally substituted with one or more halogen atoms}, a halogen atom, a cyano group, a nitro group, and a hydroxy group,
Group D is a group consisting of a C1-C3 alkyl group which may be optionally substituted with one or more halogen atoms, a halogen atom, and a cyano group,
Group E is a group consisting of a C1-C3 chain hydrocarbon group, a C3-C4 cycloalkyl group, a C1-C3 alkoxy group, and C1-C3 alkylthio group {the C3-C4 cycloalkyl group, the C1-C3 alkoxy group, and the C1-C3 alkylthio group may be optionally substituted with one or more halogen atoms}, a halogen atom, a cyano group, a nitro group, and a hydroxy group, and
Group F is a group consisting of a C3-C4 cycloalkyl group, a C1-C3 alkoxy group, and a halogen atom]
or its N-oxide or agriculturally acceptable salts.

2. The compound according to claim 1 wherein
E represents a C5-C6 cycloalkenyl group which may be optionally substituted with one or more substituents selected from Group D, a phenyl group, a five- to six- membered aromatic heterocyclic group {the phenyl group and the five- to six- membered aromatic heterocyclic group may be optionally substituted with one or more substituents selected from Group H}, R⁷—C≡C—, R⁵—O—N═C(R⁴)—, or R⁶O—,
R⁴ represents a C1-C3 alkyl group, or a hydrogen atom,
R⁵ represents a C1-C6 chain hydrocarbon group which may be optionally substituted with one or more substituents selected from Group F, a C3-C6 cycloalkyl group which may be optionally substituted with one or more halogen atoms, a phenyl group, a five- to six-membered aromatic heterocyclic group {the phenyl group and the five- to six- membered aromatic heterocyclic group may be optionally substituted with one or more substituents selected from Group G}, or R¹⁸—CH₂—,
R⁶ represents a C2-C6 which may be optionally substituted with one or more substituents selected from Group F, a C3-C4 cycloalkyl group which may be optionally substituted with one or more halogen atoms, or R¹³R⁴NC(O)—,
R⁷ represents a C1-C6 chain hydrocarbon group which may be optionally substituted with one or more substituents selected from Group F, or a C3-C6 cycloalkyl group which may be optionally substituted with one or more halogen atoms,
R¹¹ and R¹³ are identical to or different from each other and represent a C1-C6 chain hydrocarbon group which may be optionally substituted with one or more substituents selected from Group F, or a C3-C6 cycloalkyl group which may be optionally substituted with one or more halogen atoms,
R¹⁴ represents a C1-C6 chain hydrocarbon group which may be optionally substituted with one or more substituents selected from Group F, a C3-C6 cycloalkyl group which may be optionally substituted with one or more halogen atoms, or a phenyl group which may be optionally substituted with one or more substituents selected from Group G,
R¹⁸ represents a phenyl group, or a five- to six- membered aromatic heterocyclic group {the phenyl group and the five- to six- membered aromatic heterocyclic group may be optionally substituted with one or more substituents selected from Group G},
Group G is a group consisting of a C1-C3 chain hydrocarbon group, a C1-C3 alkoxy group {the C1-C3 chain hydrocarbon group and the C1-C3 alkoxy group may be optionally substituted with one or more halogen atoms}, a halogen atom, and a cyano group, and
Group H is a group consisting of a C1-C6 chain hydrocarbon group which may be optionally substituted with one or more substituents, a C3-C4 cycloalkyl group which may be optionally substituted with one or more halogen atoms, OR¹⁴, a halogen atom, a cyano group, C(R⁴)═N—OR¹¹, a phenyl group, and a five- to six- membered aromatic heterocyclic group {the phenyl group and the five- to six- membered aromatic heterocyclic group may be optionally substituted with one or more substituents selected from Group G},
or its N-oxide or agriculturally acceptable salts.

3. The compound according to claim 2 wherein
E represents R⁷—C≡C—, R⁵—O—N═C(R⁴)—, R⁶O—,a phenyl group, a pyridyl group, a thienyl group, a furanyl group, a pyrimidinyl group, a thiadiazolyl group, or a pyrazolyl group {the phenyl group, the pyridyl group, the thienyl group, the furanyl group, the pyrimidinyl group, the thiadiazolyl group, and the pyrazolyl group may be optionally substituted with one or more substituents selected from Group I},
R⁵ represents a C1-C6 chain hydrocarbon group which may be optionally substituted with one or more substituents selected from Group F, or a C3-C4 cycloalkyl group which may be optionally substituted with one or more halogen atoms, or R¹⁸—CH₂—,
R⁶ represents a C2-C6 chain hydrocarbon group which may be optionally substituted with one or more halogen atoms,
R⁷ represents a C1-C6 chain hydrocarbon group which may be optionally substituted with one or more halogen atoms, or a C3-C4 cycloalkyl group which may be optionally substituted with one or more halogen atoms,
R¹¹ represents a C1-C3 alkyl group which may be optionally substituted with one or more halogen atoms,
R¹⁴ represents a C1-C6 chain hydrocarbon group which may be optionally substituted with one or more substituents selected from Group F, a C3-C4 cycloalkyl group which may be optionally substituted with one or more halogen atoms, or a phenyl group which may be optionally substituted with one or more substituents selected from Group G,
R¹⁸ represents a phenyl group which may be optionally substituted with one or more substituents selected from Group G, and
Group I is a group consisting of a C1-C6 chain hydrocarbon group which may be optionally substituted with one or more substituents selected from Group F, a cyclopropyl group, OR¹⁴, a halogen atom, a cyano group, C(R⁴)═N—OR¹¹, a phenyl group, a pyridyl group, and a pyrazolyl group {the phenyl group, the pyridyl group, and the pyrazolyl group may be optionally substituted with one or more substituents selected from Group G},
or its N-oxide or agriculturally acceptable salts.

4. The compound according to claim 2 wherein
E represents R⁷—C≡C—, a phenyl group, a pyridyl group, a thienyl group, a furanyl group, a thiadiazolyl group, a pyrazolyl group {the phenyl group, the pyridyl group, the thienyl group, the furanyl group, the thiadiazolyl group, and the pyrazolyl group may be optionally substituted with one or more substituents selected from Group J}, or a pyrimidinyl group {the pyrimidinyl group may be optionally substituted with one or more substituents selected from Group K},
R⁷ represents a C1-C6 alkyl group which may be optionally substituted with one or more halogen atoms, or a cyclopropyl group,
Group J is a group consisting of a C1-C6 chain hydrocarbon group, a C1-C3 alkoxy group {the C1-C6 chain hydrocarbon group and the C1-C3 alkoxy group may be optionally substituted with one or more substituents selected from Group F}, a cyclopropyl group, a halogen atom, a cyano group, C(R⁴)═N—OR¹¹, a phenyl group, a pyridyl group, and a pyrazolyl group {the phenyl group, the pyridyl group, and the pyrazolyl group may be optionally substituted with one or more substituents selected from Group G}, and
Group K is a group consisting of a C1-C3 alkyl group which may be optionally substituted with one or more halogen atoms, a phenoxy group which may be optionally substituted with one or more substituents selected from Group G, and a halogen atom,
or its N-oxide or agriculturally acceptable salts.

5. The compound according to claim 1 wherein
R¹represents a C1-C3 chain hydrocarbon group which may be optionally substituted with one or more halogen atoms, or a halogen atom, and
n is 0,
or its N-oxide or agriculturally acceptable salts.

6. The compound according to claim 1 wherein
Q represents a group represented by Q1,
R¹ represents a methyl group, and
n is 0,
or its N-oxide or agriculturally acceptable salts.

7. An agricultural composition which comprises the compound according to claim 1 or its N-oxide compound or an agriculturally acceptable salt thereof and an inert carrier.

8. A composition which comprises one or more ingredients selected from the group consisting of the following Groups (a), (b), (c) and (d) and the compound according to claim 1 or its N-oxide compound or agriculturally acceptable salts:
Group (a): a group consisting of insecticidal ingredients, miticidal ingredients, and nematicidal ingredients;
Group (b): fungicidal ingredients :
Group (c): plant growth modulating ingredients; and
Group (d): repellent ingredients.

9. A method for controlling pests which comprises applying an effective amount of the compound according to claim 1 or its N-oxide compound or an agriculturally acceptable salt thereof to a plant or soil.

10. A method for controlling soybean rust fungus having an amino acid substitution of F129L on mitochondrial cytochrome b protein by applying an effective amount of the compound according to claim 1 or its N-oxide compound or an agriculturally acceptable salt thereof to soybean or soils where soybean grows.

11. (canceled)

12. A seed or vegetative reproductive organ carrying an effective amount of the compound according to claim 1 or its N-oxide thereof, .

13. A compound represented by formula (II): 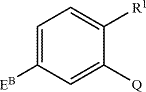
[wherein
a combination of Q and E^{B} represents a combination where Q represents a group represented by Q1, and E^{B} represents R¹²C(O)—, R¹²C(═N—OH)—, or a halogen atom, or,
a combination where Q represents a group represented by Q2, and E^{B} represents R¹²C(O)—, R¹²C(═N—OH)—, a bromine atom, or an iodine atom,
each of the group represented by Q1 and the group represented by Q2 represents the following group (where ● represents a binding site to a benzene ring), 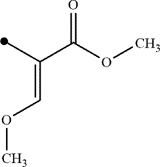 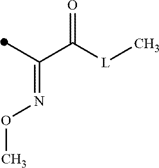
L represents an oxygen atom or NH,
R¹ represents a C1-C3 chain hydrocarbon group which may be optionally substituted with one or more halogen atoms, a methoxy group, a cyclopropyl group, or a halogen atom, and
R¹² represents a C1-C3 alkyl group].
